(19) United States
(12) Patent Application Publication
(10) Pub. No.: US 2022/0123233 A1
(43) Pub. Date: Apr. 21, 2022

(54) A PLURALITY OF HOST MATERIALS AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Su-Hyun LEE, Gyeonggi-do (KR); Hyo-Nim SHIN, Gyeonggi-do (KR); Bitnari KIM, Gyeonggi-do (KR); So-Young JUNG, Gyeonggi-do (KR)

(21) Appl. No.: 17/285,822

(22) PCT Filed: Sep. 25, 2019
(86) PCT No.: PCT/KR2019/012468

§ 371 Date: Apr. 15, 2021

(30) Foreign Application Priority Data
Oct. 17, 2018 (KR) 10-2018-0124002
Jul. 2, 2019 (KR) 10-2019-0079401
Aug. 13, 2019 (KR) 10-2019-0098864

(51) Int. Cl. H01L 51/00; C07D 405/14; C09K 11/06; C07D 405/10

(52) U.S. Cl. CPC H01L 51/0073; C07D 405/14; C09K 11/06; H01L 51/0072; H01L 51/5016; H01L 51/0052; H01L 51/0067; H01L 51/0058; H01L 2251/5384; C07D 405/10

(57) ABSTRACT

The present disclosure relates to a plurality of host materials comprising a first host material comprising a compound represented by formula 1, and a second host material comprising a compound represented by formula 2, and an organic electroluminescent device comprising the same. By comprising a specific combination of compounds as a host material, it is possible to provide an organic electroluminescent device having low driving voltage, high luminous efficiency, high power efficiency and/or improved lifespan properties, compared to conventional organic electroluminescent devices.

### TECHNICAL FIELD

The present disclosure relates to a plurality of host materials and an organic electroluminescent device comprising the same.

### BACKGROUND ART

A small molecular green organic electroluminescent device (OLED) was first developed by Tang, et al., of Eastman Kodak in 1987 by using TPD/ALq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation. In many applications such as TVs and lightings, OLED lifetime is insufficient, and high efficiency of OLEDs is still required. Typically, the higher the luminance of an OLED is, the shorter the lifetime of an OLED is. Therefore, an OLED having high luminous efficiency and/or long lifespan characteristics is required for long time use and high resolution of a display.

In order to enhance luminous efficiency, driving voltage and/or lifespan, various materials or concepts for an organic layer of an organic electroluminescent device have been proposed. However, they were not satisfactory in practical use.

U.S. Pat. No. 9,397,307 B2 discloses an organic electroluminescent device in which a compound comprising carbazole, dibenzofuran, or dibenzothiopene is used as a host. However, said reference does not specifically disclose an organic electroluminescent device using a specific combination of a plurality of host materials of the present disclosure. In addition, development of a host material for improving performances of an OLED is still required.

### DISCLOSURE OF INVENTION

### Technical Problem

The objective of the present disclosure is to provide an organic electroluminescent device having low driving voltage, high luminous efficiency, high power efficiency and/or improved lifespan properties by comprising a plurality of host materials including a specific combination of compounds.

### Solution to Problem

The present inventors found that the above objective can be achieved by using a plurality of host materials comprising a first host material comprising a compound represented by the following formula 1, and a second host material comprising a compound represented by the following formula 2:

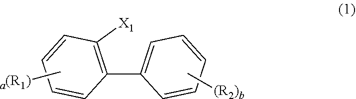

wherein

X₁ represents NR₃, CR₄R₅, O, or S;

R₁ and R₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or

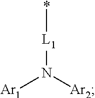

or two R₁'s, two R₂'s, or both thereof may be linked to each other to form a ring(s);

R₃ represents a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, or

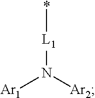

L₁, each independently, represents a single bond, a substituted or unsubstituted (C6-C30)arylene, or a substituted or unsubstituted (3- to 30-membered)heteroarylene, where if a plurality of L₁'s are present, each of L₁ may be the same or different;

Ar₁ and Ar₂, each independently, represent a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino, where if a plurality of Ar₁'s and a plurality of Ar₂'s, each independently, are present, each of Ar₁ and each of Ar₂ may be the same or different;

R₄ and R₅, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to each other to form a ring(s); and

a and b, each independently, represent an integer of 1 to 4, where if a and b are an integer of 2 or more, each of R₁ and each of R₂ may be the same or different;

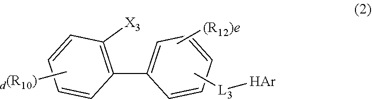

wherein

HAr represents

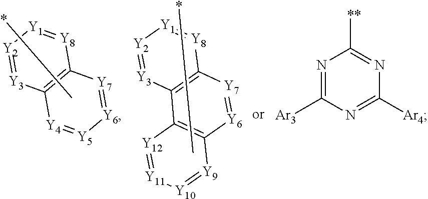

X₃ represents O or S;

L₃ represents a single bond, a substituted or unsubstituted (C6-C30)arylene, or a substituted or unsubstituted (3- to 30-membered)heteroarylene, with the proviso that if HAr represents

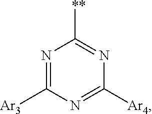

L₃ represents a substituted or unsubstituted naphthylene;

Y₁ to Y₁₂, each independently, represent CR₁₁ or N, with the proviso that at least one of Y₁ to Y₈ in

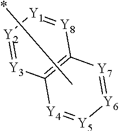

represents N, and at least one of Y₁ to Y₃, Y₆ to Y₈, and Y₉ to Y₁₂ in

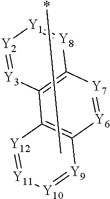

represents N;

R₁₀ and R₁₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or two R₀'s, two R₁₂'s, or both thereof may be linked to each other to form a ring(s);

R₁₁, Ar₃, and Ar₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl, where if a plurality of R₁₁'s are present, each of R₁₁ may be the same or different;

d represents an integer of 1 to 4, and e represents an integer of 1 to 3, where if d and e are an integer of 2 or more, each of R₁₀ and each of R₁₂ may be the same or different; and

* represents a bonding site.

### Advantageous Effects of Invention

By comprising a plurality of host materials according to the present disclosure, an organic electroluminescent device having low driving voltage, high luminous efficiency, high power efficiency and/or improved lifespan properties, compared to conventional organic electroluminescent devices can be provided, and it is possible to produce a display device or a lighting device using the same.

### Mode for the Invention

Hereinafter, the present disclosure will be described in detail. However, the following description is intended to explain the invention, and is not meant in any way to restrict the scope of the invention.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. Such at least two compounds may be comprised in the same layer or different layers through methods used in the art, and, for example, may be mixture-evaporated or co-evaporated, or may be individually evaporated.

The term “a plurality of host materials” in the present disclosure means a host material comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of host materials of the present disclosure may be a combination of at least two host materials, and selectively may further comprise conventional materials comprised in an organic electroluminescent material. A plurality of host materials of the present disclosure may be comprised in any light-emitting layer constituting an organic electroluminescent device, and at least two compounds comprised in the plurality of host materials may be comprised together in one light-emitting layer or may respectively be comprised in different light-emitting layers, through methods used in the art. For example, the at least two compounds may be mixture-evaporated or co-evaporated, or may be individually evaporated.

Herein, the term “(C1-C30)alkyl” is meant to be a linear or branched alkylene) having 1 to 30 carbon atoms constituting the chain, in which the number of carbon atoms is preferably 1 to 20, and more preferably 1 to 10. The above alkyl may include methyl, ethyl, n-propyl, iso-propyl, n-butyl, iso-butyl, Pert-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl. 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7, The above cycloalkyl may include cyclopropyl, cyclobutyl, cyclopentyl, cyclohexyl, etc. The term “(3- to 7-membered)heterocycloalkyl” is meant to be a cycloalkyl having 3 to 7, preferably 5 to 7, ring backbone atoms, and including at least one heteroatom selected from the group consisting of B, N, O, S, Si, and P, and preferably the group consisting of 0, 5, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms, in which the number of the ring backbone carbon atoms is preferably 6 to 20. The above aryl(ene) may be partially saturated, and may comprise a spiro structure. The above aryl may include phenyl, biphenyl, terphenyl, naphthyl, binaphthyl, phenylnaphthyl, naphthylphenyl, phenylterphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, etc. More specifically, the above aryl may include a phenyl group, a 1-naphthyl group, a 2-naphthyl group, a 1-anthryl group, a 2-anthryl group, a 9-anthryl group, a benzanthryl group, a 1-phenanthryl group, a 2-phenanthryl group, a 3-phenanthryl group, a 4-phenanthryl group, a 9-phenanthryl group, a naphthacenyl group, a pyrenyl group, a 1-chrysenyl group, a 2-chrysenyl group, a 3-chrysenyl group, a 4-chrysenyl group, a 5-chrysenyl group, a 6-chrysenyl group, a benzo[c]phenanthryl group, a benzo[g]chrysenyl group, a 1-triphenylenyl group, a 2-triphenylenyl group, a 3-triphenylenyl group, a 4-triphenylenyl group, a 1-fluorenyl group, a 2-fluorenyl group, a 3-fluorenyl group, a 4-fluorenyl group, a 9-fluorenyl group, a benzofluorenyl group, a dibenzofluorenyl group, a 2-biphenylyl group, a 3-biphenylyl group, a 4-biphenylyl group, an o-terphenyl group, an m-terphenyl-4-yl group, an m-terphenyl-3-yl group, an m-terphenyl-2-yl group, a p-terphenyl-4-yl group, a p-terphenyl-3-yl group, a p-terphenyl-2-yl group, an m-quaterphenyl group, a 3-fluoranthenyl group, a 4-fluoranthenyl group, an 8-fluoranthenyl group, a 9-fluoranthenyl group, a benzofluoranthenyl group, an o-tolyl group, an m-tolyl group, a p-tolyl group, a 2,3-xylyl group, a 3,4-xylyl group, a 2,5-xylyl group, a mesityl group, an o-cumenyl group, an m-cumenyl group, a p-cumenyl group, a p-t-butylphenyl group, a p-(2-phenylpropyl)phenyl group, a 4′-methylbiphenylyl group, a 4″-t-butyl-p-terphenyl-4-yl group, a 9,9-dimethyl-1-fluorenyl group, a 9,9-dimethyl-2-fluorenyl group, a 9,9-dimethyl-3-fluorenyl group, a 9,9-dimethyl-4-fluorenyl group, a 9,9-diphenyl-1-fluorenyl group, a 9,9-diphenyl-2-fluorenyl group, a 9,9-diphenyl-3-fluorenyl group, a 9,9-diphenyl-4-fluorenyl group, etc.

The term “(3- to 30-membered)heteroaryl(ene)” is meant to be an aryl having 3 to 30 ring backbone atoms, and including at least one, preferably 1 to 4 heteroatoms selected from the group consisting of B, N, O, S, Si, and P. The above heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, benzonaphthofuranyl, benzonaphthothiophenyl, diazadibenzofuranyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, benzoquinolyl, isoquinolyl, benzoisoquinolyl, cinnolinyl, quinazolinyl, benzoquinazolinyl, quinoxalinyl, benzoquinoxalinyl, naphthyridinyl, triazanaphthyl, benzothienopyrirnidinyl, carbazolyl, benzocarbazolyl, dibenzocarbazolyl, phenoxazinyl, phenothiazinyl, phenanthridinyl, benzodioxolyl, and dihydroacridinyl, More specifically, the above heteroaryl may include a 1-pyrrolyl group, a 2-pyrrolyl group, a 3-pyrrolyl group, a pyrazinyl group, a 2-pyridinyl group, a 2-pyrimidinyl group, a 4-pyrimidinyl group, a 5-pyrimidinyl group, a 6-pyrimidinyl group, a 1,2,3-triazin-4-yl group, a 1,2,4-triazin-3-yl group, a 1,3,5-triazin-2-yl group, a 1-imidazolyl group, a 2-imidazolyl group, a 1-pyrazolyl group, a 1-indolidinyl group, a 2-indolidinyl group, a 3-indolidinyl group, a 5-indolidinyl group, a 6-indolidinyl group, a 7-indolidinyl group, an 8-indolidinyl group, a 2-imidazopyridinyl group, a 3-imidazopyridinyl group, a 5-imidazopyridinyl group, a 6-imidazopyridinyl group, a 7-imidazopyridinyl group, an 8-imidazopyridinyl group, a 3-pyridinyl group, a 4-pyridinyl group, a 1-indolyl group, a 2-indolyl group, a 3-indolyl group, a 4-indolyl group, a 5-indolyl group, a 6-indolyl group, a 7-indolyl group, a 1-isoindolyl group, a 2-isoindolyl group, a 3-isoindolyl group, a 4-isoindolyl group, a 5-isoindolyl group, a 6-isoindolyl group, a 7-isoindolyl group, a 2-furyl group, a 3-furyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 3-isobenzofuranyl group, a 4-isobenzofuranyl group, a 5-isobenzofuranyl group, a 6-isobenzofuranyl group, a 7-isobenzofuranyl group, a 2-quinolyl group, a 3-quinolyl group, a 4-quinolyl group, a 5-quinolyl group, a 6-quinolyl group, a 7-quinolyl group, an 8-quinolyl group, a 1-isoquinolyl group, a 3-isoquinolyl group, a 4-isoquinolyl group, a 5-isoquinolyl group, a 6-isoquinolyl group, a 7-isoquinolyl group, an 8-isoquinolyl group, a 2-quinoxalinyl group, a 5-quinoxalinyl group, a 6-quinoxalinyl group, a 1-carbazolyl group, a 2-carbazolyl group, a 3-carbazolyl group, a 4-carbazolyl group, a 9-carbazolyl group, an azacarbazolyl-1-yl group, an azacarbazolyl-2-yl group, an azacarbazolyl-3-yl group, an azacarbazolyl-4-yl group, an azacarbazolyl-5-yl group, an azacarbazolyl-6-yl group, an azacarbazolyl-7-yl group, an azacarbazolyl-8-yl group, an azacarbazolyl-9-yl group, a 1-phenanthridinyl group, a 2-phenanthridinyl group, a 3-phenanthridinyl group, a 4-phenanthridinyl group, a 6-phenanthridinyl group, a 7-phenanthridinyl group, an 8-phenanthridinyl group, a 9-phenanthridinyl group, a 10-phenanthridinyl group, a 1-acridinyl group, a 2-acridinyl group, a 3-acridinyl group, a 4-acridinyl group, a 9-acridinyl group, a 2-oxazolyl group, a 4-oxazolyl group, a 5-oxazolyl group, a 2-oxadiazolyl group, a 5-oxadiazolyl group, a 3-furazanyl group, a 2-thienyl group, a 3-thienyl group, a 2-methylpyrrol-1-yl group, a 2-methylpyrrol-3-yl group, a 2-methylpyrrol-4-yl group, a 2-methylpyrrol-5-yl group, a 3-methylpyrrol-1-yl group, a 3-methylpyrrol-2-yl group, a 3-methylpyrrol-4-yl group, a 3-methylpyrrol-5-yl group, a 2-t-butylpyrrol-4-yl group, a 3-(2-phenylpropyl)pyrrol-1-yl group, a 2-methyl-1-indolyl group, a 4-methyl-1-indolyl group, a 2-methyl-3-indolyl group, a 4-methyl-3-indolyl group, a 2-t-butyl-1-indolyl group, a 4-t-butyl-1-indolyl group, a 2-t-butyl-3-indolyl group, a 4-t-butyl-3-indolyl group, a 1-dibenzofuranyl group, a 2-dibenzofuranyl group, a 3-dibenzofuranyl group, a 4-dibenzofuranyl group, a 1-dibenzothiophenyl group, a 2-dibenzothiophenyl group, a 3-dibenzothiophenyl group, a 4-dibenzothiophenyl group, a 1-silafluorenyl group, a 2-silafluorenyl group, a 3-silafluorenyl group, a 4-silafluorenyl group, a 1-germafluorenyl group, a 2-germafluorenyl group, a 3-germafluorenyl group, and a 4-germafluorenyl group. Furthermore, “halogen” includes F, Cl, Br, and I.

Herein, “substituted” in the expression “substituted or unsubstituted” means that a hydrogen atom in a certain functional group is replaced with another atom or another functional group, i.e., a substituent. The substituents of the substituted alkyl, the substituted aryl, the substituted arylene, the substituted heteroaryl, the substituted heteroarylene, the substituted cycloalkyl, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-acylamino, and the substituted alkylarylamino in the formulas of the present disclosure, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C8-C30)arylthio; a (3- to 30-membered)heteroaryl unsubstituted or substituted with a (C6-C30)aryl(s); a (C6-C30)aryl unsubstituted or substituted with at least one of a (C1-C30)alkyl(s) and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino: a mono- or di-(C6-C30)arylamino; a (C1-C30)alkyl(C6-C30)arylamino; a (C1-C30)alkylcarbonyl; a (C1-C30)alkoxycarbonyl: a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl; preferably, at least one selected from the group consisting of a (C1-C6)alkyl, a substituted or unsubstituted (C6-C20)aryl, a substituted or unsubstituted (3- to 15-membered)heteroaryl, and a di(C6-C12)arylamino; more preferably, at least one selected from the group consisting of a (C1-C6)alkyl, a (C6-C20)aryl unsubstituted or substituted with a (C1-C6)alkyl(s), a (3- to 15-membered)heteroaryl unsubstituted or substituted with a (C6-C12)aryl(s), and a di(C6-C12)arylamino; and for example, at least one selected from the group consisting of a methyl, a phenyl, a naphthyl, a terphenyl, a dimethylfluorenyl, a phenylquinoxalinyl, a carbazolyl, a dibenzofuranyl, a dibenzothiophenyl, and a diphenylamino.

In the formulas of the present disclosure, if a substituent is linked to an adjacent substituent to form a ring or two adjacent substituents are linked to each other to form a ring, the ring may be a substituted or unsubstituted, mono- or polycyclic, (3- to 30-membered) alicyclic or aromatic ring, or the combination thereof, in which the formed ring may contain at least one heteroatom selected from B, N, O, S, Si, and P, preferably at least one heteroatom selected from N, O, and S. According to one embodiment of the present disclosure, the number of the ring backbone atoms is 5 to 20. According to another embodiment of the present disclosure, the number of the ring backbone atoms is 5 to 15. For example, the fused ring may be a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring.

In the formulas of the present disclosure, heteroaryl or heteroarylene may, each independently, contain at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-030)arylamino.

Hereinafter, the compounds represented by formulas 1 and 2 will be described in more detail.

In formula 1, X₁ represents NR₃, CR₄R₅, O, or S.

Herein, R₃ represents a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, or

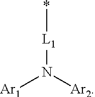

According to one embodiment of the present disclosure, R₃ represents a substituted or unsubstituted (C6-C30)aryl, or

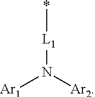

According to another embodiment of the present disclosure. R₃ represents a (C6-C30)aryl unsubstituted or substituted with a (C6-C20)aryl(s) or a (5- to 15-membered)heteroaryl(s); or

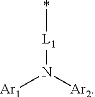

Specifically, R₃ may be a naphthylphenyl, a terphenylnaphthyl, a dibenzofuranylnaphthyl,

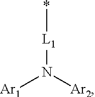

etc.

R₄ and R₅, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to each other to form a ring(s). According to one embodiment of the present disclosure, R₄ and R₅, each independently, represent a substituted or unsubstituted (C1-C6)alkyl, or a substituted or unsubstituted (C6-C12)aryl. According to another embodiment of the present disclosure, R₄ and R₅, each independently, represent an unsubstituted (C1-C6)alkyl, or an unsubstituted (C6-C12)aryl. Specifically, R₄ and R₅, each independently, may be a methyl, a phenyl, etc. R₄ and R₅ may be the same or different. According to one embodiment of the present disclosure, R₄ and R₅ are the same.

In formula 1, R₁ and R₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C36)arylsilyl, or

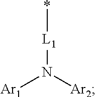

or two R₁'s, two R₂'s, or both thereof may be linked to each other to form a ring(s). According to one embodiment of the present disclosure, R₁ and R₂, each independently, represent hydrogen, a substituted or unsubstituted (C6-C12)aryl, a substituted or unsubstituted (5- to 15-membered)heteroaryl, or

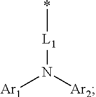

or two R₁'s, two R₂'s, or both thereof may be linked to each other to form a ring(s). According to another embodiment of the present disclosure, R₁ and R₂, each independently, represent hydrogen, an unsubstituted (C6-C12)aryl, an unsubstituted (5- to 15-membered)heteroaryl, or

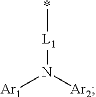

or two R₁'s, two R₂'s, or both thereof may be linked to each other to form a ring(s). Specifically, R₁ and R₂, each independently, may be hydrogen, a phenyl, a dibenzothiophenyl, etc.; or two R₁'s, two R₂'s, or both thereof may be linked to each other to form

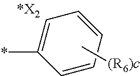

or a benzene ring(s), in which X₂ represents NR₇, CR₈R₉, O, or S; R₆ is the same as the definition of R₁ and R₂; R₇ is the same as the definition of R₃; R₈ and R₉, each independently, are the same as the definition of R₄ and R₅; c represents an integer of 1 to 4, where if c is an integer of 2 or more, each of R₆ may be the same or different; and * represents a bonding site. According to one embodiment of the present disclosure, R₆ is hydrogen.

In

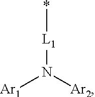

L₁, each independently, represents a single bond, a substituted or unsubstituted (C6-C30)arylene, or a substituted or unsubstituted (3- to 30-membered)heteroarylene, where if a plurality of L₁'s are present, each of L₁ may be the same or different. According to one embodiment of the present disclosure, L₁, each independently, represents a single bond, or a substituted or unsubstituted (C6-C15)arylene. According to another embodiment of the present disclosure, L₁, each independently, represents a single bond; or a (C6-C15)arylene unsubstituted or substituted with a (3- to 30-membered)heteroarylene(s) or a di(C6-C12)arylamino(s). Specifically, L₁, each independently, may be a single bond, a phenylene, a naphthylene, a biphenylene, a dibenzothiophenylphenylene, a phenylene substituted with a diphenylamino, etc.

In

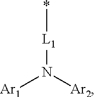

Ar₁ and Ar₂, each independently, represent a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino, where if a plurality of AR₁'s and a plurality of Arₜ's, each independently, are present, each of Ar₁ and each of Ar₂ may be the same or different. According to one embodiment of the present disclosure, Ar₁ and Ar₂, each independently, represent a substituted or unsubstituted (C6-C25)aryl, or a substituted or unsubstituted (5- to 15-membered)heteroaryl. According to another embodiment of the present disclosure, Ar₁ and Ar₂, each independently, represent a (C6-C25)aryl unsubstituted or substituted with at least one of a (C1-C6)alkyl(s) and a (C6-C12)aryl(s); or an unsubstituted (5- to 15-membered)heteroaryl. Specifically, Ar₁ and Ar₂, each independently, may be a phenyl, a naphthyl, a biphenyl, a naphthylphenyl, a dimethylfluorenyl, a diphenylfluorenyl, a phenyl substituted with a dimethylfluorenyl, a dibenzofuranyl, etc.

In formula 1, a and b, each independently, represent an integer of 1 to 4, where if a and b are an integer of 2 or more, each of R₁ and each of R₂ may be the same or different.

According to one embodiment of the present disclosure, formula 1 may be represented by at least one of the following formulas 1-1 to 1-3.

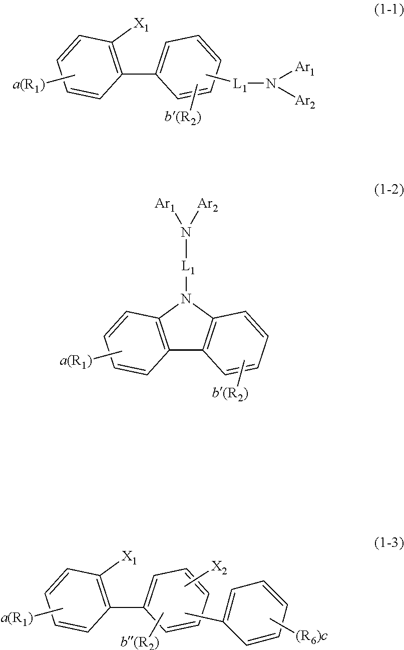

wherein

X₁, R₁, R₂, L₁, Ar₁, Ar₂, a, and b are as defined in formula 1;

X₂ represents NR₇, CR₈R₉, O, or S;

R₆ is the same as the definition of R₁ and R₂;

R₇ is the same as the definition of R₃;

R₈ and R₉, each independently, are the same as the definition of R₄ and R₅;

b′ represents an integer of 1 to 3, b″ represents 1 or 2, and c represents an integer of 1 to 4, where if b′, b″, and c are an integer of 2 or more, each of R₂ and each of R₅ may be the same or different; and

* represents a bonding site.

In formula 2, HAr represents

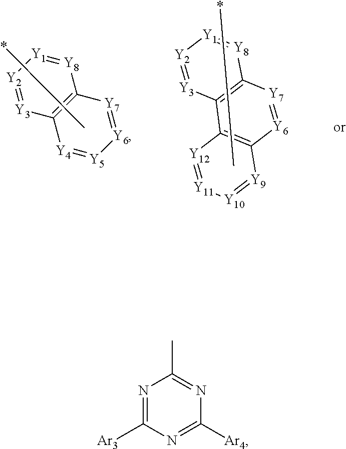

and X₃ represents O or S.

In formula 2, L₃ represents a single bond, a substituted or unsubstituted (C6-C30)arylene, or a substituted or unsubstituted (3- to 30-membered)heteroarylene, with the proviso that if HAr represents

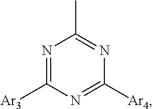

L₃ represents a substituted or unsubstituted naphthylene. According to one embodiment of the present disclosure, L₃ represents a single bond, or a substituted or unsubstituted (C6-C15)arylene. According to another embodiment of the present disclosure, L₃ represents a single bond, or an unsubstituted (C6-C15)arylene. Specifically, Ls may be a single bond, a phenylene, a naphthylene, a biphenylene, etc.

In formula 2, Y₁ to Y₁₂, each independently, represent CR₁₁ or N, with the proviso that at least one of Y₁ to Y₈ in

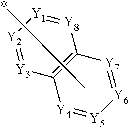

represents N, and at least one of Y₁ to Y₃, Y₆ to Y₈, and Y₉ to Y₁₂ in

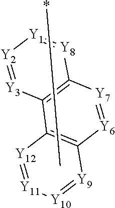

represents N. According to one embodiment of the present disclosure, at least two of Y₁ to Y₈ in

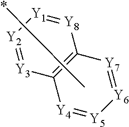

represent N, and at least two of Y₁ to Y₃, Y₆ to Y₈, and Y₉ to Y₁₂ in

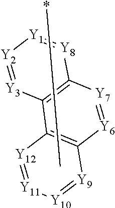

represent N.

In formula 2. R₁₀ and R₁₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or two R₀'s, two R₁₂'s, or both thereof may be linked to each other to form a ring(s). According to one embodiment of the present disclosure, R₁₀ and R₁₂, each independently, represent hydrogen.

In formula 2, R₁₁, Ar₃, and Ar₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl, where if a plurality of R₁₁'s are present, each of R₁₁ may be the same or different. According to one embodiment of the present disclosure, R₁₁ represents hydrogen, a substituted or unsubstituted (C6-C20)aryl, or a substituted or unsubstituted (5- to 15-membered)heteroaryl. According to another embodiment of the present disclosure, R₁₁ represents hydrogen; a (C6-C20)aryl unsubstituted or substituted with at least one of a (C1-C6)alkyl(s), a (C6-C12)aryl(s), a (5- to 20-membered)heteroaryl(s), and a di(C6-C12)arylamino(s); or a (5- to 15-membered)heteroaryl unsubstituted or substituted with a (C6-C12)aryl(s), Specifically; R₁₁ may be hydrogen, a phenyl, a naphthyl, a biphenyl, a terphenyl, a phenanthrenyl, a triphenylenyl, a naphthylphenyl, a phenylnaphthyl, a dimethylfluorenyl, a dimethylbenzofluorenyl, a phenyl substituted with a phenylquinoxalinyl, a carbazolylphenyl, a dibenzofuranylphenyl, a phenyl substituted with a diphenylamino, a dibenzofuranyl, a phenylcarbazolyl, etc. According to one embodiment of the present disclosure, Ar₃ and Ar₄, each independently, represent a substituted or unsubstituted (C6-C20)aryl. According to another embodiment of the present disclosure, Ar₃ and Ar₄, each independently, represent an unsubstituted (C6-C20)aryl. Specifically, Ar₃ and Ar₄, each independently, may be an unsubstituted phenyl, an unsubstituted naphthyl, an unsubstituted biphenyl, an unsubstituted terphenyl, etc.

In formula 2, d represents an integer of 1 to 4, and e represents an integer of 1 to 3, where if d and e are an integer of 2 or more, each of R₁₀ and each of R₁₂ may be the same or different.

In formulas 1 and 2, * represents a bonding site.

According to one embodiment of the present disclosure, formula 2 may be represented by at least one of the following formulas 2-1 to 2-1a

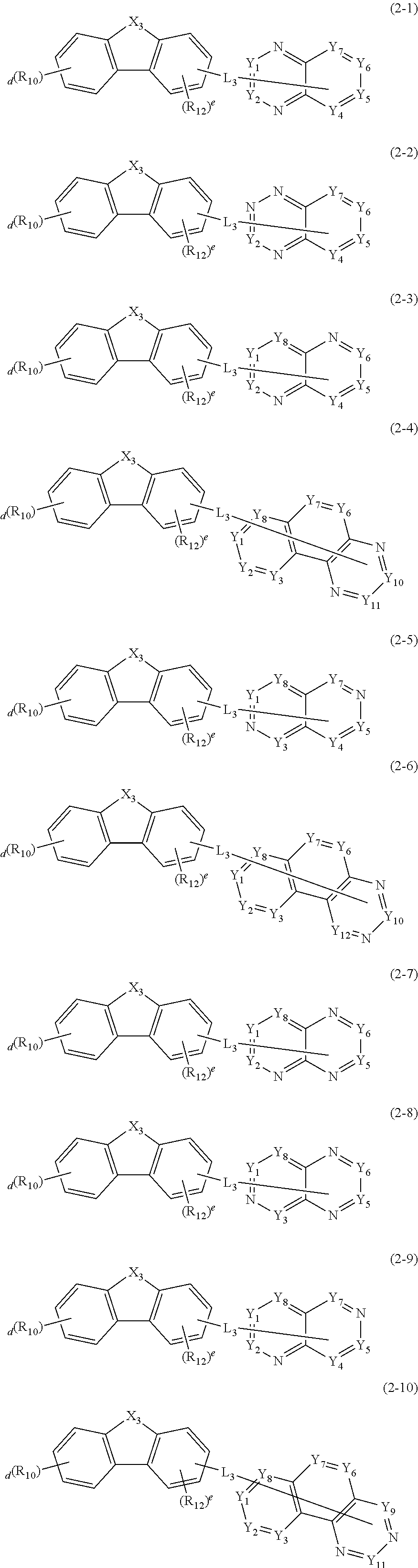

wherein

Y₁ to Y₈, Y₁₀, and Y₁₁, each independently, represent CR₁₁ or N; and

X₃, L₃, R₁₀ to R₁₂, d, and e are as defined in formula 2.

According to one embodiment of the present disclosure, formula 2 may be represented by the following formula 2-11.

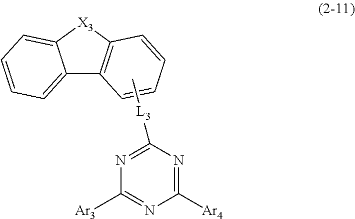

wherein

X; represents O or S;

L₃ represents an unsubstituted naphthylene; and

Ar₃ and Ar₄, each independently, represent an unsubstituted phenyl, an unsubstituted naphthyl, an unsubstituted biphenyl, or an unsubstituted terphenyl.

The compound represented by formula 1 includes the following compounds, but is not limited thereto.

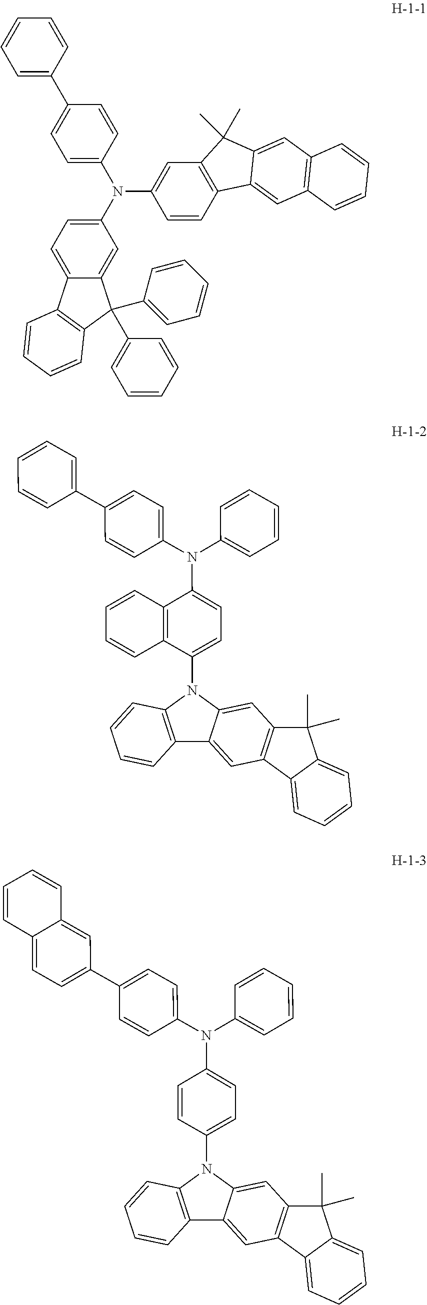 [SMILES: CC1(C)c2cc(N(c3ccc(-c4ccccc4)cc3)c3ccc4c(c3)C(c3ccccc3)(c3ccccc3)c3ccccc3-4)ccc2-c2cc3ccccc3cc21 ; CC1(C)c2ccccc2-c2cc3c4ccccc4n(-c4ccc(N(c5ccccc5)c5ccc(-c6ccccc6)cc5)c5ccccc45)c3cc21 ; CC1(C)c2ccccc2-c2cc3c4ccccc4n(-c4ccc(N(c5ccccc5)c5ccc(-c6ccc7ccccc7c6)cc5)cc4)c3cc21]

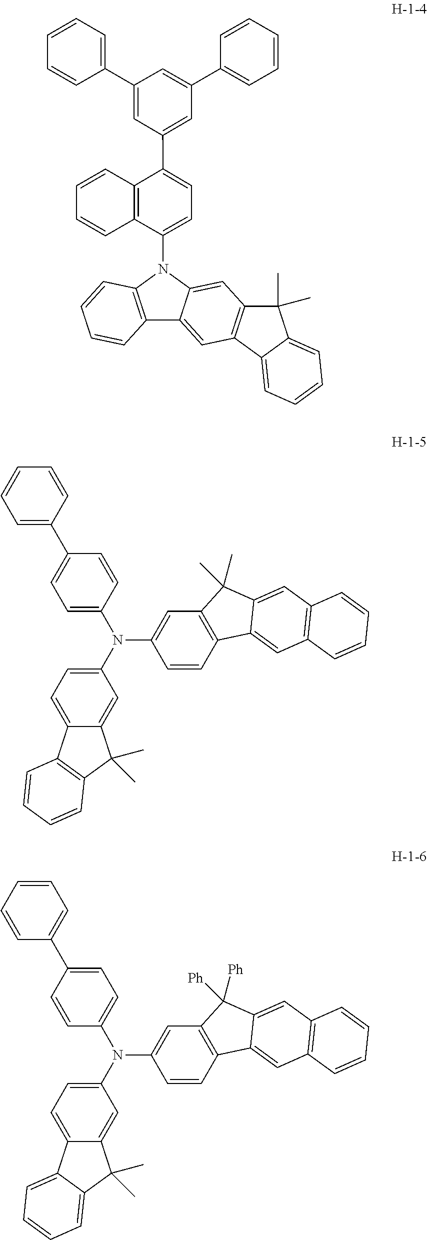 [SMILES: CC1(C)c2ccccc2-c2cc3c4ccccc4n(-c4ccc(-c5cc(-c6ccccc6)cc(-c6ccccc6)c5)c5ccccc45)c3cc21 ; CC1(C)c2ccccc2-c2ccc(N(c3ccc(-c4ccccc4)cc3)c3ccc4c(c3)C(C)(C)c3cc5ccccc5cc3-4)cc21 ; CC1(C)c2ccccc2-c2ccc(N(c3ccc(-c4ccccc4)cc3)c3ccc4c(c3)C(c3ccccc3)(c3ccccc3)c3cc5ccccc5cc3-4)cc21]

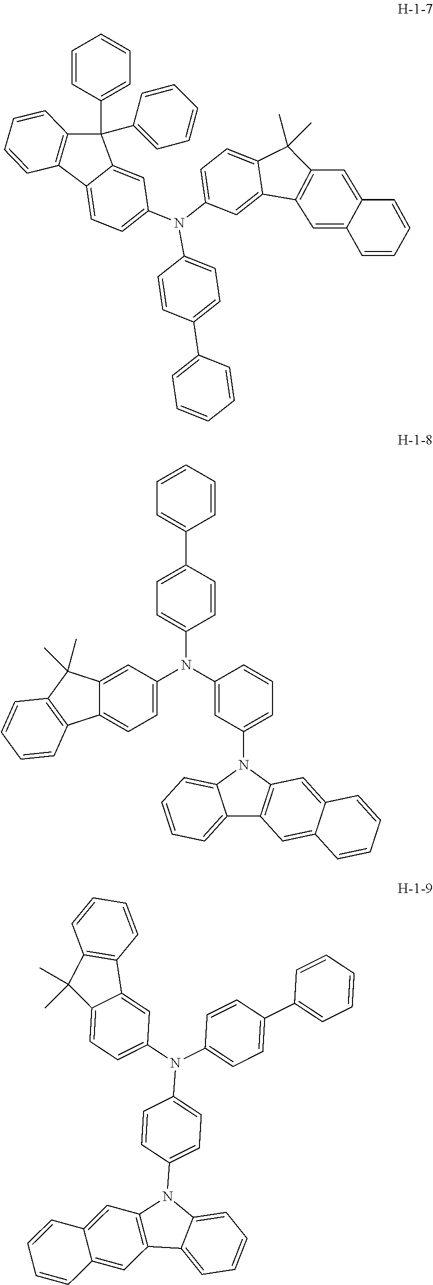 [SMILES: CC1(C)c2ccc(N(c3ccc(-c4ccccc4)cc3)c3ccc4c(c3)C(c3ccccc3)(c3ccccc3)c3ccccc3-4)cc2-c2cc3ccccc3cc21 ; CC1(C)c2ccccc2-c2ccc(N(c3ccc(-c4ccccc4)cc3)c3cccc(-n4c5ccccc5c5cc6ccccc6cc54)c3)cc21 ; CC1(C)c2ccccc2-c2cc(N(c3ccc(-c4ccccc4)cc3)c3ccc(-n4c5ccccc5c5cc6ccccc6cc54)cc3)ccc21]

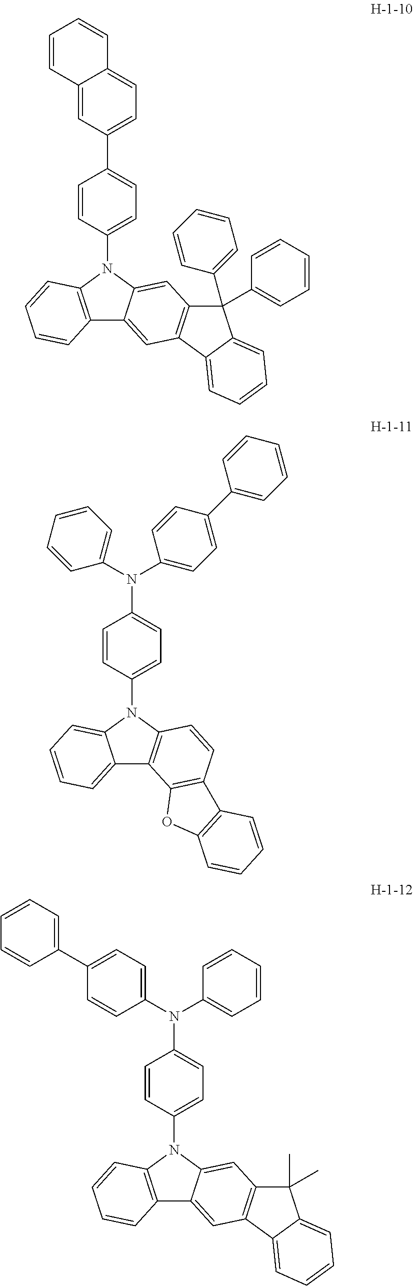

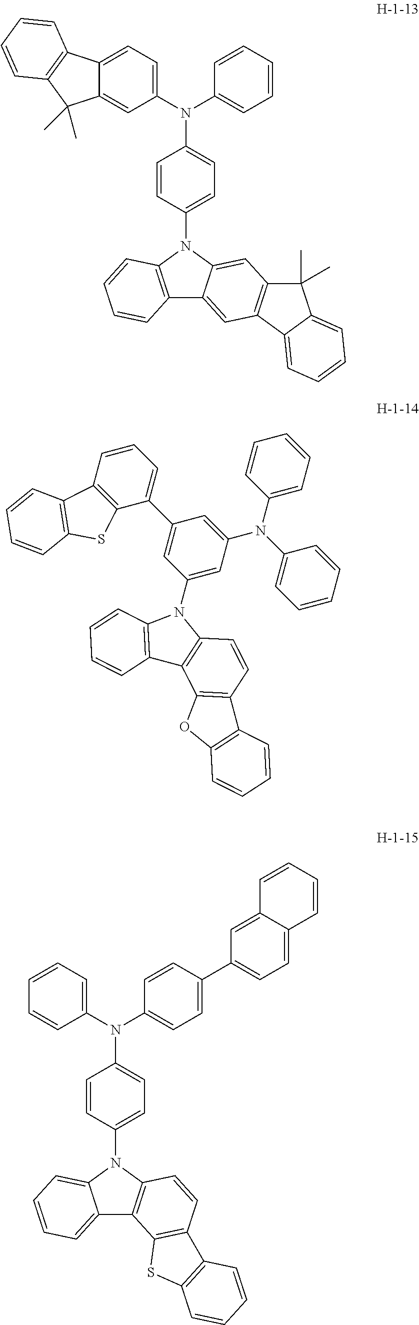 [SMILES: CC1(C)c2ccccc2-c2ccc(N(c3ccccc3)c3ccc(-n4c5ccccc5c5cc6c(cc54)C(C)(C)c4ccccc4-6)cc3)cc21 ; c1ccc(N(c2ccccc2)c2cc(-c3cccc4c3sc3ccccc34)cc(-n3c4ccccc4c4c5oc6ccccc6c5ccc43)c2)cc1 ; c1ccc(N(c2ccc(-c3ccc4ccccc4c3)cc2)c2ccc(-n3c4ccccc4c4c5sc6ccccc6c5ccc43)cc2)cc1]

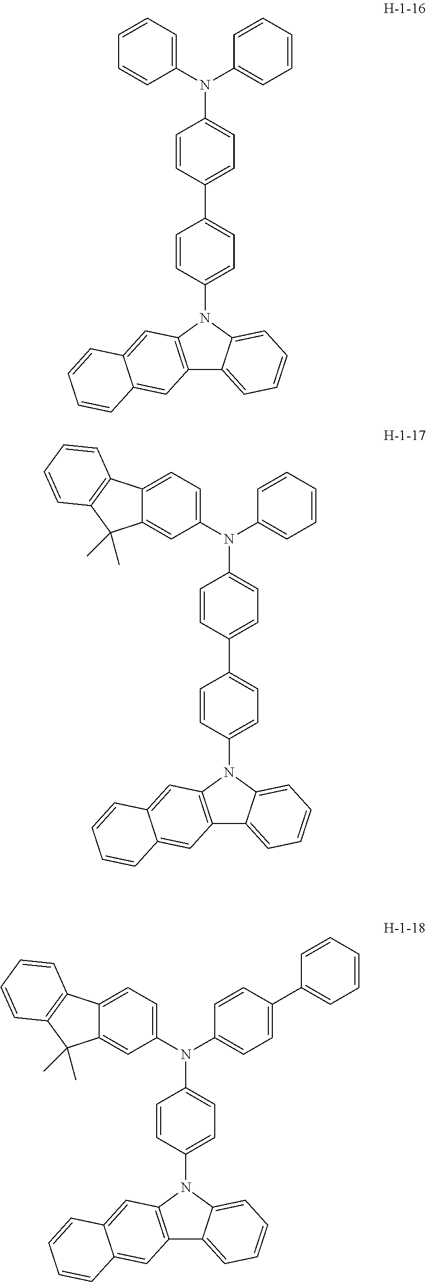

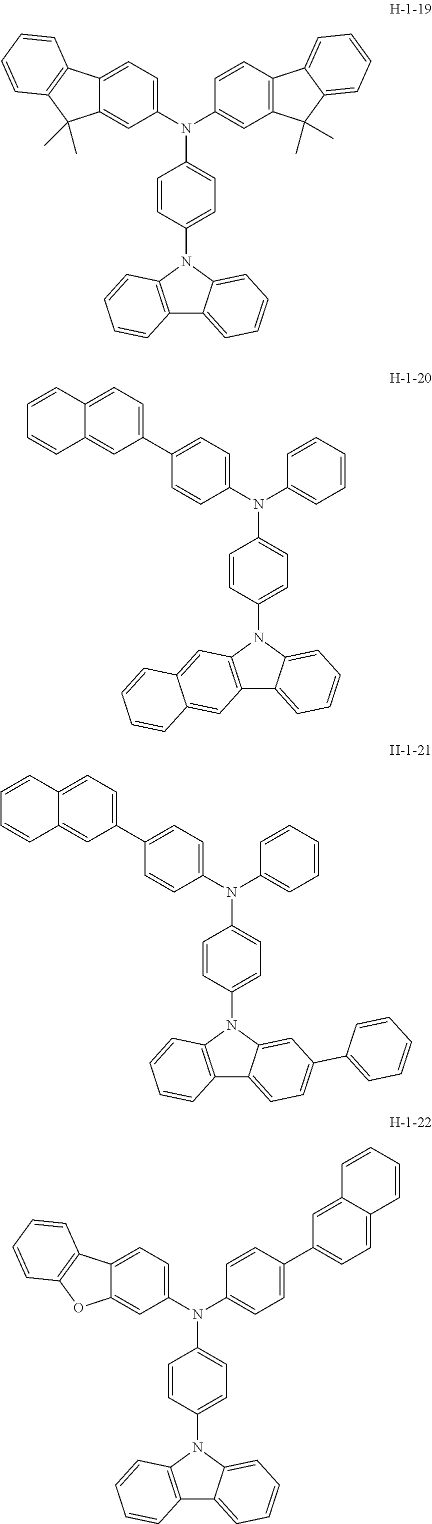 [SMILES: CC1(C)c2ccccc2-c2ccc(N(c3ccc(-n4c5ccccc5c5ccccc54)cc3)c3ccc4c(c3)C(C)(C)c3ccccc3-4)cc21 ; c1ccc(N(c2ccc(-c3ccc4ccccc4c3)cc2)c2ccc(-n3c4ccccc4c4cc5ccccc5cc43)cc2)cc1 ; c1ccc(-c2ccc3c4ccccc4n(-c4ccc(N(c5ccccc5)c5ccc(-c6ccc7ccccc7c6)cc5)cc4)c3c2)cc1 ; c1ccc2cc(-c3ccc(N(c4ccc(-n5c6ccccc6c6ccccc65)cc4)c4ccc5c(c4)oc4ccccc45)cc3)ccc2c1]

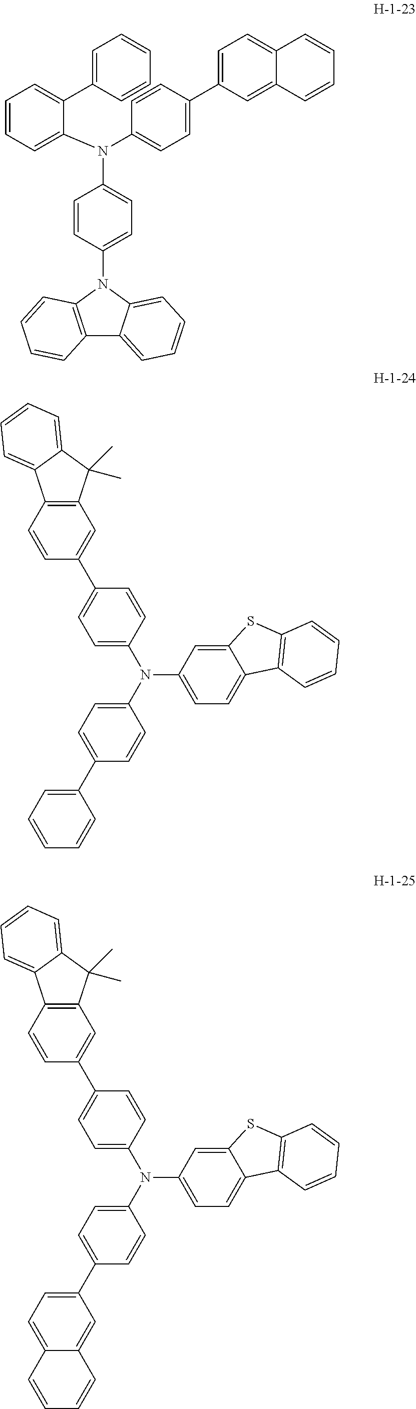 [SMILES: c1ccc(-c2ccccc2N(c2ccc(-c3ccc4ccccc4c3)cc2)c2ccc(-n3c4ccccc4c4ccccc43)cc2)cc1 ; CC1(C)c2ccccc2-c2ccc(-c3ccc(N(c4ccc(-c5ccccc5)cc4)c4ccc5c(c4)sc4ccccc45)cc3)cc21 ; CC1(C)c2ccccc2-c2ccc(-c3ccc(N(c4ccc(-c5ccc6ccccc6c5)cc4)c4ccc5c(c4)sc4ccccc45)cc3)cc21]

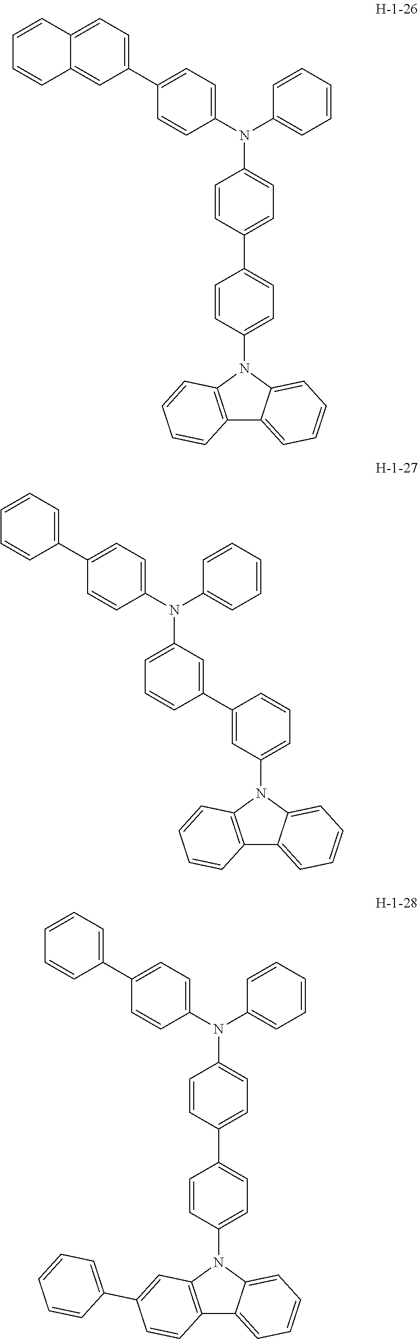 [SMILES: c1ccc(N(c2ccc(-c3ccc(-n4c5ccccc5c5ccccc54)cc3)cc2)c2ccc(-c3ccc4ccccc4c3)cc2)cc1 ; c1ccc(-c2ccc(N(c3ccccc3)c3cccc(-c4cccc(-n5c6ccccc6c6ccccc65)c4)c3)cc2)cc1 ; c1ccc(-c2ccc(N(c3ccccc3)c3ccc(-c4ccc(-n5c6ccccc6c6ccc(-c7ccccc7)cc65)cc4)cc3)cc2)cc1]

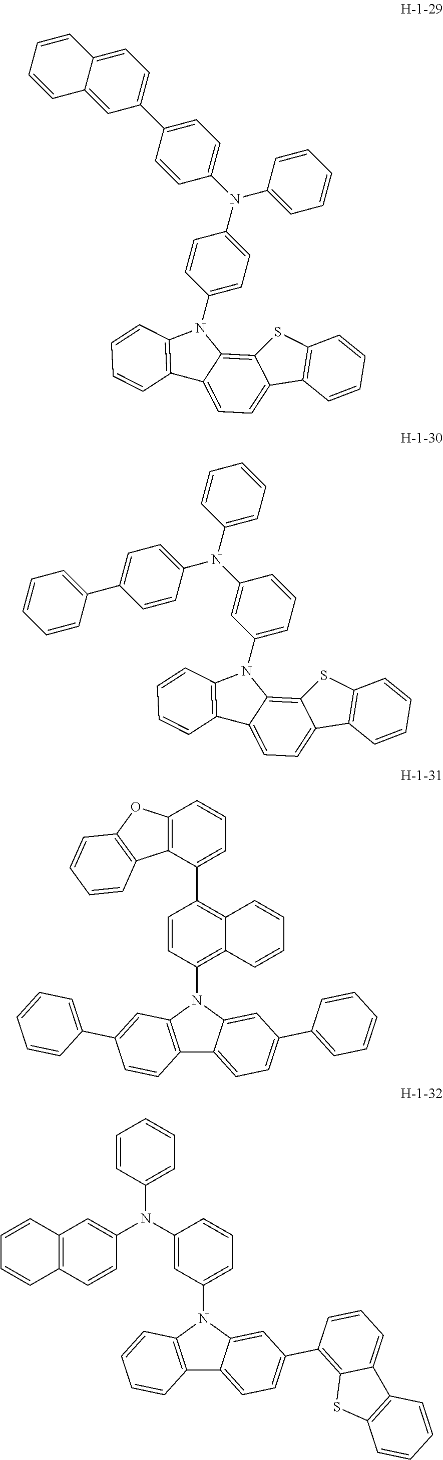

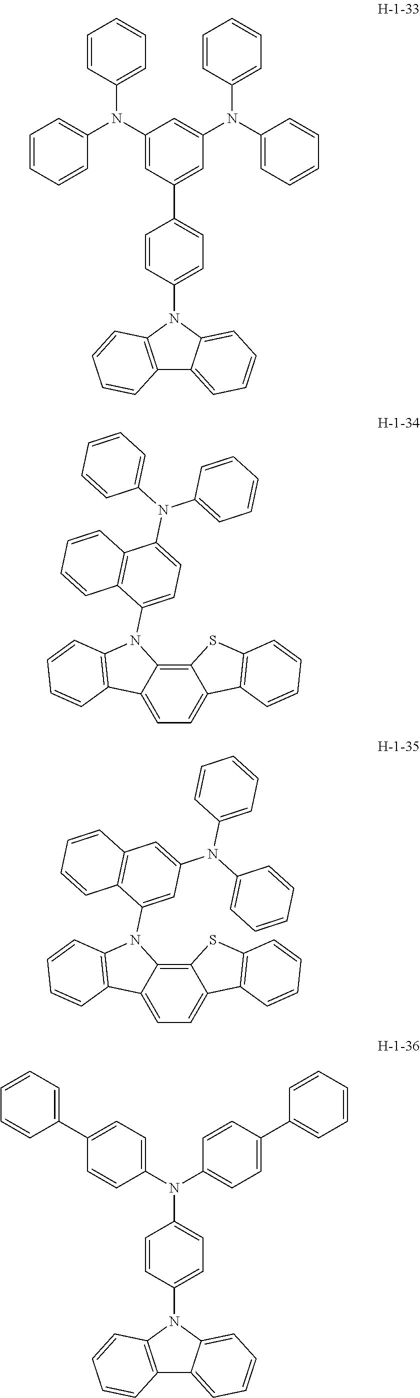

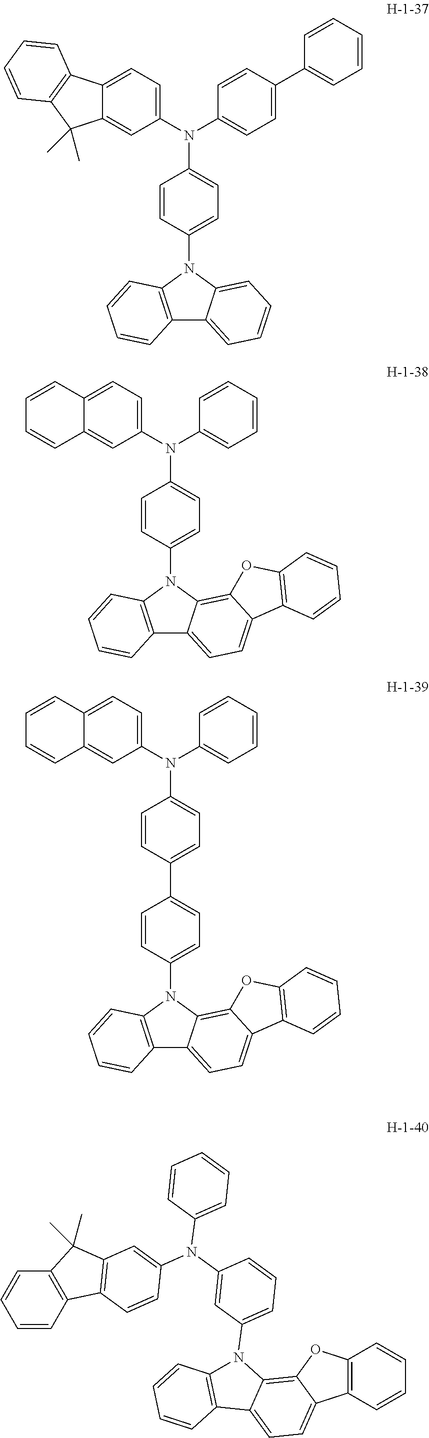

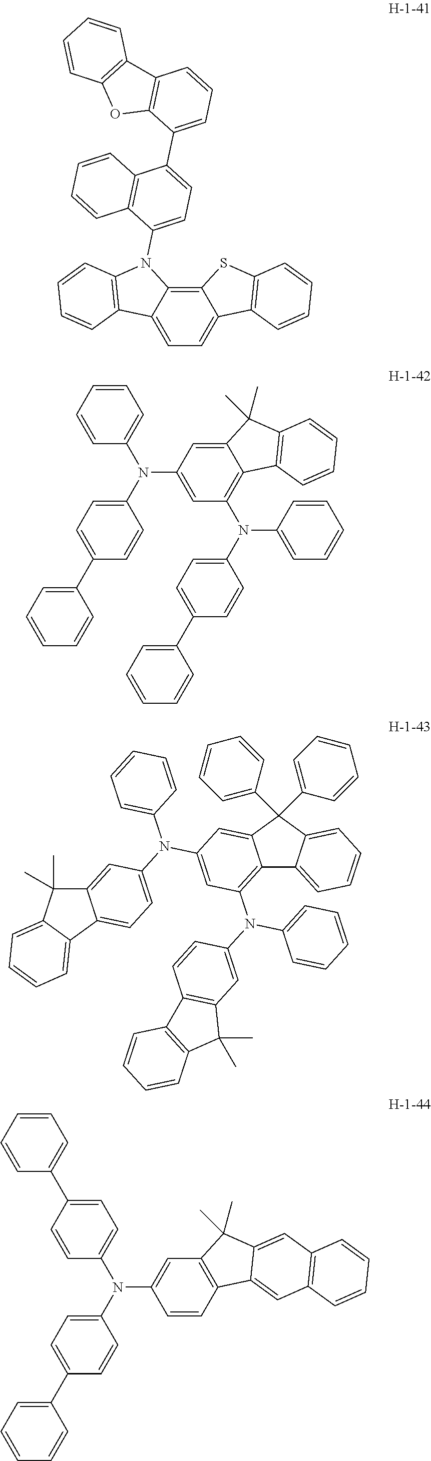 [SMILES: c1ccc2c(c1)oc1c(-c3ccc(-n4c5ccccc5c5ccc6c7ccccc7sc6c54)c4ccccc34)cccc12 ; CC1(C)c2ccccc2-c2c(N(c3ccccc3)c3ccc(-c4ccccc4)cc3)cc(N(c3ccccc3)c3ccc(-c4ccccc4)cc3)cc21 ; CC1(C)c2ccccc2-c2ccc(N(c3ccccc3)c3cc(N(c4ccccc4)c4ccc5c(c4)C(C)(C)c4ccccc4-5)c4c(c3)C(c3ccccc3)(c3ccccc3)c3ccccc3-4)cc21 ; CC1(C)c2cc(N(c3ccc(-c4ccccc4)cc3)c3ccc(-c4ccccc4)cc3)ccc2-c2cc3ccccc3cc21]

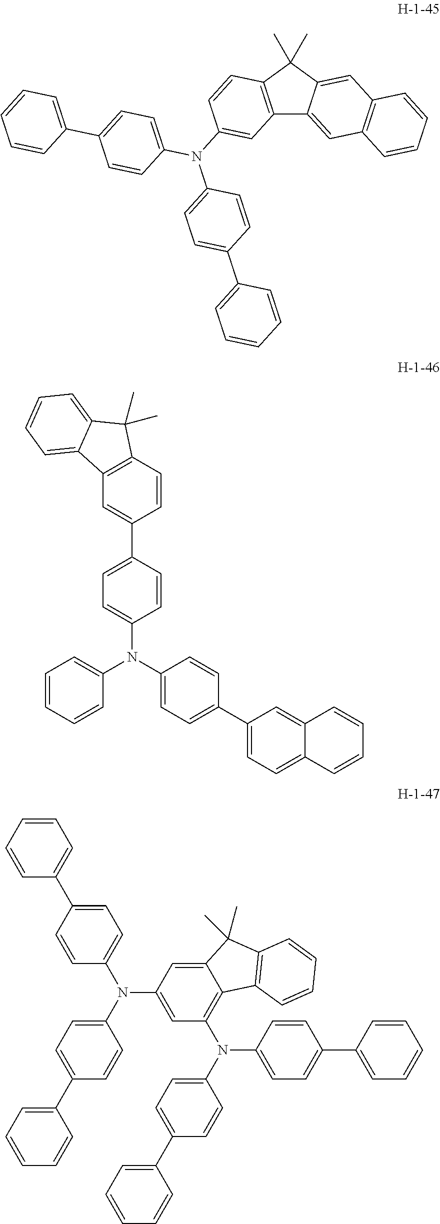 [SMILES: CC1(C)c2ccc(N(c3ccc(-c4ccccc4)cc3)c3ccc(-c4ccccc4)cc3)cc2-c2cc3ccccc3cc21 ; CC1(C)c2ccccc2-c2cc(-c3ccc(N(c4ccccc4)c4ccc(-c5ccc6ccccc6c5)cc4)cc3)ccc21 ; CC1(C)c2ccccc2-c2c(N(c3ccc(-c4ccccc4)cc3)c3ccc(-c4ccccc4)cc3)cc(N(c3ccc(-c4ccccc4)cc3)c3ccc(-c4ccccc4)cc3)cc21]

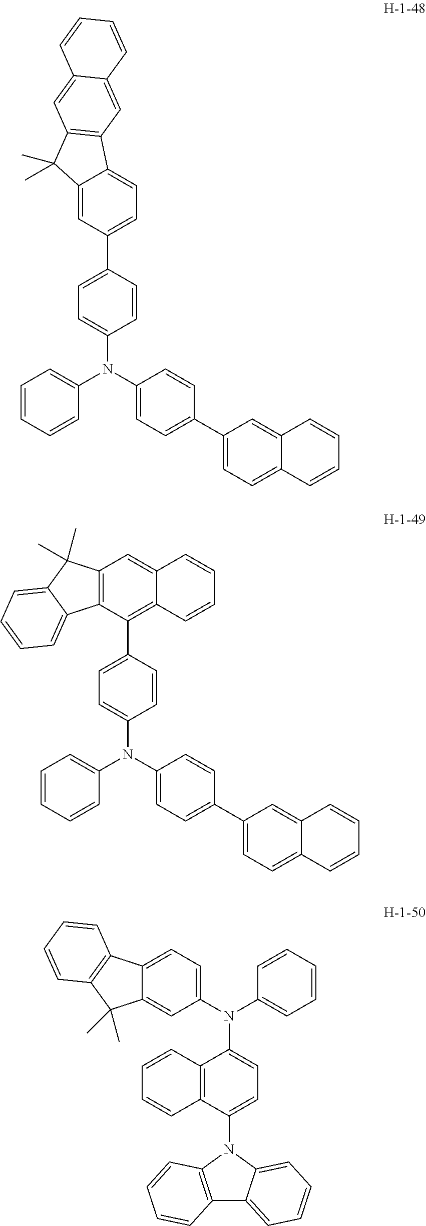 [SMILES: CC1(C)c2cc(-c3ccc(N(c4ccccc4)c4ccc(-c5ccc6ccccc6c5)cc4)cc3)ccc2-c2cc3ccccc3cc21 ; CC1(C)c2ccccc2-c2c1cc1ccccc1c2-c1ccc(N(c2ccccc2)c2ccc(-c3ccc4ccccc4c3)cc2)cc1 ; CC1(C)c2ccccc2-c2ccc(N(c3ccccc3)c3ccc(-n4c5ccccc5c5ccccc54)c4ccccc34)cc21]

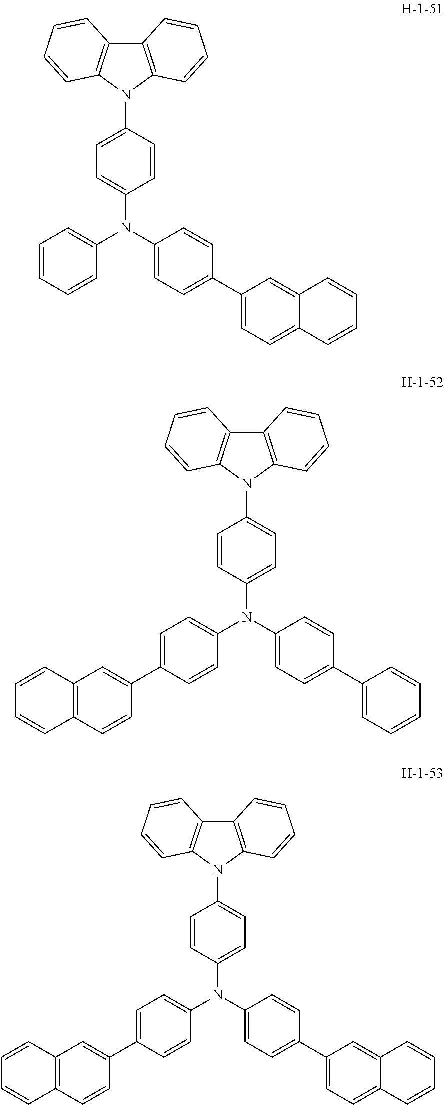 [SMILES: c1ccc(N(c2ccc(-c3ccc4ccccc4c3)cc2)c2ccc(-n3c4ccccc4c4ccccc43)cc2)cc1 ; c1ccc(-c2ccc(N(c3ccc(-c4ccc5ccccc5c4)cc3)c3ccc(-n4c5ccccc5c5ccccc54)cc3)cc2)cc1 ; c1ccc2cc(-c3ccc(N(c4ccc(-c5ccc6ccccc6c5)cc4)c4ccc(-n5c6ccccc6c6ccccc65)cc4)cc3)ccc2c1]

The compound represented by formula 2 includes the following compounds, but is not limited thereto,

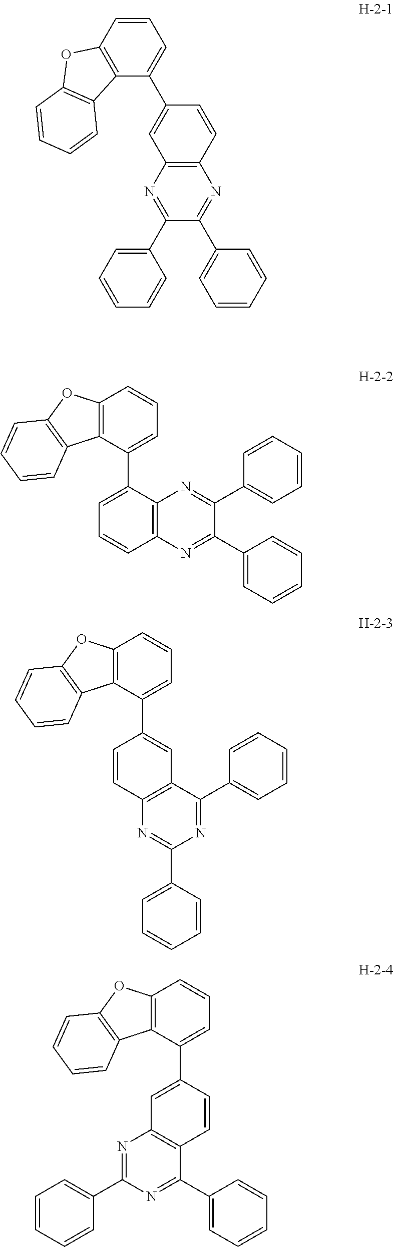

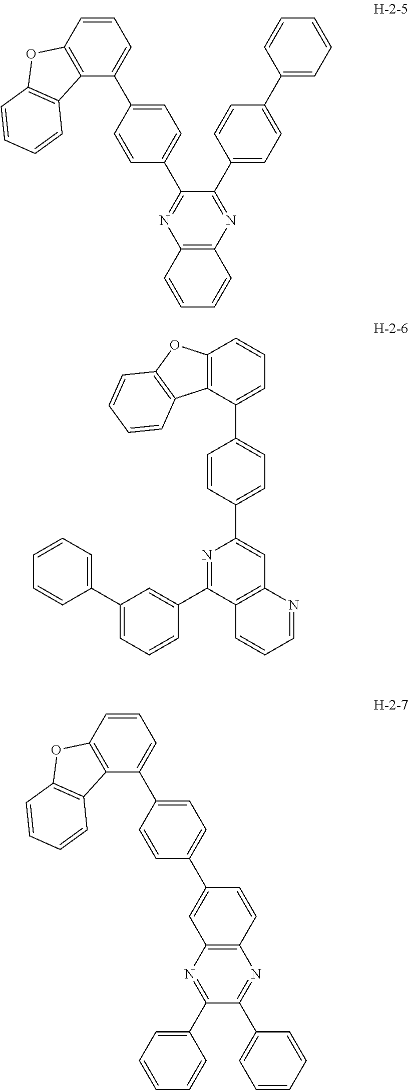

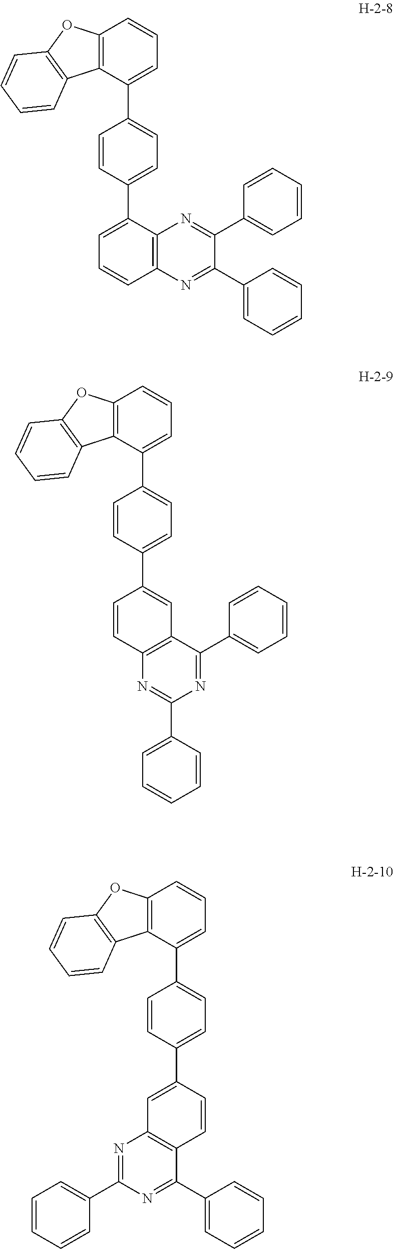 [SMILES: c1ccc(-c2nc3cccc(-c4ccc(-c5cccc6oc7ccccc7c56)cc4)c3nc2-c2ccccc2)cc1 ; c1ccc(-c2nc(-c3ccccc3)c3cc(-c4ccc(-c5cccc6oc7ccccc7c56)cc4)ccc3n2)cc1 ; c1ccc(-c2nc(-c3ccccc3)c3ccc(-c4ccc(-c5cccc6oc7ccccc7c56)cc4)cc3n2)cc1]

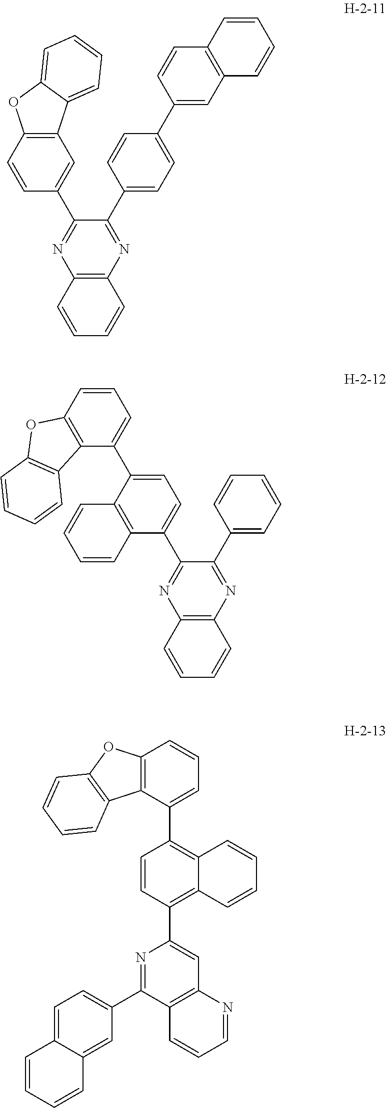 [SMILES: c1ccc2cc(-c3ccc(-c4nc5ccccc5nc4-c4ccc5oc6ccccc6c5c4)cc3)ccc2c1 ; c1ccc(-c2nc3ccccc3nc2-c2ccc(-c3cccc4oc5ccccc5c34)c3ccccc23)cc1 ; c1ccc2cc(-c3nc(-c4ccc(-c5cccc6oc7ccccc7c56)c5ccccc45)cc4ncccc34)ccc2c1]

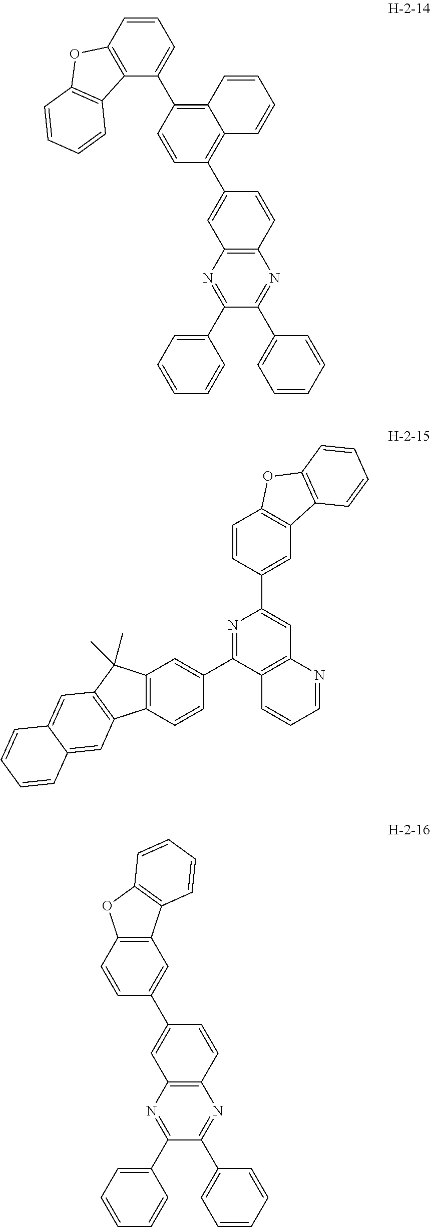 [SMILES: c1ccc(-c2nc3ccc(-c4ccc(-c5cccc6oc7ccccc7c56)c5ccccc45)cc3nc2-c2ccccc2)cc1 ; CC1(C)c2cc(-c3nc(-c4ccc5oc6ccccc6c5c4)cc4ncccc34)ccc2-c2cc3ccccc3cc21 ; c1ccc(-c2nc3ccc(-c4ccc5oc6ccccc6c5c4)cc3nc2-c2ccccc2)cc1]

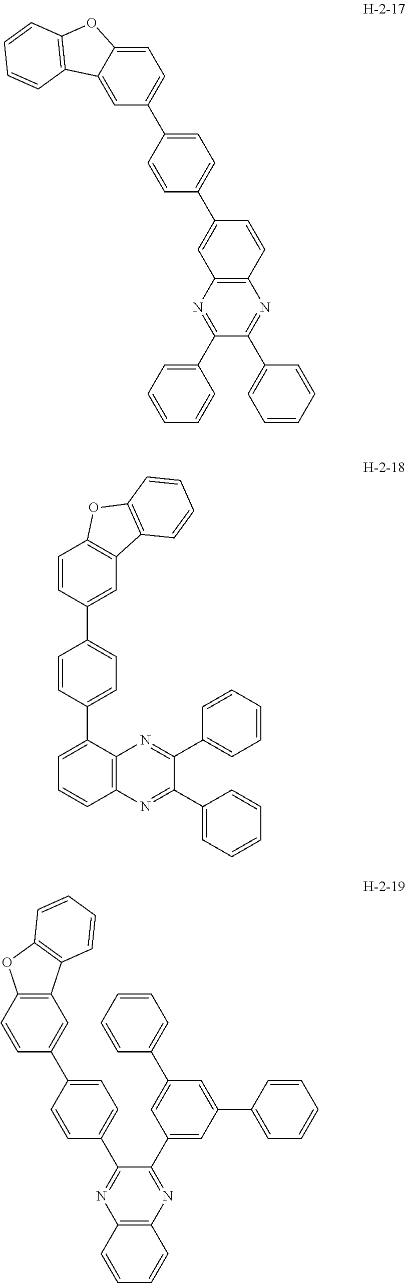 [SMILES: c1ccc(-c2nc3ccc(-c4ccc(-c5ccc6oc7ccccc7c6c5)cc4)cc3nc2-c2ccccc2)cc1 ; c1ccc(-c2nc3cccc(-c4ccc(-c5ccc6oc7ccccc7c6c5)cc4)c3nc2-c2ccccc2)cc1 ; c1ccc(-c2cc(-c3ccccc3)cc(-c3nc4ccccc4nc3-c3ccc(-c4ccc5oc6ccccc6c5c4)cc3)c2)cc1]

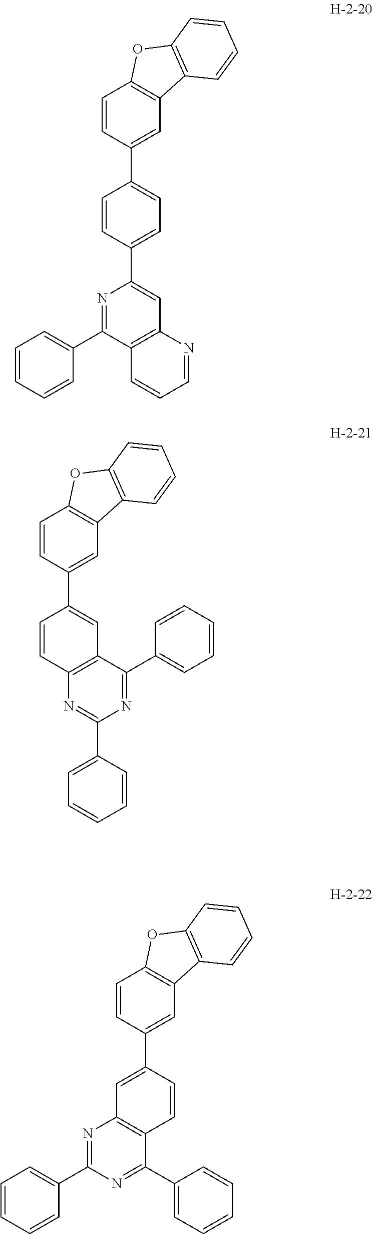 [SMILES: c1ccc(-c2nc(-c3ccc(-c4ccc5oc6ccccc6c5c4)cc3)cc3ncccc23)cc1 ; c1ccc(-c2nc(-c3ccccc3)c3cc(-c4ccc5oc6ccccc6c5c4)ccc3n2)cc1 ; c1ccc(-c2nc(-c3ccccc3)c3ccc(-c4ccc5oc6ccccc6c5c4)cc3n2)cc1]

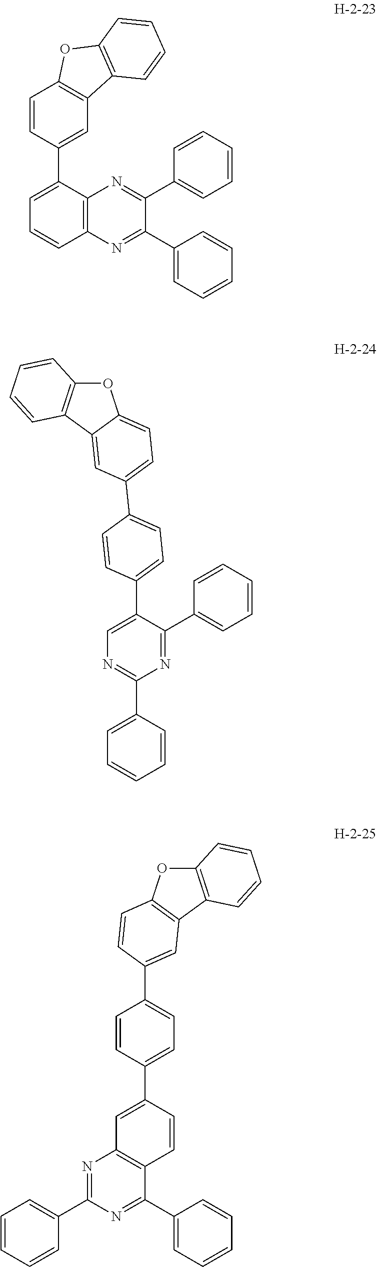 [SMILES: c1ccc(-c2nc3cccc(-c4ccc5oc6ccccc6c5c4)c3nc2-c2ccccc2)cc1 ; c1ccc(-c2ncc(-c3ccc(-c4ccc5oc6ccccc6c5c4)cc3)c(-c3ccccc3)n2)cc1 ; c1ccc(-c2nc(-c3ccccc3)c3ccc(-c4ccc(-c5ccc6oc7ccccc7c6c5)cc4)cc3n2)cc1]

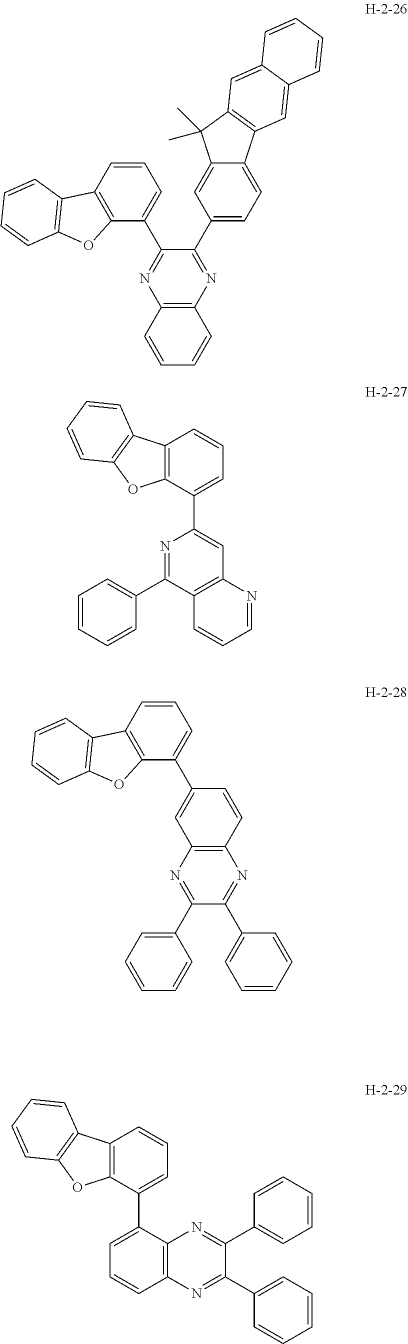 [SMILES: CC1(C)c2cc(-c3nc4ccccc4nc3-c3cccc4c3oc3ccccc34)ccc2-c2cc3ccccc3cc21 ; c1ccc(-c2nc(-c3cccc4c3oc3ccccc34)cc3ncccc23)cc1 ; c1ccc(-c2nc3ccc(-c4cccc5c4oc4ccccc45)cc3nc2-c2ccccc2)cc1 ; c1ccc(-c2nc3cccc(-c4cccc5c4oc4ccccc45)c3nc2-c2ccccc2)cc1]

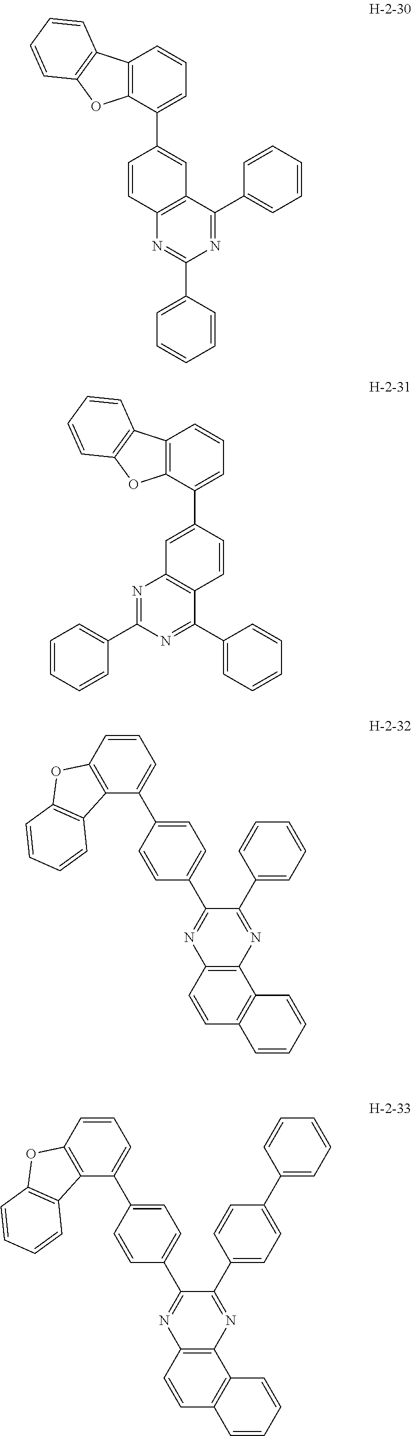 [SMILES: c1ccc(-c2nc(-c3ccccc3)c3cc(-c4cccc5c4oc4ccccc45)ccc3n2)cc1 ; c1ccc(-c2nc(-c3ccccc3)c3ccc(-c4cccc5c4oc4ccccc45)cc3n2)cc1 ; c1ccc(-c2nc3c(ccc4ccccc43)nc2-c2ccc(-c3cccc4oc5ccccc5c34)cc2)cc1 ; c1ccc(-c2ccc(-c3nc4c(ccc5ccccc54)nc3-c3ccc(-c4cccc5oc6ccccc6c45)cc3)cc2)cc1]

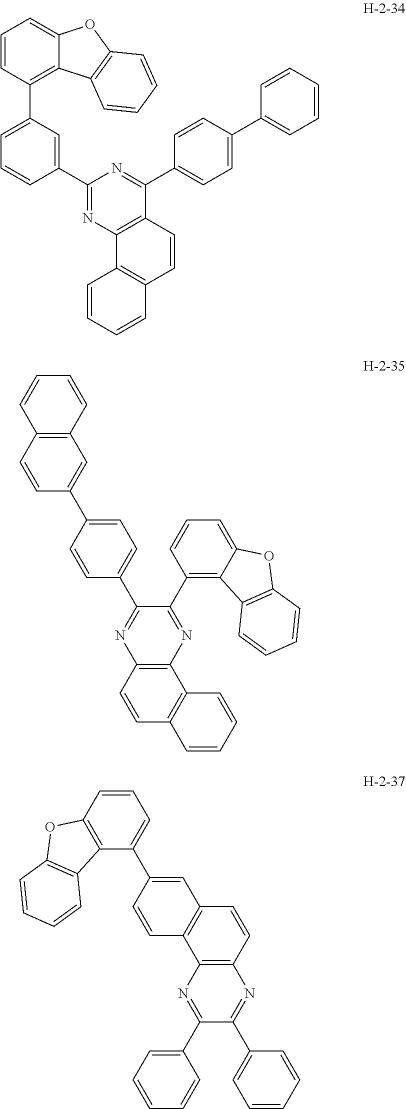 [SMILES: c1ccc(-c2ccc(-c3nc(-c4cccc(-c5cccc6oc7ccccc7c56)c4)nc4c3ccc3ccccc34)cc2)cc1 ; c1ccc2cc(-c3ccc(-c4nc5ccc6ccccc6c5nc4-c4cccc5oc6ccccc6c45)cc3)ccc2c1 ; c1ccc(-c2nc3ccc4cc(-c5cccc6oc7ccccc7c56)ccc4c3nc2-c2ccccc2)cc1]

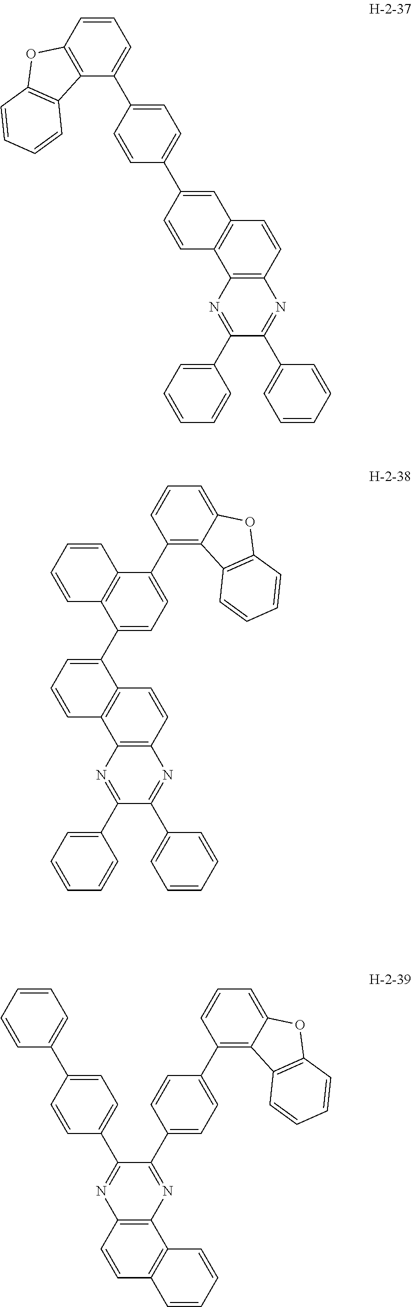 [SMILES: c1ccc(-c2nc3ccc4cc(-c5ccc(-c6cccc7oc8ccccc8c67)cc5)ccc4c3nc2-c2ccccc2)cc1 ; c1ccc(-c2nc3ccc4c(-c5ccc(-c6cccc7oc8ccccc8c67)c6ccccc56)cccc4c3nc2-c2ccccc2)cc1 ; c1ccc(-c2ccc(-c3nc4ccc5ccccc5c4nc3-c3ccc(-c4cccc5oc6ccccc6c45)cc3)cc2)cc1]

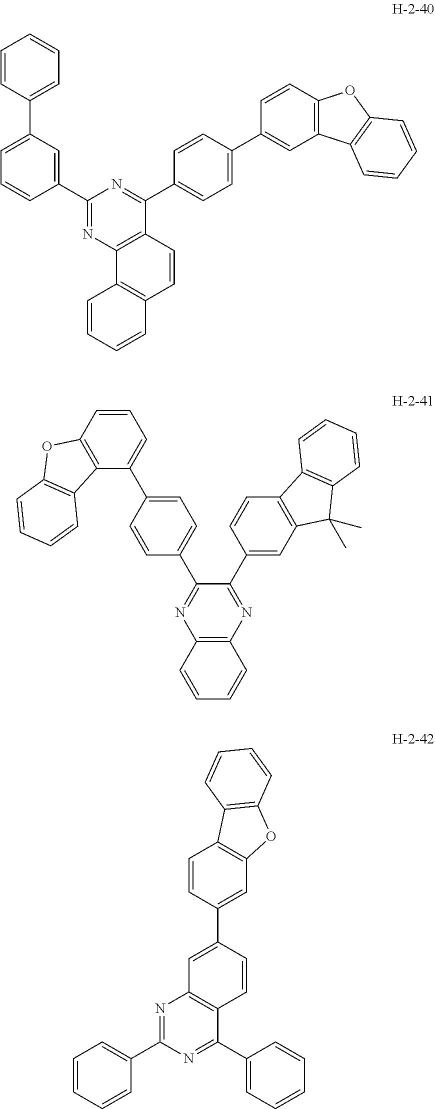 [SMILES: c1ccc(-c2cccc(-c3nc(-c4ccc(-c5ccc6oc7ccccc7c6c5)cc4)c4ccc5ccccc5c4n3)c2)cc1 ; CC1(C)c2ccccc2-c2ccc(-c3nc4ccccc4nc3-c3ccc(-c4cccc5oc6ccccc6c45)cc3)cc21 ; c1ccc(-c2nc(-c3ccccc3)c3ccc(-c4ccc5c(c4)oc4ccccc45)cc3n2)cc1]

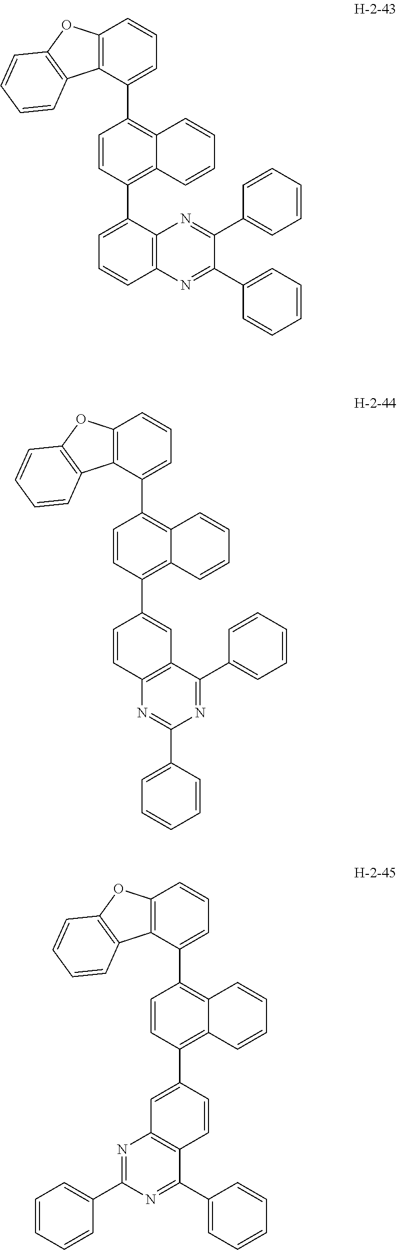 [SMILES: c1ccc(-c2nc3cccc(-c4ccc(-c5cccc6oc7ccccc7c56)c5ccccc45)c3nc2-c2ccccc2)cc1 ; c1ccc(-c2nc(-c3ccccc3)c3cc(-c4ccc(-c5cccc6oc7ccccc7c56)c5ccccc45)ccc3n2)cc1 ; c1ccc(-c2nc(-c3ccccc3)c3ccc(-c4ccc(-c5cccc6oc7ccccc7c56)c5ccccc45)cc3n2)cc1]

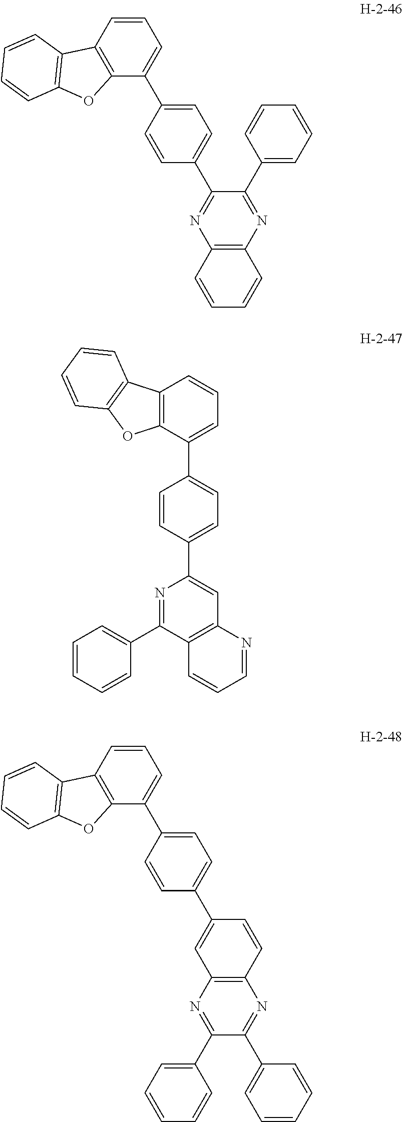 [SMILES: c1ccc(-c2nc3ccccc3nc2-c2ccc(-c3cccc4c3oc3ccccc34)cc2)cc1 ; c1ccc(-c2nc(-c3ccc(-c4cccc5c4oc4ccccc45)cc3)cc3ncccc23)cc1 ; c1ccc(-c2nc3ccc(-c4ccc(-c5cccc6c5oc5ccccc56)cc4)cc3nc2-c2ccccc2)cc1]

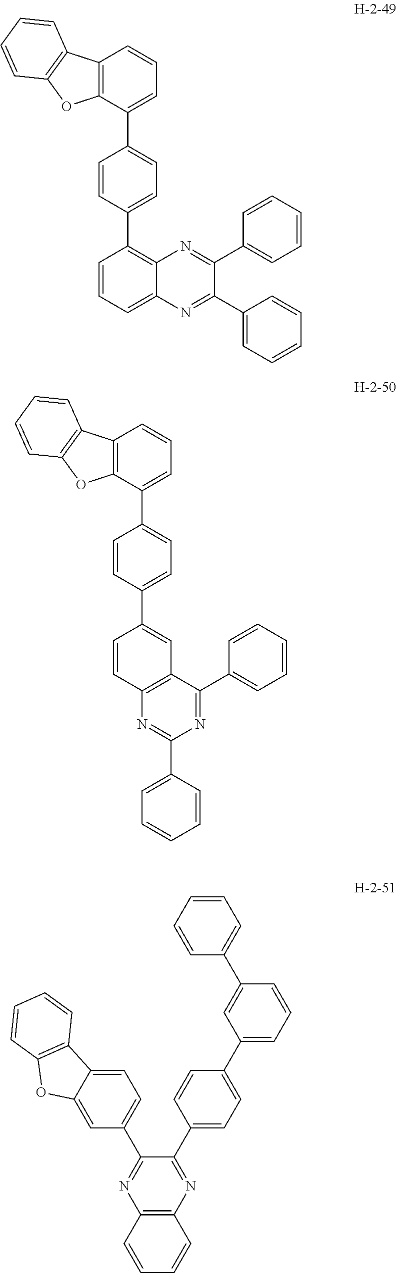 [SMILES: c1ccc(-c2nc3cccc(-c4ccc(-c5cccc6c5oc5ccccc56)cc4)c3nc2-c2ccccc2)cc1 ; c1ccc(-c2nc(-c3ccccc3)c3cc(-c4ccc(-c5cccc6c5oc5ccccc56)cc4)ccc3n2)cc1 ; c1ccc(-c2cccc(-c3ccc(-c4nc5ccccc5nc4-c4ccc5c(c4)oc4ccccc45)cc3)c2)cc1]

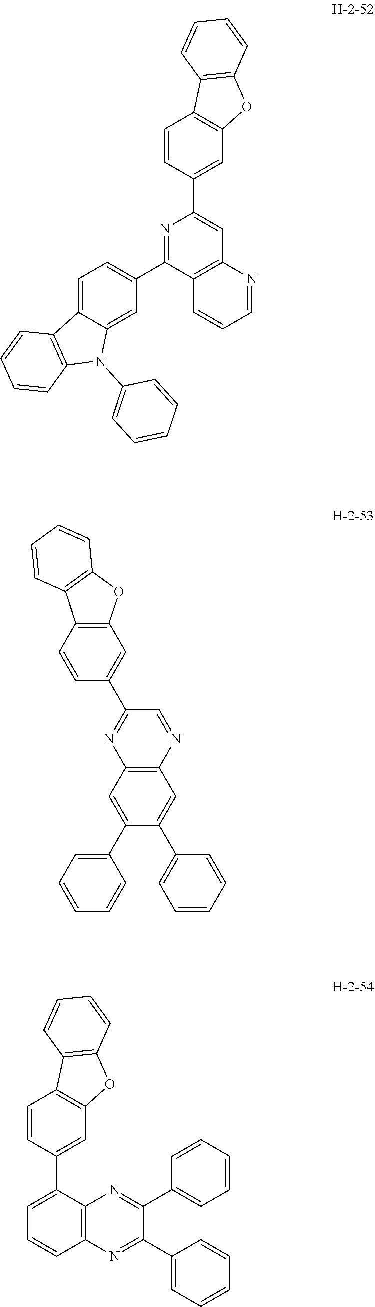 [SMILES: c1ccc(-n2c3ccccc3c3ccc(-c4nc(-c5ccc6c(c5)oc5ccccc56)cc5ncccc45)cc32)cc1 ; c1ccc(-c2cc3ncc(-c4ccc5c(c4)oc4ccccc45)nc3cc2-c2ccccc2)cc1 ; c1ccc(-c2nc3cccc(-c4ccc5c(c4)oc4ccccc45)c3nc2-c2ccccc2)cc1]

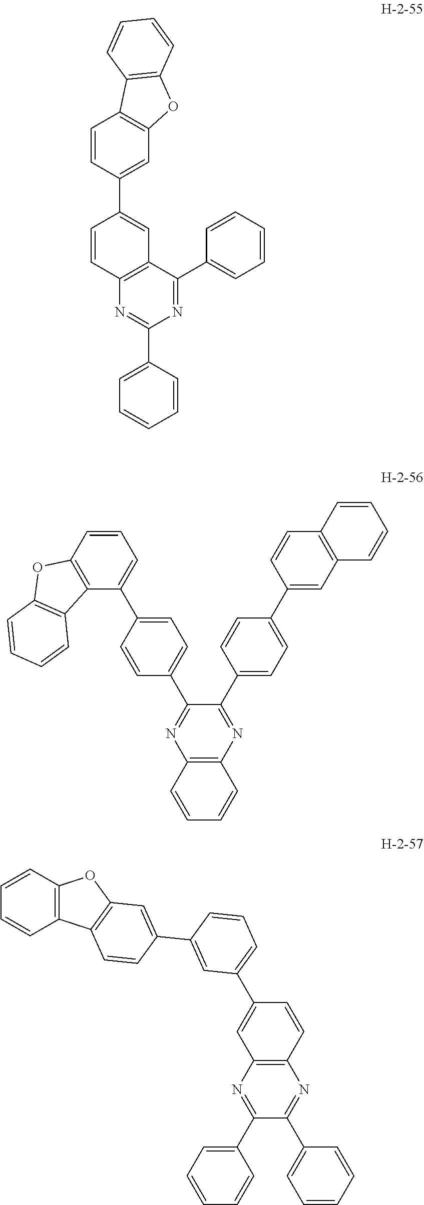 [SMILES: c1ccc(-c2nc(-c3ccccc3)c3cc(-c4ccc5c(c4)oc4ccccc45)ccc3n2)cc1 ; c1ccc2cc(-c3ccc(-c4nc5ccccc5nc4-c4ccc(-c5cccc6oc7ccccc7c56)cc4)cc3)ccc2c1 ; c1ccc(-c2nc3ccc(-c4cccc(-c5ccc6c(c5)oc5ccccc56)c4)cc3nc2-c2ccccc2)cc1]

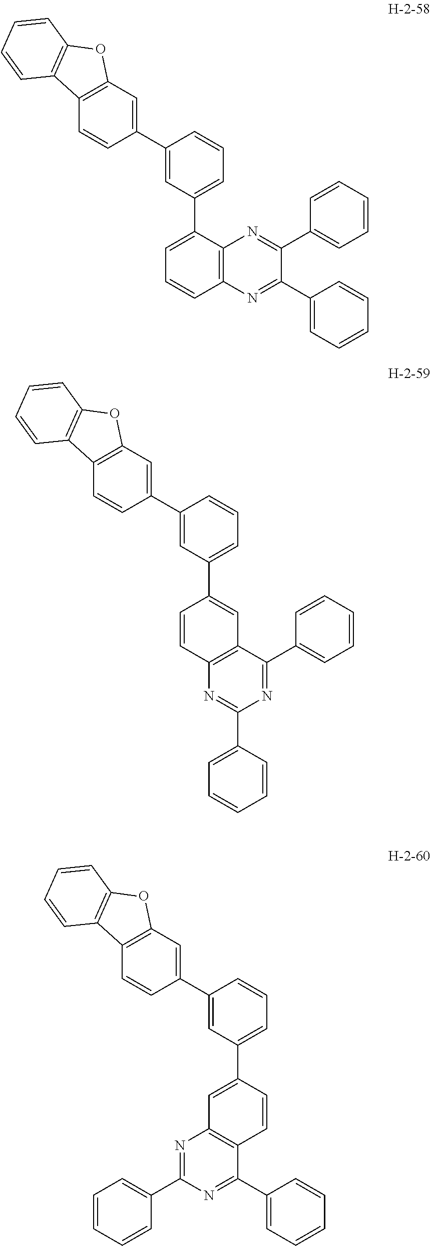 [SMILES: c1ccc(-c2nc3cccc(-c4cccc(-c5ccc6c(c5)oc5ccccc56)c4)c3nc2-c2ccccc2)cc1 ; c1ccc(-c2nc(-c3ccccc3)c3cc(-c4cccc(-c5ccc6c(c5)oc5ccccc56)c4)ccc3n2)cc1 ; c1ccc(-c2nc(-c3ccccc3)c3ccc(-c4cccc(-c5ccc6c(c5)oc5ccccc56)c4)cc3n2)cc1]

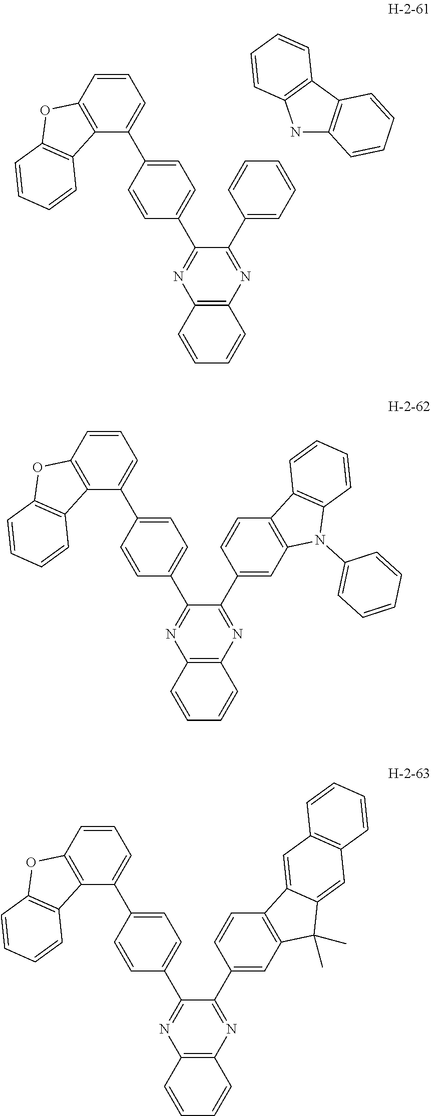

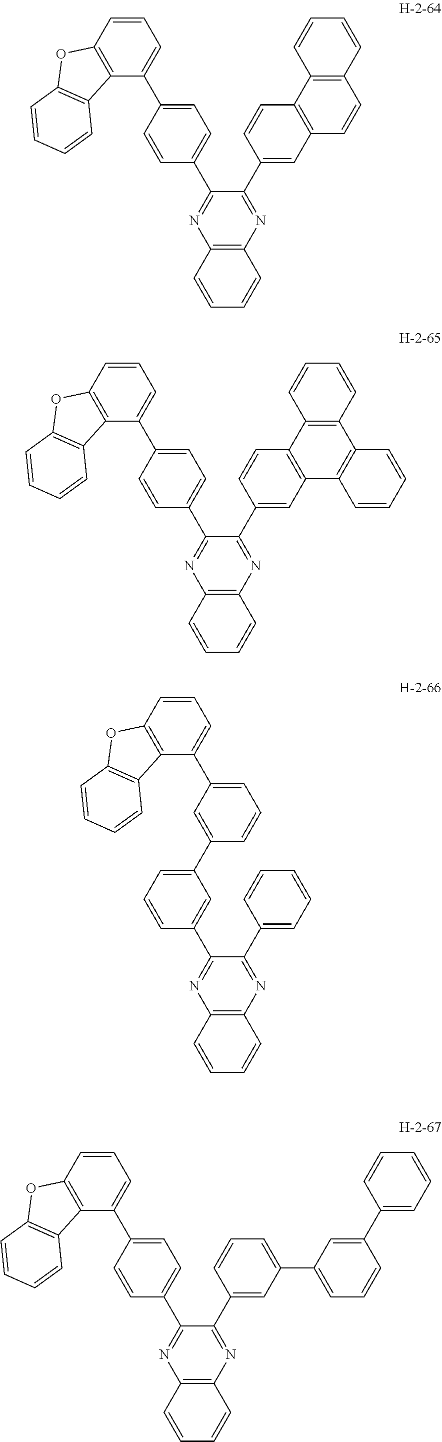

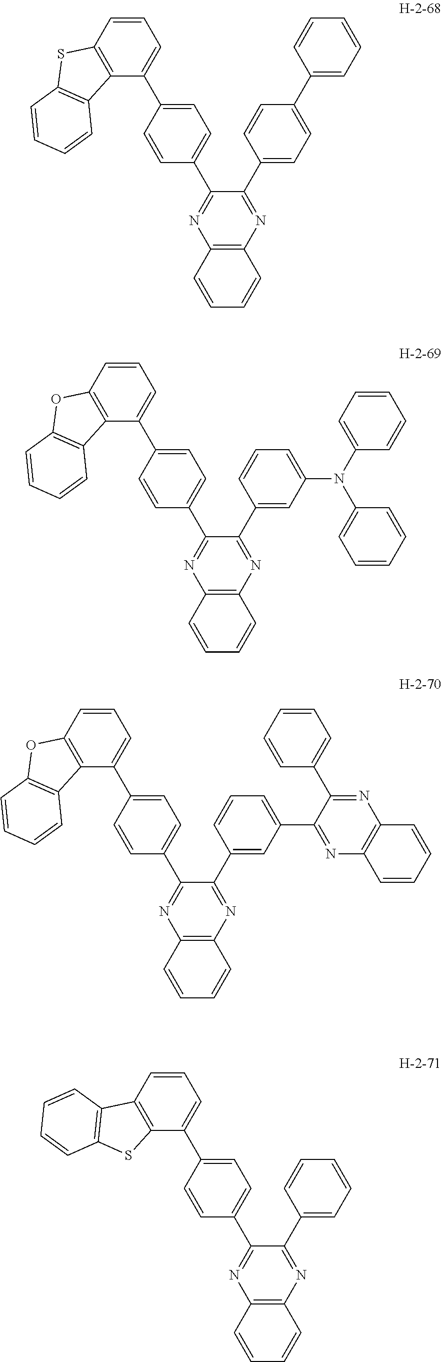 [SMILES: c1ccc(-c2ccc(-c3nc4ccccc4nc3-c3ccc(-c4cccc5sc6ccccc6c45)cc3)cc2)cc1 ; c1ccc(N(c2ccccc2)c2cccc(-c3nc4ccccc4nc3-c3ccc(-c4cccc5oc6ccccc6c45)cc3)c2)cc1 ; c1ccc(-c2nc3ccccc3nc2-c2cccc(-c3nc4ccccc4nc3-c3ccc(-c4cccc5oc6ccccc6c45)cc3)c2)cc1 ; c1ccc(-c2nc3ccccc3nc2-c2ccc(-c3cccc4c3sc3ccccc34)cc2)cc1]

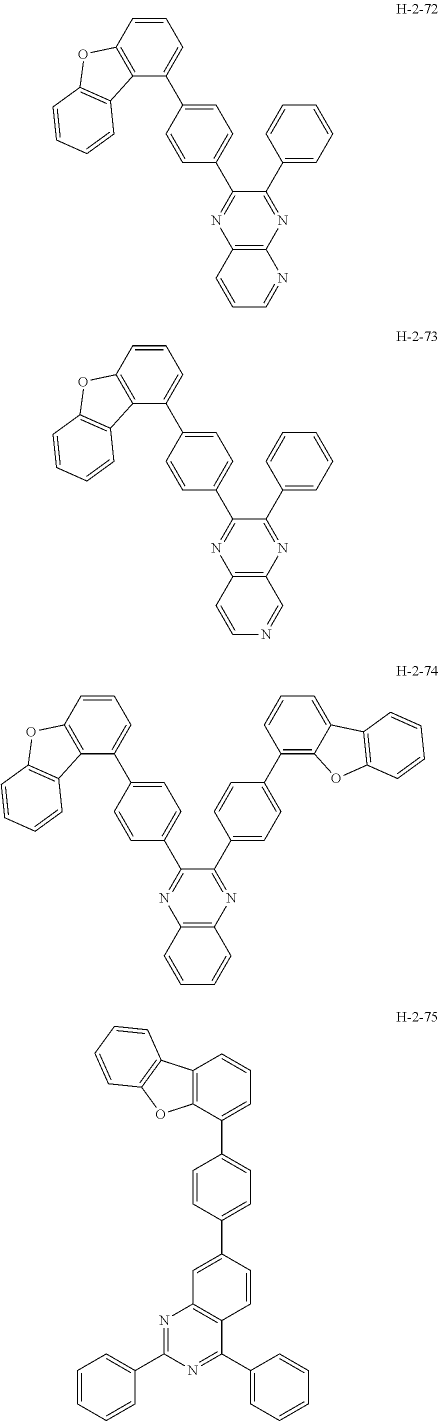 [SMILES: c1ccc(-c2nc3ncccc3nc2-c2ccc(-c3cccc4oc5ccccc5c34)cc2)cc1 ; c1ccc(-c2nc3cnccc3nc2-c2ccc(-c3cccc4oc5ccccc5c34)cc2)cc1 ; c1ccc2nc(-c3ccc(-c4cccc5oc6ccccc6c45)cc3)c(-c3ccc(-c4cccc5c4oc4ccccc45)cc3)nc2c1 ; c1ccc(-c2nc(-c3ccccc3)c3ccc(-c4ccc(-c5cccc6c5oc5ccccc56)cc4)cc3n2)cc1]

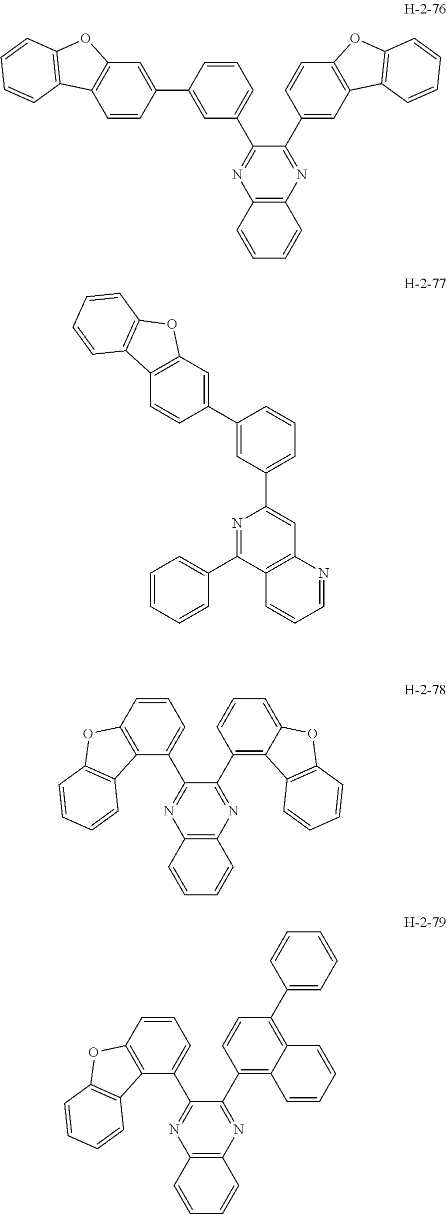 [SMILES: c1cc(-c2ccc3c(c2)oc2ccccc23)cc(-c2nc3ccccc3nc2-c2ccc3oc4ccccc4c3c2)c1 ; c1ccc(-c2nc(-c3cccc(-c4ccc5c(c4)oc4ccccc45)c3)cc3ncccc23)cc1 ; c1ccc2nc(-c3cccc4oc5ccccc5c34)c(-c3cccc4oc5ccccc5c34)nc2c1 ; c1ccc(-c2ccc(-c3nc4ccccc4nc3-c3cccc4oc5ccccc5c34)c3ccccc23)cc1]

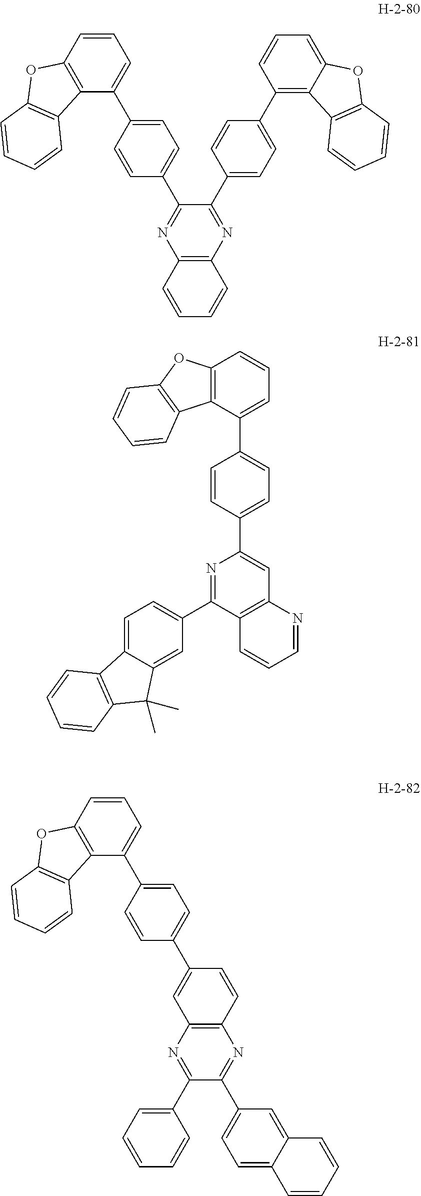

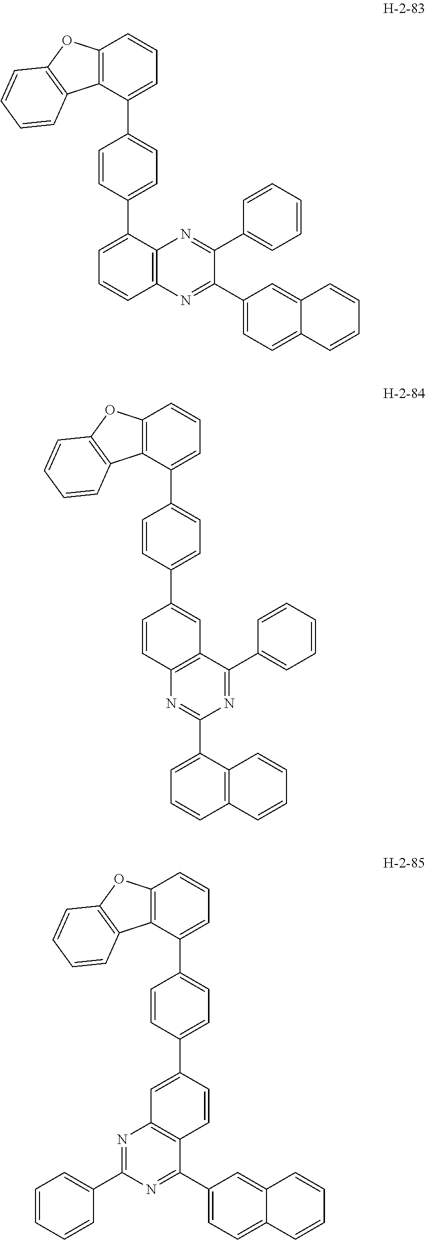 [SMILES: c1ccc(-c2nc3c(-c4ccc(-c5cccc6oc7ccccc7c56)cc4)cccc3nc2-c2ccc3ccccc3c2)cc1 ; c1ccc(-c2nc(-c3cccc4ccccc34)nc3ccc(-c4ccc(-c5cccc6oc7ccccc7c56)cc4)cc23)cc1 ; c1ccc(-c2nc(-c3ccc4ccccc4c3)c3ccc(-c4ccc(-c5cccc6oc7ccccc7c56)cc4)cc3n2)cc1]

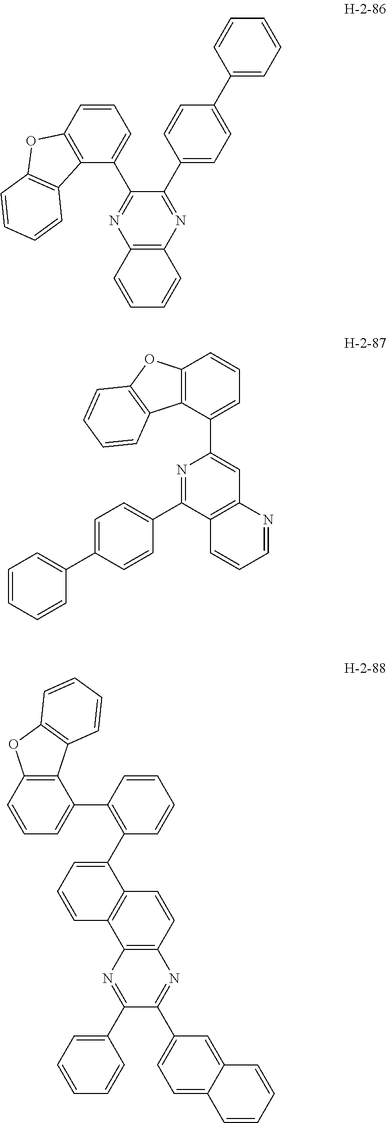 [SMILES: c1ccc(-c2ccc(-c3nc4ccccc4nc3-c3cccc4oc5ccccc5c34)cc2)cc1 ; c1ccc(-c2ccc(-c3nc(-c4cccc5oc6ccccc6c45)cc4ncccc34)cc2)cc1 ; c1ccc(-c2nc3c(ccc4c(-c5ccccc5-c5cccc6oc7ccccc7c56)cccc43)nc2-c2ccc3ccccc3c2)cc1]

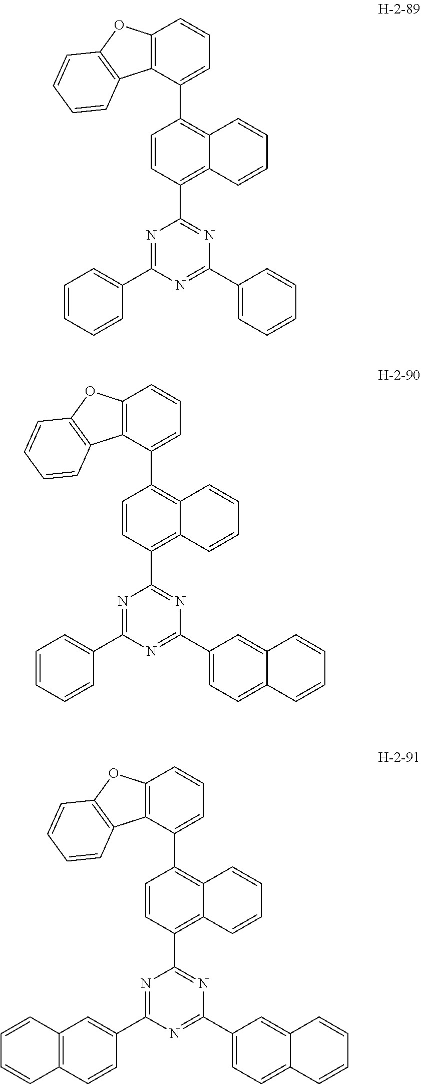 [SMILES: c1ccc(-c2nc(-c3ccccc3)nc(-c3ccc(-c4cccc5oc6ccccc6c45)c4ccccc34)n2)cc1 ; c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1]

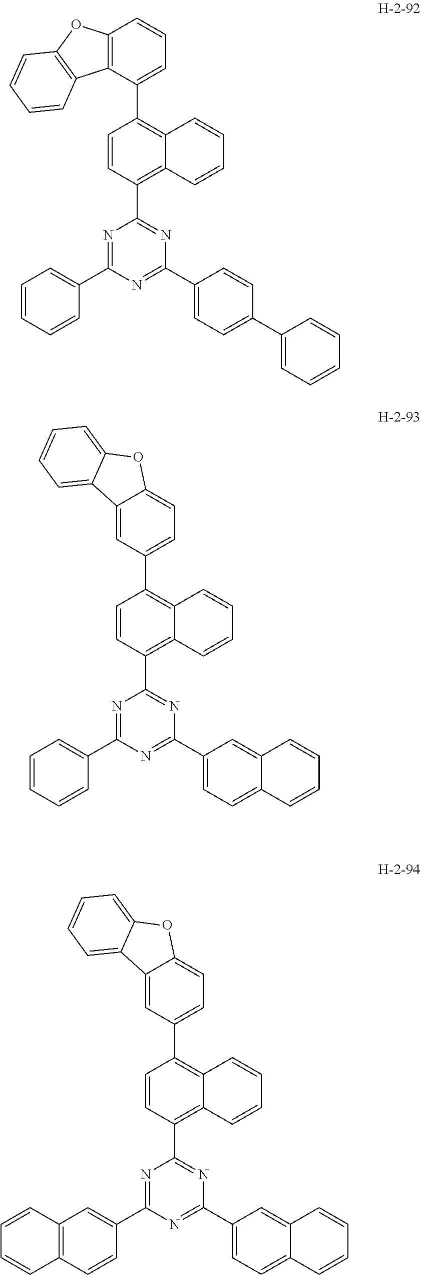 [SMILES: c1ccc(-c2ccc(-c3nc(-c4ccccc4)nc(-c4ccc(-c5cccc6oc7ccccc7c56)c5ccccc45)n3)cc2)cc1 ; c1ccc(-c2nc(-c3ccc4ccccc4c3)nc(-c3ccc(-c4ccc5oc6ccccc6c5c4)c4ccccc34)n2)cc1 ; c1ccc2cc(-c3nc(-c4ccc5ccccc5c4)nc(-c4ccc(-c5ccc6oc7ccccc7c6c5)c5ccccc45)n3)ccc2c1]

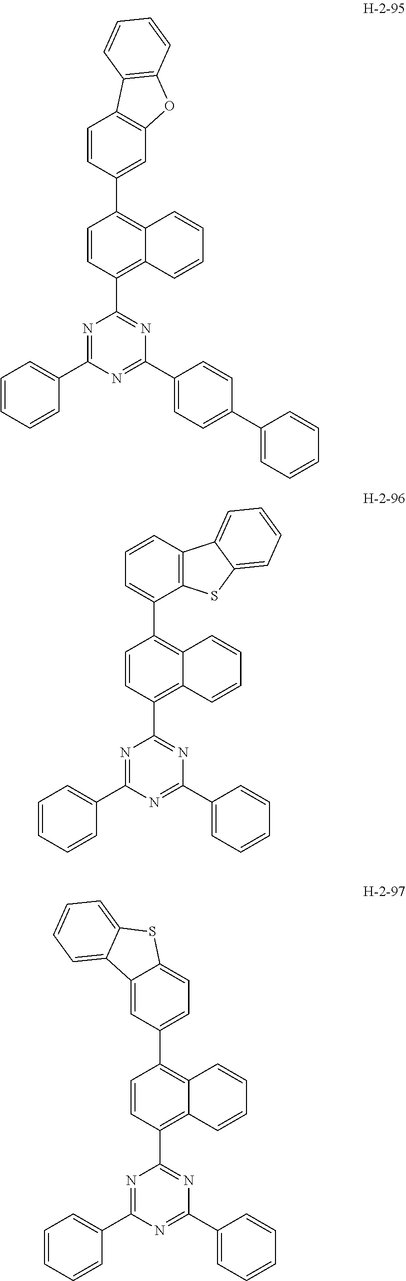

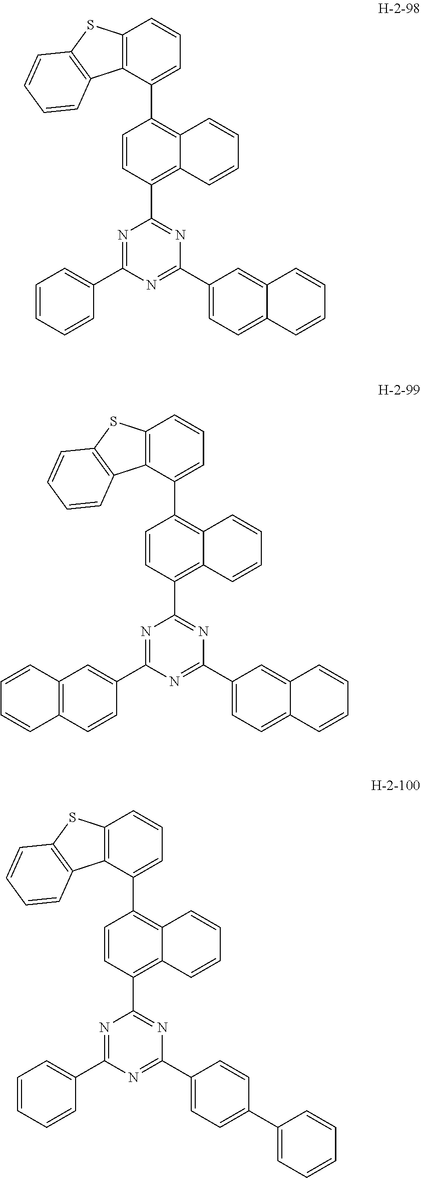 [SMILES: c1ccc(-c2nc(-c3ccc4ccccc4c3)nc(-c3ccc(-c4cccc5sc6ccccc6c45)c4ccccc34)n2)cc1 ; c1ccc2cc(-c3nc(-c4ccc5ccccc5c4)nc(-c4ccc(-c5cccc6sc7ccccc7c56)c5ccccc45)n3)ccc2c1 ; c1ccc(-c2ccc(-c3nc(-c4ccccc4)nc(-c4ccc(-c5cccc6sc7ccccc7c56)c5ccccc45)n3)cc2)cc1]

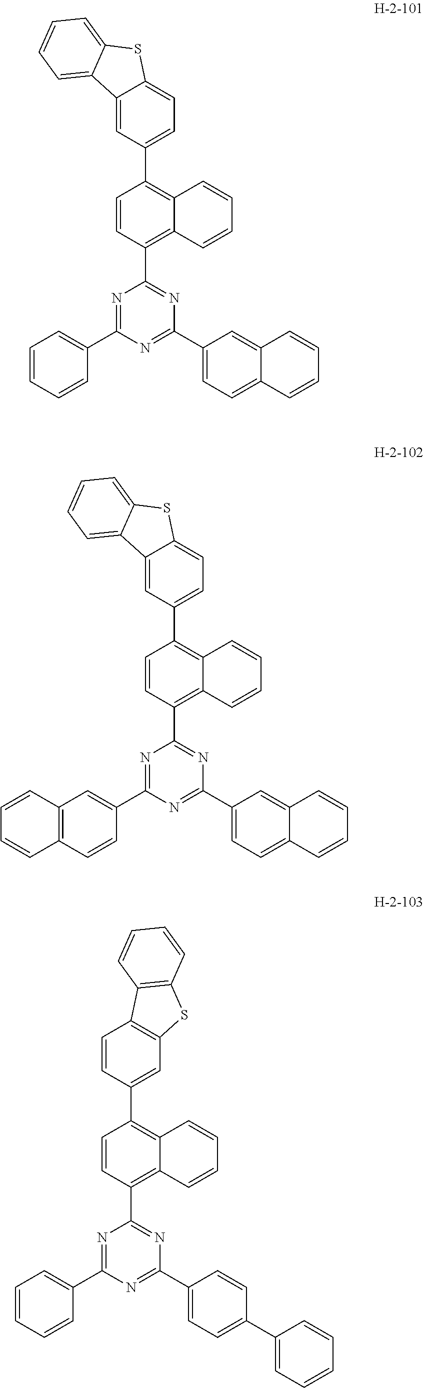 [SMILES: c1ccc(-c2nc(-c3ccc4ccccc4c3)nc(-c3ccc(-c4ccc5sc6ccccc6c5c4)c4ccccc34)n2)cc1 ; c1ccc2cc(-c3nc(-c4ccc5ccccc5c4)nc(-c4ccc(-c5ccc6sc7ccccc7c6c5)c5ccccc45)n3)ccc2c1 ; c1ccc(-c2ccc(-c3nc(-c4ccccc4)nc(-c4ccc(-c5ccc6c(c5)sc5ccccc56)c5ccccc45)n3)cc2)cc1]

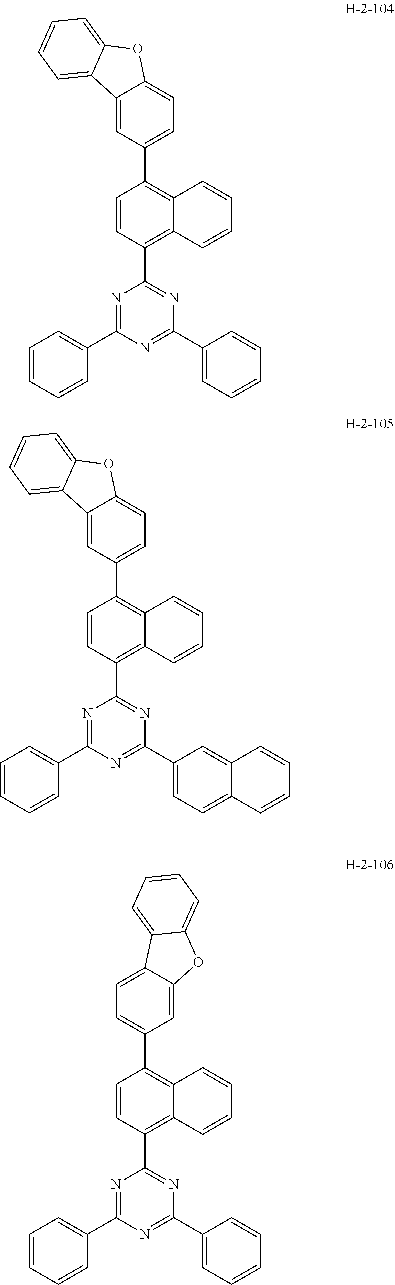

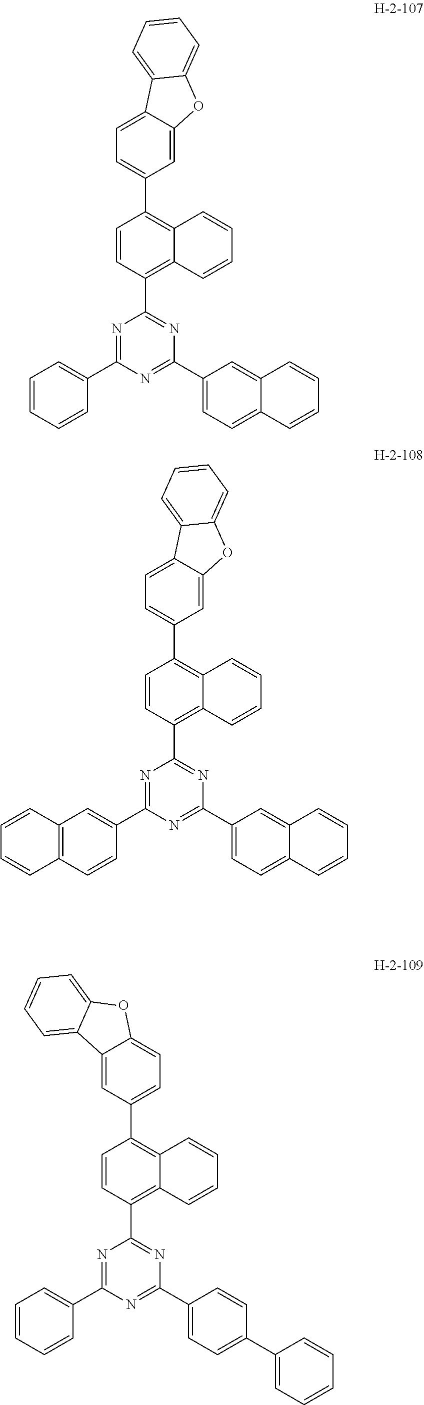

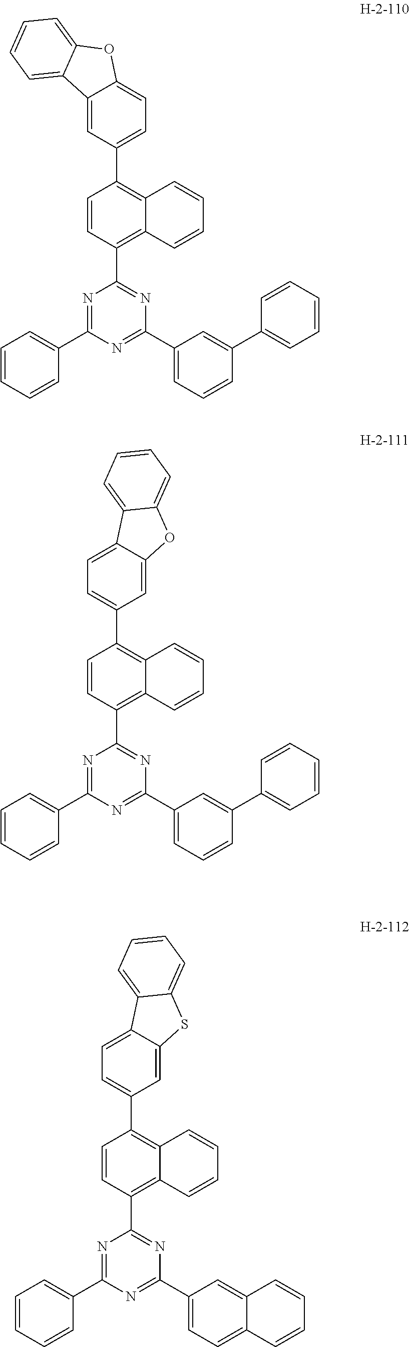 [SMILES: c1ccc(-c2cccc(-c3nc(-c4ccccc4)nc(-c4ccc(-c5ccc6oc7ccccc7c6c5)c5ccccc45)n3)c2)cc1 ; c1ccc(-c2cccc(-c3nc(-c4ccccc4)nc(-c4ccc(-c5ccc6c(c5)oc5ccccc56)c5ccccc45)n3)c2)cc1 ; c1ccc(-c2nc(-c3ccc4ccccc4c3)nc(-c3ccc(-c4ccc5c(c4)sc4ccccc45)c4ccccc34)n2)cc1]

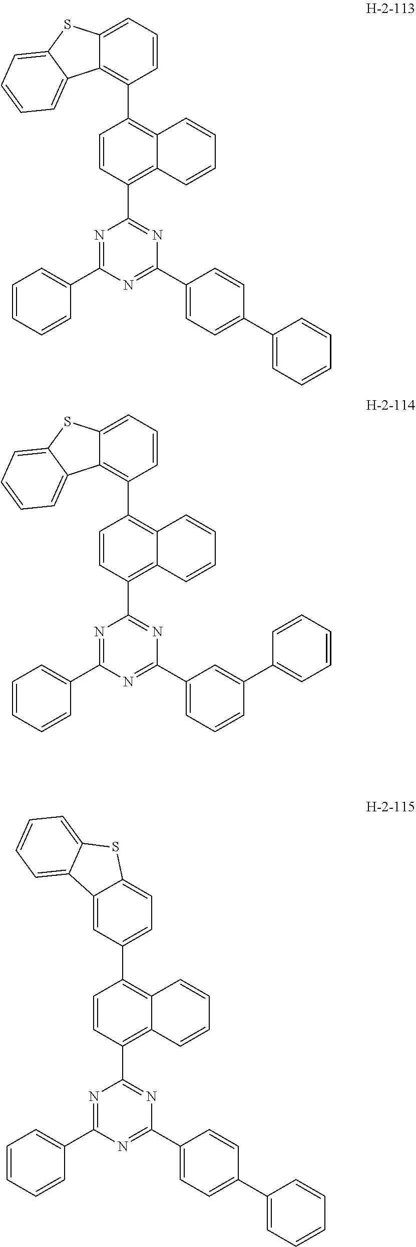 [SMILES: c1ccc(-c2ccc(-c3nc(-c4ccccc4)nc(-c4ccc(-c5cccc6sc7ccccc7c56)c5ccccc45)n3)cc2)cc1 ; c1ccc(-c2cccc(-c3nc(-c4ccccc4)nc(-c4ccc(-c5cccc6sc7ccccc7c56)c5ccccc45)n3)c2)cc1 ; c1ccc(-c2ccc(-c3nc(-c4ccccc4)nc(-c4ccc(-c5ccc6sc7ccccc7c6c5)c5ccccc45)n3)cc2)cc1]

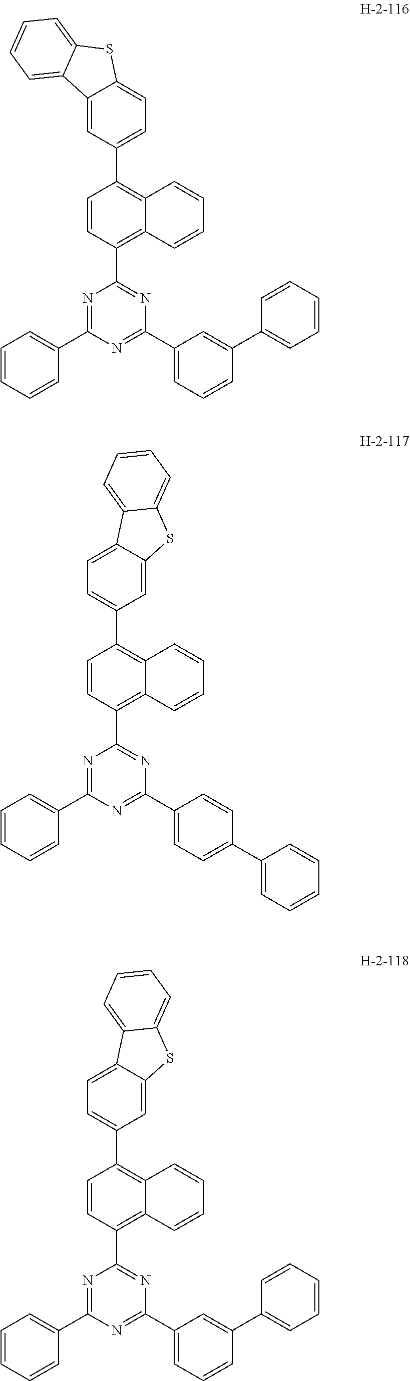 [SMILES: c1ccc(-c2cccc(-c3nc(-c4ccccc4)nc(-c4ccc(-c5ccc6sc7ccccc7c6c5)c5ccccc45)n3)c2)cc1 ; c1ccc(-c2ccc(-c3nc(-c4ccccc4)nc(-c4ccc(-c5ccc6c(c5)sc5ccccc56)c5ccccc45)n3)cc2)cc1 ; c1ccc(-c2cccc(-c3nc(-c4ccccc4)nc(-c4ccc(-c5ccc6c(c5)sc5ccccc56)c5ccccc45)n3)c2)cc1]

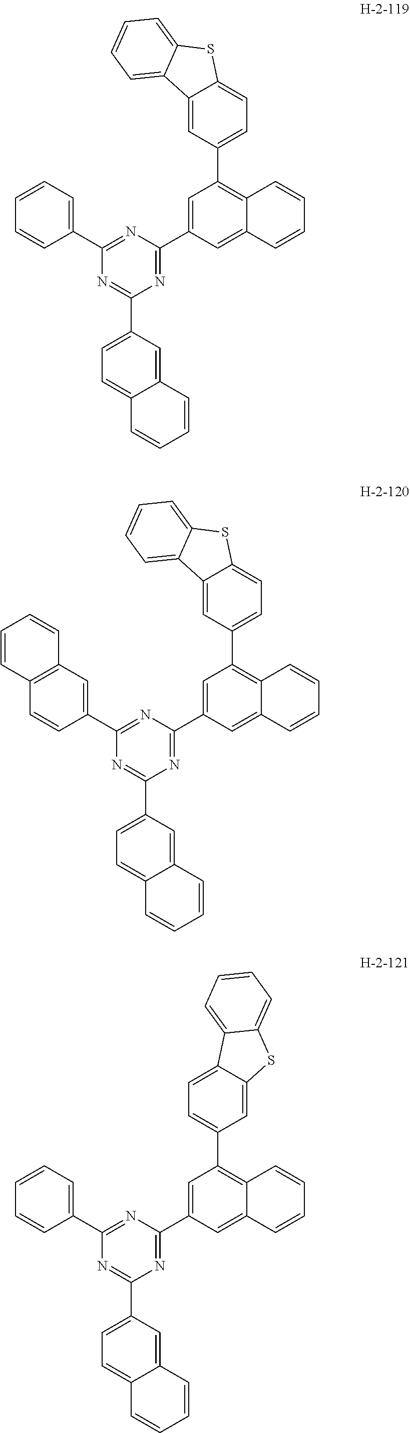 [SMILES: c1ccc(-c2nc(-c3ccc4ccccc4c3)nc(-c3cc(-c4ccc5sc6ccccc6c5c4)c4ccccc4c3)n2)cc1 ; c1ccc2cc(-c3nc(-c4ccc5ccccc5c4)nc(-c4cc(-c5ccc6sc7ccccc7c6c5)c5ccccc5c4)n3)ccc2c1 ; c1ccc(-c2nc(-c3ccc4ccccc4c3)nc(-c3cc(-c4ccc5c(c4)sc4ccccc45)c4ccccc4c3)n2)cc1]

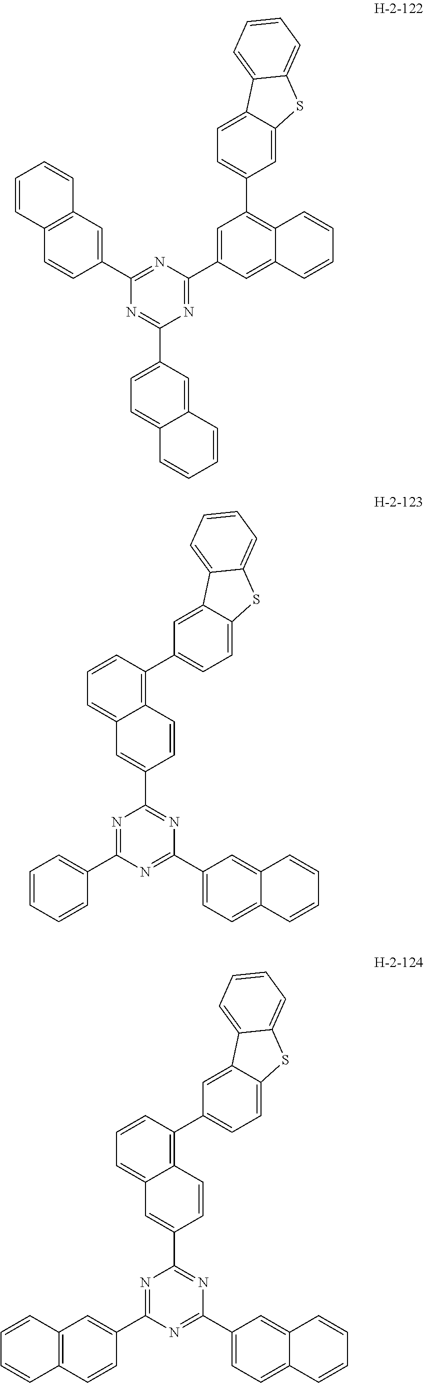

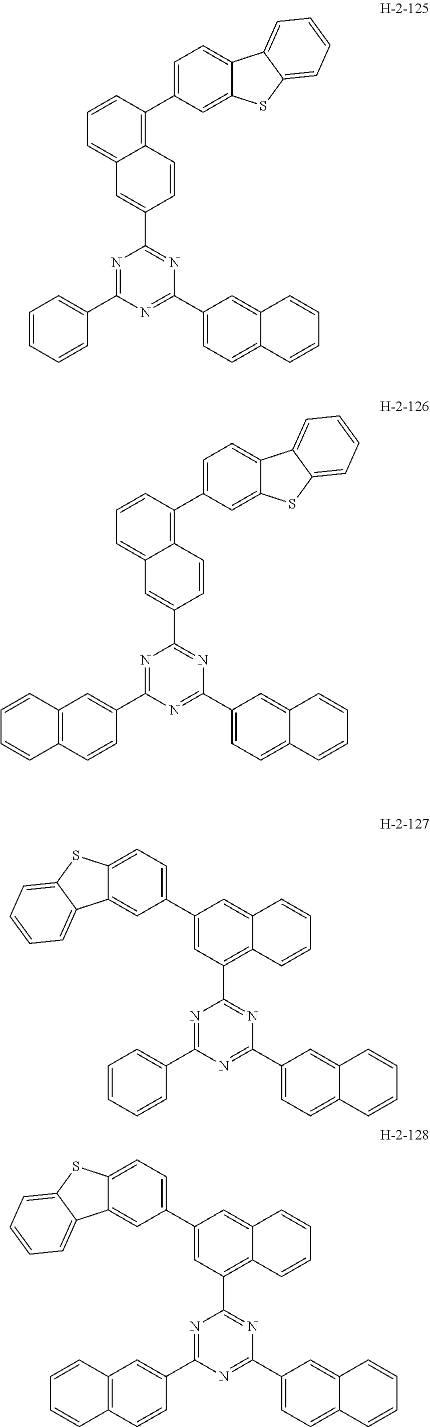

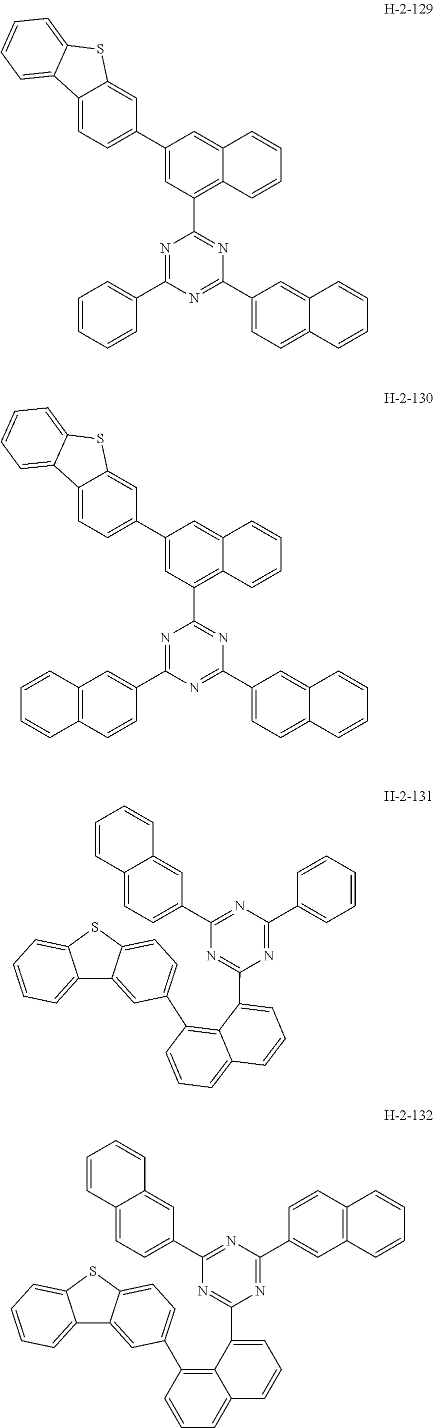 [SMILES: c1ccc(-c2nc(-c3ccc4ccccc4c3)nc(-c3cc(-c4ccc5c(c4)sc4ccccc45)cc4ccccc34)n2)cc1 ; c1ccc2cc(-c3nc(-c4ccc5ccccc5c4)nc(-c4cc(-c5ccc6c(c5)sc5ccccc56)cc5ccccc45)n3)ccc2c1 ; c1ccc(-c2nc(-c3ccc4ccccc4c3)nc(-c3cccc4cccc(-c5ccc6sc7ccccc7c6c5)c34)n2)cc1 ; c1ccc2cc(-c3nc(-c4ccc5ccccc5c4)nc(-c4cccc5cccc(-c6ccc7sc8ccccc8c7c6)c45)n3)ccc2c1]

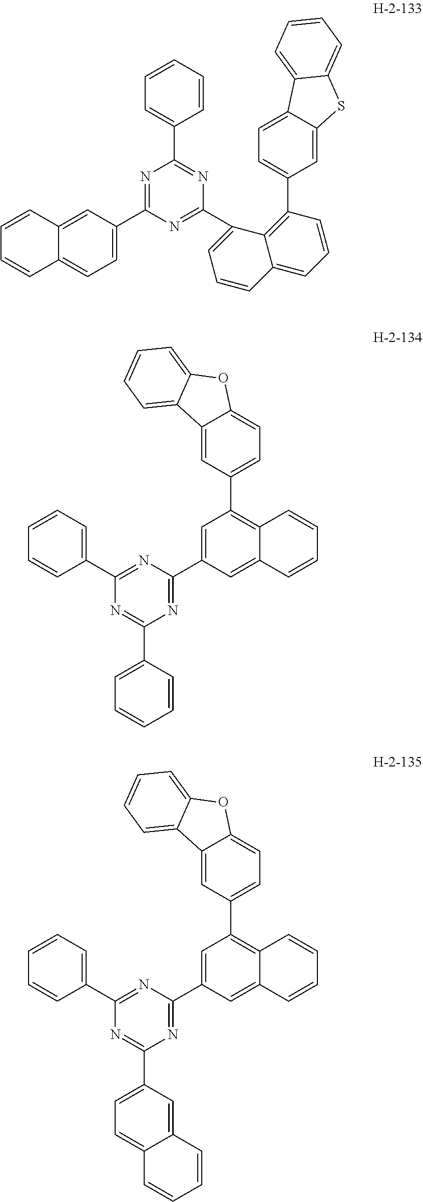 [SMILES: c1ccc(-c2nc(-c3ccc4ccccc4c3)nc(-c3cccc4cccc(-c5ccc6c(c5)sc5ccccc56)c34)n2)cc1 ; c1ccc(-c2nc(-c3ccccc3)nc(-c3cc(-c4ccc5oc6ccccc6c5c4)c4ccccc4c3)n2)cc1 ; c1ccc(-c2nc(-c3ccc4ccccc4c3)nc(-c3cc(-c4ccc5oc6ccccc6c5c4)c4ccccc4c3)n2)cc1]

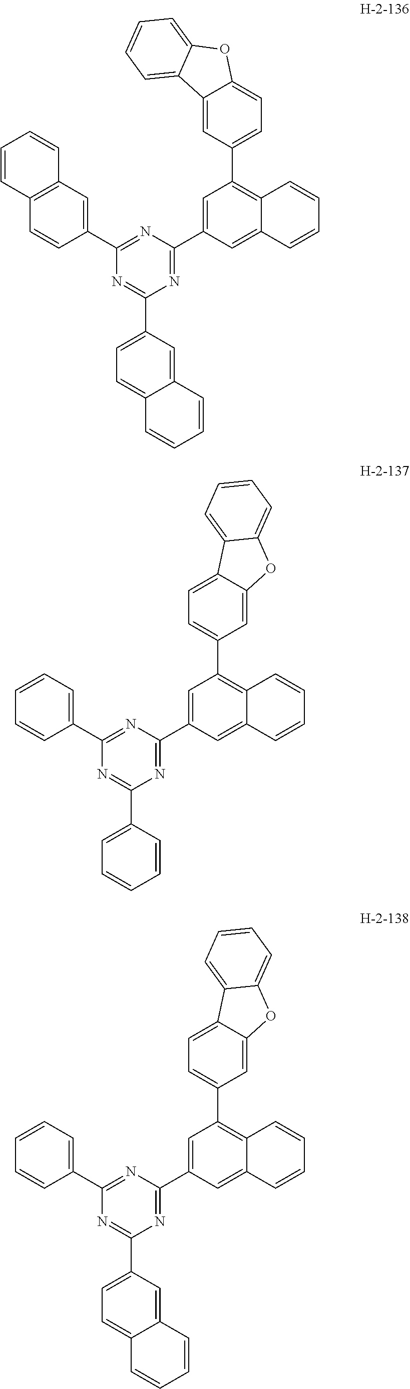 [SMILES: c1ccc2cc(-c3nc(-c4ccc5ccccc5c4)nc(-c4cc(-c5ccc6oc7ccccc7c6c5)c5ccccc5c4)n3)ccc2c1 ; c1ccc(-c2nc(-c3ccccc3)nc(-c3cc(-c4ccc5c(c4)oc4ccccc45)c4ccccc4c3)n2)cc1 ; c1ccc(-c2nc(-c3ccc4ccccc4c3)nc(-c3cc(-c4ccc5c(c4)oc4ccccc45)c4ccccc4c3)n2)cc1]

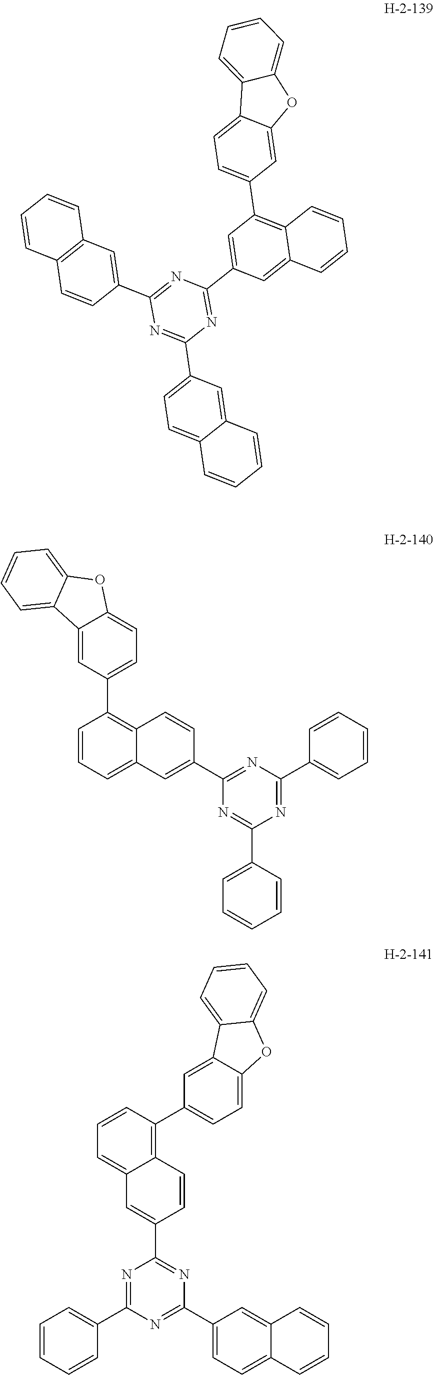

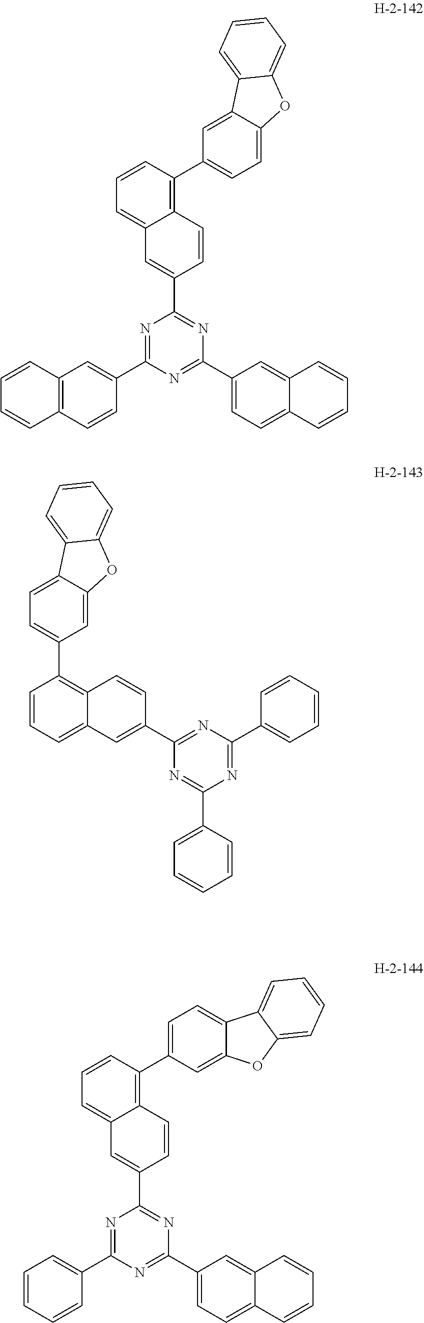 [SMILES: c1ccc2cc(-c3nc(-c4ccc5ccccc5c4)nc(-c4ccc5c(-c6ccc7oc8ccccc8c7c6)cccc5c4)n3)ccc2c1 ; c1ccc(-c2nc(-c3ccccc3)nc(-c3ccc4c(-c5ccc6c(c5)oc5ccccc56)cccc4c3)n2)cc1 ; c1ccc(-c2nc(-c3ccc4ccccc4c3)nc(-c3ccc4c(-c5ccc6c(c5)oc5ccccc56)cccc4c3)n2)cc1]

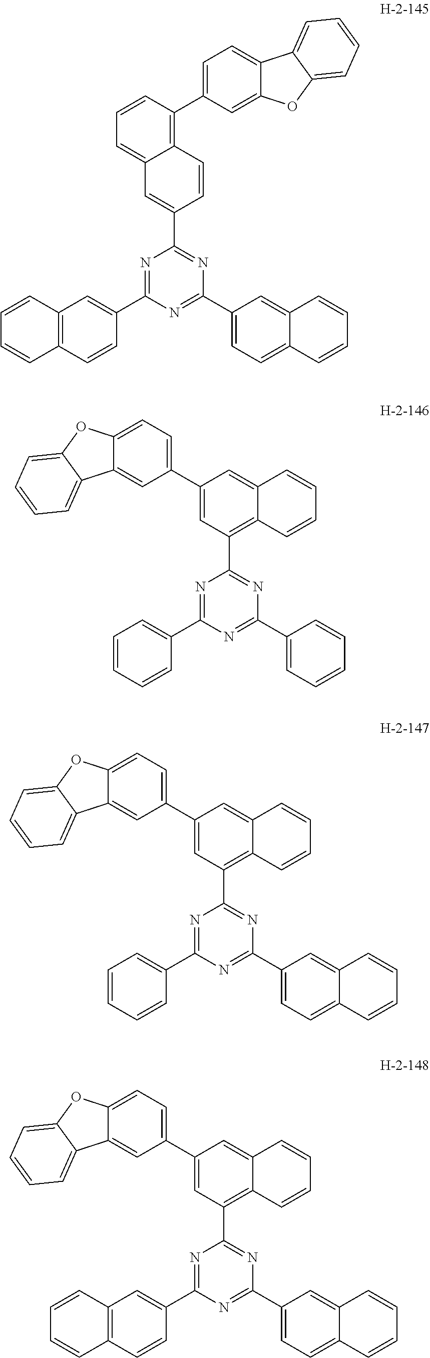 [SMILES: c1ccc2cc(-c3nc(-c4ccc5ccccc5c4)nc(-c4ccc5c(-c6ccc7c(c6)oc6ccccc67)cccc5c4)n3)ccc2c1 ; c1ccc(-c2nc(-c3ccccc3)nc(-c3cc(-c4ccc5oc6ccccc6c5c4)cc4ccccc34)n2)cc1 ; c1ccc(-c2nc(-c3ccc4ccccc4c3)nc(-c3cc(-c4ccc5oc6ccccc6c5c4)cc4ccccc34)n2)cc1 ; c1ccc2cc(-c3nc(-c4ccc5ccccc5c4)nc(-c4cc(-c5ccc6oc7ccccc7c6c5)cc5ccccc45)n3)ccc2c1]

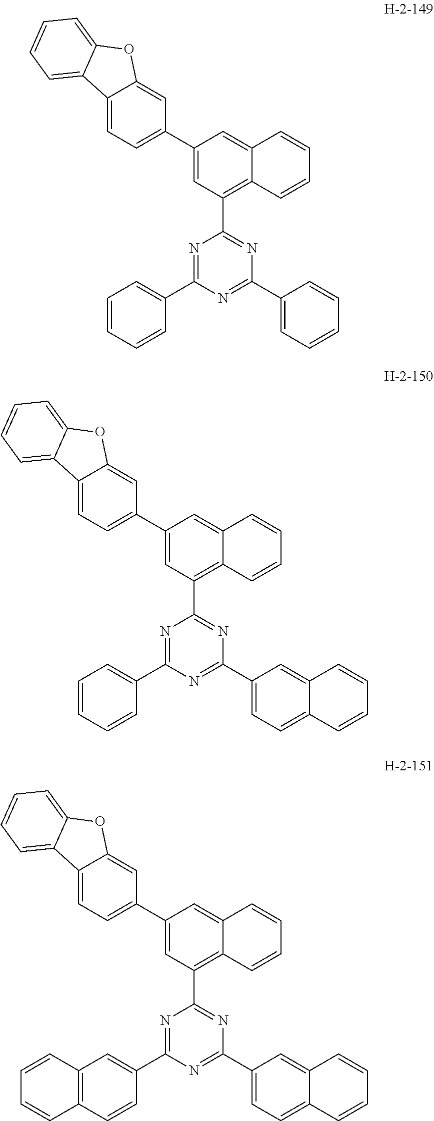 [SMILES: c1ccc(-c2nc(-c3ccccc3)nc(-c3cc(-c4ccc5c(c4)oc4ccccc45)cc4ccccc34)n2)cc1 ; c1ccc(-c2nc(-c3ccc4ccccc4c3)nc(-c3cc(-c4ccc5c(c4)oc4ccccc45)cc4ccccc34)n2)cc1 ; c1ccc2cc(-c3nc(-c4ccc5ccccc5c4)nc(-c4cc(-c5ccc6c(c5)oc5ccccc56)cc5ccccc45)n3)ccc2c1]

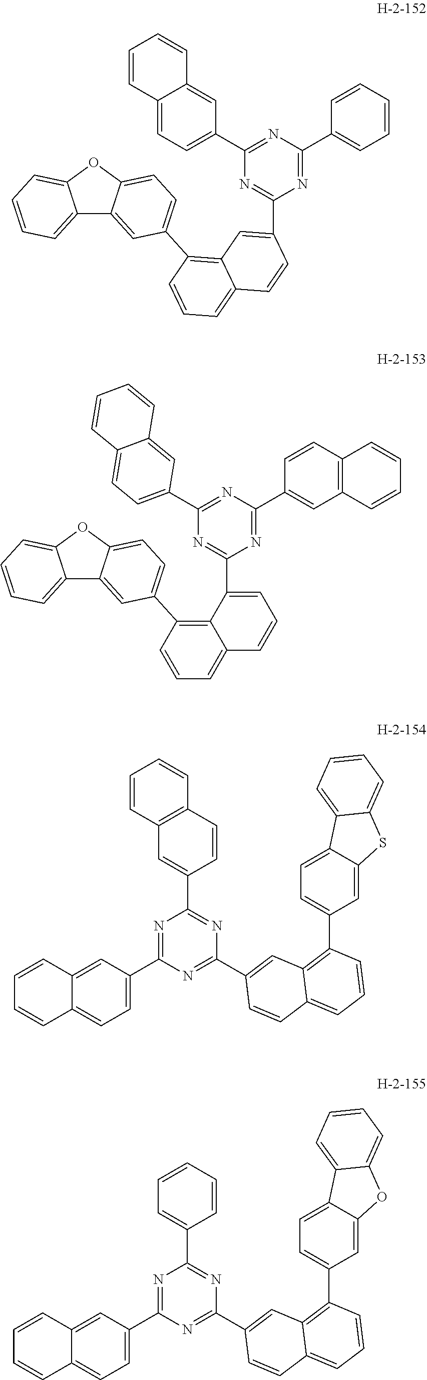 [SMILES: c1ccc(-c2nc(-c3ccc4ccccc4c3)nc(-c3ccc4cccc(-c5ccc6oc7ccccc7c6c5)c4c3)n2)cc1 ; c1ccc2cc(-c3nc(-c4ccc5ccccc5c4)nc(-c4cccc5cccc(-c6ccc7oc8ccccc8c7c6)c45)n3)ccc2c1 ; c1ccc2cc(-c3nc(-c4ccc5ccccc5c4)nc(-c4ccc5cccc(-c6ccc7c(c6)sc6ccccc67)c5c4)n3)ccc2c1 ; c1ccc(-c2nc(-c3ccc4ccccc4c3)nc(-c3ccc4cccc(-c5ccc6c(c5)oc5ccccc56)c4c3)n2)cc1]

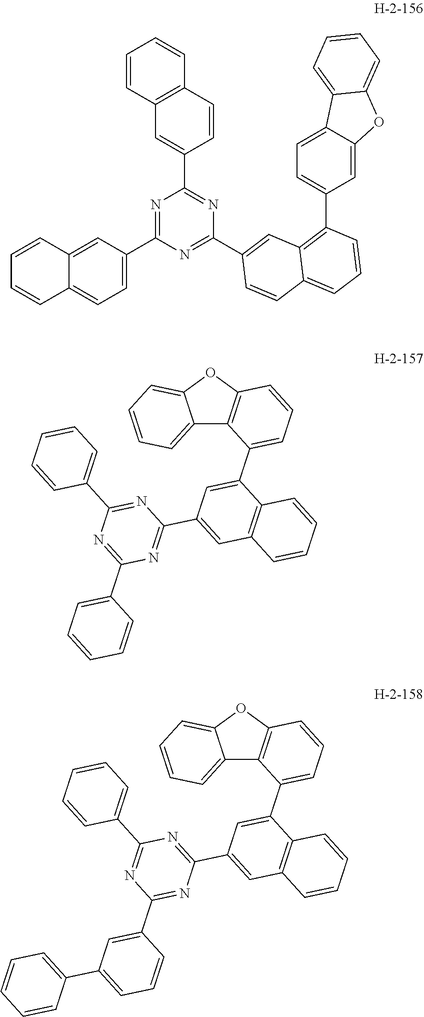 [SMILES: c1ccc2cc(-c3nc(-c4ccc5ccccc5c4)nc(-c4ccc5cccc(-c6ccc7c(c6)oc6ccccc67)c5c4)n3)ccc2c1 ; c1ccc(-c2nc(-c3ccccc3)nc(-c3cc(-c4cccc5oc6ccccc6c45)c4ccccc4c3)n2)cc1 ; c1ccc(-c2cccc(-c3nc(-c4ccccc4)nc(-c4cc(-c5cccc6oc7ccccc7c56)c5ccccc5c4)n3)c2)cc1]

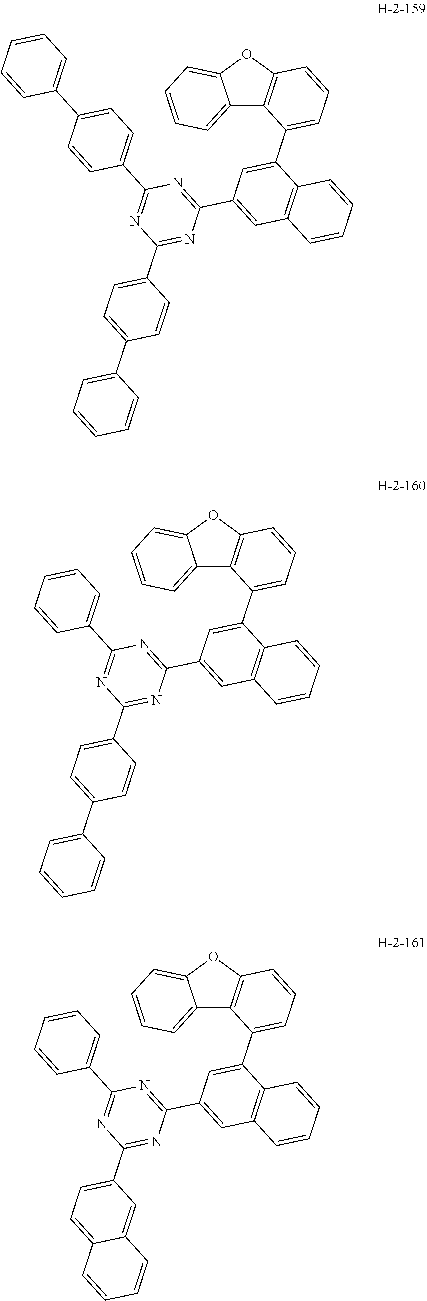 [SMILES: c1ccc(-c2ccc(-c3nc(-c4ccc(-c5ccccc5)cc4)nc(-c4cc(-c5cccc6oc7ccccc7c56)c5ccccc5c4)n3)cc2)cc1 ; c1ccc(-c2ccc(-c3nc(-c4ccccc4)nc(-c4cc(-c5cccc6oc7ccccc7c56)c5ccccc5c4)n3)cc2)cc1 ; c1ccc(-c2nc(-c3ccc4ccccc4c3)nc(-c3cc(-c4cccc5oc6ccccc6c45)c4ccccc4c3)n2)cc1]

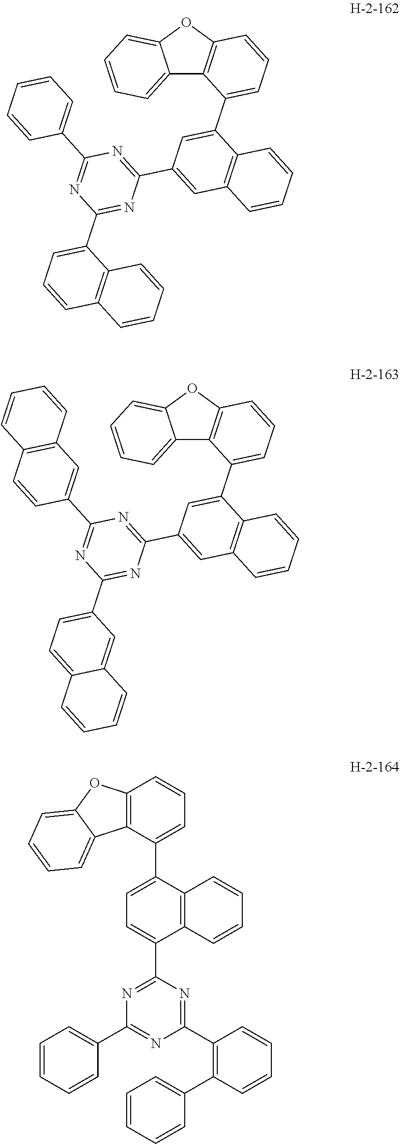 [SMILES: c1ccc(-c2nc(-c3cc(-c4cccc5oc6ccccc6c45)c4ccccc4c3)nc(-c3cccc4ccccc34)n2)cc1 ; c1ccc2cc(-c3nc(-c4ccc5ccccc5c4)nc(-c4cc(-c5cccc6oc7ccccc7c56)c5ccccc5c4)n3)ccc2c1 ; c1ccc(-c2nc(-c3ccccc3-c3ccccc3)nc(-c3ccc(-c4cccc5oc6ccccc6c45)c4ccccc34)n2)cc1]

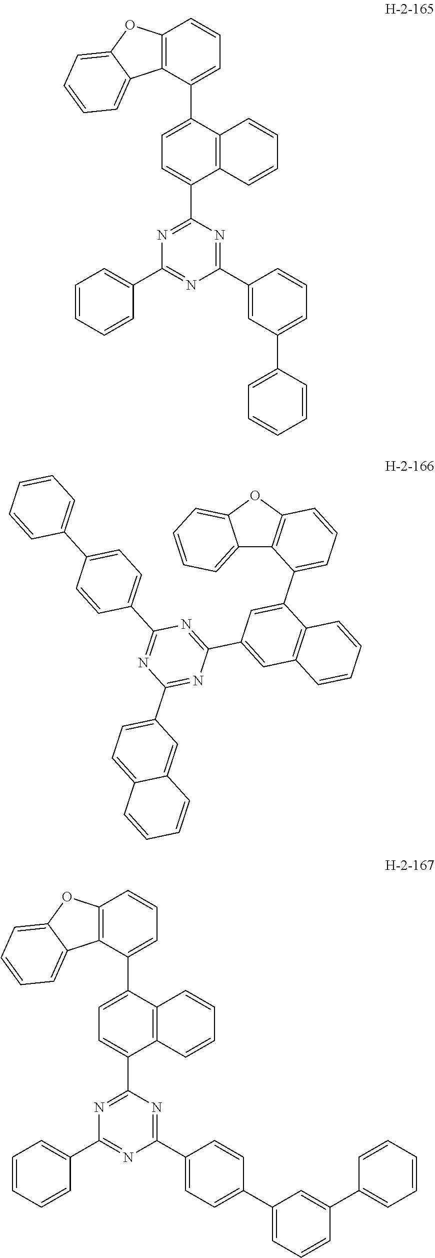 [SMILES: c1ccc(-c2cccc(-c3nc(-c4ccccc4)nc(-c4ccc(-c5cccc6oc7ccccc7c56)c5ccccc45)n3)c2)cc1 ; c1ccc(-c2ccc(-c3nc(-c4ccc5ccccc5c4)nc(-c4cc(-c5cccc6oc7ccccc7c56)c5ccccc5c4)n3)cc2)cc1 ; c1ccc(-c2cccc(-c3ccc(-c4nc(-c5ccccc5)nc(-c5ccc(-c6cccc7oc8ccccc8c67)c6ccccc56)n4)cc3)c2)cc1]

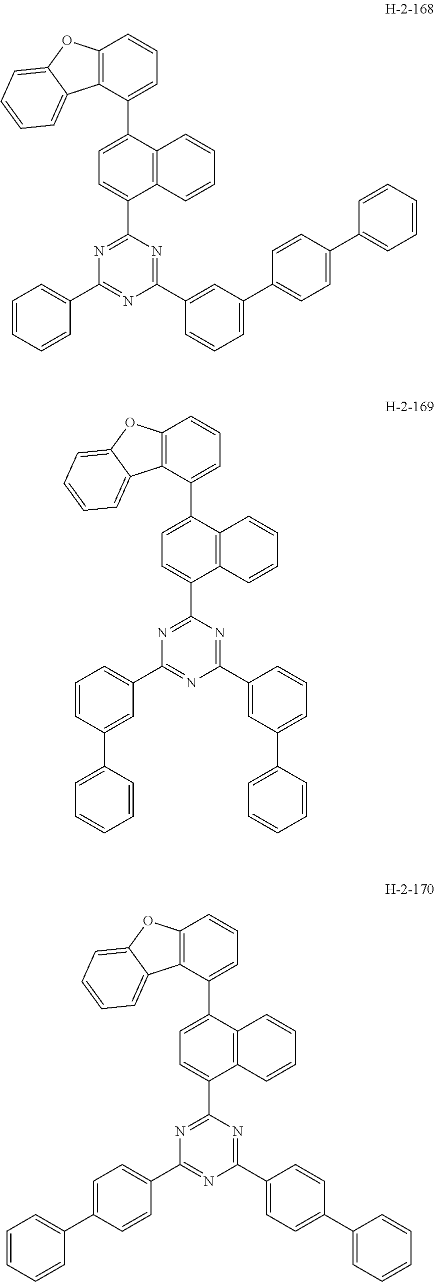 [SMILES: c1ccc(-c2ccc(-c3cccc(-c4nc(-c5ccccc5)nc(-c5ccc(-c6cccc7oc8ccccc8c67)c6ccccc56)n4)c3)cc2)cc1 ; c1ccc(-c2cccc(-c3nc(-c4cccc(-c5ccccc5)c4)nc(-c4ccc(-c5cccc6oc7ccccc7c56)c5ccccc45)n3)c2)cc1 ; c1ccc(-c2ccc(-c3nc(-c4ccc(-c5ccccc5)cc4)nc(-c4ccc(-c5cccc6oc7ccccc7c56)c5ccccc45)n3)cc2)cc1]

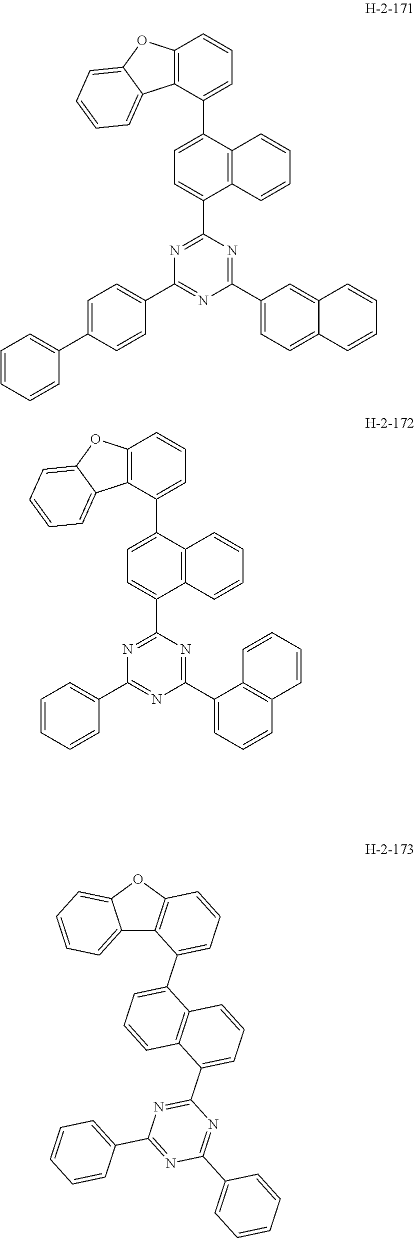 [SMILES: c1ccc(-c2ccc(-c3nc(-c4ccc5ccccc5c4)nc(-c4ccc(-c5cccc6oc7ccccc7c56)c5ccccc45)n3)cc2)cc1 ; c1ccc(-c2nc(-c3cccc4ccccc34)nc(-c3ccc(-c4cccc5oc6ccccc6c45)c4ccccc34)n2)cc1 ; c1ccc(-c2nc(-c3ccccc3)nc(-c3cccc4c(-c5cccc6oc7ccccc7c56)cccc34)n2)cc1]

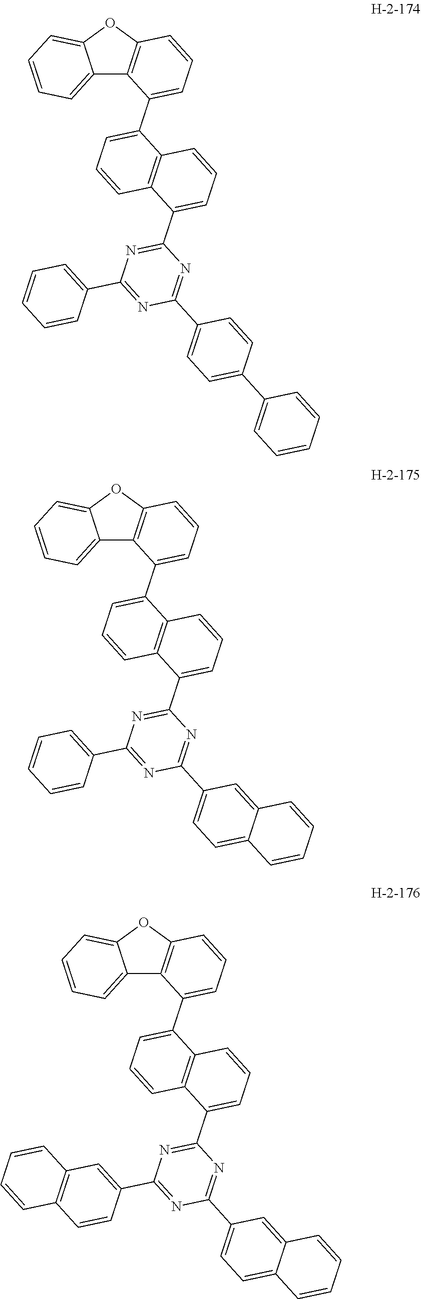 [SMILES: c1ccc(-c2ccc(-c3nc(-c4ccccc4)nc(-c4cccc5c(-c6cccc7oc8ccccc8c67)cccc45)n3)cc2)cc1 ; c1ccc(-c2nc(-c3ccc4ccccc4c3)nc(-c3cccc4c(-c5cccc6oc7ccccc7c56)cccc34)n2)cc1 ; c1ccc2cc(-c3nc(-c4ccc5ccccc5c4)nc(-c4cccc5c(-c6cccc7oc8ccccc8c67)cccc45)n3)ccc2c1]

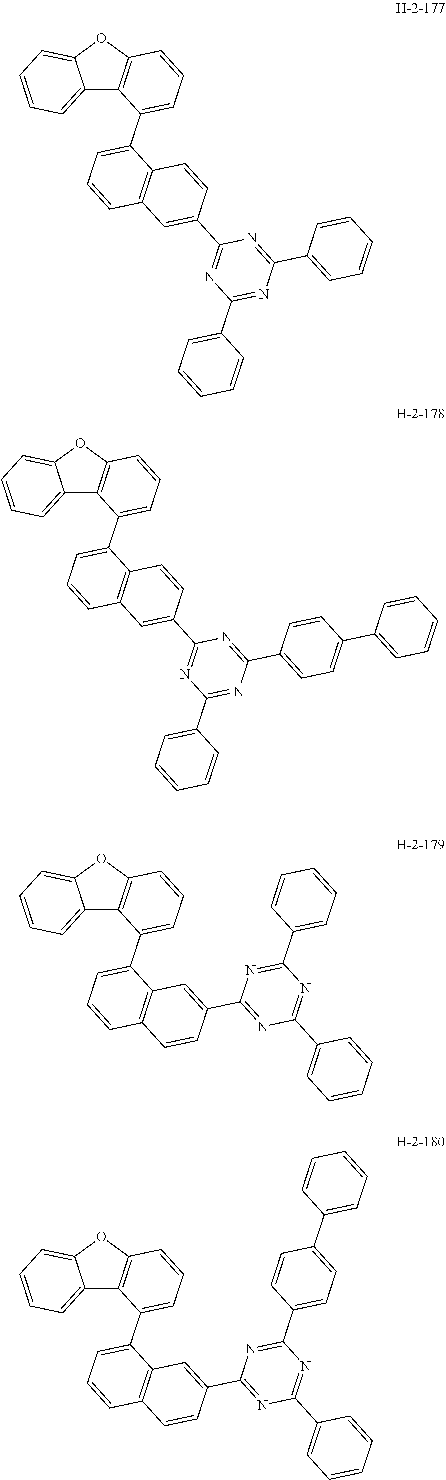 [SMILES: c1ccc(-c2nc(-c3ccccc3)nc(-c3ccc4c(-c5cccc6oc7ccccc7c56)cccc4c3)n2)cc1 ; c1ccc(-c2ccc(-c3nc(-c4ccccc4)nc(-c4ccc5c(-c6cccc7oc8ccccc8c67)cccc5c4)n3)cc2)cc1 ; c1ccc(-c2nc(-c3ccccc3)nc(-c3ccc4cccc(-c5cccc6oc7ccccc7c56)c4c3)n2)cc1 ; c1ccc(-c2ccc(-c3nc(-c4ccccc4)nc(-c4ccc5cccc(-c6cccc7oc8ccccc8c67)c5c4)n3)cc2)cc1]

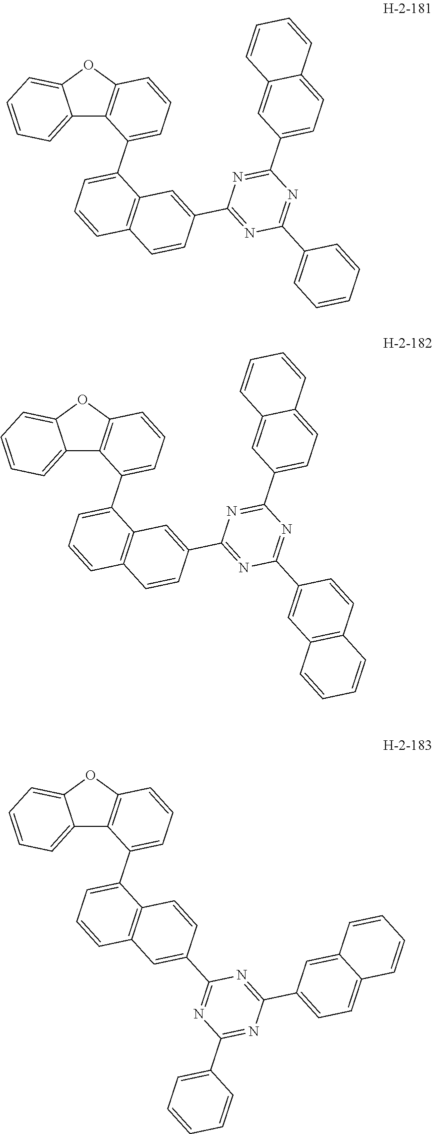 [SMILES: c1ccc(-c2nc(-c3ccc4ccccc4c3)nc(-c3ccc4cccc(-c5cccc6oc7ccccc7c56)c4c3)n2)cc1 ; c1ccc2cc(-c3nc(-c4ccc5ccccc5c4)nc(-c4ccc5cccc(-c6cccc7oc8ccccc8c67)c5c4)n3)ccc2c1 ; c1ccc(-c2nc(-c3ccc4ccccc4c3)nc(-c3ccc4c(-c5cccc6oc7ccccc7c56)cccc4c3)n2)cc1]

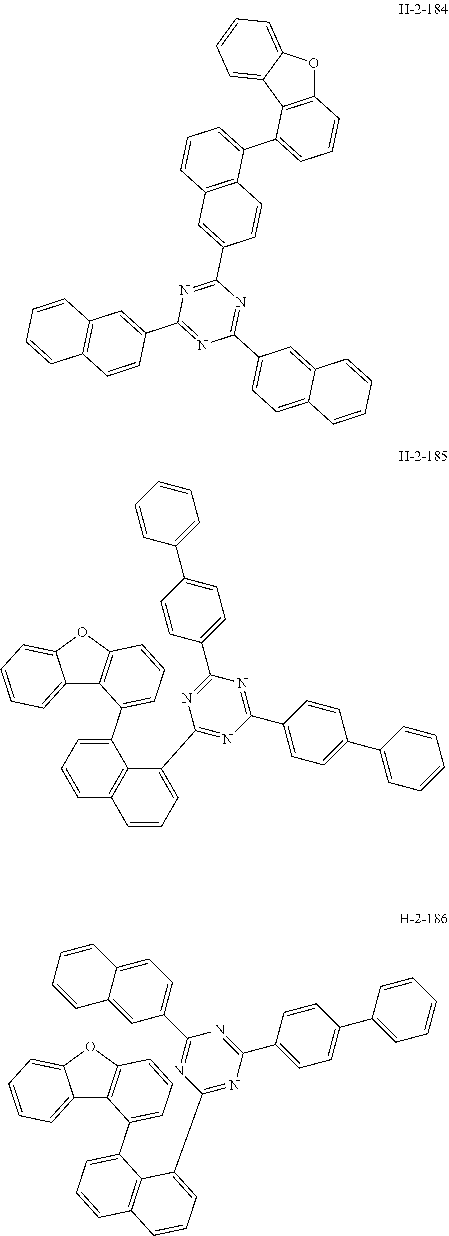 [SMILES: c1ccc2cc(-c3nc(-c4ccc5ccccc5c4)nc(-c4ccc5c(-c6cccc7oc8ccccc8c67)cccc5c4)n3)ccc2c1 ; c1ccc(-c2ccc(-c3nc(-c4ccc(-c5ccccc5)cc4)nc(-c4cccc5cccc(-c6cccc7oc8ccccc8c67)c45)n3)cc2)cc1 ; c1ccc(-c2ccc(-c3nc(-c4ccc5ccccc5c4)nc(-c4cccc5cccc(-c6cccc7oc8ccccc8c67)c45)n3)cc2)cc1]

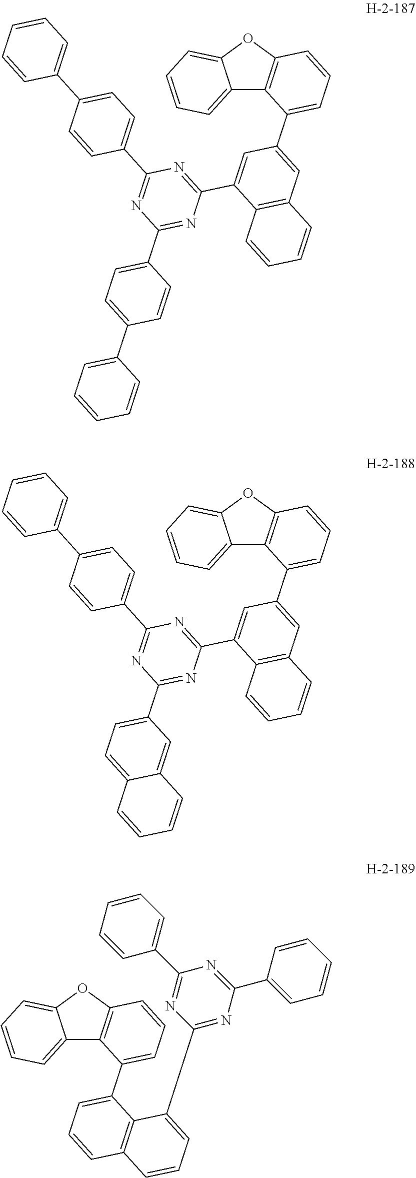 [SMILES: c1ccc(-c2ccc(-c3nc(-c4ccc(-c5ccccc5)cc4)nc(-c4cc(-c5cccc6oc7ccccc7c56)cc5ccccc45)n3)cc2)cc1 ; c1ccc(-c2ccc(-c3nc(-c4ccc5ccccc5c4)nc(-c4cc(-c5cccc6oc7ccccc7c56)cc5ccccc45)n3)cc2)cc1 ; c1ccc(-c2nc(-c3ccccc3)nc(-c3cccc4cccc(-c5cccc6oc7ccccc7c56)c34)n2)cc1]

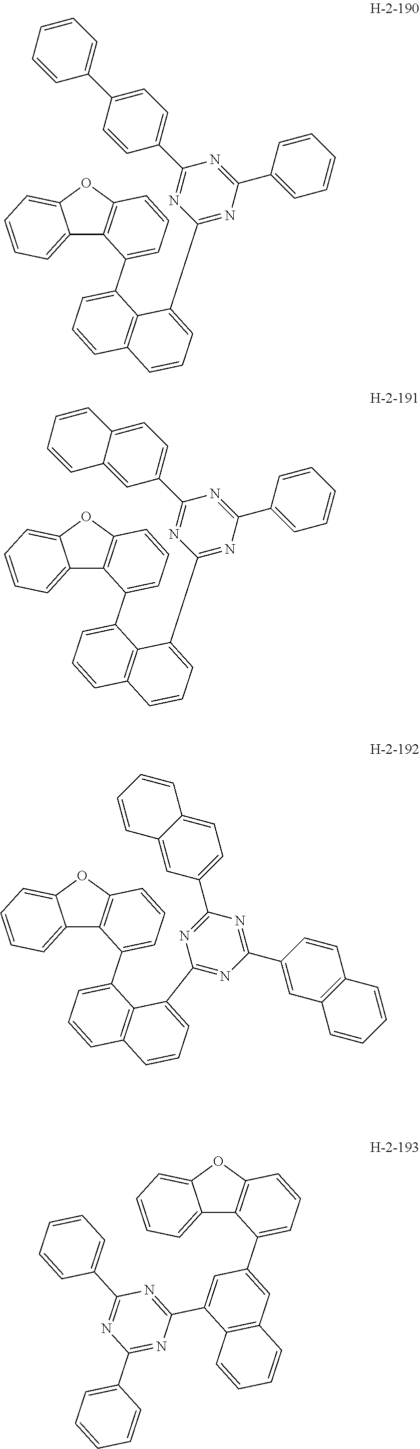 [SMILES: c1ccc(-c2ccc(-c3nc(-c4ccccc4)nc(-c4cccc5cccc(-c6cccc7oc8ccccc8c67)c45)n3)cc2)cc1 ; c1ccc(-c2nc(-c3ccc4ccccc4c3)nc(-c3cccc4cccc(-c5cccc6oc7ccccc7c56)c34)n2)cc1 ; c1ccc2cc(-c3nc(-c4ccc5ccccc5c4)nc(-c4cccc5cccc(-c6cccc7oc8ccccc8c67)c45)n3)ccc2c1 ; c1ccc(-c2nc(-c3ccccc3)nc(-c3cc(-c4cccc5oc6ccccc6c45)cc4ccccc34)n2)cc1]

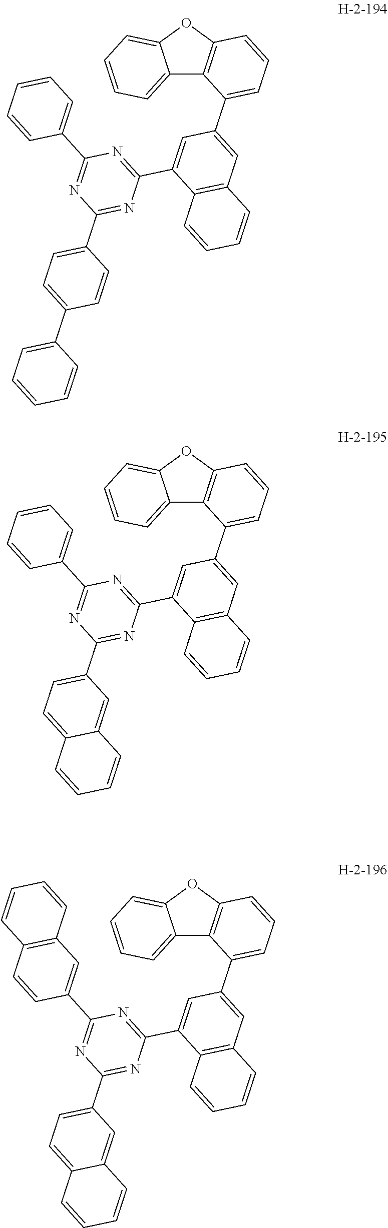 [SMILES: c1ccc(-c2ccc(-c3nc(-c4ccccc4)nc(-c4cc(-c5cccc6oc7ccccc7c56)cc5ccccc45)n3)cc2)cc1 ; c1ccc(-c2nc(-c3ccc4ccccc4c3)nc(-c3cc(-c4cccc5oc6ccccc6c45)cc4ccccc34)n2)cc1 ; c1ccc2cc(-c3nc(-c4ccc5ccccc5c4)nc(-c4cc(-c5cccc6oc7ccccc7c56)cc5ccccc45)n3)ccc2c1]

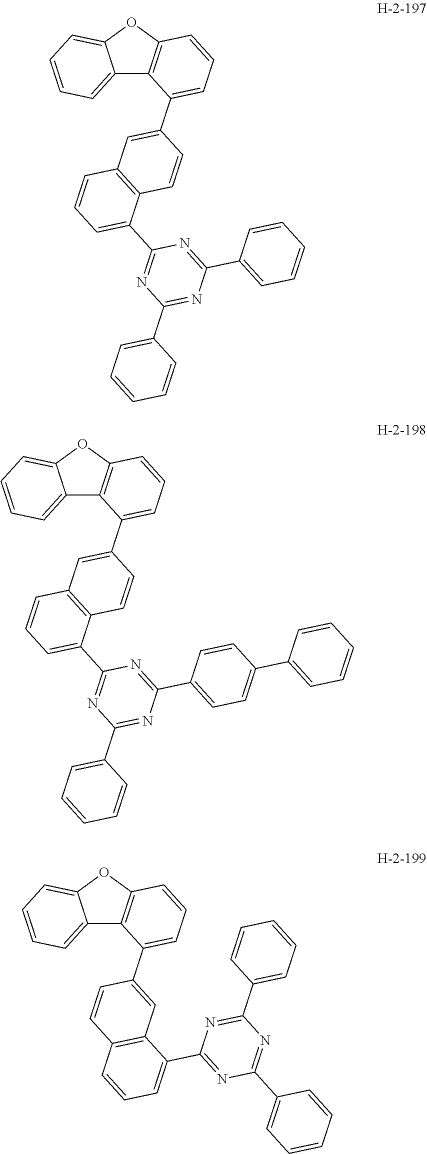 [SMILES: c1ccc(-c2nc(-c3ccccc3)nc(-c3cccc4cc(-c5cccc6oc7ccccc7c56)ccc34)n2)cc1 ; c1ccc(-c2ccc(-c3nc(-c4ccccc4)nc(-c4cccc5cc(-c6cccc7oc8ccccc8c67)ccc45)n3)cc2)cc1 ; c1ccc(-c2nc(-c3ccccc3)nc(-c3cccc4ccc(-c5cccc6oc7ccccc7c56)cc34)n2)cc1]

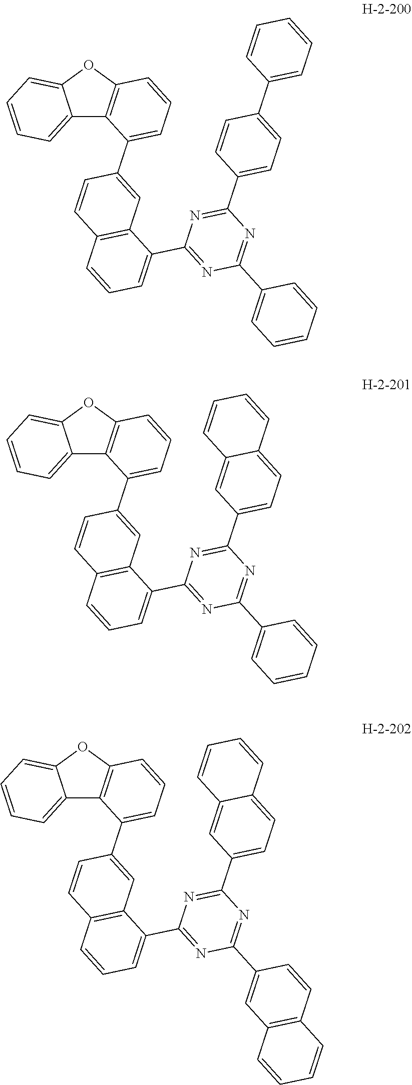 [SMILES: c1ccc(-c2ccc(-c3nc(-c4ccccc4)nc(-c4cccc5ccc(-c6cccc7oc8ccccc8c67)cc45)n3)cc2)cc1 ; c1ccc(-c2nc(-c3ccc4ccccc4c3)nc(-c3cccc4ccc(-c5cccc6oc7ccccc7c56)cc34)n2)cc1 ; c1ccc2cc(-c3nc(-c4ccc5ccccc5c4)nc(-c4cccc5ccc(-c6cccc7oc8ccccc8c67)cc45)n3)ccc2c1]

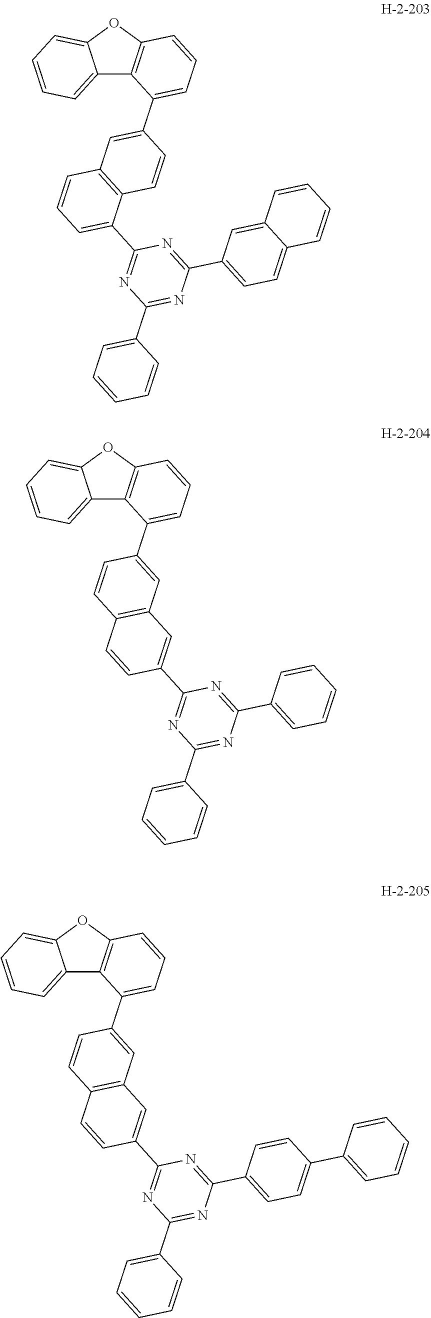

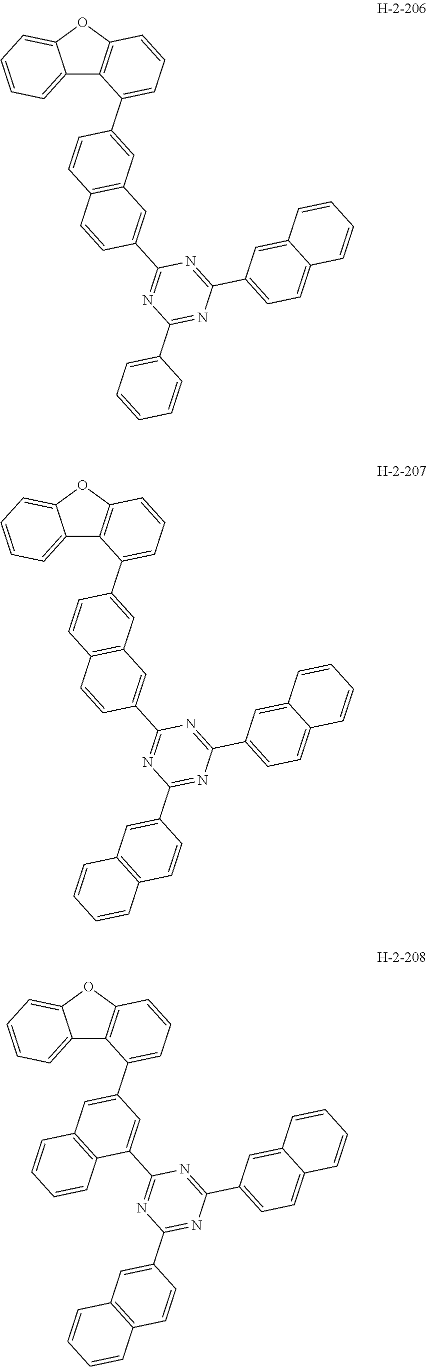 [SMILES: c1ccc(-c2nc(-c3ccc4ccccc4c3)nc(-c3ccc4ccc(-c5cccc6oc7ccccc7c56)cc4c3)n2)cc1 ; c1ccc2cc(-c3nc(-c4ccc5ccccc5c4)nc(-c4ccc5ccc(-c6cccc7oc8ccccc8c67)cc5c4)n3)ccc2c1 ; c1ccc2cc(-c3nc(-c4ccc5ccccc5c4)nc(-c4cc(-c5cccc6oc7ccccc7c56)cc5ccccc45)n3)ccc2c1]

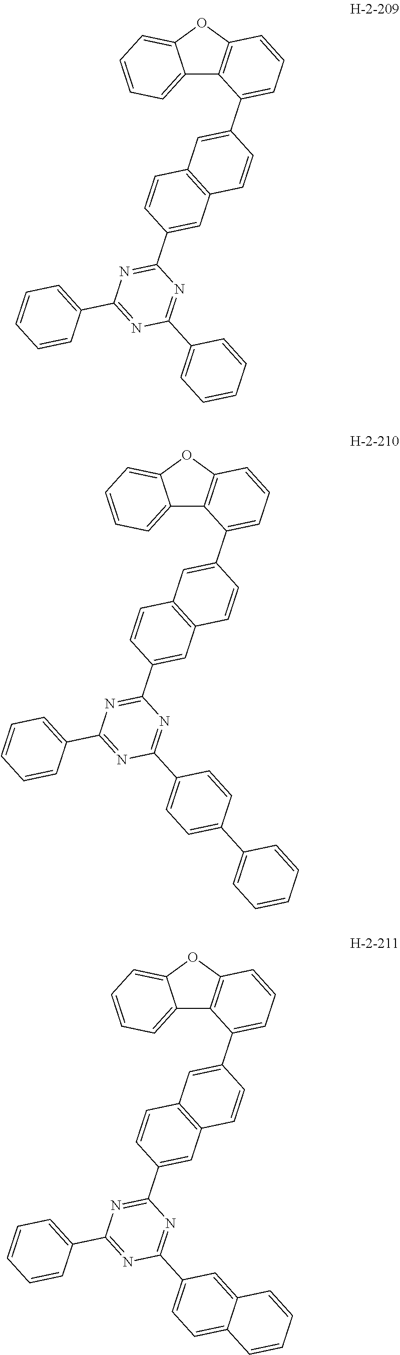 [SMILES: c1ccc(-c2nc(-c3ccccc3)nc(-c3ccc4cc(-c5cccc6oc7ccccc7c56)ccc4c3)n2)cc1 ; c1ccc(-c2ccc(-c3nc(-c4ccccc4)nc(-c4ccc5cc(-c6cccc7oc8ccccc8c67)ccc5c4)n3)cc2)cc1 ; c1ccc(-c2nc(-c3ccc4ccccc4c3)nc(-c3ccc4cc(-c5cccc6oc7ccccc7c56)ccc4c3)n2)cc1]

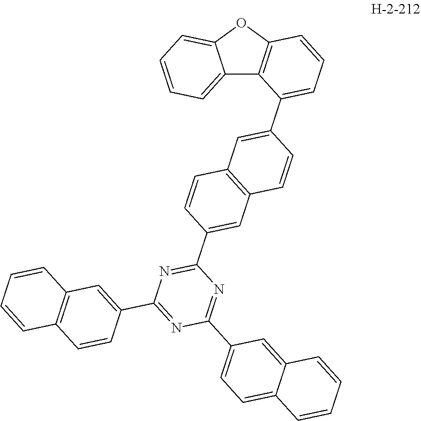 [SMILES: c1ccc2cc(-c3nc(-c4ccc5ccccc5c4)nc(-c4ccc5cc(-c6cccc7oc8ccccc8c67)ccc5c4)n3)ccc2c1]

At least one of compounds H-1-1 to H-1-53 and at least one of compounds H-2-1 to H-2-212 may be combined and used in an organic electroluminescent device.

The compound represented by formula 1 according to the present disclosure may be produced by a synthetic method known to one skilled in the art, and for example, according to the methods disclosed in Korean Patent Application Laying-Open Nos. 2013-0106255 (Sep. 27, 2013), 2012-0042633 (May 3, 2012), and 2015-0066202 (Jun. 16, 2015), but is not limited thereto.

The compound represented by formula 2 according to the present disclosure may be produced by a synthetic method known to one skilled in the art, and for example, according to the following reaction scheme 1, but is not limited thereto.

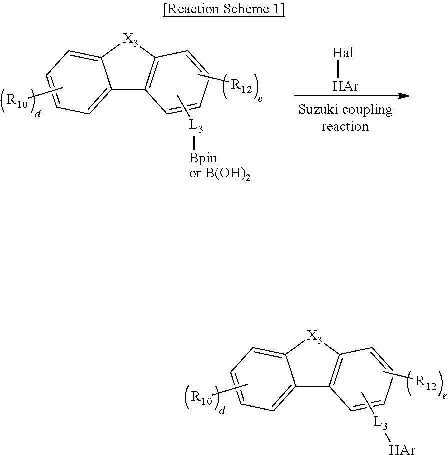

In reaction scheme 1, X₃, L₃, HAr, R₁₀, R₁₂, d, and e are as defined in formula 2.

The organic electroluminescent device of the present disclosure may comprise a first electrode, a second electrode, and at least one organic layer between the first and second electrodes.

One of the first and second electrodes may be an anode, and the other may be a cathode. The organic layer may comprise a light-emitting layer, and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. The second electrode may be a transflective electrode or a reflective electrode, and may be a top emission type, a bottom emission type, or both-sides emission type according to the kinds of the material. In addition, the hole injection layer may be further doped with a p-dopant, and the electron injection layer may be further doped with an n-dopant.

The organic electroluminescent device according to the present disclosure may comprise an anode, a cathode, and at least one organic layer between the anode and cathode, in which the organic layer may comprise a plurality of organic electroluminescent materials including the compound represented by formula 1 as the first organic electroluminescent material, and the compound represented by formula 2 as the second organic electroluminescent material. According to one embodiment of the present disclosure, the organic electroluminescent device according to the present disclosure may comprise an anode, a cathode, and at least one light-emitting layer between the anode and cathode, in which the light-emitting layer may comprise the compound represented by formula 1 and the compound represented by formula 2.

The light-emitting layer includes a host and a dopant, in which the host includes a plurality of host materials, and the compound represented by formula 1 may be included as the first host compound of the plurality of host materials, and the compound represented by formula 2 may be included as the second host compound of the plurality of host materials. The weight ratio of the first host compound and the second host compound is about 1:99 to about 99:1, preferably about 10:90 to about 90:10, more preferably about 30:70 to about 70:30, even more preferably about 40:60 to about 60:40, and further more preferably about 50:50.

Herein, the light-emitting layer is a layer from which light is emitted, and may be a single layer or a multi-layer of which two or more layers are stacked. All of the first host material and the second host material may be included in one layer, or the first host material and the second host material may be included in respective different light-emitting layers. According to one embodiment of the present disclosure, the doping concentration of the dopant compound with respect to the host compound in the light-emitting layer may be less than 20 wt %.

The organic electroluminescent device of the present disclosure may further comprise at least one layer selected from a hole injection layer, a hole transport layer, a hole auxiliary layer, a light-emitting auxiliary layer, an electron transport layer, an electron injection layer, an interlayer, an electron buffer layer, a hole blocking layer, and an electron blocking layer. According to one embodiment of the present disclosure, the organic electroluminescent device of the present disclosure may further comprise an amine-based compound besides the plurality of host materials of the present disclosure as at least one of a hole injection material, a hole transport material, a hole auxiliary material, a light-emitting material, a light-emitting auxiliary material, and an electron blocking material. Further; according to one embodiment of the present disclosure; the organic electroluminescent device of the present disclosure may further comprise an azine-based compound besides the plurality of host materials of the present disclosure as at least one of an electron transport material, an electron injection material; an electron buffer material, and a hole blocking material.

The dopant comprised in the organic electroluminescent device of the present disclosure may be at least one phosphorescent or fluorescent dopont, and is preferably at least one phosphorescent dopant. The phosphorescent dopant material applied to the organic electroluminescent device of the present disclosure is not particulary limited, but may be preferably selected from the metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may comprise a compound represented by the following formula 101, but is not limited thereto.

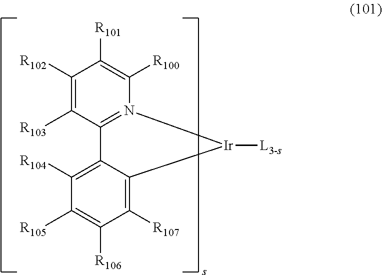

In formula 101, L is selected from the following structures 1 and 2:

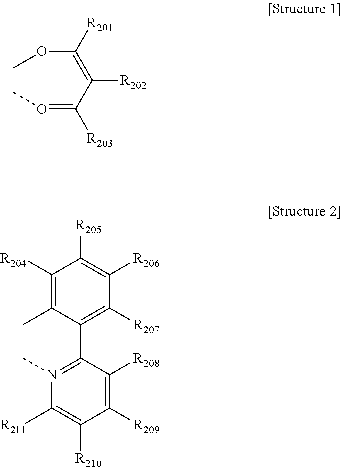

R₁₀₀ to R₁₀₃, each independently, represent hydrogen, deuterium, a halogen, a (C1-C30)alkyl unsubstituted or substituted with a halogen(s); a substituted or unsubstituted (C3-C30)cycloalkyl; a substituted or unsubstituted (C6-C30)aryl, a cyano; a substituted or unsubstituted (3- to 30-membered)heteroaryl, or a substituted or unsubstituted C30)alkoxy; or may be linked to an adjacent substituent to form a ring(s), e.g.; a substituted or unsubstituted, quinoline; benzofuropyridine, benzothienopyridine, indenopyridine, benzofuroquinoline, benzothienoquinoline, or indenoquinoline ring, together with pyridine;

R₁₀₄ to R₁₀₇, each independently, represent hydrogen, deuterium, a halogen, a (C1-C30)alkyl unsubstituted or substituted with a halogen(s), a substituted or unsubstituted (C3-C30)cycloalkyl; a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a ring(s), e.g., a substituted or unsubstituted, naphthyl, fluorene, dibenzothiophene, dibenzofuran, indenopyridine; benzofuropyridine, or benzothienopyridine ring, together with benzene;

R₂₀₁ to R₂₁₁ each independently, represent hydrogen, deuterium, a halogen, a (C1-C30)alkyl unsubstituted or substituted with a halogen(s), a substituted or unsubstituted (C3-C30)cycloalkyl, or a substituted or unsubstituted (C6-C30)aryl; or may be linked to an adjacent substituent to form a ring(s); and

s represents an integer of 1 to 3.

The specific examples of the dopant compound are as follows, but are not limited thereto,

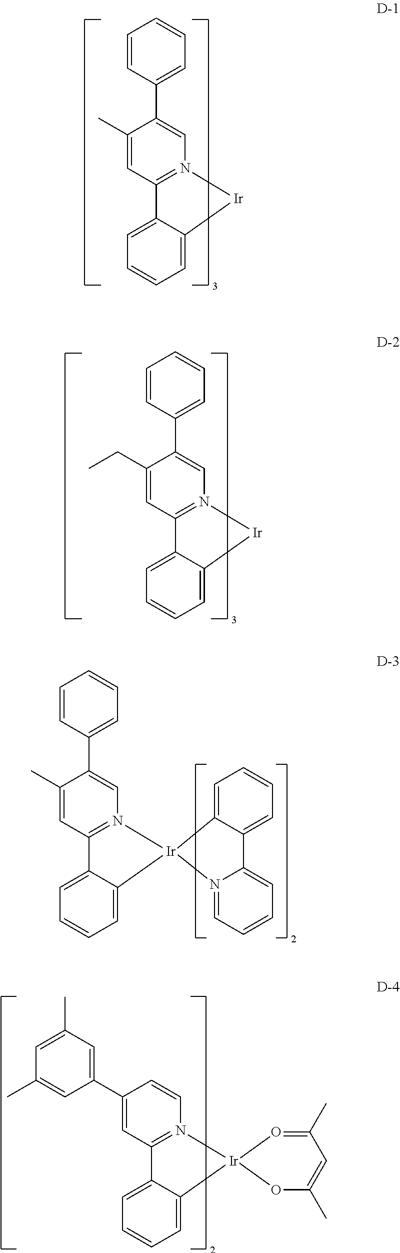

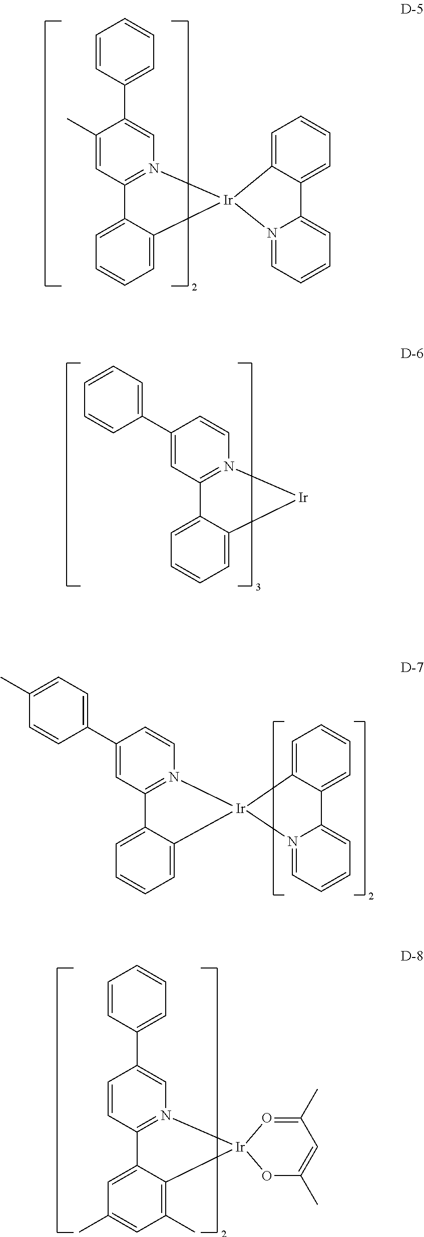

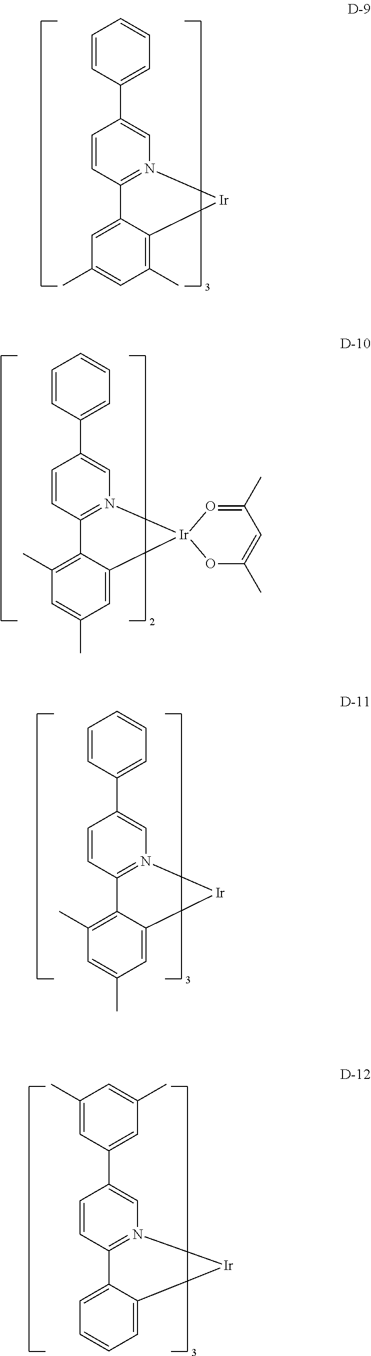

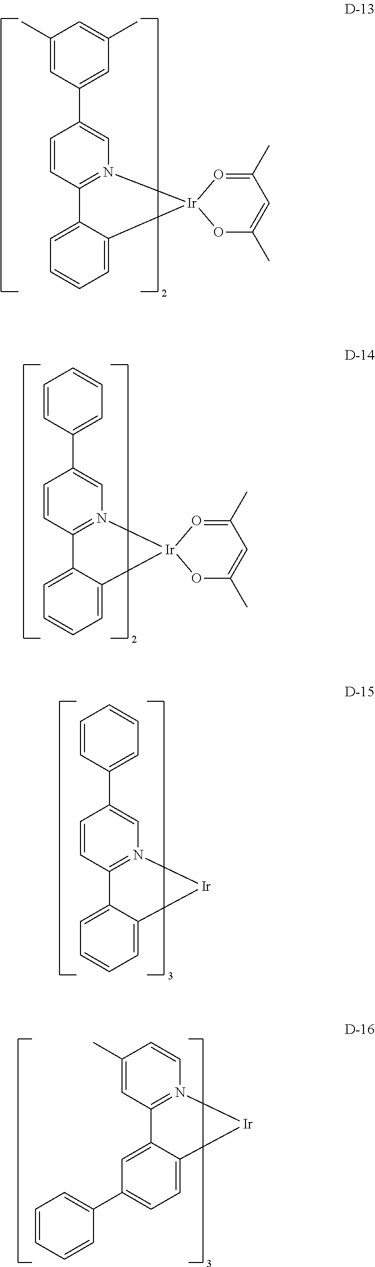

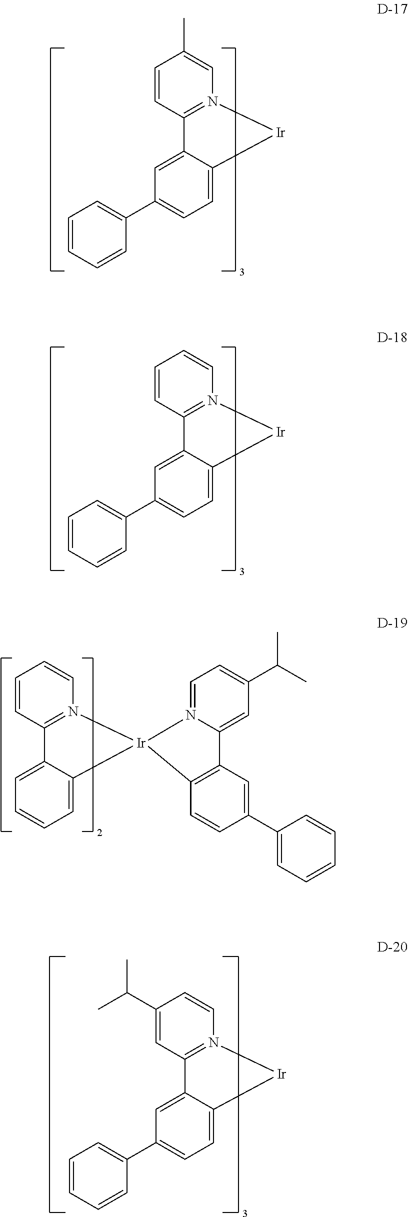

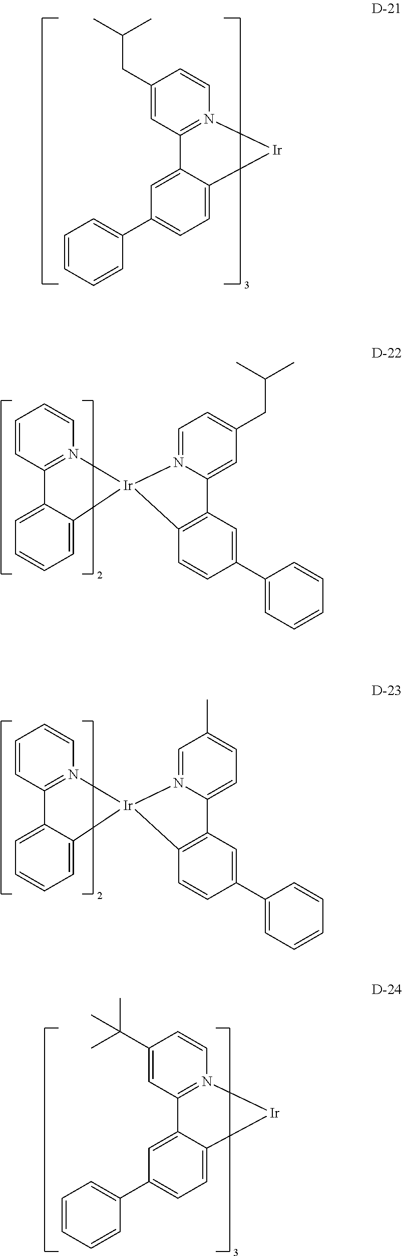

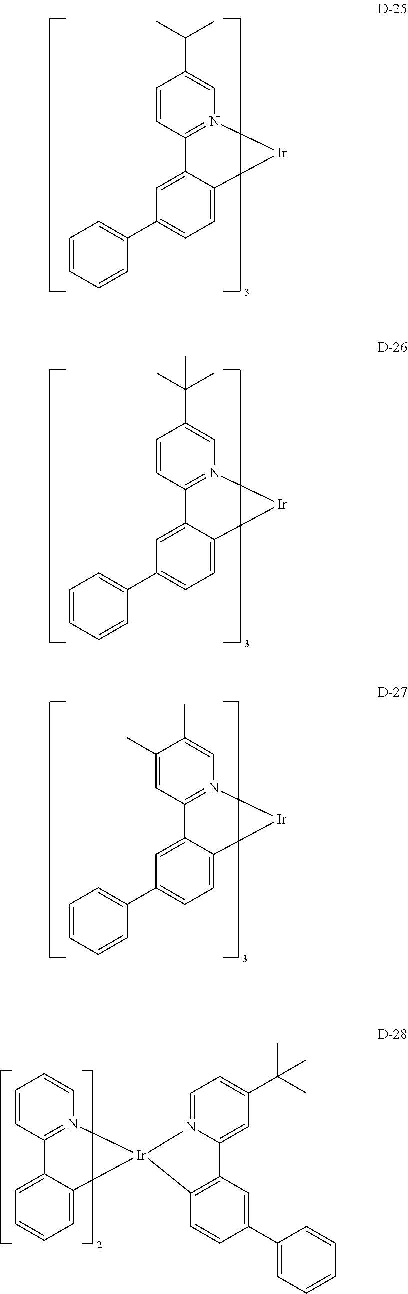

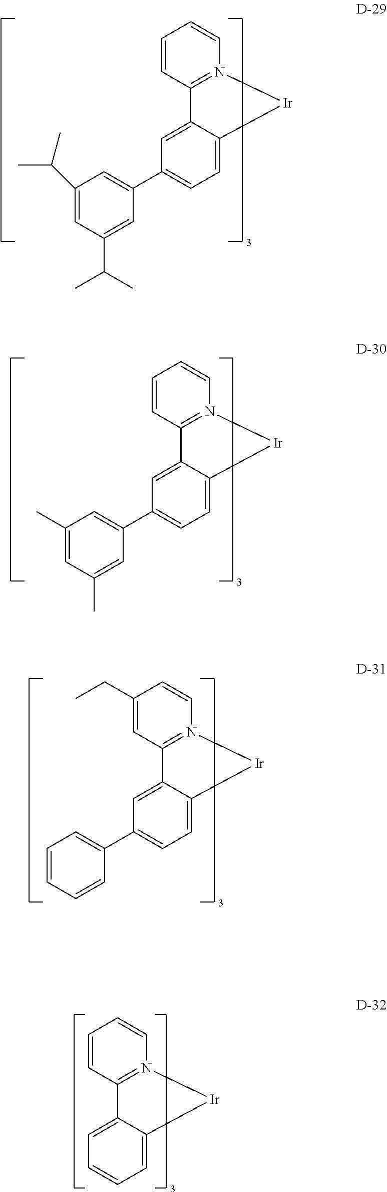

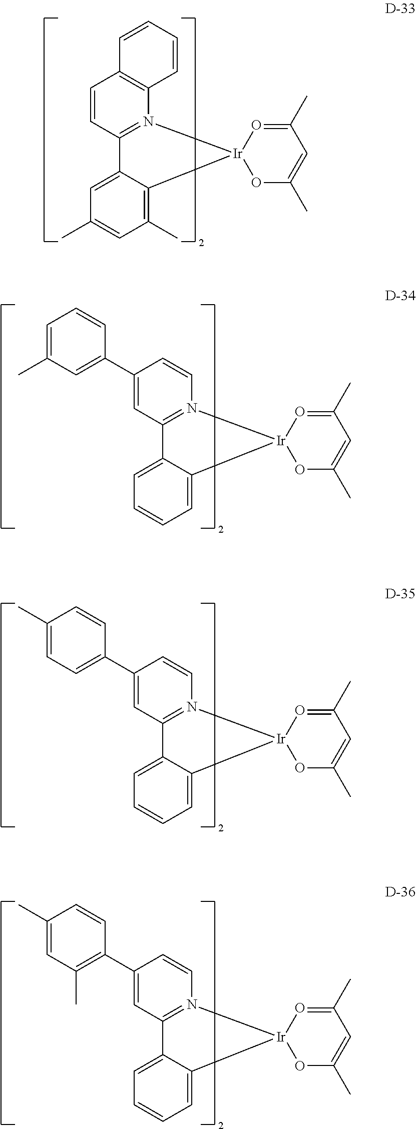

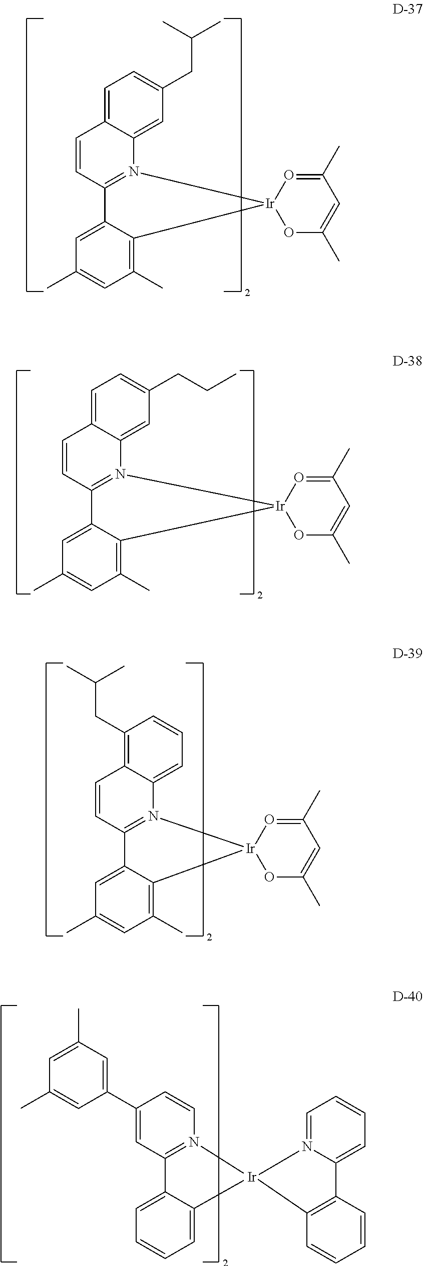

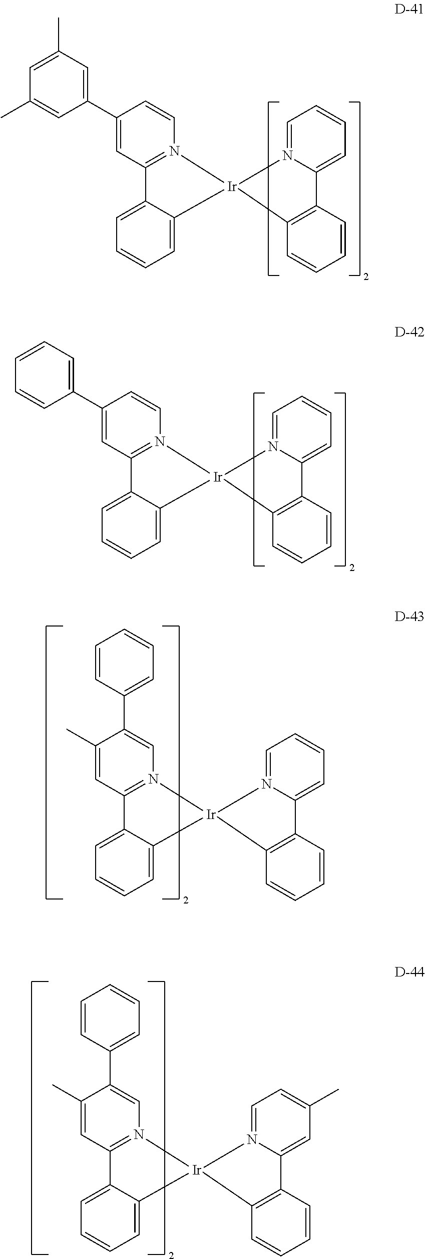

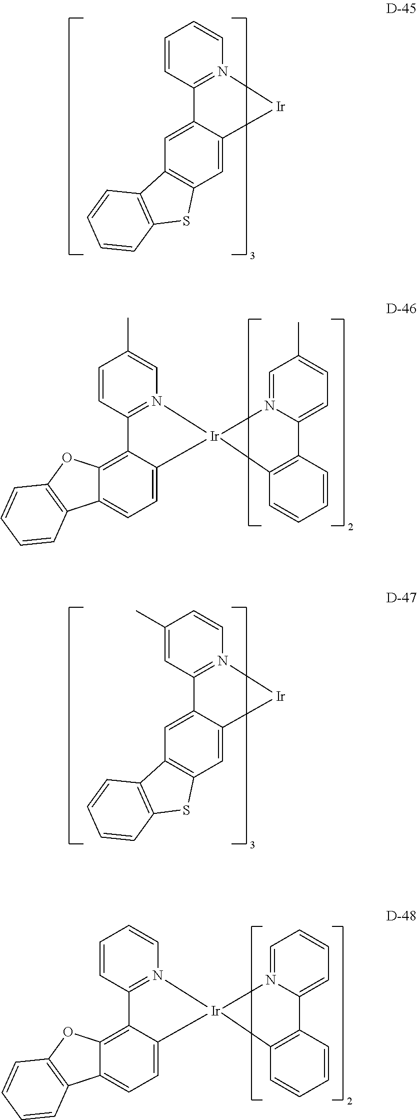

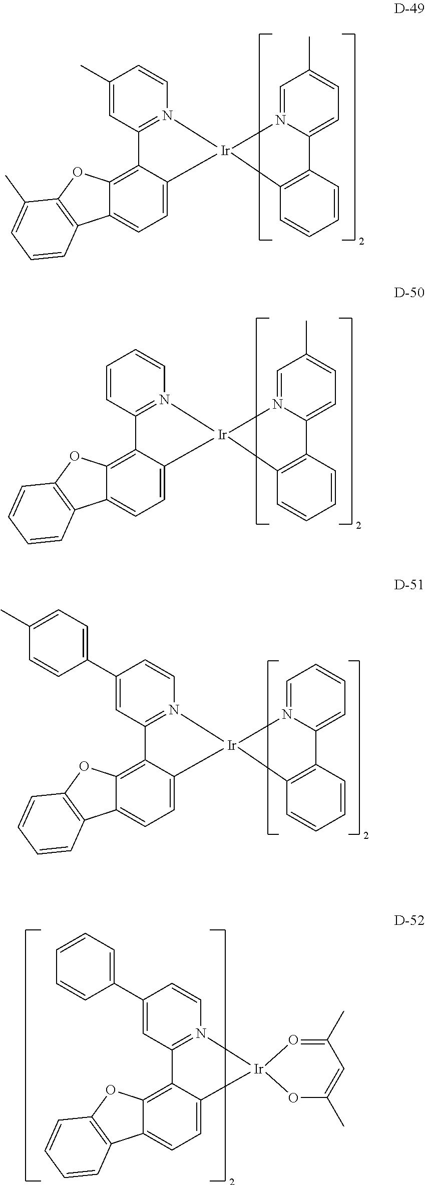

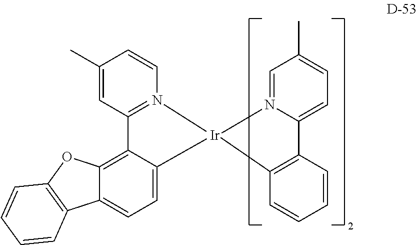

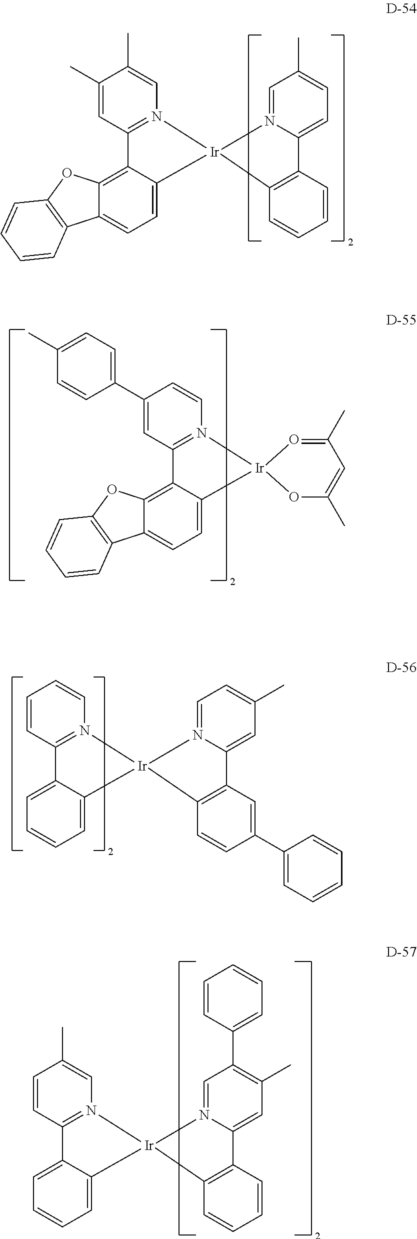

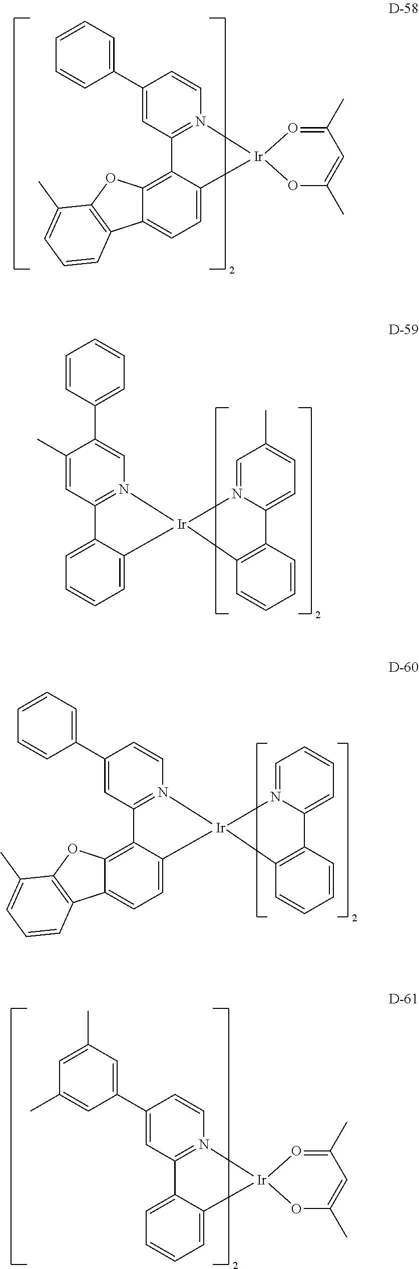

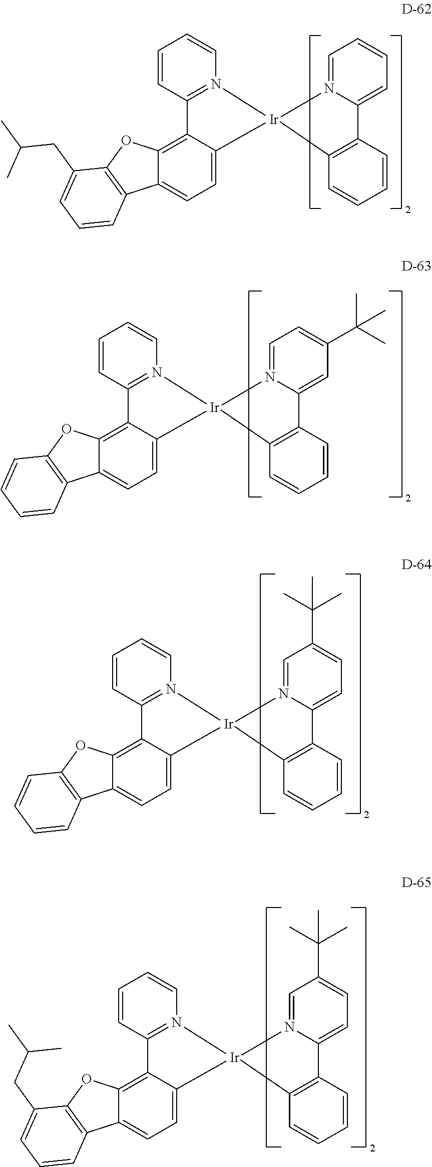

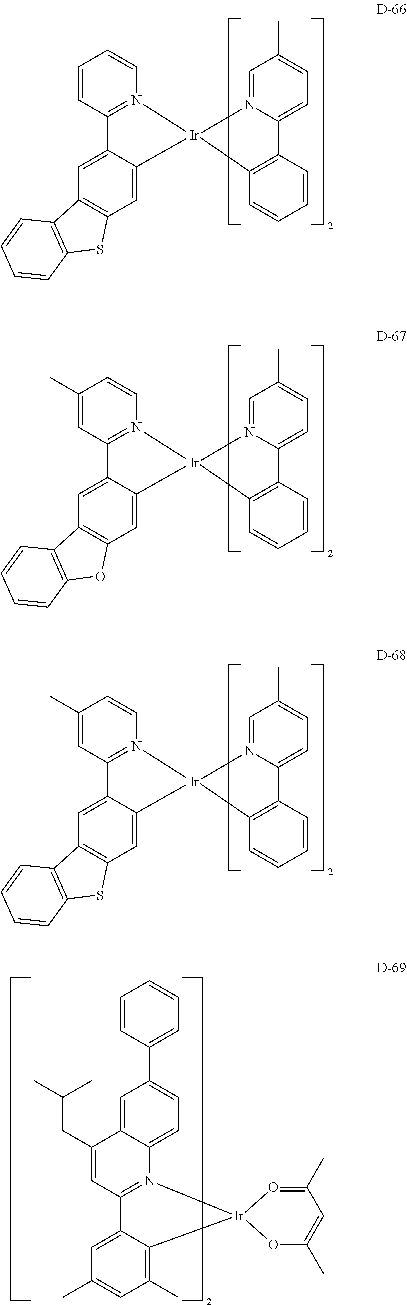

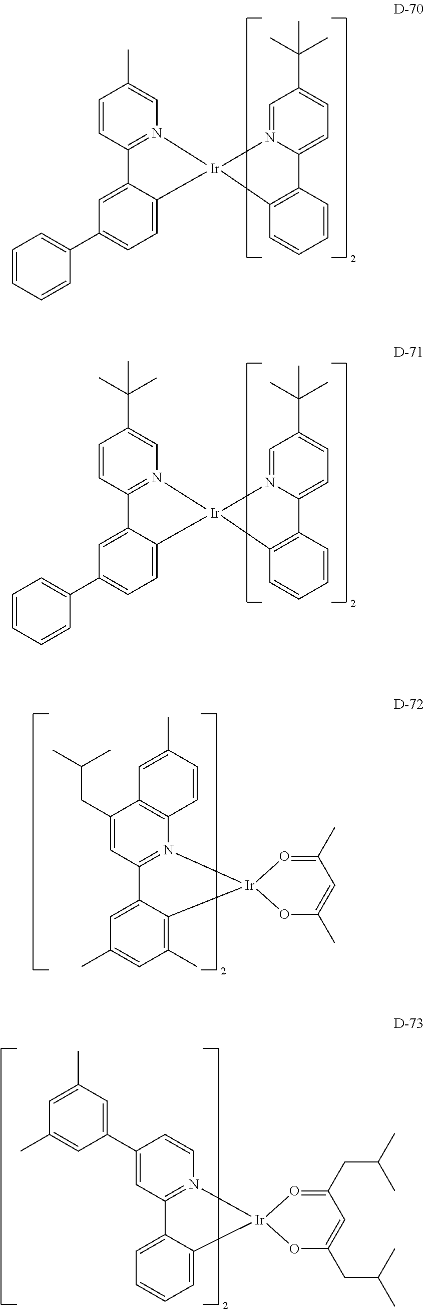

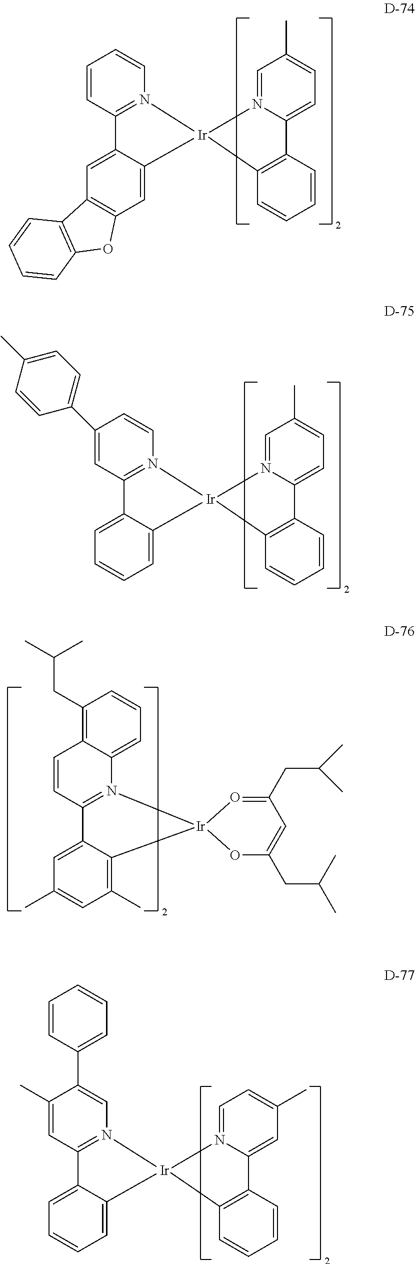

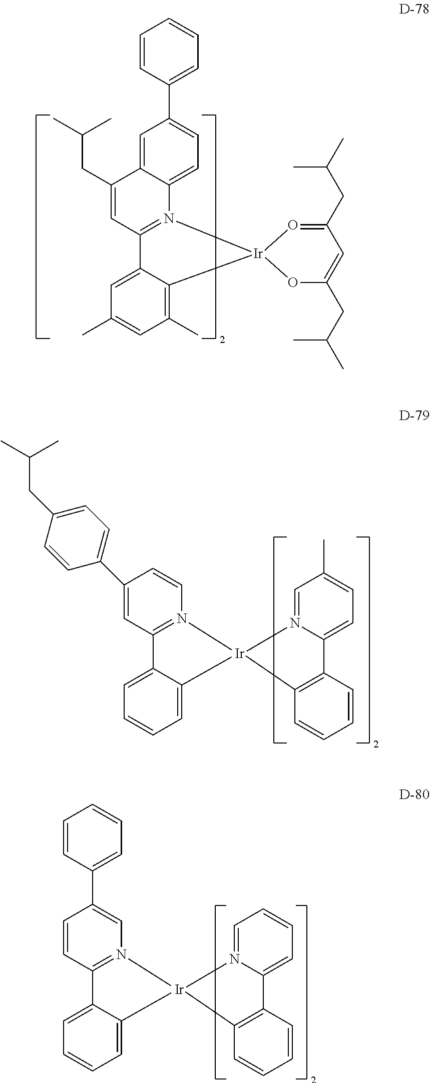

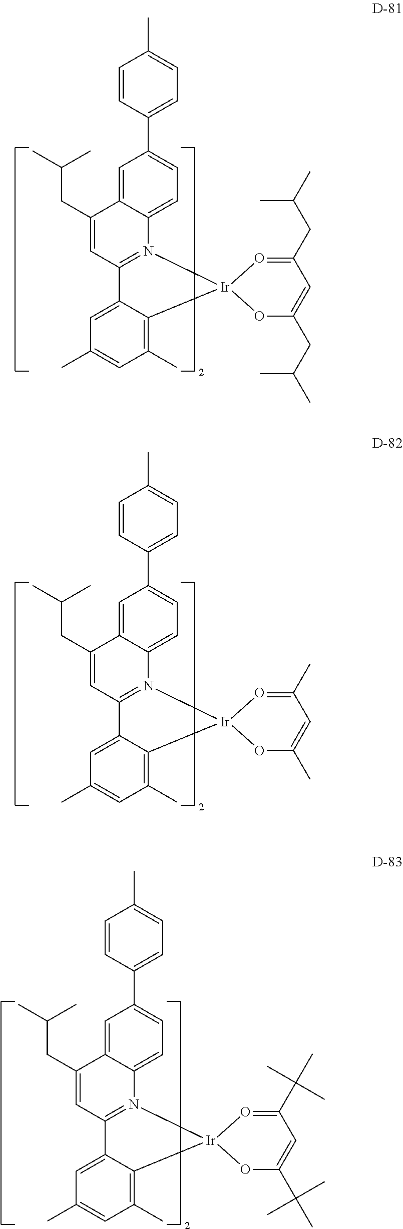

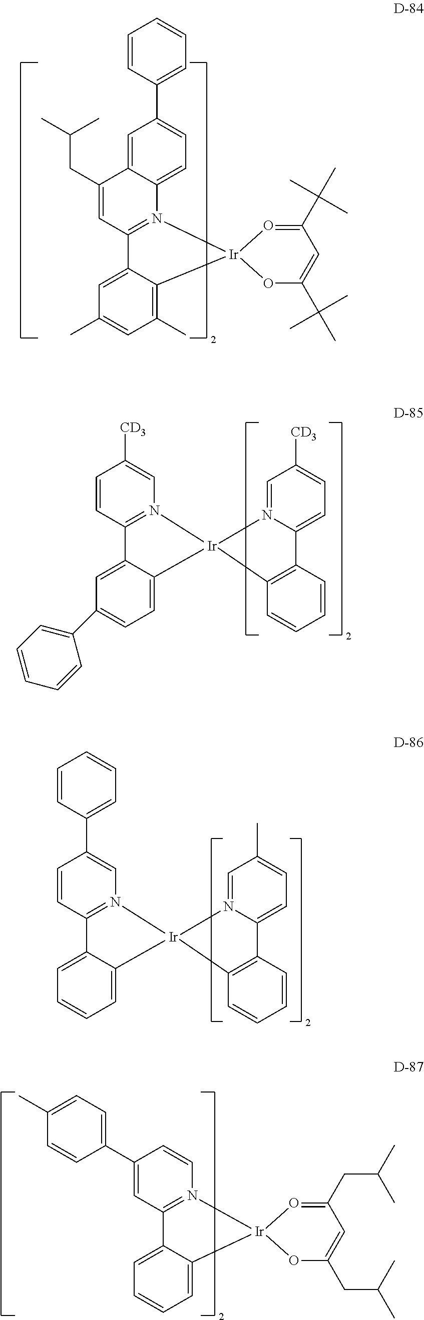

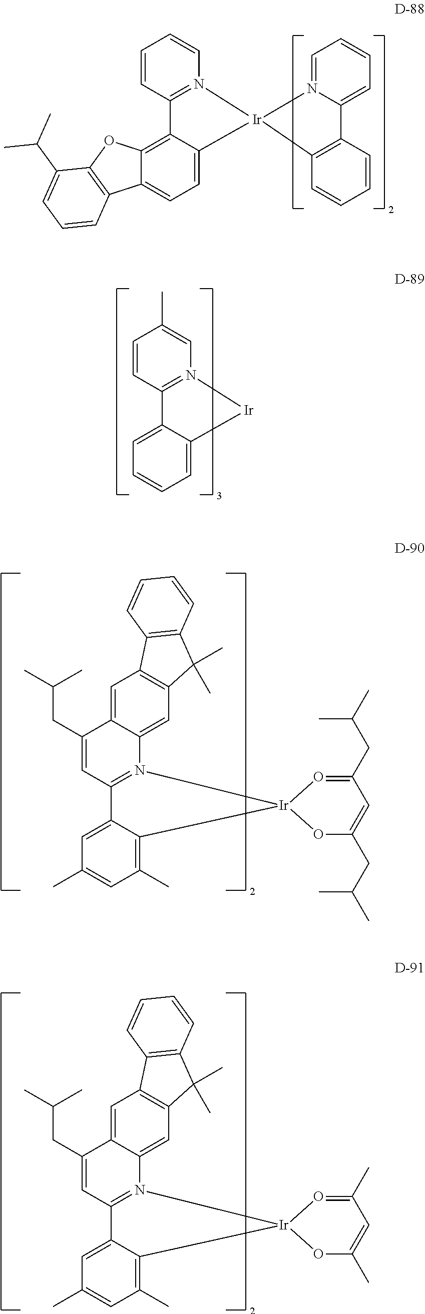

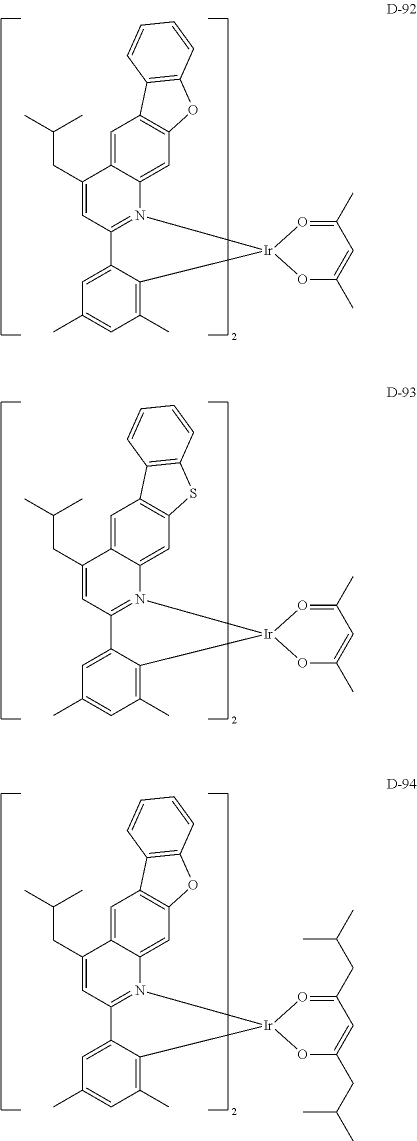

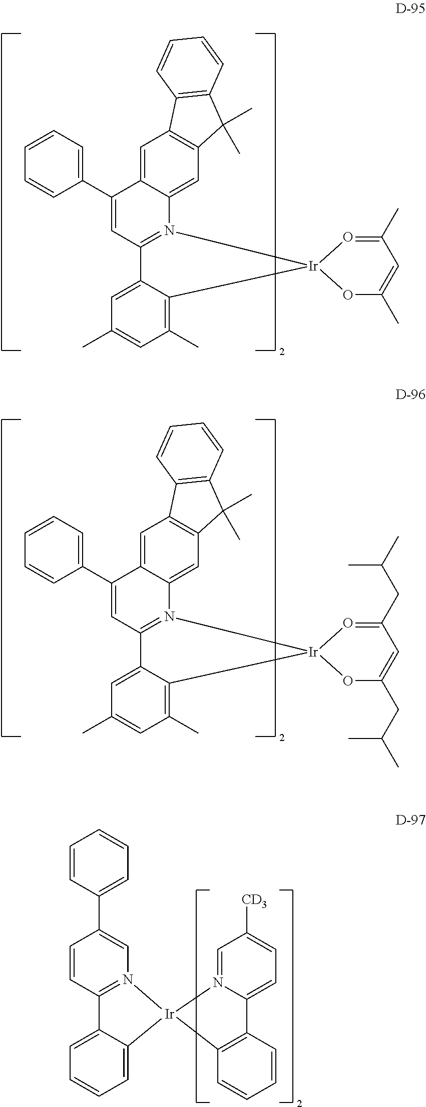

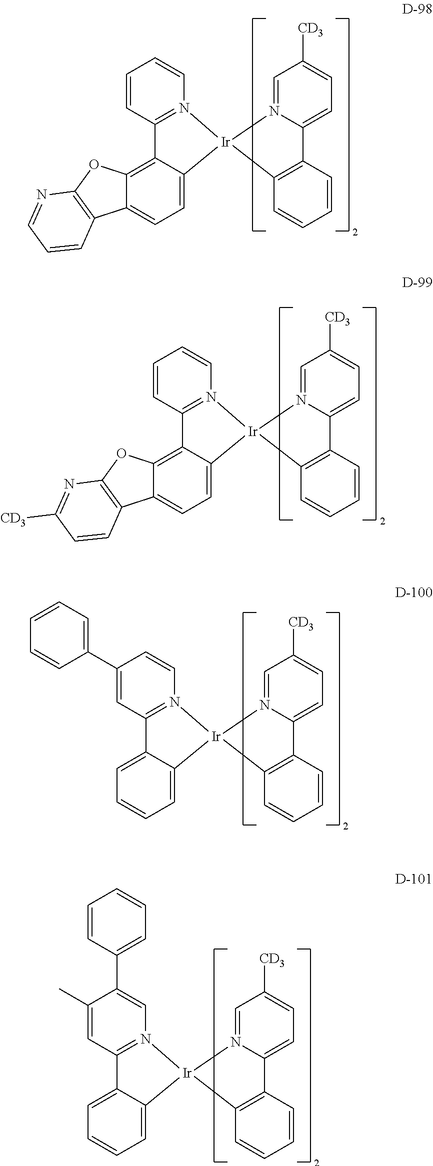

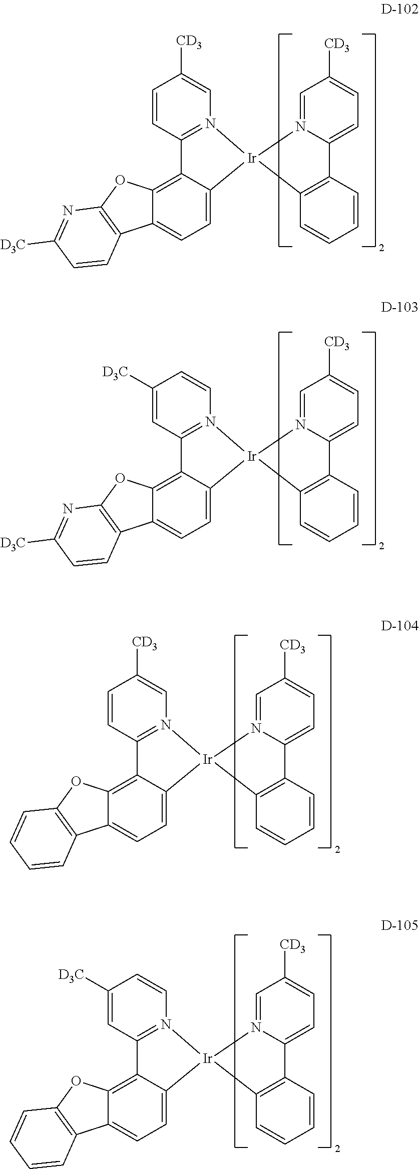

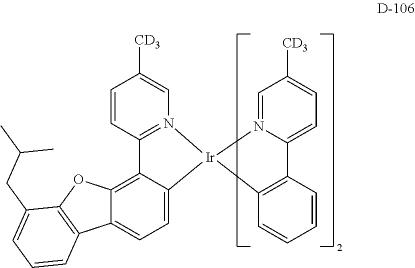

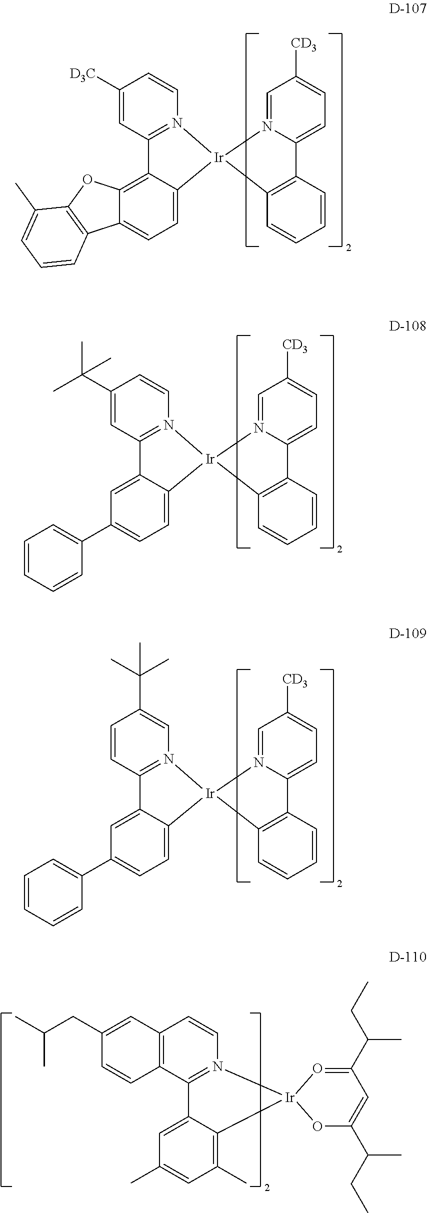

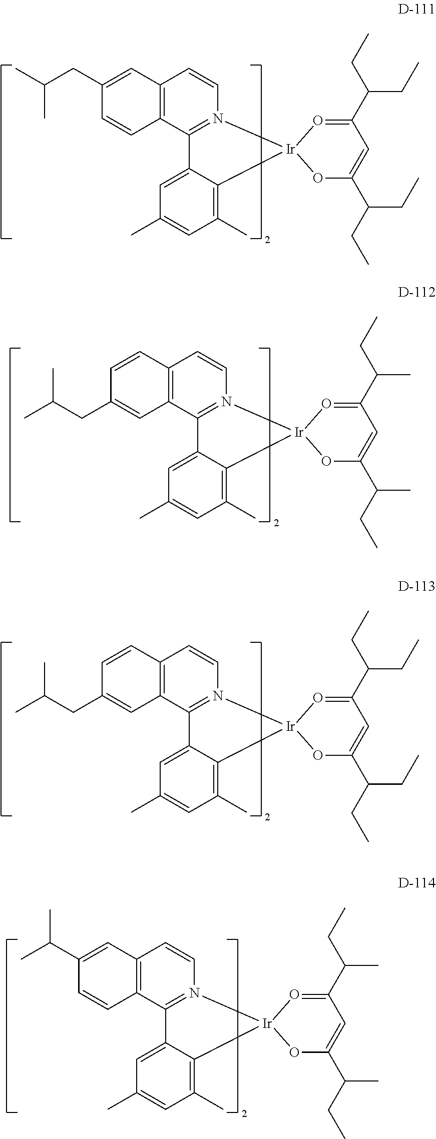

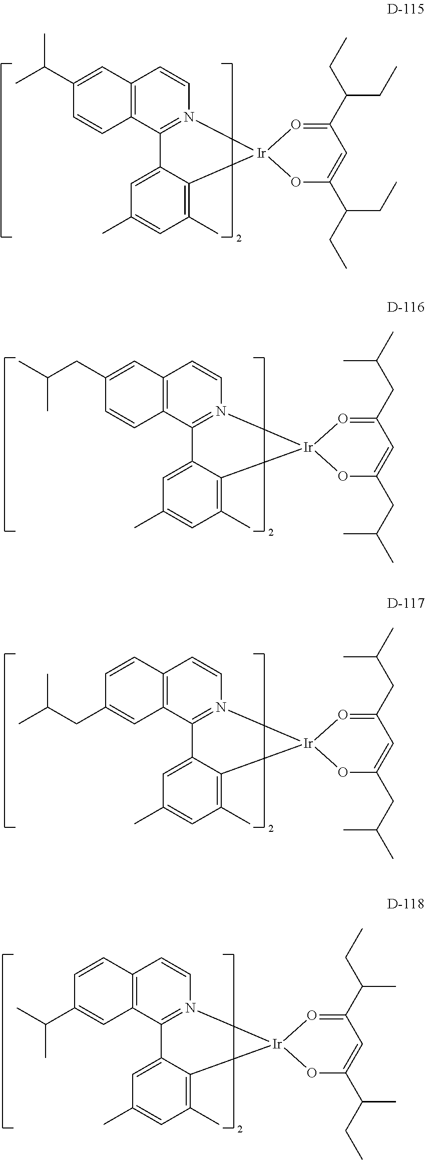

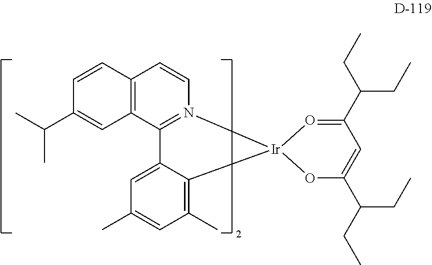

In the organic electroluminescent device of the present disclosure, a hole injection layer, a hole transport layer, an electron blocking layer, or a combination thereof can be used between the anode and the light-emitting layer. The hole injection layer may be multilayers in order to lower the hole injection barrier (or hole injection voltage) from the anode to the hole transport layer or the electron blocking layer, wherein each of the multilayers may use two compounds simultaneously. The hole transport layer or the electron blocking layer may also be multilayers.

An electron buffer layer, a hole blocking layer, an electron transport layer, an electron injection layer, or a combination thereof can be used between the light-emitting layer and the cathode. The electron buffer layer may be multilayers in order to control the injection of the electron and improve the interfacial properties between the light-emitting layer and the electron injection layer, wherein each of the multilayers may use two compounds simultaneously. The hole blocking layer or the electron transport layer may also be multilayers, wherein each of the multilayers may use a plurality of compounds.

In addition, the organic electroluminescent compound or the plurality of host materials according to the present disclosure may also be used in an organic electroluminescent device comprising a QD (quantum dot).

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

The first and the second host compounds of the present disclosure may be film-formed by the above-listed methods, commonly by a co-evaporation process or a mixture-evaporation process. The co-evaporation is a mixed deposition method in which two or more materials are placed in a respective individual crucible source and a current is applied to both cells at the same time to evaporate the materials. The mixture-evaporation is a mixed deposition method in which two or more materials are mixed in one crucible source before evaporating them, and a current is applied to the cell to evaporate the materials. Further, if the first and the second host compounds are present in the same layer or different layers in an organic electroluminescent device, the two host compounds may individualy form films. For example, the second host compound may be deposited after depositing the first host compound.

The present disclosure may provide a display device by using the plurality of host materials including the compound represented by formula 1 and the compound represented by formula 2. That is, by using the plurality of host materials of the present disclosure, it is possible to manufacture a display system or a lighting system. Specifically, by using the plurality of host materials of the present disclosure, a display system, for example, for white organic light emitting devices, smart phones, tablets, notebooks, PCs, TVs, or cars; or a lighting system, for example an outdoor or indoor lighting system, can be produced.

Hereinafter, the preparation method of the compound of the present disclosure and the properties thereof, and the properties of an organic electroluminescent device comprising the plurality of host materials of the present disclosure will be explained in detail with reference to the representative compounds of the present disclosure. However, the present disclosure is not limited by the following examples.

### EXAMPLE 1: PREPARATION OF COMPOUND H-2-80

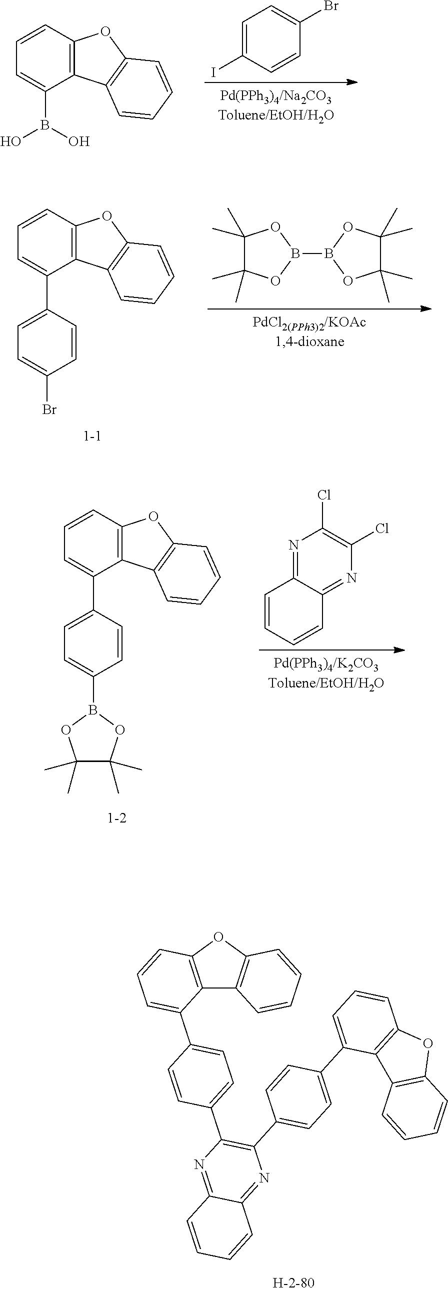

Synthesis of Compound 1-1

550 mL of toluene, 200 mL of EtOH, and 200 mL of H₂O were added dropwise to 40.0 g of dibenzo[b,d]furan-1-yl boronic acid (189 mmol), 80.06 g of 1-bromo-4-iodobenzene (283 mmol), 10.90 g of Pd(PPhs)₄ (9 mmol), and 49.99 g of Na₂CO₂ (472 mmol) in a flask, and the mixture was stirred under reflux at 150° C. for 2 hours, After completion of the reaction, the organic layer was extracted with ethyl acetate (EA), and dried with MgSO₄. The residue was separated by column chromatography, and MeOH was added thereto. The resulting solid was filtered under reduced pressure to obtain 30.1 g of compound 1-1 (yield: 49.3%).

Synthesis of Compound 1-2

150 mL of 1,4-dioxane was added dropwise to 9.0 g of compound 1-(28 mmol), 10.61 g of 4,4,4′,4′,5,5,5′,5′-octamethyl-2,2′-bi(1,3,2-dioxaborolane) (42 mmol), 0.977 g of PdCl₂(PPh₃)₂ (1 mmol), and 6.832 g of KOAc (70 mmol) in a flask, and the mixture was stirred under reflux at 140° C. for 1 hour. After completion of the reaction, the organic layer was extracted with EA, and dried with MgSO₄, The residue was separated by column chromatography, and MeOH was added thereto. The resulting solid was filtered under reduced pressure to obtain 10.2 g of compound 1-2 (yield: 98,93%).

Synthesis of Compound H-2-80

10 mL of toluene, 3 mL of EtOH, and 3 mL of H₂O were added dropwise to 2.50 g of 2,3-dichloroquinoxaline (13 mmol), 10.23 g of compound 1-2 (28 mmol), 1.451 g of Pd(PPh₃)₄ (1 mmol), and 8.680 g of K₂CO₃ (63 mmol) in a flask, and the mixture was stirred under reflux at 150° C. for 2 hours. After completion of the reaction, the organic layer was extracted with EA, and dried with MgSO₄. The residue was separated by column chromatography, and MeOH was added thereto. The resulting solid was filtered under reduced pressure to obtain 1.6 g of compound H-2-80 (yield: 20.0%).

¹H NMR (600 MHz, DMSO-d6, δ) 8.28 (dd, J=6, 3, 3.4 Hz, 2H), 7.98 (dd, J=6.3, 3.4 Hz, 2H), 7.85-7.80 (m, 4H), 7.77 (dd, J=8.3, 0.9 Hz, 2H), 7.73-7.68 (m, 4H), 7.66 (d, J=8.1 Hz, 2H), 7.63 (dd, J=8.2, 7.4 Hz, 2H), 7.42 (dt, J=7.9, 0.9 Hz, 2H), 7.37 (dd, J=7.4, 0.9 Hz, 2H), 7.30 (ddd, J=8.4, 7.2, 1.3 Hz, 2H), 6.91 (td, J=7.6, 1.0 Hz, 2H)

| Compound | MW | M.P. |
|---|---|---|
| H-2-80 | 614.70 | 231° C. |

### EXAMPLE 2: PREPARATION OF COMPOUND H-2-12

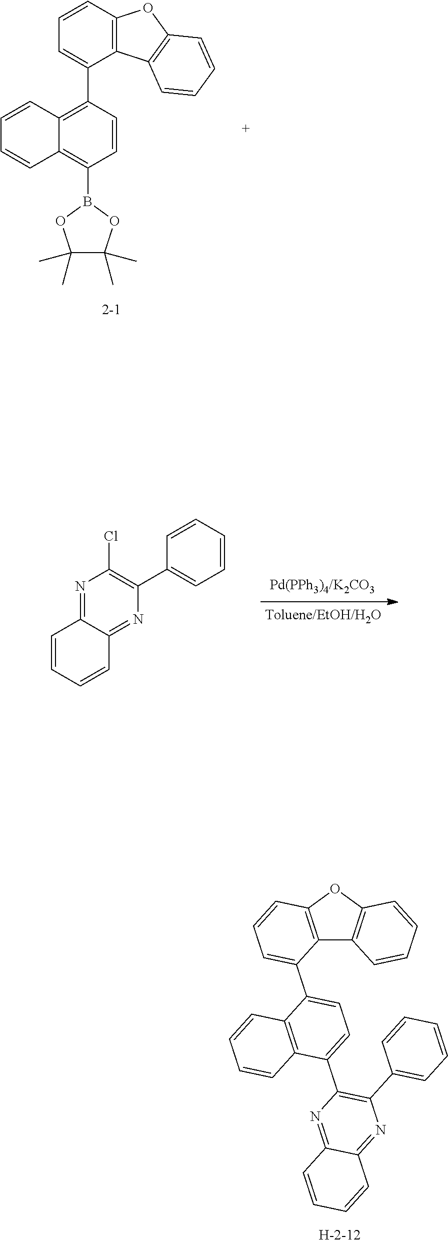 [SMILES: CC1(C)OB(c2ccc(-c3cccc4oc5ccccc5c34)c3ccccc23)OC1(C)C ; Clc1nc2ccccc2nc1-c1ccccc1 ; c1ccc(-c2nc3ccccc3nc2-c2ccc(-c3cccc4oc5ccccc5c34)c3ccccc23)cc1]

50 mL of toluene, 20 ml of EtOH, and 20 mL of H₂O were added dropwise to 4.0 g of compound 2-1 (17 mmol), 8.38 g of 2-chloro-3-phenylquinoxaline (20 mmol), 0.960 g of Pd(PPh₃)₄ (0.83 mmol), and 6.89 g of K₂CO₃ (50 mmol) in a flask, and the mixture was stirred under reflux at 140° C. for 2 hours. After completion of the reaction, the organic layer was extracted with EA, and dried with MgSO₄. The residue was separated by column chromatography, and MeOH was added thereto. The resulting solid was filtered under reduced pressure to obtain 3.2 g of compound H-2-12 (yield: 38.6%).

¹H NMR (600 MHz, DMSO-d6, δ) 8.34-8.29 (m, 1H), 8.25 (d, J=7.8 Hz, 1H), 8.04-7.95 (m, 2H), 7.87 (dd, J=8.3, 0.9 Hz, 1H), 7.76-7.69 (m, 4H), 7.62 (d, J=7.2 Hz, 1H), 7.54 (d, J=7.5 Hz, 2H), 7.48-7.39 (m, 4H), 7.37 (s, 1H), 7.30 (dt, J=26.1, 7.6 Hz, 3H), 7.19 (s, 1H), 7.03 (t, J=7.5 Hz, 1H)

| Compound | MW | M.P. |
|---|---|---|
| H-2-12 | 498.59 | 245° C. |

### EXAMPLE 3: PREPARATION OF COMPOUND H-2-9

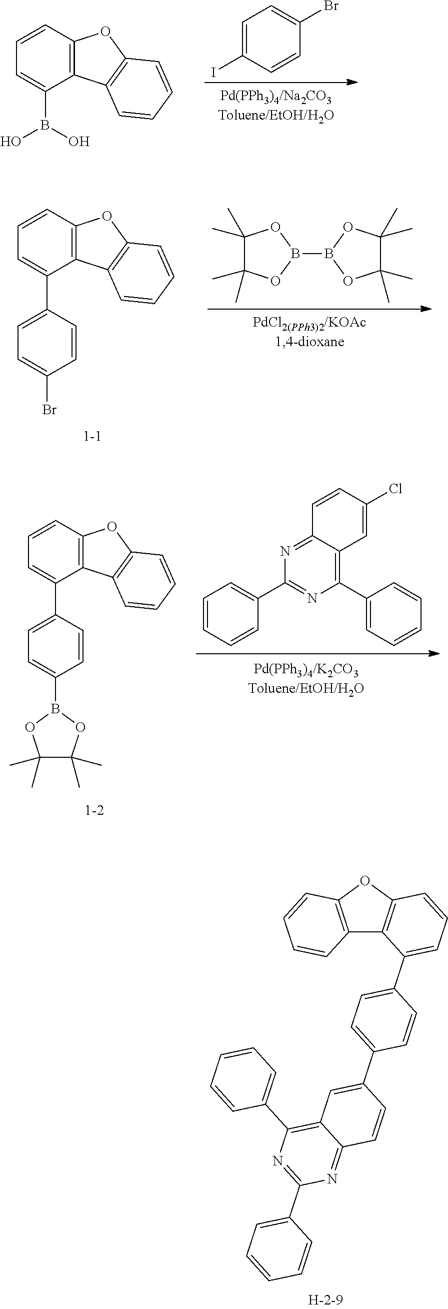

Synthesis of Compound 1-1

550 mL of toluene, 200 mL of EtOH, and 200 mL of H₂O were added dropwise to 80.0 g of dibenzo[b,d]furan-1-yl boronic acid (377 mmol), 160.13 g of 1-bromo-4-iodobenzene (566 mmol), 21.80 g of Pd(PPh₃)₄ (19 mmol), and 99.99 g of Na₂CO₃ (943 mmol) in a flask, and the mixture was stirred under reflux at 150° C. for 2.5 hours. After completion of the reaction, the organic layer was extracted with EA, and dried with MgSO₄. The residue was separated by column chromatography, and MeOH was added thereto. The resulting solid was filtered under reduced pressure to obtain 51.8 g of compound 1-1 (yield: 42.5%).

Synthesis of Compound 1-2

150 mL of 1,4-dioxane was added dropwise to 30.0 g of compound 1-1 (93 mmol), 35.4 g of 4,4,4′,4′,5,5,5′,5′-octamethyl-2,2′-bi(1,3,2-dioxaborolane) (139 mmol), 3.26 g of PdCl₂(PPh₃)₂ (5 mmol), and 22.77 g of KOAc (232 mmol) in a flask, and the mixture was stirred under reflux at 140° C. for 1 hour. After completion of the reaction, the organic layer was extracted with EA, and dried with MgSO₄. The residue was separated by column chromatography, and MeOH was added thereto. The resulting solid was filtered under reduced pressure to obtain 23.3 g of compound 1-2 (yield: 67.8%).

Synthesis of Compound H-2-9

40 mL of toluene, 15 mL of EtOH, and 15 mL of H₂O were added dropwise to 4.28 g of 6-chloro-2,4-diphenylquinazoline (14 mmol), 6.00 g of compound 1-2 (16 mmol), 0.780 g of Pd(PPh₃)₄ (0.675 mmol), and 4.67 g of K₂CO₃ (34 mmol) in a flask, and the mixture was stirred under reflux at 150° C. for 2 hours. After completion of the reaction, the organic layer was extracted with EA, and dried with MgSO₄. The residue was separated by column chromatography, and MeOH was added thereto. The resulting solid was filtered under reduced pressure to obtain 4.3 g of compound H-2-9 (yield: 60.7%).

¹H NMR (600 MHz, DMSO-d6, δ) 8.69-8.64 (m, 2H), 8.54 (dd, J=8.7, 2.0 Hz, 1H), 8.42 (d, J=2.0 Hz, 1H), 8.31 (d, J=8.7 Hz, 1H), 8.03 (dd, J=21.1, 7.3 Hz, 4H), 7.83-7.69 (m, 7H), 7.66-7.56 (m, 5H), 7.51 (t, J=7.7 Hz, 1H), 7.37 (d, J=7.4 Hz, 1H), 7.25 (t, J=7.6 Hz, 1H)

| Compound | MW | M.P. |
|---|---|---|
| H-2-9 | 524.62 | 242° C. |

### EXAMPLE 4: PREPARATION OF COMPOUND H-2-7

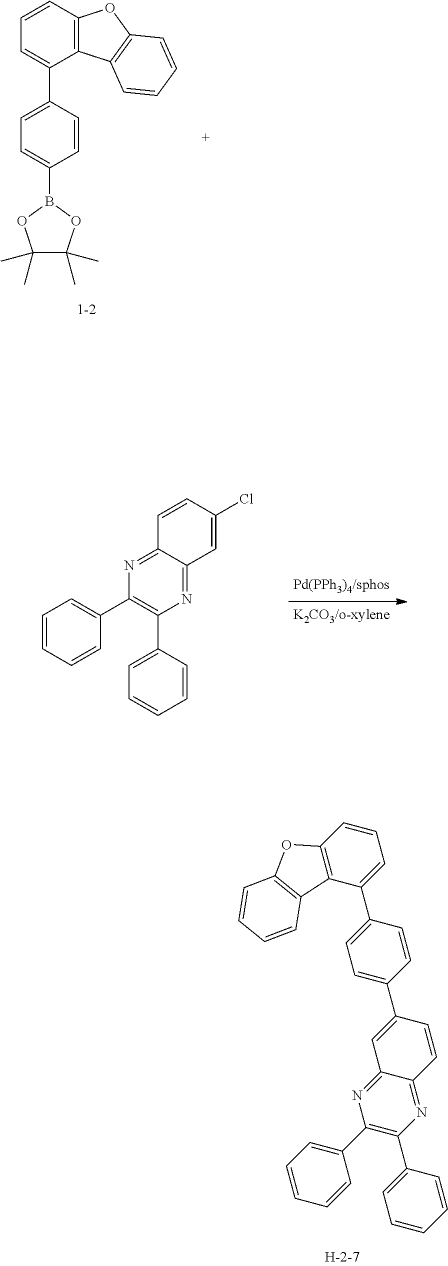 [SMILES: CC1(C)OB(c2ccc(-c3cccc4oc5ccccc5c34)cc2)OC1(C)C ; Clc1ccc2nc(-c3ccccc3)c(-c3ccccc3)nc2c1 ; c1ccc(-c2nc3ccc(-c4ccc(-c5cccc6oc7ccccc7c56)cc4)cc3nc2-c2ccccc2)cc1]

70 mL of o-xylene was added dropwise to 6.00 g of compound 1-2 (16 mmol), 4.28 g of 6-chloro-2,3-diphenylquinoxaline (14 mmol), 0.618 g of Pd(PPh₃)₄ (0.83 mmol), 0.554 g of 2-dicyclohexylphosphino-2′,6′-dimethoxybiphenyl (sphos) (1 mmol), and 3.24 g of K₂CO₃ (34 mmol) in a flask, and the mixture was stirred under reflux at 140° C. for 2 hours. After completion of the reaction, the organic layer was extracted with EA, and dried with MgSO₄. The residue was separated by column chromatography, and MeOH was added thereto. The resulting solid was filtered under reduced pressure to obtain 4.6 g of compound H-2-7 (yield: 64.9%).

¹H NMR (600 MHz, DMSO-d6, δ) 8.59 (d, J=2.1 Hz, 1H), 8.41 (dd, J=8.7, 2.1 Hz, 1H), 8.30 (d, J=8.7 Hz, 1H), 8.21 (d, J=8.2 Hz, 2H), 7.87-7.83 (m, 2H), 7.81-7.74 (m, 2H), 7.68-7.62 (m, 2H), 7.57-7.50 (m, 5H), 7.45-7.36 (m, 7H), 7.27 (t, J=7.6 Hz, 1H)

| Compound | MW | M.P. |
|---|---|---|
| H-2-7 | 524.62 | 225° C. |

### EXAMPLE 5: PREPARATION OF COMPOUND H-2-89

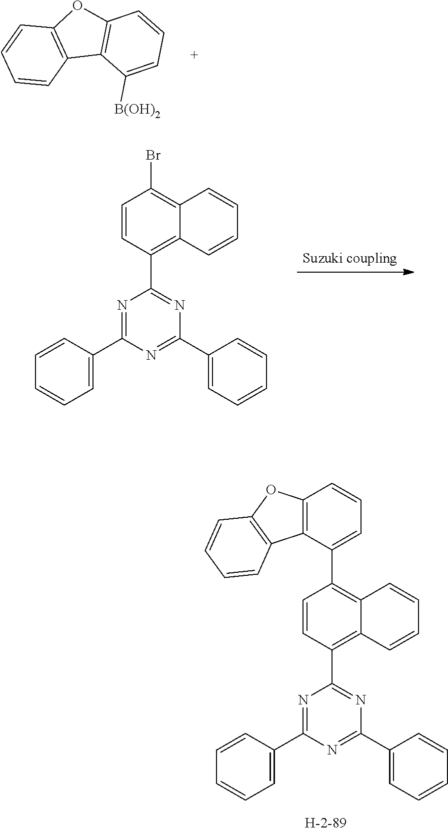 [SMILES: OB(O)c1cccc2oc3ccccc3c12 ; Brc1ccc(-c2nc(-c3ccccc3)nc(-c3ccccc3)n2)c2ccccc12 ; c1ccc(-c2nc(-c3ccccc3)nc(-c3ccc(-c4cccc5oc6ccccc6c45)c4ccccc34)n2)cc1]

3.0 g of dibenzo[b,d]furan-1-yl boronic acid (14.2 mmol), 6.3 g of 2-(4-bromonaphthalen-1-yl)-4,6-diphenyl-1,3,5-triazine (14.2 mmol), 0.82 g of tetrakis(triphenylphosphine)palladium (0) (0.71 mmol), and 3.9 g of sodium carbonate (28.4 mmol) were dissolved in 30 mL of toluene, 8 mL of ethanol, and 15 mL of water in a flask, and the mixture was refluxed for 2 hours. After completion of the reaction, the organic layer was extracted with ethyl acetate, and the residual moisture was removed with magnesium sulfate. The residue was dried and separated by column chromatography to obtain 1.9 g of compound H-2-89 (yield: 26%).

| Compound | MW | M.P. |
|---|---|---|
| H-2-89 | 525.6 | 203° C. |

### EXAMPLE 6: PREPARATION OF COMPOUND H-2-91

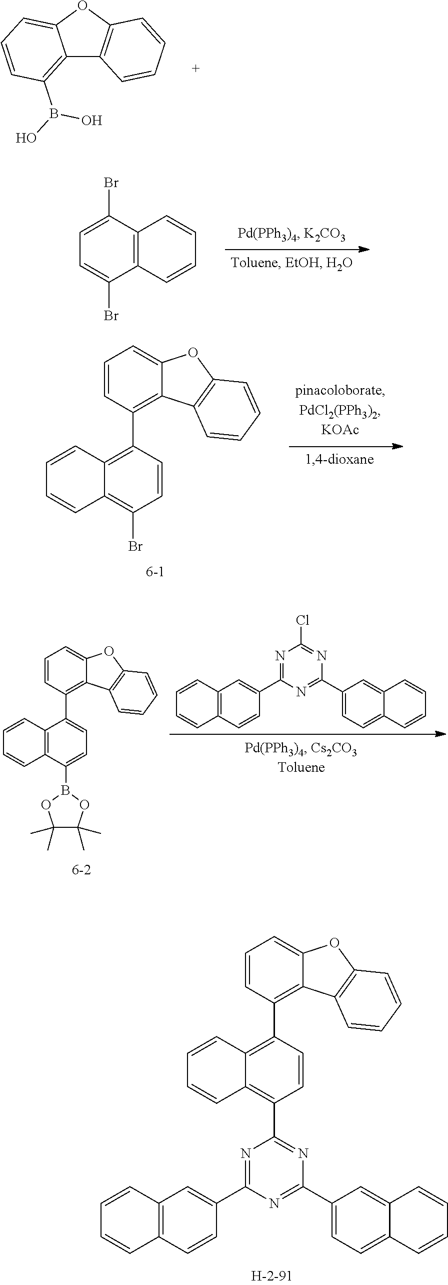

Synthesis of Compound 6-1

20 g of dibenzo[b,d]furan-1-yl boronic acid (94.3 mmol), 53.9 g of 1; 4-dibromonaphthalene (188.67 mmol), 32.6 g of K₂CO₃ (235.75 mmol), and 5.4 g of Pd(PPh₃)₄ (4.7 nmol) were dissolved in 470 mL of toluene, 235 mL of ethanol, and 235 mL of water in a flask, and the mixture was refluxed at 140° C. for 4 hours. After completion of the reaction, the organic layer was extracted with ethyl acetate, and the residual moisture was removed with magnesium sulfate. The residue was dried and separated by column chromatography to obtain 20 g of compound 6-1 (yield: 56.8%).

Synthesis of Compound 6-2

20 g of compound 6-1 (53.6 mmol), 16.3 g of 4,4,4′,4′,5,5,5′,5′-octamethyl-2,2′-bi(1,3,2-dioxaborolane) (64.3 mmol), 3.76 g of PdCl₂(PPh₃)₂ (5.36 mmol), and 10.5 g of KOAc (107.2 mmol) were dissolved in 270 mL of 1,4-dioxane in a flask, and the mixture was refluxed at 150° C. for 4 hours. After completion of the reaction, the organic layer was extracted with ethyl acetate, and the residual moisture was removed with magnesium sulfate. The residue was dried and separated by column chromatography to obtain 23 g of compound 6-2 (yield: 100%).

Synthesis of Compound H-2-91

7 g of compound 6-2 (16.6 mmol), 7.35 g of 2-chloro-4,6-di(naphthalen-2-yl)-1,3,5-triazine (19.9 mmol), 13.5 g of Cs₂CO₃ (41.5 mmol), and 959 mg of Pd(PPh₃)₄ (0.83 mmol) were dissolved in 83 mL of toluene in a flask, and the mixture was refluxed at 130° C. for 18 hours. After completion of the reaction, the organic layer was extracted with ethyl acetate, and the residual moisture was removed with magnesium sulfate. The residue was dried and separated by column chromatography to obtain 2 g of compound H-2-91 (yield: 19.2%).

| Compound | MW | M.P. |
|---|---|---|
| H-2-91 | 625.73 | 15C° C. |

### EXAMPLE 7: PREPARATION OF COMPOUND H-2-94

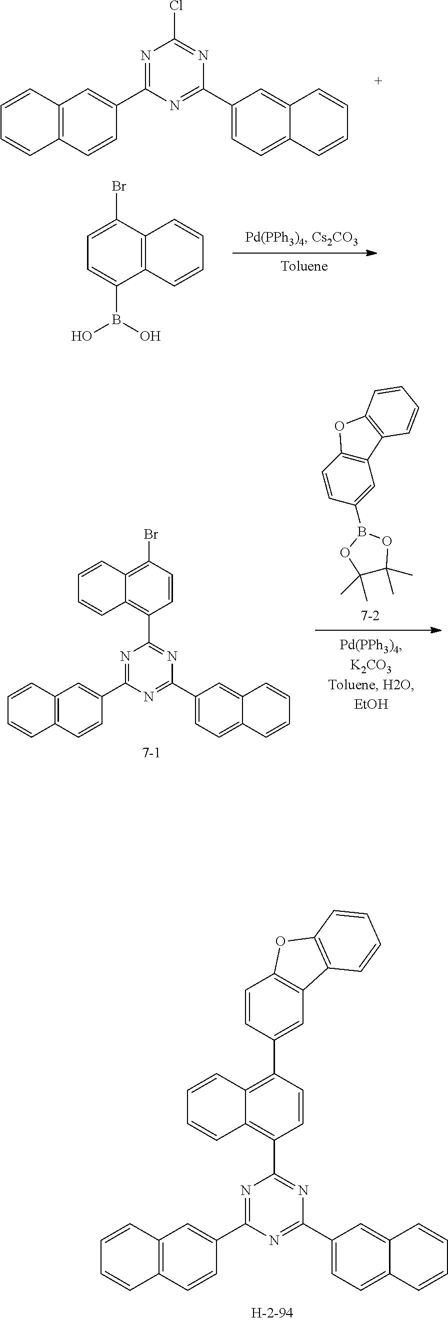

Synthesis of Compound 7-1

32.2 g of 2-chloro-4,6-di(naphthalen-2-yl)-1,3,5-triazine (87.7 mmol), 20 g of (4-bromonaphthalen-1-yl) boronic acid (79.7 mmol), 65 g of Cs₂CO₃ (199.25 mmol), and 4.6 g of Pd(PPh₃)₄ (3.985 mmol) were dissolved in 400 mL of toluene in a flask, and the mixture was refluxed at 140° C. for 4 hours. After completion of the reaction, the organic layer was extracted with ethyl acetate, and the residual moisture was removed with magnesium sulfate. The residue was dried and separated by column chromatography to obtain 20 g of compound 7-1 (yield: 46.6%).

Synthesis of Compound H-2-94

7 g of compound 7-1 (13 mmol), 4.6 g of compound 2-2 (15.6 mmol), 4.5 g of K₂CO₃, (32.5 mmol), and 0.75 g of Pd(PPh₃)₄ (0.65 mmol) were dissolved in 65 mL of toluene, 32.5 mL of ethanol, and 32.5 mL of H₂O in a flask, and the mixture was refluxed at 130° C. for 3 hours. After completion of the reaction, the organic layer was extracted with ethyl acetate, and the residual moisture was removed with magnesium sulfate. The residue was dried and separated by column chromatography to obtain 3.4 g of compound H-2-94 (yield: 41%).

| Compound | MW | MP. |
|---|---|---|
| H-2-94 | 625.73 | 250° C. |

### EXAMPLE 8: PREPARATION OF COMPOUND H-2-108

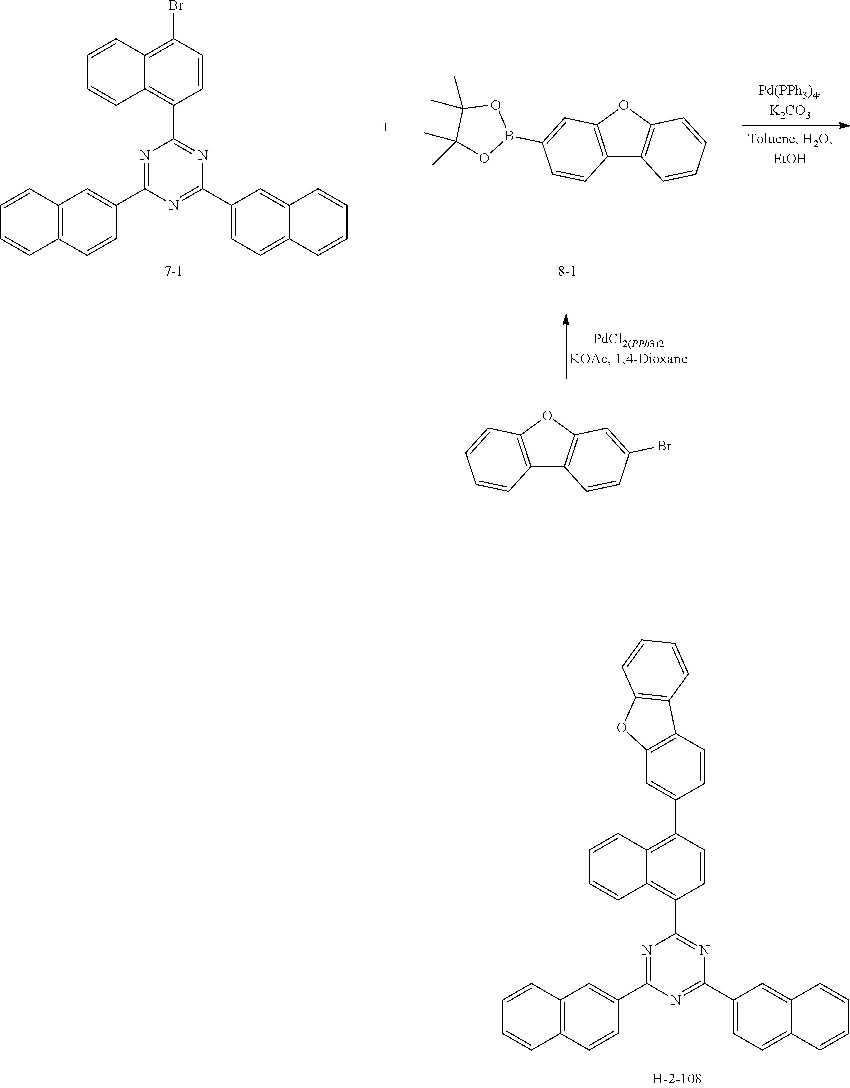 [SMILES: Brc1ccc(-c2nc(-c3ccc4ccccc4c3)nc(-c3ccc4ccccc4c3)n2)c2ccccc12 ; CC1(C)OB(c2ccc3c(c2)oc2ccccc23)OC1(C)C ; Brc1ccc2c(c1)oc1ccccc12 ; c1ccc2cc(-c3nc(-c4ccc5ccccc5c4)nc(-c4ccc(-c5ccc6c(c5)oc5ccccc56)c5ccccc45)n3)ccc2c1]

Synthesis of Compound 8-1

5 g of 3-bromodibenzofuran (20 mmol), 7.6 g of 4,4,4′,4′,5,5,5″,5′-octamethyl-2,2′-bi(1,3,2-dioxaborolane) (30 mmol), 1.4 g of PdCl₂(PPh₃)₂ (2 mmol), and 3.9 g of KOAc (50 mmol) were dissolved in 100 mL of 1,4-dioxane in a flask, and the mixture was refluxed at 150° C. for 4 hours. After completion of the reaction, the organic layer was extracted with ethyl acetate, and the residual moisture was removed with magnesium sulfate. The residue was dried and separated by column chromatography to obtain 5 g of compound 8-1 (yield: 85%).

Synthesis of Compound H-2-108

4.4 g of compound 7-1 (12.3 mmol), 5 g of compound 84 (13.5 mmol), 4.5 g of K₂CO₃ (32.5 mmol), and 0.75 g of Pd(PPh₃)₄ (0.65 mmol) were dissolved in 60 mL of toluene, 30 mL of ethanol, and 30 mL of H₂O in a flask, and the mixture was refluxed at 130° C. for 3 hours. After completion of the reaction, the organic layer was extracted with ethyl acetate, and the residual moisture was removed with magnesium sulfate. The residue was dried and separated by column chromatography to obtain 4 g of compound H-2-108 (yield: 49%).

### EXAMPLE 9: PREPARATION OF COMPOUND H-2-90

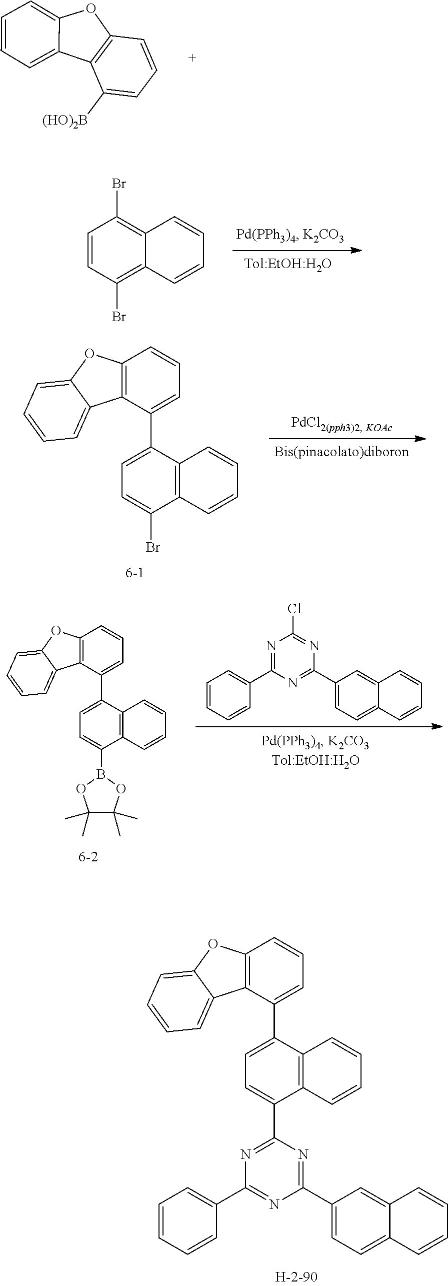

Synthesis of Compound 6-1

20 g of dibenzo[b,d]furan-1-yl boronic acid (94.33 mmol), 54 g of 1,4-dibromonaphthalene (188.6 mmol), 5.4 g of Pd(PPh₃)₄ (4,716 mmol), and 26 g of K₂CO₃ (188.6 mmol) were added to 380 mL of toluene, 95 mL of EtOH, and 95 mL of purified water in a flask, and the mixture was stirred under reflux for 3 hours. After completion of the reaction, the mixture was cooled to room temperature, and extracted with distilled water and EA. The organic layer was distilled under reduced pressure, and separated by column chromatography using MC/Hex to obtain 20 g of compound 6-1 (yield: 55%),

Synthesis of Compound 6-2

3.7 g of PdCl₂(PPh₃)₂ (53.59 mmol), 10.5 g of KOAc (107.1 mmol), 17.7 g of bis(pinacolato)diboron (69.66 mmol), and 270 mL of 1,4-dioxane were added to 20 g of compound 6-1 (53.59 mmol) in a flask, and the mixture was stirred under reflux for 2 hours. After completion of the reaction, the mixture was filtered with cellite, and extracted with MC. The organic layer was concentrated, and separated by column chromatography using MC/Hex to obtain 20 g of compound 6-2 (yield: 88%).

Synthesis of Compound H-2-90

6 g of compound 6-2 (14.16 mmol), 5 g of 2-chloro-4-(naphthalen-2-yl)-6-phenyl-1,3,5-triazine (15.73 mmol), 0.9 g of Pd(PPh₃)₄ (0.786 mmol), and 4.3 g of K₂CO₃ (31.47 mmol) were added to 64 mL of toluene, 16 mL of EtOH, and 16 mL of purified water in a flask, and the mixture was stirred under reflux for 2 hours, After completion of the reaction, the mixture was cooled to room temperature, and extracted with distilled water and EA. The organic layer was distilled under reduced pressure, and separated by column chromatography using MC/Hex to obtain 4 g of compound H-2-90 (yield: 44%).

| Compound | MW | M.P. |
|---|---|---|
| H-2-90 | 575.6 | 131.3° C. |

### DEVICE EXAMPLES 1 TO 5: PRODUCING AN OLED COMPRISING THE PLURALITY OF HOST MATERIALS ACCORDING TO THE PRESENT DISCLOSURE

OLEDs according to the present disclosure were produced. A transparent electrode indium tin oxide (ITO) thin film (10 Ω/sq) on a glass substrate for an OLED (GEOMATEC CO., LTD., Japan) was subjected to an ultrasonic washing with acetone, trichloroethylene, acetone, ethanol, and distilled water, sequentially, and then was stored in isopropanol. The ITO substrate was then mounted on a substrate holder of a vacuum vapor deposition apparatus. Compound HI-1 was introduced into a cell of the vacuum vapor deposition apparatus, and then the pressure in the chamber of the apparatus was controlled to 10⁻⁶ torr. Thereafter, an electric current was applied to the cell to evaporate the above-introduced material, thereby forming a first hole injection layer having a thickness of 80 nm on the ITO substrate. Next, compound HI-2 was introduced into another cell of the vacuum vapor deposition apparatus and was evaporated by applying an electric current to the cell, thereby forming a second hole injection layer having a thickness of 5 nm on the first hole injection layer. Compound HT-1 was then introduced into a cell of the vacuum vapor deposition apparatus and was evaporated by applying an electric current to the cell, thereby forming a first hole transport layer having a thickness of 10 nm on the second hole injection layer. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layers and the hole transport layers, a light-emitting layer was formed thereon as follows: The first host compound and the second host compound shown in Table 1 or 2 below were introduced into two cells of the vacuum vapor depositing apparatus as a host, and compound D-39 was introduced into another cell as a dopant. The two host materials were evaporated at a rate of 1:1 and the dopant material was simulataneously evaporated at a different rate, and the dopant was deposited in a doping amount of 3 wt % based on the total amount of the host and the dopant to form a light-emitting layer having a thickness of 40 nm on the second hole transport layer. Compound ET-1 and compound EI-1 were introduced into two cells and evaporated at a rate of 1:1 to form an electron transport layer having a thickness of 35 nm on the light-emitting layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced.

### COMPARATIVE EXAMPLES 1 TO 5: PRODUCING AN OLED NOT ACCORDING TO THE PRESENT DISCLOSURE

OLEDs were produced in the same manner as in Device Examples 1 to 5, except that the host materials shown in Table 1 or 2 below were used, instead of the host combination of the present disclosure.

The driving voltage, the luminous efficiency; the increase rate of the luminous efficiency, and the power efficiency at a luminance of 5,000 nit, and/or the time taken for luminance to decrease from 100% to 90% at a luminance of 5,000 nit (lifespan; T90) of the OLEDs produced in Device Examples 1 and 2, and Comparative Examples 1 and 2 are provided in Table 1 below.

TABLE 1

| | | | Driving | Luminous | Increase Rate of | Power | |
|---|---|---|---|---|---|---|---|
| | First | Second | Voltage | Efficiency | Luminous Efficiency | Efficiency | Lifespan |
| | Host | Host | (V) | (cd/A) | (%) | (lm/W) | (T90, hr) |
| Comparative Example 1 | — | H-2-80 | 6.0 | 21.3 | — | 11.2 | 3 |
| Device Example 1 | H-1-26 | H-2-80 | 5.0 | 27.3 | 28.2 | 17.1 | 48 |
| Comparative Example 2 | — | H-2-12 | 5.0 | 21.4 | — | 13.4 | 3 |
| Device Example 2 | H-1-26 | H-2-12 | 4.6 | 26.9 | 25.7 | 18.6 | 29 |

In addition, the power efficiency at a luminance of 1,000 nit, and/or the time taken for luminance to decrease from 100% to 98% at a luminance of 5,000 nit and at a constant current (lifespan; T98) of the OLEDs produced in Device Examples 3 to 5, and Comparative Examples 3 to 5 are provided in Table 2 below.

TABLE 2

| | | | Power | |
|---|---|---|---|---|
| | | | Efficiency | Lifespan |
| | First Host | Second Host | (Im/W) | (T98 · hr) |
| Device Example | H-1-26 | H-2-91 | 26.2 | 105 |
| 3 | | | | |
| Device Example | H-1-26 | H-2-90 | 26.9 | 120 |
| 4 | | | | |
| Device Example | H-1-52 | H-2-91 | 31.3 | 120 |
| 5 | | | | |
| Comparative | H-1-26 | Comparative | 23.9 | 27 |
| Example 3 | | Compound 1 | | |
| Comparative | H-1-26 | Comparative | 25.8 | 57 |
| Example 4 | | Compound 2 | | |
| Comparative | H-1-26 | Comparative | 23.8 | 38 |
| Example 5 | | Compound 3 | | |

From Table 1 above, it can be seen that the OLEDs comprising the plurality of host materials comprising a specific combination of compounds according to the present disclosure have low driving voltage and remarkably improved luminous efficiency, power efficiency and lifespan properties, compared to the conventional OLEDs.

Further, from Table 2 above, it can be seen that the OLEDs comprising the plurality of host materials comprising a specific combination of compounds according to the present disclosure show remarkably improved lifespan property, while having equivalent or higher power efficiency, compared to the conventional OLEDs.

The compounds used in the Device Examples and the Comparative Example are shown in Table 3 below,

TABLE 3

| | |
|---|---|
| Hole Injection Layer/ Hole Transport Layer | 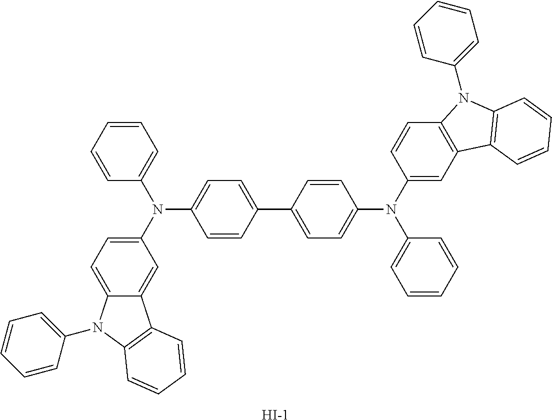 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; [I-]] |
| | 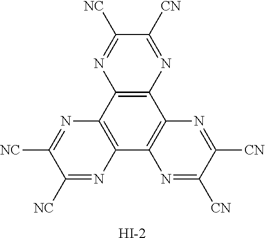 |
| | 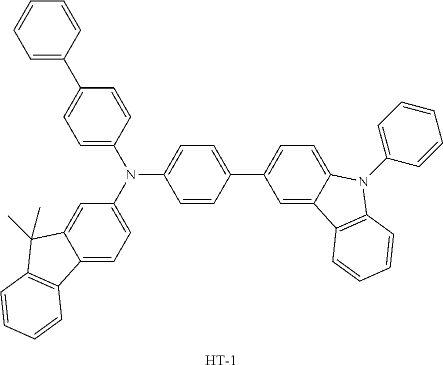 |
| | 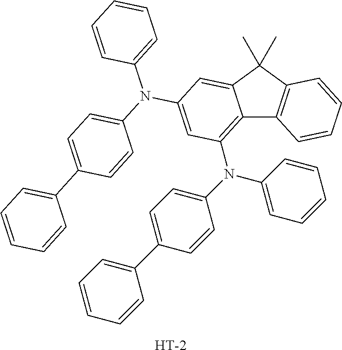 |
| Light-Emitting Layer | 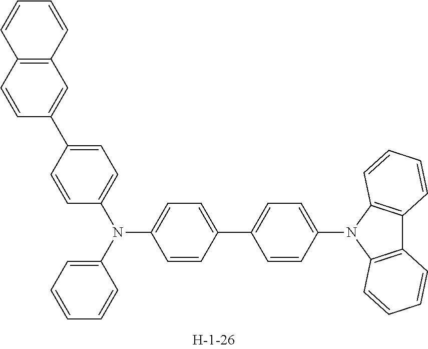 [SMILES: c1ccc(N(c2ccc(-c3ccc(-n4c5ccccc5c5ccccc54)cc3)cc2)c2ccc(-c3ccc4ccccc4c3)cc2)cc1] |
| | 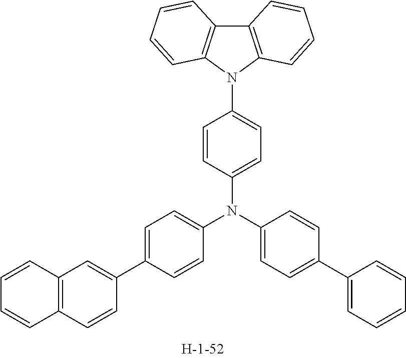 [SMILES: c1ccc(-c2ccc(N(c3ccc(-c4ccc5ccccc5c4)cc3)c3ccc(-n4c5ccccc5c5ccccc54)cc3)cc2)cc1] |
| | 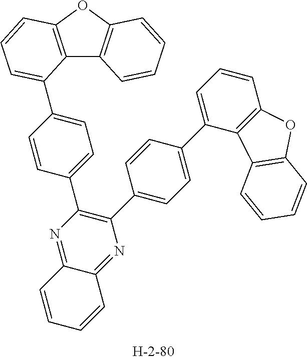 [SMILES: c1ccc2nc(-c3ccc(-c4cccc5oc6ccccc6c45)cc3)c(-c3ccc(-c4cccc5oc6ccccc6c45)cc3)nc2c1] |
| | 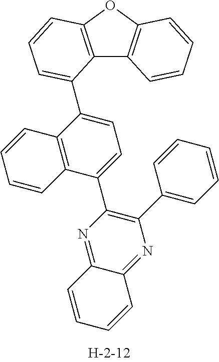 [SMILES: c1ccc(-c2nc3ccccc3nc2-c2ccc(-c3cccc4oc5ccccc5c34)c3ccccc23)cc1] |
| | 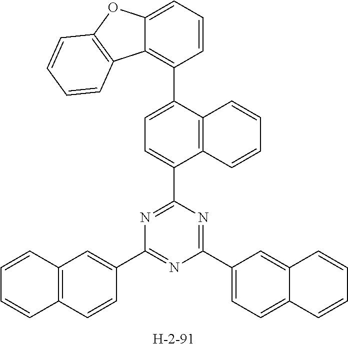 [SMILES: c1ccc2cc(-c3nc(-c4ccc5ccccc5c4)nc(-c4ccc(-c5cccc6oc7ccccc7c56)c5ccccc45)n3)ccc2c1] |
| | 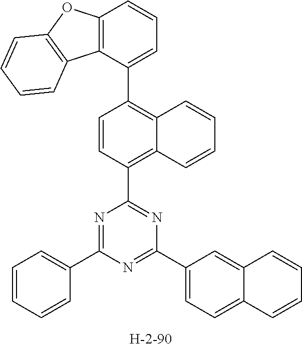 [SMILES: c1ccc(-c2nc(-c3ccc4ccccc4c3)nc(-c3ccc(-c4cccc5oc6ccccc6c45)c4ccccc34)n2)cc1] |
| | 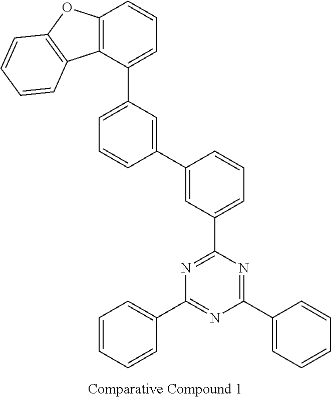 [SMILES: c1ccc(-c2nc(-c3ccccc3)nc(-c3cccc(-c4cccc(-c5cccc6oc7ccccc7c56)c4)c3)n2)cc1] |
| | 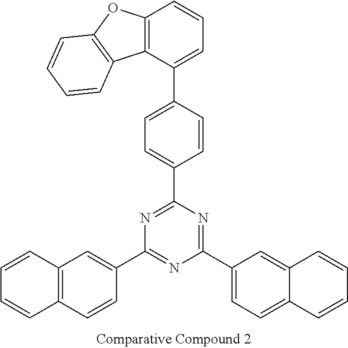 [SMILES: c1ccc2cc(-c3nc(-c4ccc(-c5cccc6oc7ccccc7c56)cc4)nc(-c4ccc5ccccc5c4)n3)ccc2c1] |
| | 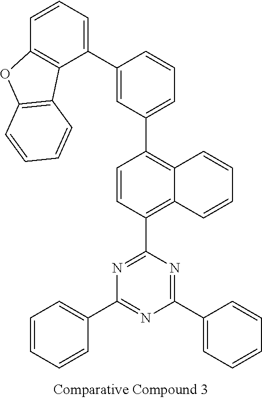 [SMILES: c1ccc(-c2nc(-c3ccccc3)nc(-c3ccc(-c4cccc(-c5cccc6oc7ccccc7c56)c4)c4ccccc34)n2)cc1] |
| | 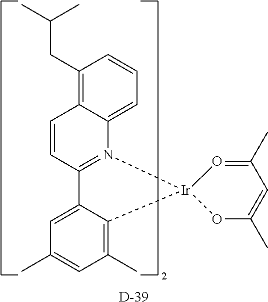 |
| Electron Transport Layer/ Electron Injection Layer | 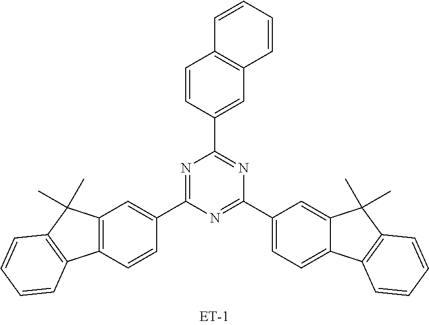 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 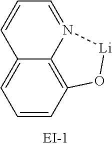 |

1. A plurality of host materials comprising a first host material comprising a compound represented by the following formula 1, and a second host material comprising a compound represented by the following formula 2: 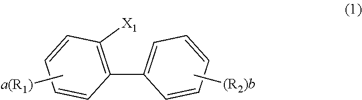
wherein
X₁ represents NR₃, CR₄R₅, O, or S;
R₁ and R₂, each independently, represent hydrogen, deuterium, a halogen, a cyano, a substituted or unsubstituted (C1-C30)alkyl, a substituted or unsubstituted (C6-C30)ayl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or 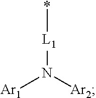
or two R₁'s, two R₂'s, or both thereof may be linked to each other to form a ring(s);
R₃ represents a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, or 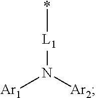
L₁, each independently, represents a single bond, a substituted or unsubstituted (C6-C30)arylene, or a substituted or unsubstituted (3- to 30-membered)heteroarylene, where if a plurality of L₁'s are present, each of L₁ may be the same or different;
Ar₁ and Ar₂, each independently, represent a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino, where if a plurality of Ar₁'s and a plurality of Ar₂'s, each independently, are present, each of Ar₁ and each of Ar₂ may be the same or different;
R₄ and R₅, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to each other to form a ring(s); and
a and b, each independently, represent an integer of 1 to 4, where if a and b are an integer of 2 or more, each of R₁ and each of R₂ may be the same or different; 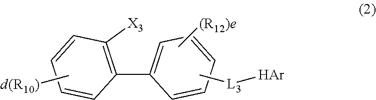
wherein
HAr represents 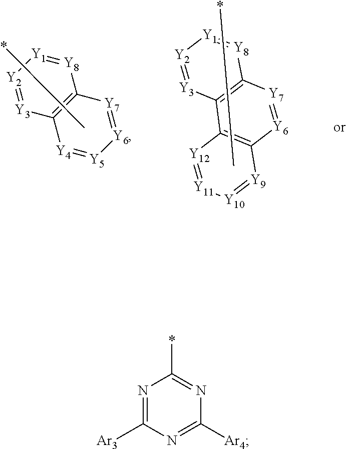
X₃ represents O or S;
L₃ represents a single bond, a substituted or unsubstituted (C6-C30)arylene, or a substituted or unsubstituted (3- to 30-membered)heteroarylene, with the proviso that if HAr represents 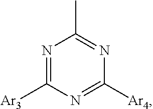
L₃ represents a substituted or unsubstituted naphthylene;
Y₁ to Y₁₂, each independently, represent CR₁₁ or N, with the proviso that at least one of Y₁ to Y₈ in 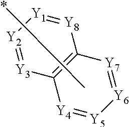
represents N, and at least one of Y₁ to Y₃, Y₆ to Y₈, and Y₉ to
Y₁₂ in 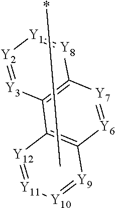
represents N;
R₁₀ and R₁₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or two R₁₀'s, two R₁₂'s, or both thereof may be linked to each other to form a ring(s);
R₁₁, Ar₃, and Ar₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)aylsilyl, where if a plurality of R₁₁'s are present, each of R₁₁ may be the same or different;
d represents an integer of 1 to 4, and e represents an integer of 1 to 3, where if d and e are an integer of 2 or more, each of R₁₀ and each of R₁₂ may be the same or different; and
* represents a bonding site.

2. The plurality of host materials according to claim 1, wherein the substituents of the substituted alkyl, the substituted aryl, the substituted arylene, the substituted heteroaryl, the substituted heteroarylene, the substituted cycloalkyl, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-arylamino, and the substituted alkylarylamino in R₁ to R₅, R₁₀ to R₁₂, L₁, L₃, and Ar₁ to Ar₄, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-030)alkynyl; a (C1-C30)alkoxy; a (C1-C30)alkylthio; a (C3-C30)cycloalkyl: a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of a (C1-C30)alkyl(s) and a (3- to 30-membered)heteroaryl(s); a tri(C1-C30)alkylsilyl; a tri(C6-C30)arylsilyl; a di(C1-030)alkyl(C6-030)arylsilyl; a (C1-C30)alkyldi(C6-C30)arylsilyl; an amino; a mono- or di-(C1-C30)alkylamino; a mono- or di-(C6-C30)arylamino; a (C1-C30)alkyl(C6-C30)arylamino; a (C1-C30)alkylcarbonyl; a (C1-C30)alkoxycarbonyl; a (C6-C30)arylcarbonyl: a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The plurality of host materials according to claim 1, wherein in the definition of R₁ and R₂, if two R₁'s, two R₂'s, or both thereof are linked to each other to form a ring(s), the formed ring is 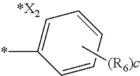
or a benzene ring(s);
X₂ represents NR₇, CR₈R₀, O, or S;
R₆ is the same as the definition of R₁ and R₂;
R₇ is the same as the definition of R₃;
R₅ and R₉, each independently, are the same as the definition of R₄ and R₅;
c represents an integer of 1 to 4, where if c is an integer of 2 or more, each of R₅ may be the same or different; and
* represents a bonding site.

4. The plurality of host materials according to claim 1, wherein formula 1 is represented by at least one of the following formulas 1-1 to 1-3: 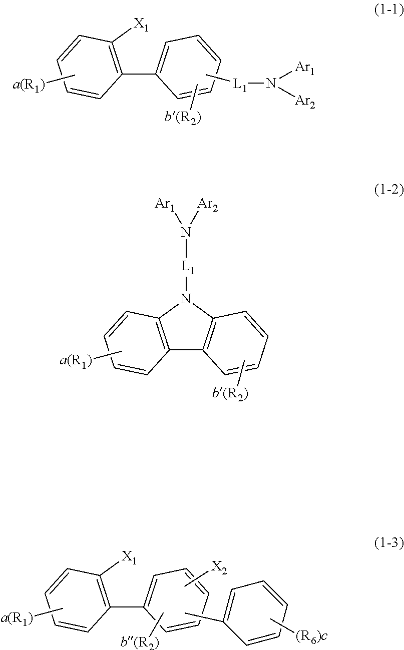
wherein
X₁, R₁, R₂, L₁, Ar₁, Ar₂, a, and b are as defined in claim 1:
X₂ represents NR₇, CR₈R₉, O, or S;
R₆ is the same as the definition of R₁ and R₂;
R₇ is the same as the definition of R₃;
R₅ and R₉, each independently, are the same as the definition of R₄ and R₅;
b′ represents an integer of 1 to 3, b″ represents 1 or 2, and c represents an integer of 1 to 4, where if b′, b″, and c are an integer of 2 or more, each of R₂ and each of R₆ may be the same or different; and
* represents a bonding site.

5. The plurality of host materials according to claim 1, wherein formula 2 is represented by at least one of the following formulas 2-1 to 2-10: 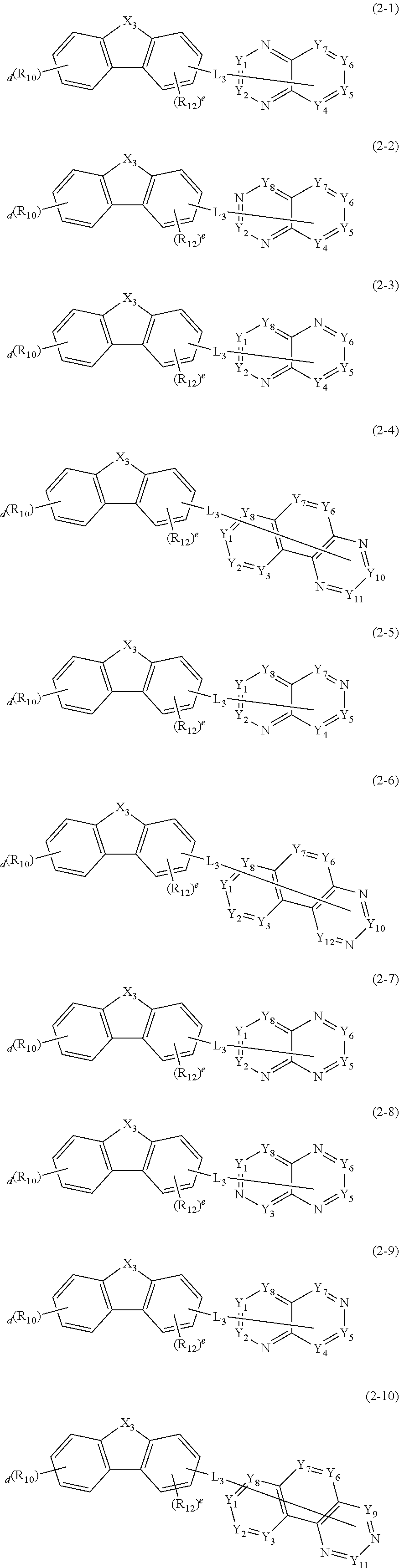
wherein
Y₁ to Y₈, Y₁₀, and Y₁₁, each independently, represent CR₁₁ or N; and
X₃, L₃, R₁₀ to R₁₂, d, and e are as defined in claim 1.

6. The plurality of host materials according to claim 1, wherein formula 2 is represented by the following formula 2-11; 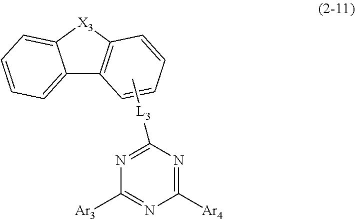
wherein
X₃ represents O or S;
L₃ represents an unsubstituted naphthylene; and
Ar₃ and Ar₄, each independently, represent an unsubstituted phenyl, an unsubstituted naphthyl, an unsubstituted biphenyl, or an unsubstituted terphenyl.

7. The plurality of host materials according to claim 1, wherein the compound represented by formula 1 is at least one selected from the following compounds:

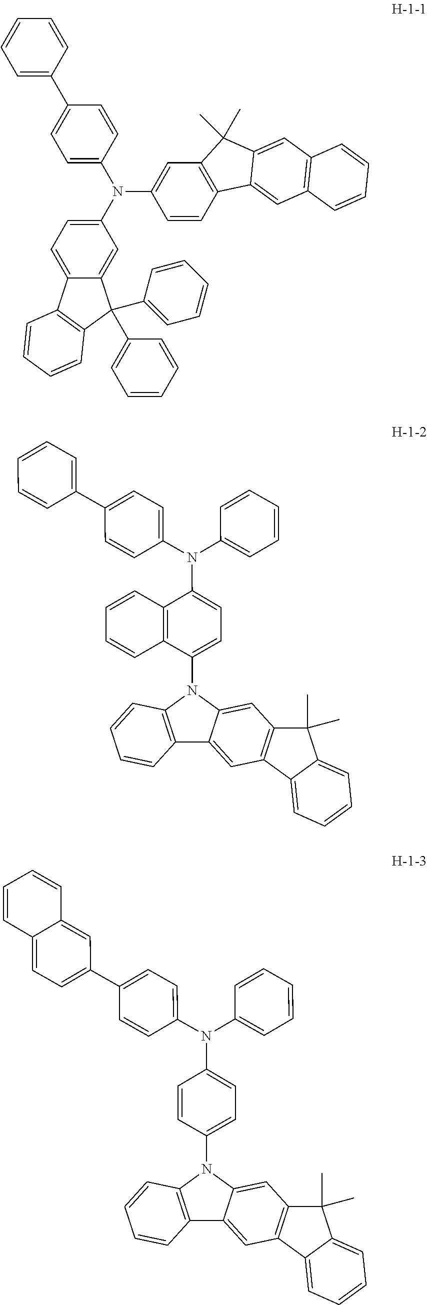 [SMILES: CC1(C)c2cc(N(c3ccc(-c4ccccc4)cc3)c3ccc4c(c3)C(c3ccccc3)(c3ccccc3)c3ccccc3-4)ccc2-c2cc3ccccc3cc21 ; CC1(C)c2ccccc2-c2cc3c4ccccc4n(-c4ccc(N(c5ccccc5)c5ccc(-c6ccccc6)cc5)c5ccccc45)c3cc21 ; CC1(C)c2ccccc2-c2cc3c4ccccc4n(-c4ccc(N(c5ccccc5)c5ccc(-c6ccc7ccccc7c6)cc5)cc4)c3cc21]

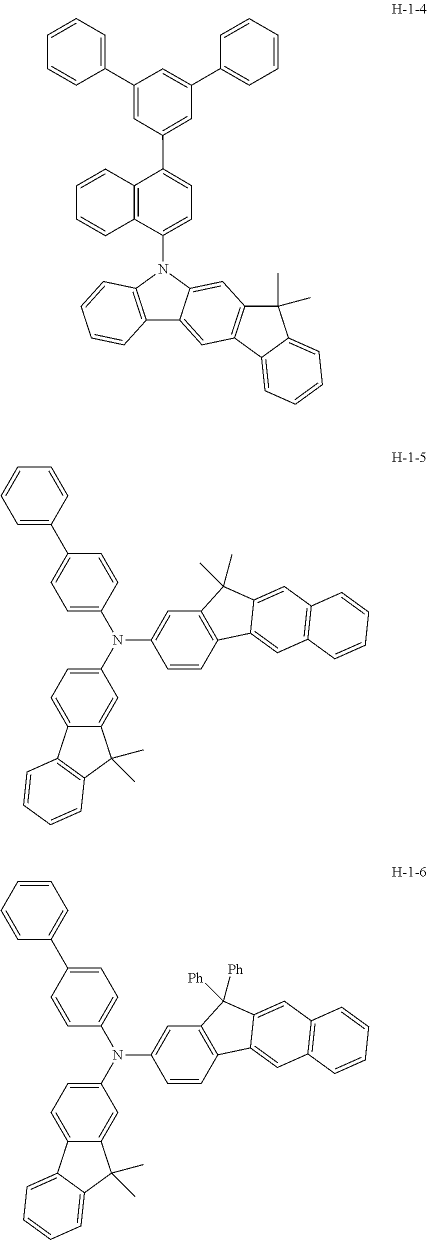 [SMILES: CC1(C)c2ccccc2-c2cc3c4ccccc4n(-c4ccc(-c5cc(-c6ccccc6)cc(-c6ccccc6)c5)c5ccccc45)c3cc21 ; CC1(C)c2ccccc2-c2ccc(N(c3ccc(-c4ccccc4)cc3)c3ccc4c(c3)C(C)(C)c3cc5ccccc5cc3-4)cc21 ; CC1(C)c2ccccc2-c2ccc(N(c3ccc(-c4ccccc4)cc3)c3ccc4c(c3)C(c3ccccc3)(c3ccccc3)c3cc5ccccc5cc3-4)cc21]

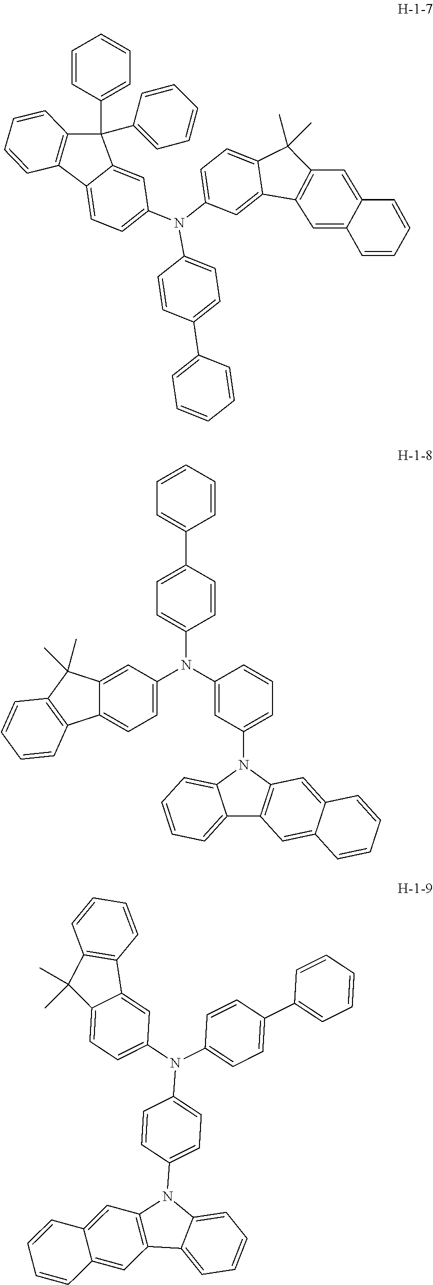 [SMILES: CC1(C)c2ccc(N(c3ccc(-c4ccccc4)cc3)c3ccc4c(c3)C(c3ccccc3)(c3ccccc3)c3ccccc3-4)cc2-c2cc3ccccc3cc21 ; CC1(C)c2ccccc2-c2ccc(N(c3ccc(-c4ccccc4)cc3)c3cccc(-n4c5ccccc5c5cc6ccccc6cc54)c3)cc21 ; CC1(C)c2ccccc2-c2cc(N(c3ccc(-c4ccccc4)cc3)c3ccc(-n4c5ccccc5c5cc6ccccc6cc54)cc3)ccc21]

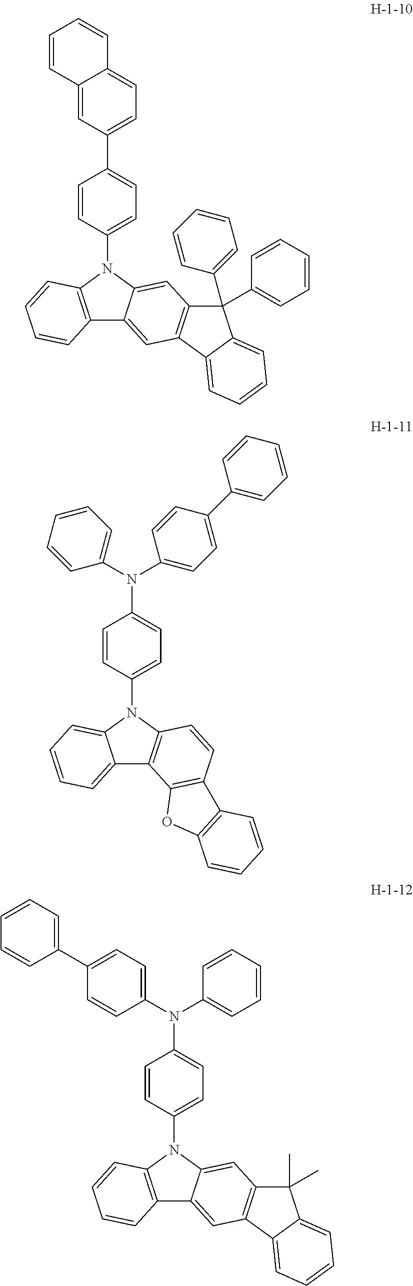

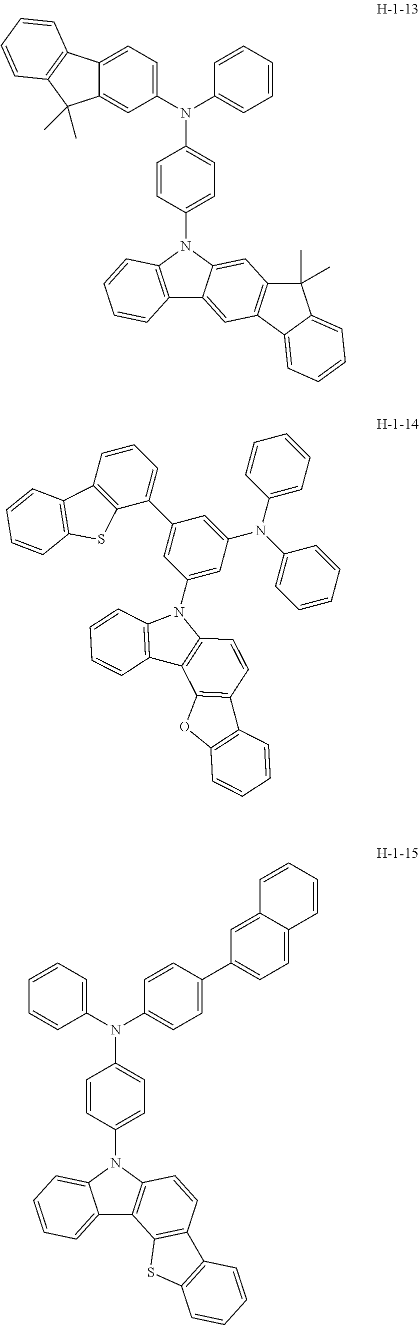 [SMILES: CC1(C)c2ccccc2-c2ccc(N(c3ccccc3)c3ccc(-n4c5ccccc5c5cc6c(cc54)C(C)(C)c4ccccc4-6)cc3)cc21 ; c1ccc(N(c2ccccc2)c2cc(-c3cccc4c3sc3ccccc34)cc(-n3c4ccccc4c4c5oc6ccccc6c5ccc43)c2)cc1 ; c1ccc(N(c2ccc(-c3ccc4ccccc4c3)cc2)c2ccc(-n3c4ccccc4c4c5sc6ccccc6c5ccc43)cc2)cc1]

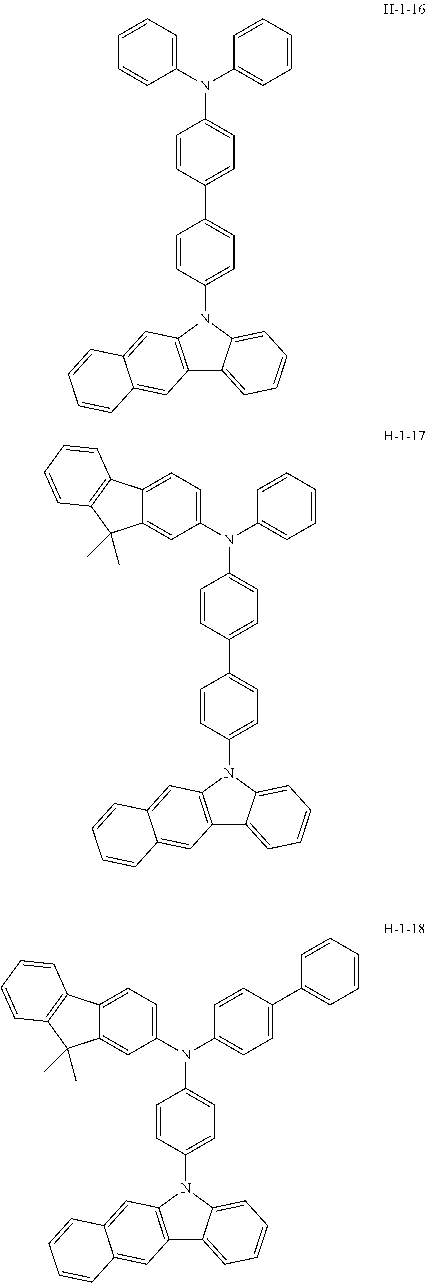

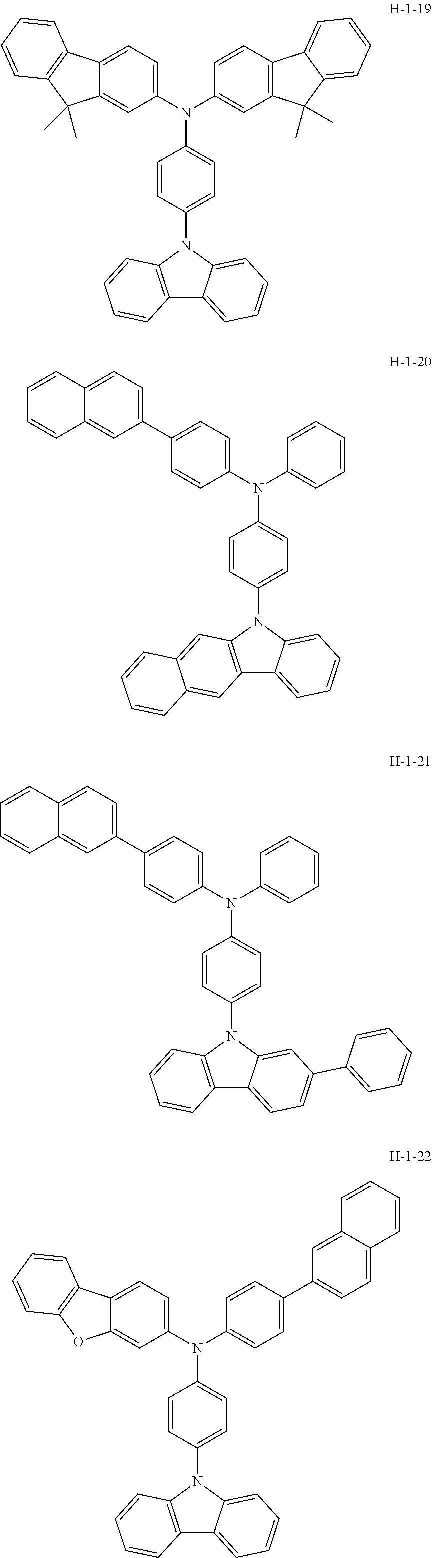 [SMILES: CC1(C)c2ccccc2-c2ccc(N(c3ccc(-n4c5ccccc5c5ccccc54)cc3)c3ccc4c(c3)C(C)(C)c3ccccc3-4)cc21 ; c1ccc(N(c2ccc(-c3ccc4ccccc4c3)cc2)c2ccc(-n3c4ccccc4c4cc5ccccc5cc43)cc2)cc1 ; c1ccc(-c2ccc3c4ccccc4n(-c4ccc(N(c5ccccc5)c5ccc(-c6ccc7ccccc7c6)cc5)cc4)c3c2)cc1 ; c1ccc2cc(-c3ccc(N(c4ccc(-n5c6ccccc6c6ccccc65)cc4)c4ccc5c(c4)oc4ccccc45)cc3)ccc2c1]

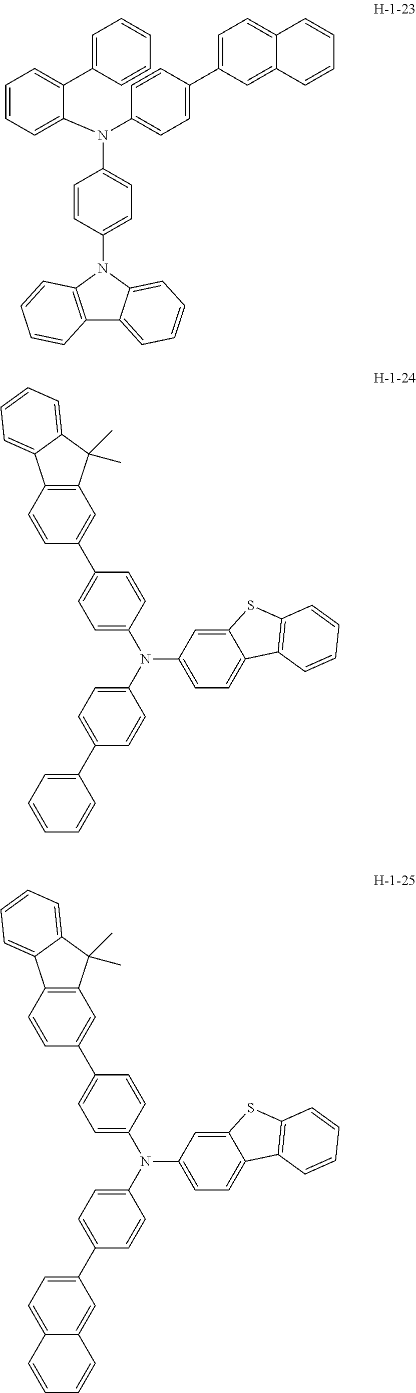 [SMILES: c1ccc(-c2ccccc2N(c2ccc(-c3ccc4ccccc4c3)cc2)c2ccc(-n3c4ccccc4c4ccccc43)cc2)cc1 ; CC1(C)c2ccccc2-c2ccc(-c3ccc(N(c4ccc(-c5ccccc5)cc4)c4ccc5c(c4)sc4ccccc45)cc3)cc21 ; CC1(C)c2ccccc2-c2ccc(-c3ccc(N(c4ccc(-c5ccc6ccccc6c5)cc4)c4ccc5c(c4)sc4ccccc45)cc3)cc21]

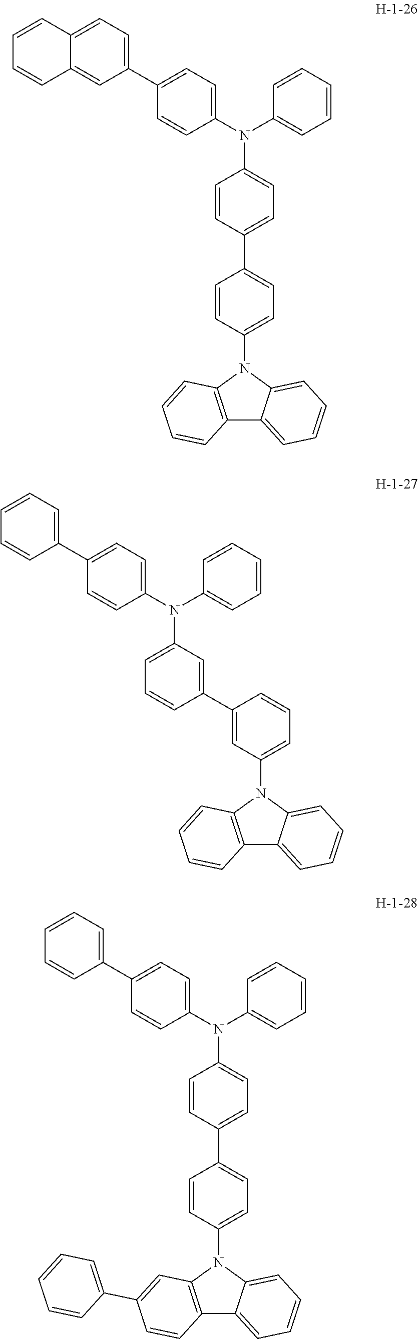 [SMILES: c1ccc(N(c2ccc(-c3ccc(-n4c5ccccc5c5ccccc54)cc3)cc2)c2ccc(-c3ccc4ccccc4c3)cc2)cc1 ; c1ccc(-c2ccc(N(c3ccccc3)c3cccc(-c4cccc(-n5c6ccccc6c6ccccc65)c4)c3)cc2)cc1 ; c1ccc(-c2ccc(N(c3ccccc3)c3ccc(-c4ccc(-n5c6ccccc6c6ccc(-c7ccccc7)cc65)cc4)cc3)cc2)cc1]

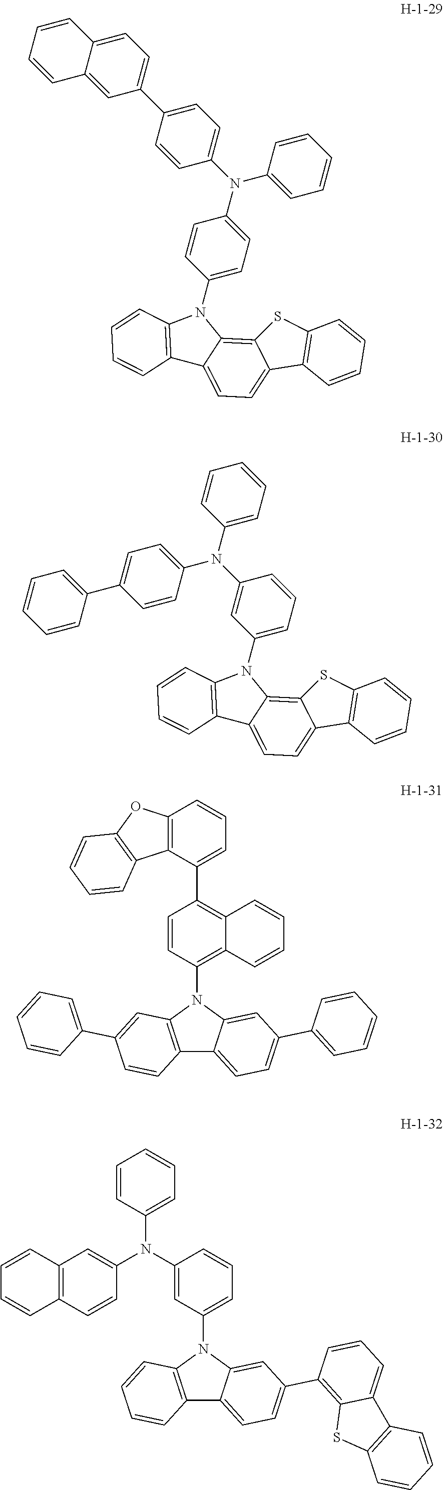 [SMILES: c1ccc(N(c2ccc(-c3ccc4ccccc4c3)cc2)c2ccc(-n3c4ccccc4c4ccc5c6ccccc6sc5c43)cc2)cc1 ; c1ccc(-c2ccc(N(c3ccccc3)c3cccc(-n4c5ccccc5c5ccc6c7ccccc7sc6c54)c3)cc2)cc1 ; c1ccc(-c2ccc3c4ccc(-c5ccccc5)cc4n(-c4ccc(-c5cccc6oc7ccccc7c56)c5ccccc45)c3c2)cc1 ; c1ccc(N(c2cccc(-n3c4ccccc4c4ccc(-c5cccc6c5sc5ccccc56)cc43)c2)c2ccc3ccccc3c2)cc1]

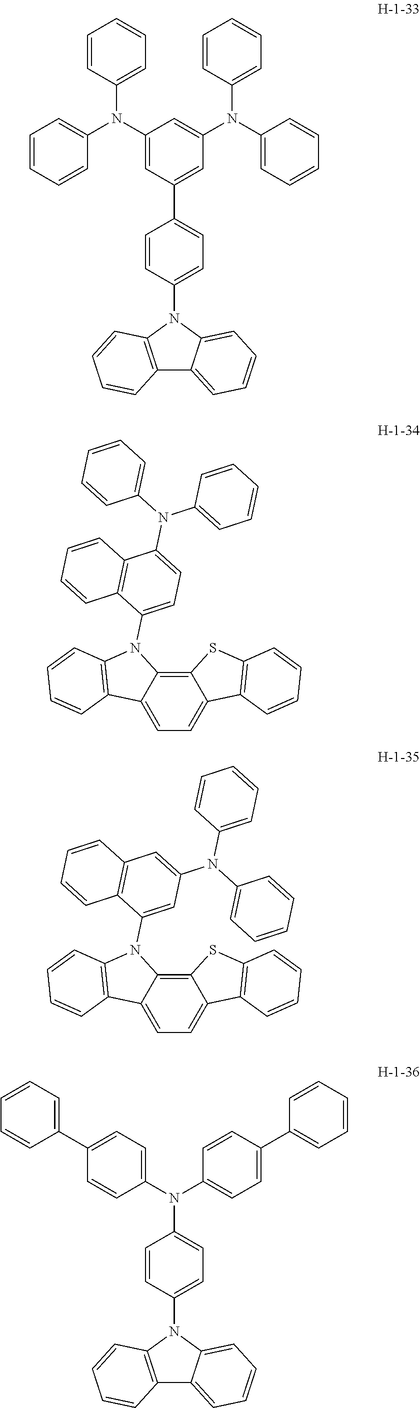

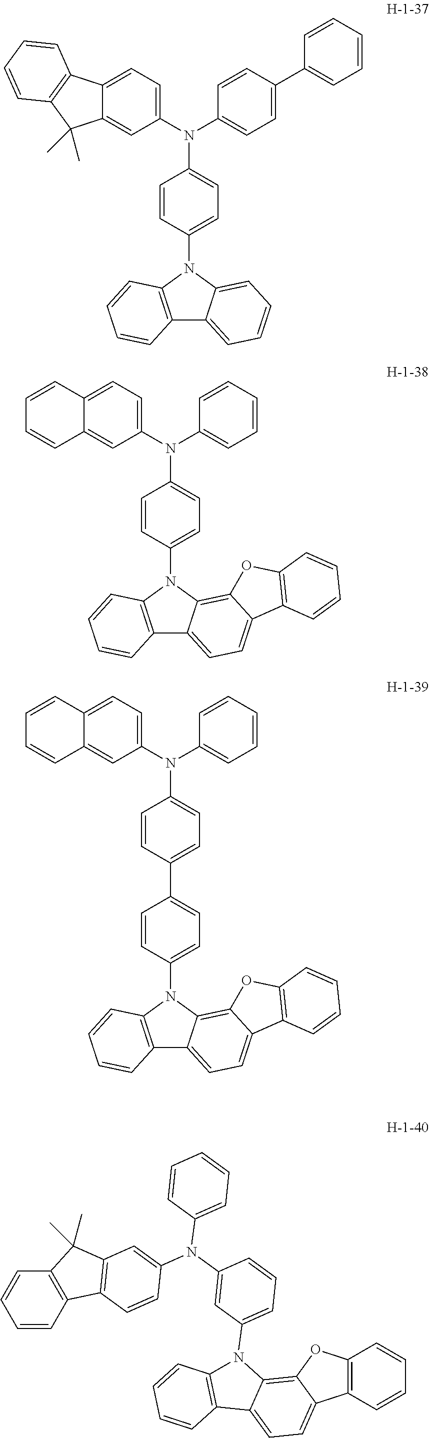

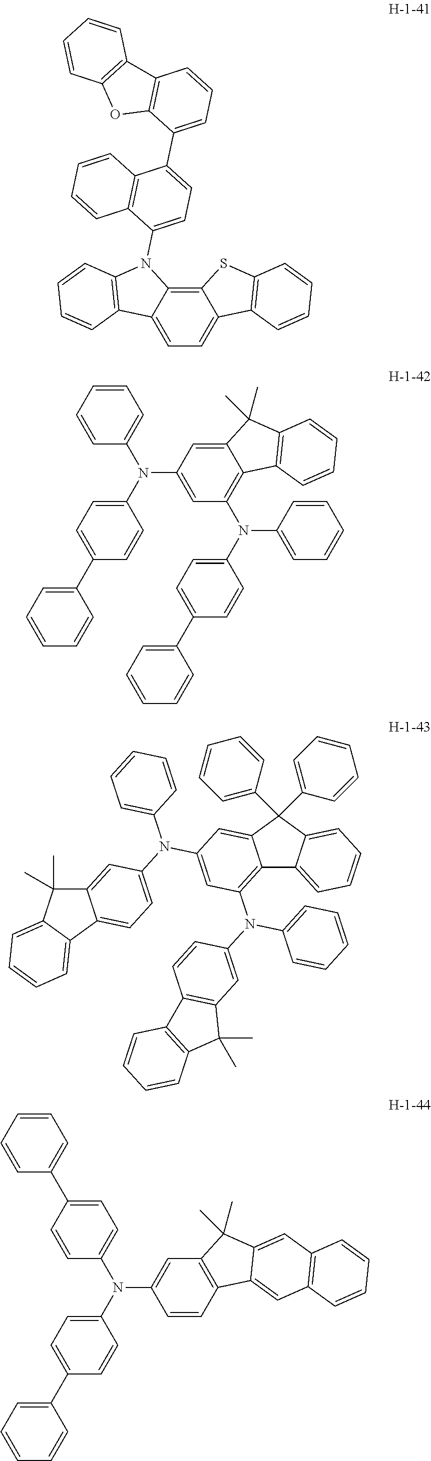 [SMILES: c1ccc2c(c1)oc1c(-c3ccc(-n4c5ccccc5c5ccc6c7ccccc7sc6c54)c4ccccc34)cccc12 ; CC1(C)c2ccccc2-c2c(N(c3ccccc3)c3ccc(-c4ccccc4)cc3)cc(N(c3ccccc3)c3ccc(-c4ccccc4)cc3)cc21 ; CC1(C)c2ccccc2-c2ccc(N(c3ccccc3)c3cc(N(c4ccccc4)c4ccc5c(c4)C(C)(C)c4ccccc4-5)c4c(c3)C(c3ccccc3)(c3ccccc3)c3ccccc3-4)cc21 ; CC1(C)c2cc(N(c3ccc(-c4ccccc4)cc3)c3ccc(-c4ccccc4)cc3)ccc2-c2cc3ccccc3cc21]

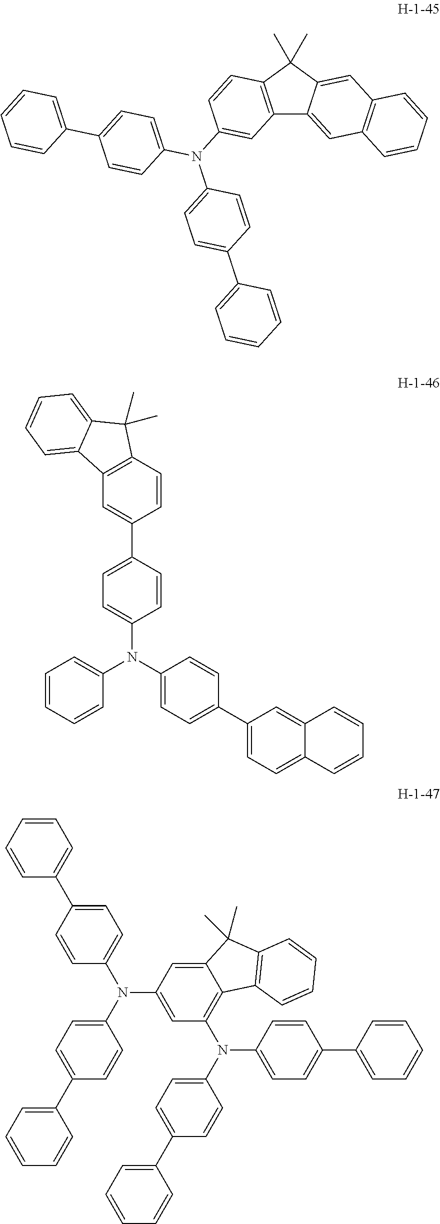 [SMILES: CC1(C)c2ccc(N(c3ccc(-c4ccccc4)cc3)c3ccc(-c4ccccc4)cc3)cc2-c2cc3ccccc3cc21 ; CC1(C)c2ccccc2-c2cc(-c3ccc(N(c4ccccc4)c4ccc(-c5ccc6ccccc6c5)cc4)cc3)ccc21 ; CC1(C)c2ccccc2-c2c(N(c3ccc(-c4ccccc4)cc3)c3ccc(-c4ccccc4)cc3)cc(N(c3ccc(-c4ccccc4)cc3)c3ccc(-c4ccccc4)cc3)cc21]

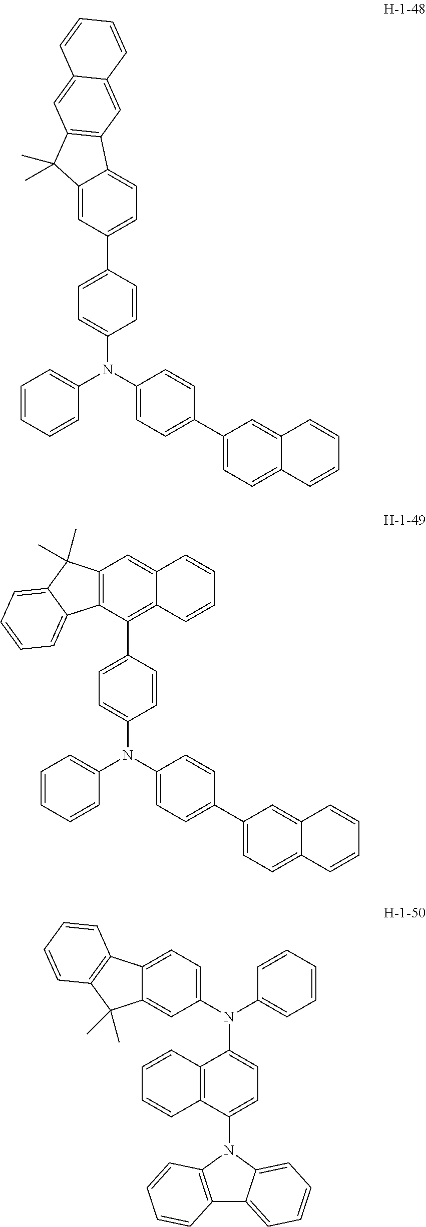 [SMILES: CC1(C)c2cc(-c3ccc(N(c4ccccc4)c4ccc(-c5ccc6ccccc6c5)cc4)cc3)ccc2-c2cc3ccccc3cc21 ; CC1(C)c2ccccc2-c2c1cc1ccccc1c2-c1ccc(N(c2ccccc2)c2ccc(-c3ccc4ccccc4c3)cc2)cc1 ; CC1(C)c2ccccc2-c2ccc(N(c3ccccc3)c3ccc(-n4c5ccccc5c5ccccc54)c4ccccc34)cc21]

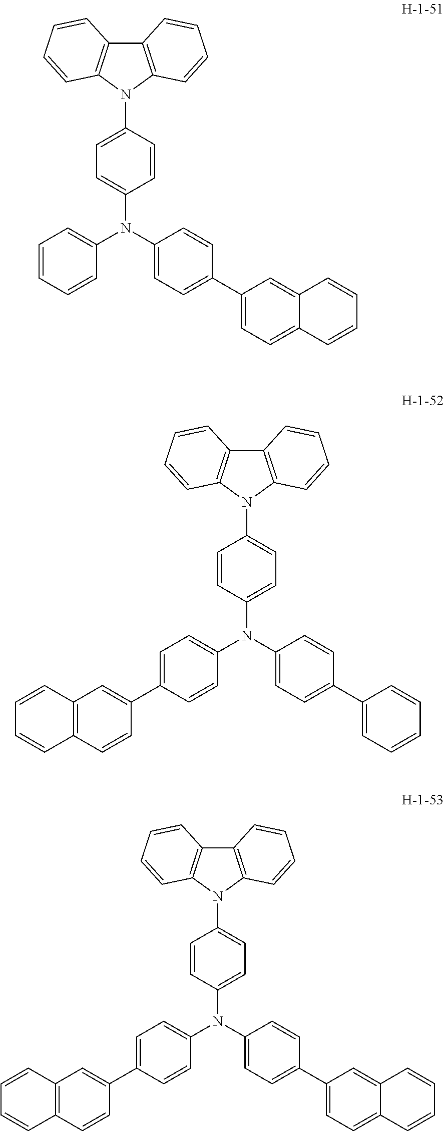 [SMILES: c1ccc(N(c2ccc(-c3ccc4ccccc4c3)cc2)c2ccc(-n3c4ccccc4c4ccccc43)cc2)cc1 ; c1ccc(-c2ccc(N(c3ccc(-c4ccc5ccccc5c4)cc3)c3ccc(-n4c5ccccc5c5ccccc54)cc3)cc2)cc1 ; c1ccc2cc(-c3ccc(N(c4ccc(-c5ccc6ccccc6c5)cc4)c4ccc(-n5c6ccccc6c6ccccc65)cc4)cc3)ccc2c1]

8. The plurality of host materials according to claim 1, wherein the compound represented by formula 2 is at least one selected from the following compounds:

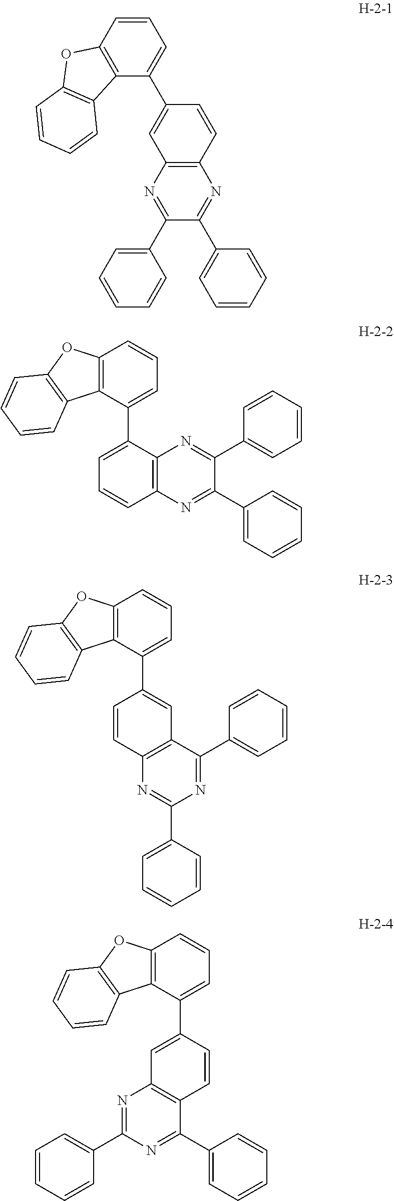

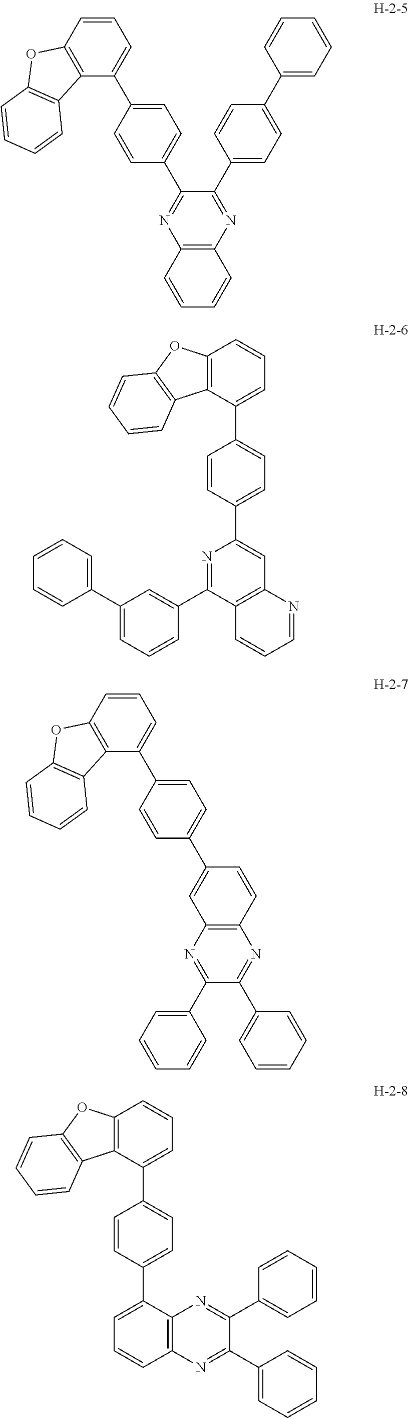

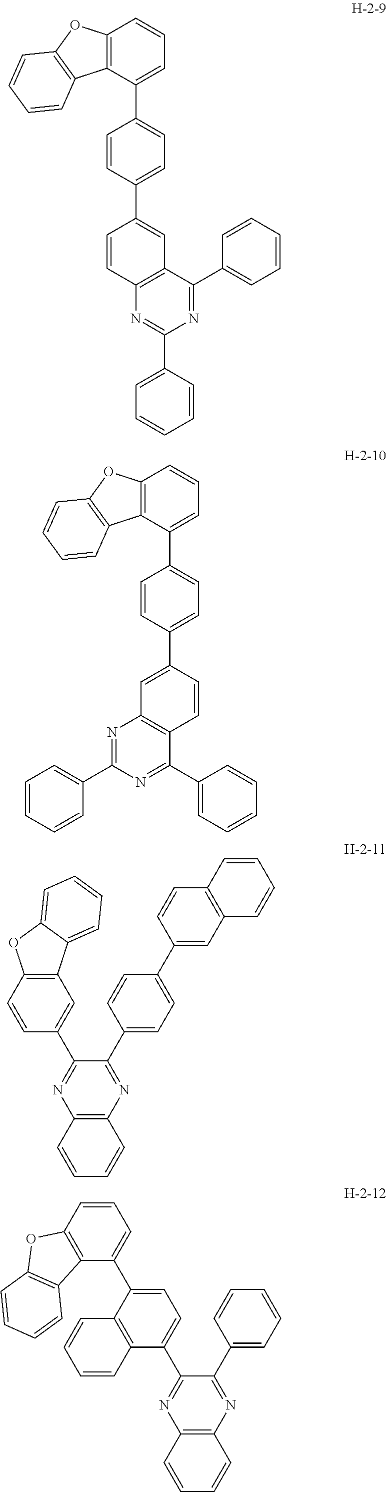

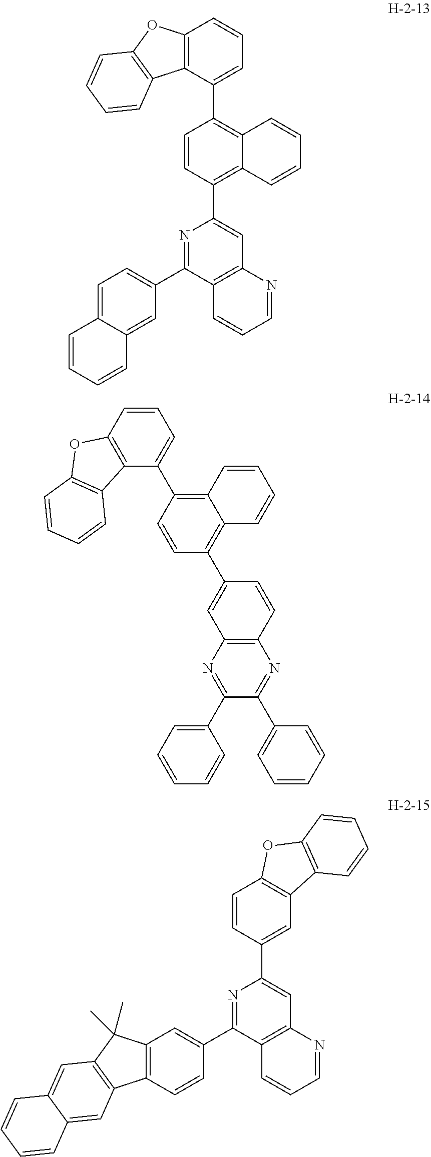

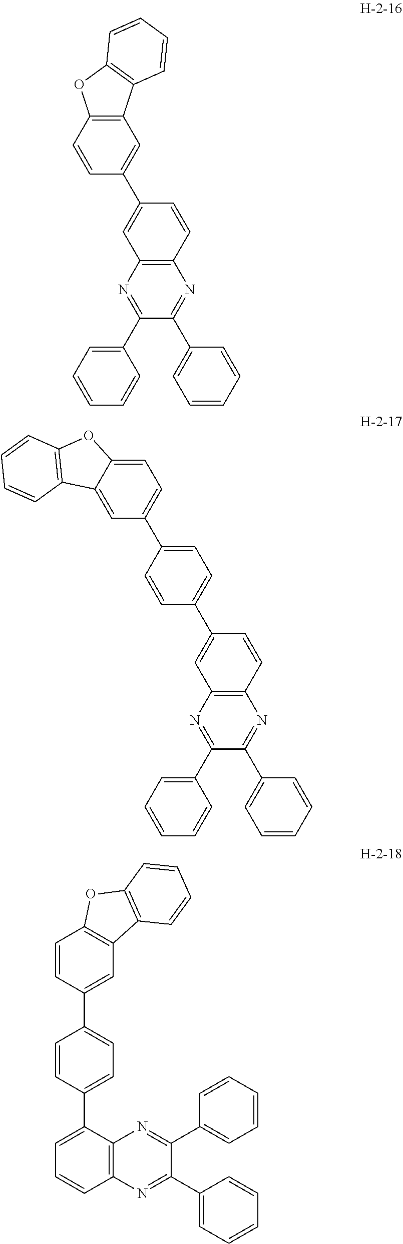

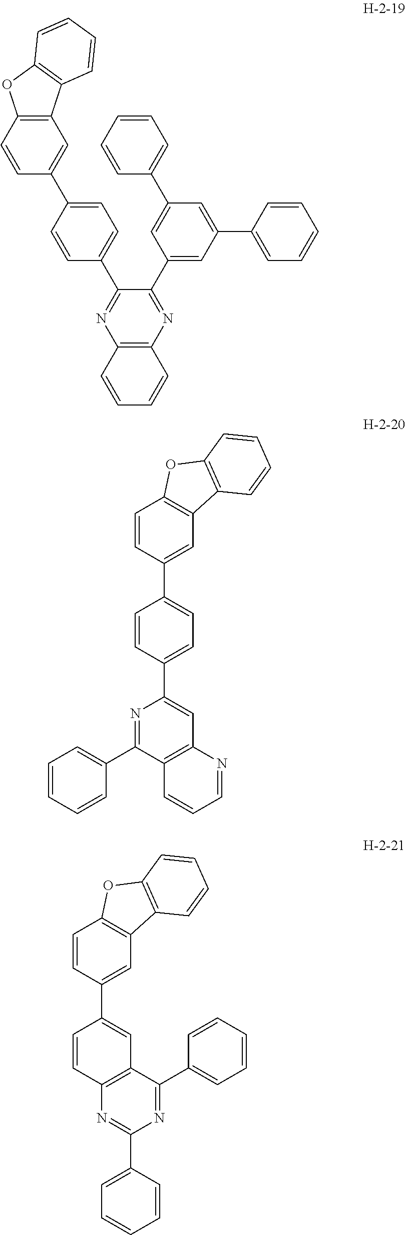

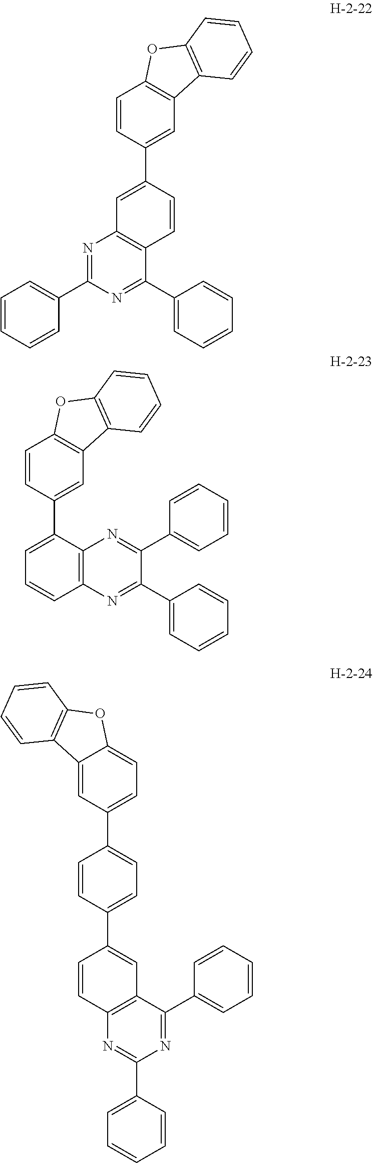 [SMILES: c1ccc(-c2nc(-c3ccccc3)c3ccc(-c4ccc5oc6ccccc6c5c4)cc3n2)cc1 ; c1ccc(-c2nc3cccc(-c4ccc5oc6ccccc6c5c4)c3nc2-c2ccccc2)cc1 ; c1ccc(-c2nc(-c3ccccc3)c3cc(-c4ccc(-c5ccc6oc7ccccc7c6c5)cc4)ccc3n2)cc1]

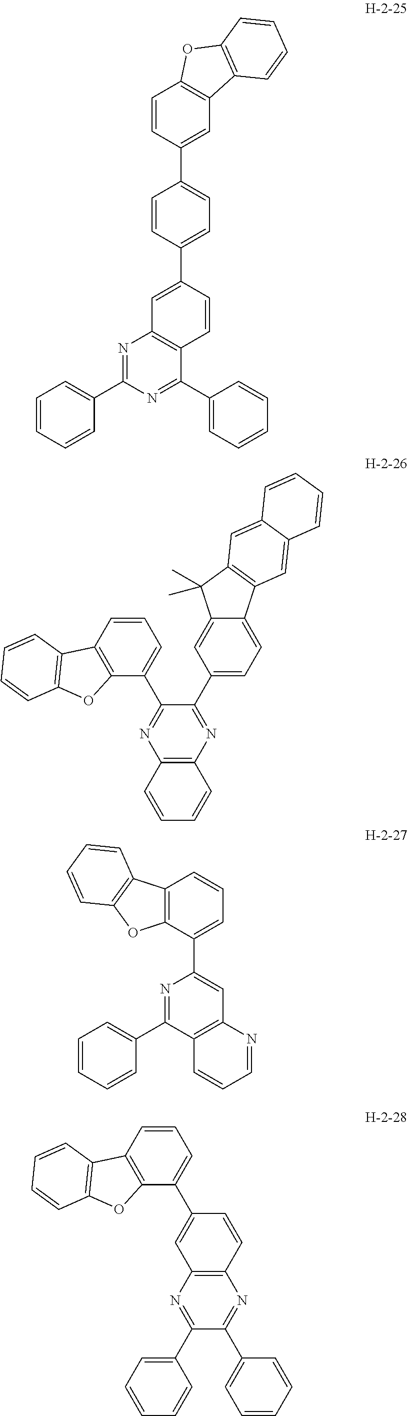 [SMILES: c1ccc(-c2nc(-c3ccccc3)c3ccc(-c4ccc(-c5ccc6oc7ccccc7c6c5)cc4)cc3n2)cc1 ; CC1(C)c2cc(-c3nc4ccccc4nc3-c3cccc4c3oc3ccccc34)ccc2-c2cc3ccccc3cc21 ; c1ccc(-c2nc(-c3cccc4c3oc3ccccc34)cc3ncccc23)cc1 ; c1ccc(-c2nc3ccc(-c4cccc5c4oc4ccccc45)cc3nc2-c2ccccc2)cc1]

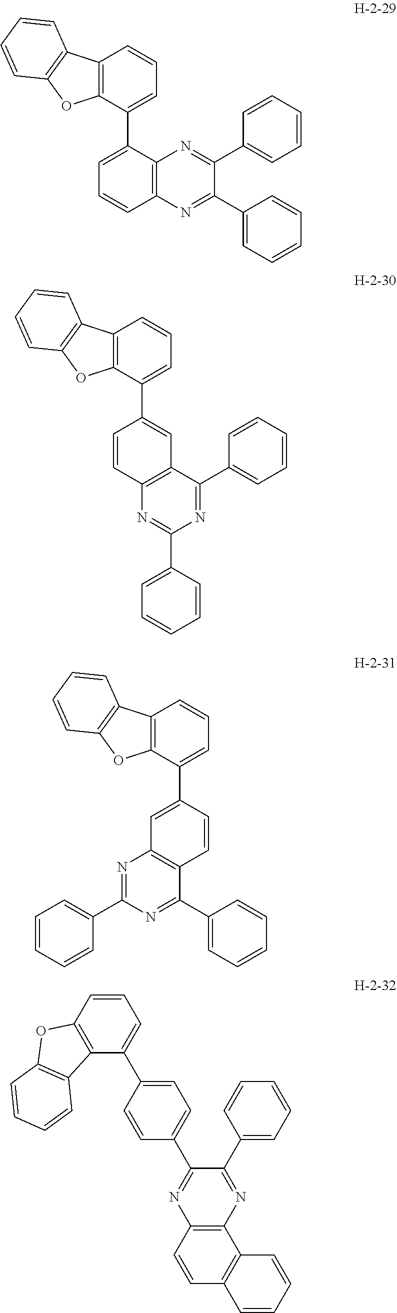

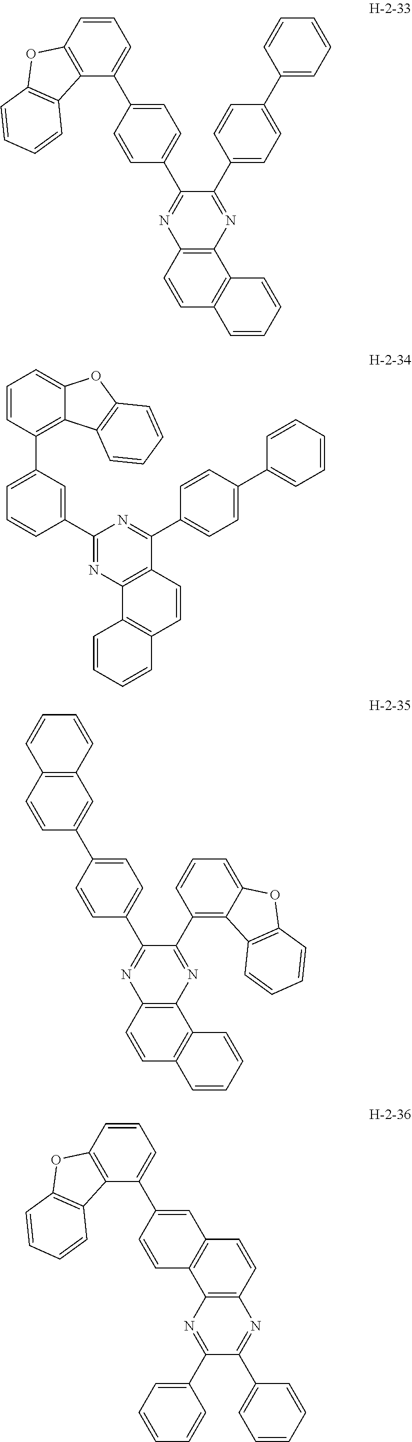 [SMILES: c1ccc(-c2ccc(-c3nc4c(ccc5ccccc54)nc3-c3ccc(-c4cccc5oc6ccccc6c45)cc3)cc2)cc1 ; c1ccc(-c2ccc(-c3nc(-c4cccc(-c5cccc6oc7ccccc7c56)c4)nc4c3ccc3ccccc34)cc2)cc1 ; c1ccc2cc(-c3ccc(-c4nc5ccc6ccccc6c5nc4-c4cccc5oc6ccccc6c45)cc3)ccc2c1 ; c1ccc(-c2nc3ccc4cc(-c5cccc6oc7ccccc7c56)ccc4c3nc2-c2ccccc2)cc1]

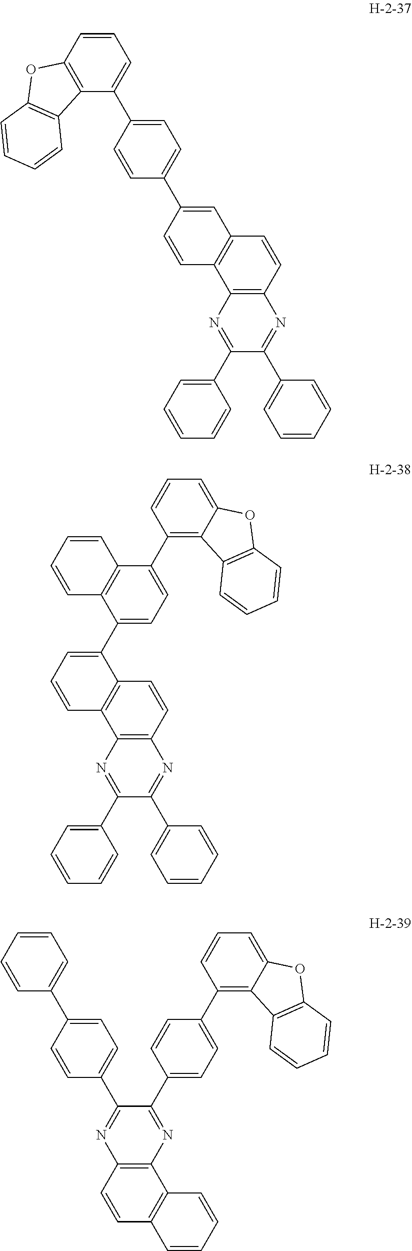

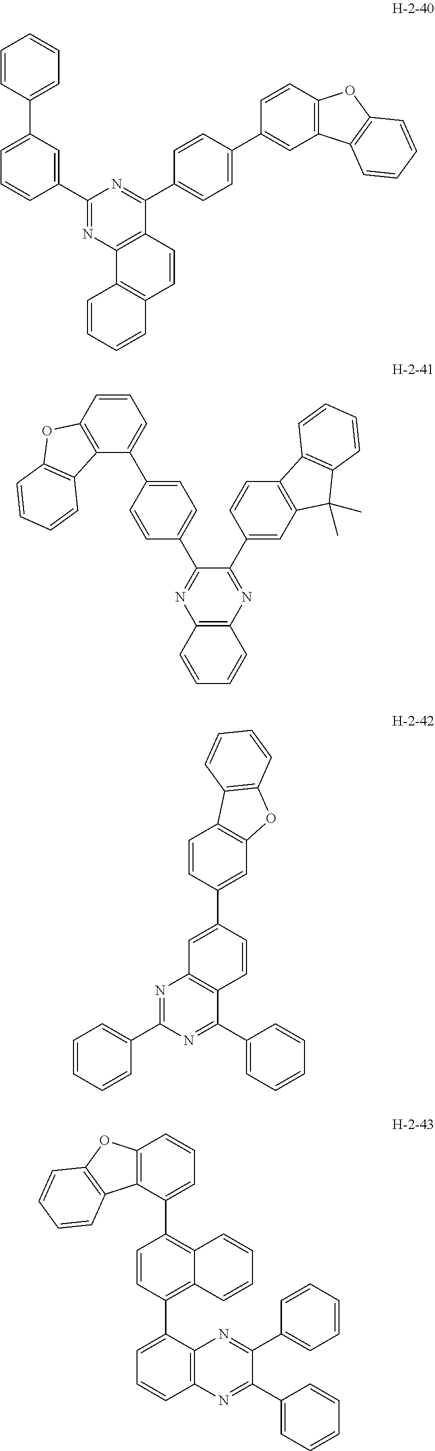

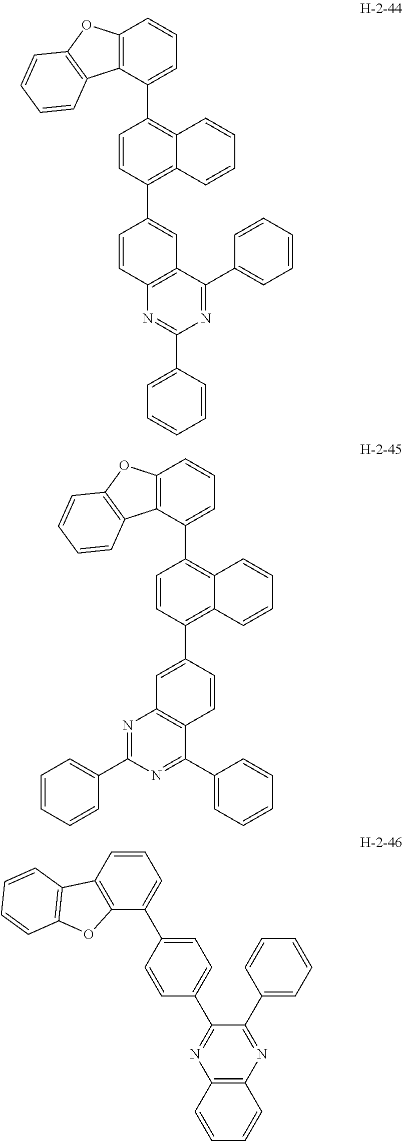

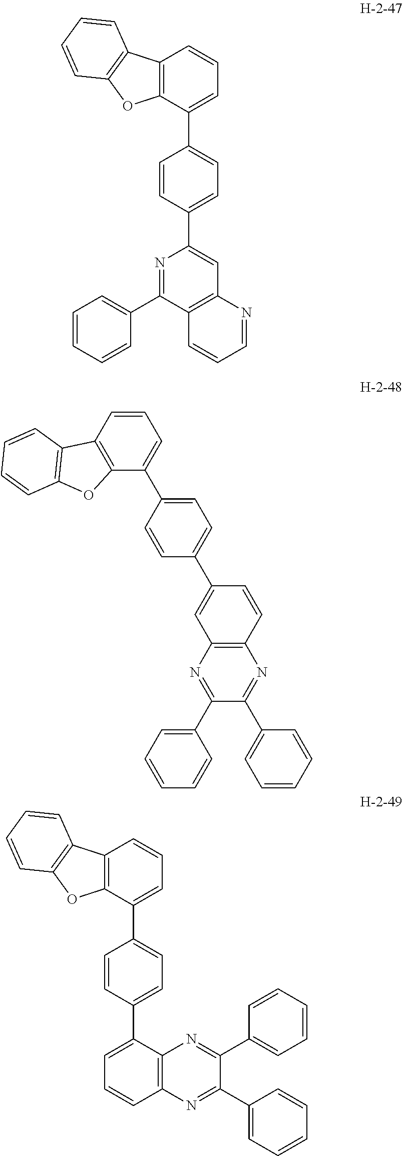 [SMILES: c1ccc(-c2nc(-c3ccc(-c4cccc5c4oc4ccccc45)cc3)cc3ncccc23)cc1 ; c1ccc(-c2nc3ccc(-c4ccc(-c5cccc6c5oc5ccccc56)cc4)cc3nc2-c2ccccc2)cc1 ; c1ccc(-c2nc3cccc(-c4ccc(-c5cccc6c5oc5ccccc56)cc4)c3nc2-c2ccccc2)cc1]

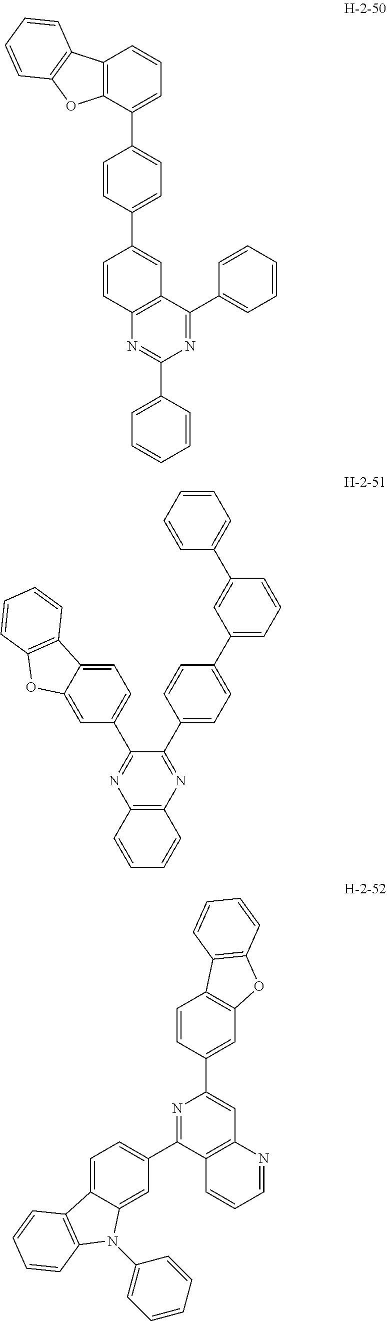

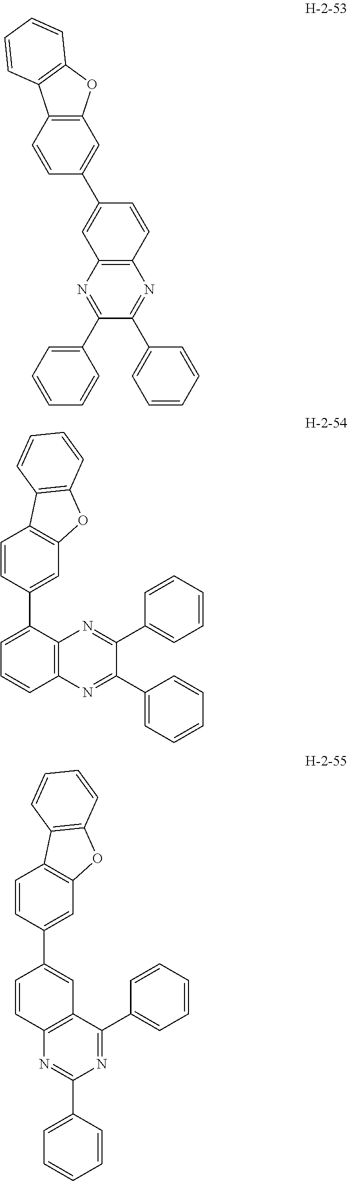

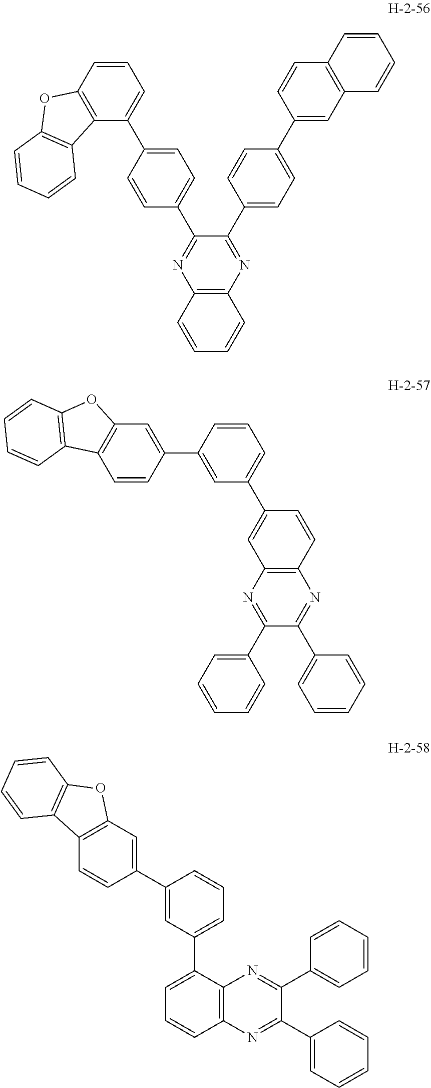 [SMILES: c1ccc2cc(-c3ccc(-c4nc5ccccc5nc4-c4ccc(-c5cccc6oc7ccccc7c56)cc4)cc3)ccc2c1 ; c1ccc(-c2nc3ccc(-c4cccc(-c5ccc6c(c5)oc5ccccc56)c4)cc3nc2-c2ccccc2)cc1 ; c1ccc(-c2nc3cccc(-c4cccc(-c5ccc6c(c5)oc5ccccc56)c4)c3nc2-c2ccccc2)cc1]

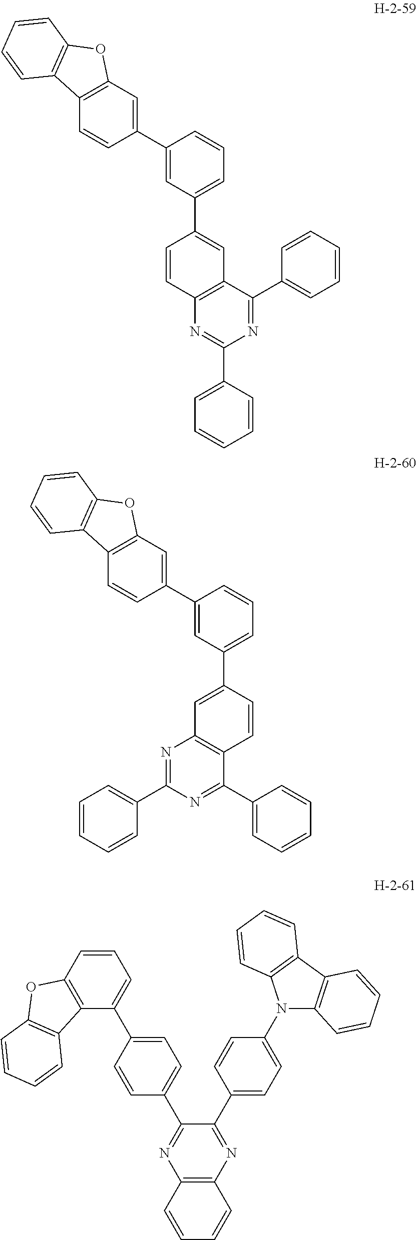 [SMILES: c1ccc(-c2nc(-c3ccccc3)c3cc(-c4cccc(-c5ccc6c(c5)oc5ccccc56)c4)ccc3n2)cc1 ; c1ccc(-c2nc(-c3ccccc3)c3ccc(-c4cccc(-c5ccc6c(c5)oc5ccccc56)c4)cc3n2)cc1 ; c1ccc2nc(-c3ccc(-n4c5ccccc5c5ccccc54)cc3)c(-c3ccc(-c4cccc5oc6ccccc6c45)cc3)nc2c1]

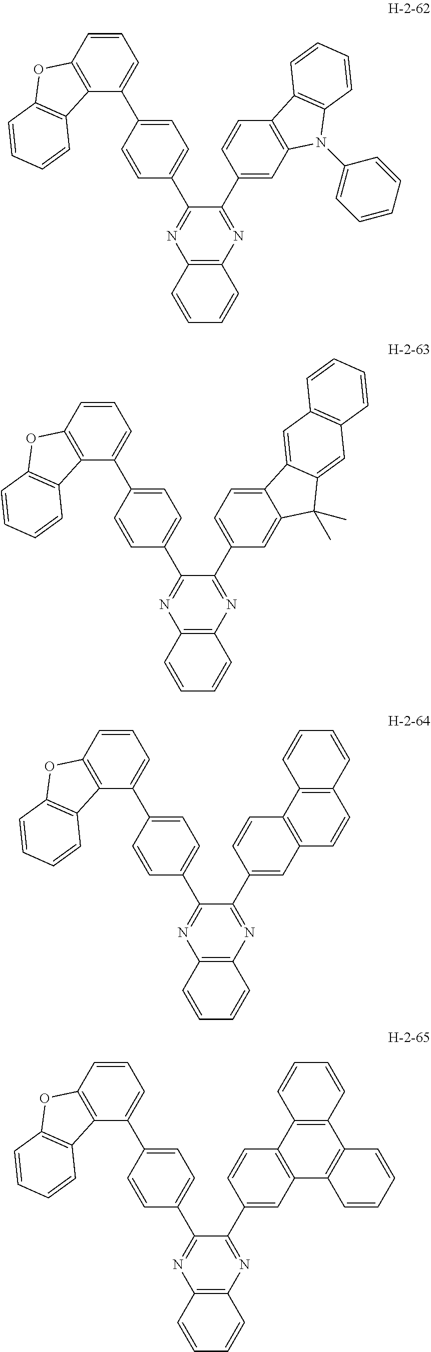 [SMILES: c1ccc(-n2c3ccccc3c3ccc(-c4nc5ccccc5nc4-c4ccc(-c5cccc6oc7ccccc7c56)cc4)cc32)cc1 ; CC1(C)c2cc(-c3nc4ccccc4nc3-c3ccc(-c4cccc5oc6ccccc6c45)cc3)ccc2-c2cc3ccccc3cc21 ; c1ccc2c(c1)ccc1cc(-c3nc4ccccc4nc3-c3ccc(-c4cccc5oc6ccccc6c45)cc3)ccc12 ; c1ccc2nc(-c3ccc4c5ccccc5c5ccccc5c4c3)c(-c3ccc(-c4cccc5oc6ccccc6c45)cc3)nc2c1]

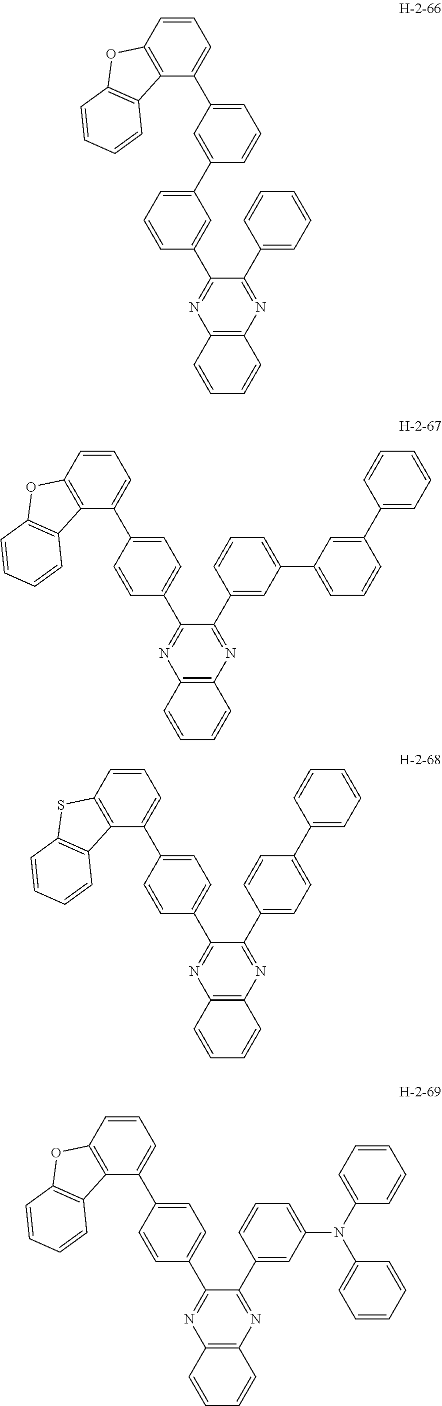 [SMILES: c1ccc(-c2nc3ccccc3nc2-c2cccc(-c3cccc(-c4cccc5oc6ccccc6c45)c3)c2)cc1 ; c1ccc(-c2cccc(-c3cccc(-c4nc5ccccc5nc4-c4ccc(-c5cccc6oc7ccccc7c56)cc4)c3)c2)cc1 ; c1ccc(-c2ccc(-c3nc4ccccc4nc3-c3ccc(-c4cccc5sc6ccccc6c45)cc3)cc2)cc1 ; c1ccc(N(c2ccccc2)c2cccc(-c3nc4ccccc4nc3-c3ccc(-c4cccc5oc6ccccc6c45)cc3)c2)cc1]

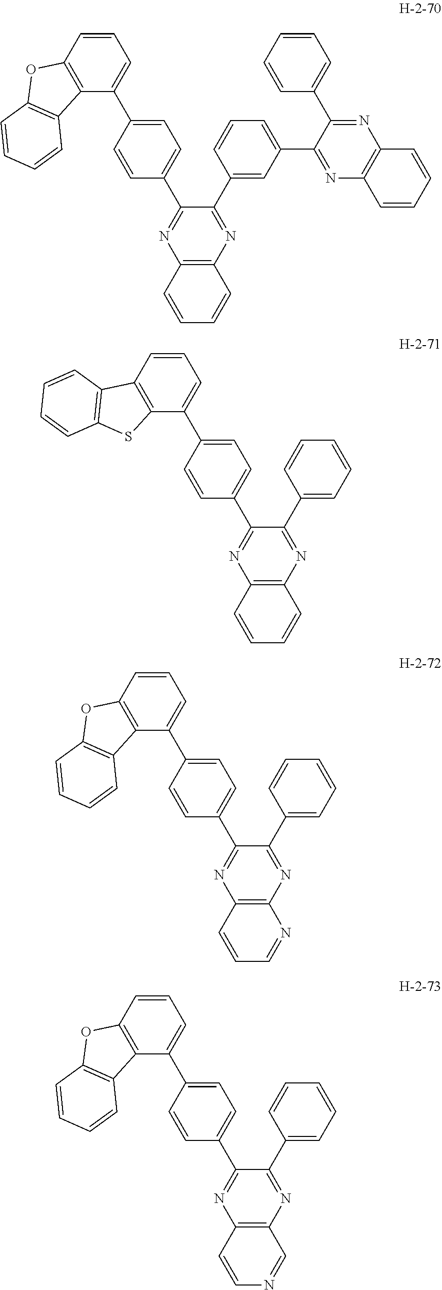

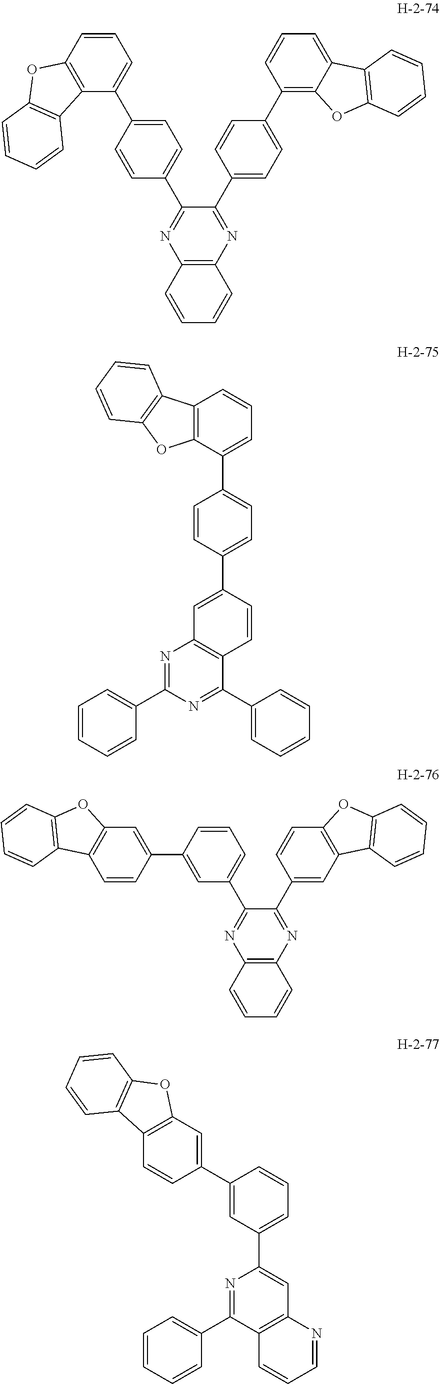 [SMILES: c1ccc2nc(-c3ccc(-c4cccc5oc6ccccc6c45)cc3)c(-c3ccc(-c4cccc5c4oc4ccccc45)cc3)nc2c1 ; c1ccc(-c2nc(-c3ccccc3)c3ccc(-c4ccc(-c5cccc6c5oc5ccccc56)cc4)cc3n2)cc1 ; c1cc(-c2ccc3c(c2)oc2ccccc23)cc(-c2nc3ccccc3nc2-c2ccc3oc4ccccc4c3c2)c1 ; c1ccc(-c2nc(-c3cccc(-c4ccc5c(c4)oc4ccccc45)c3)cc3ncccc23)cc1]

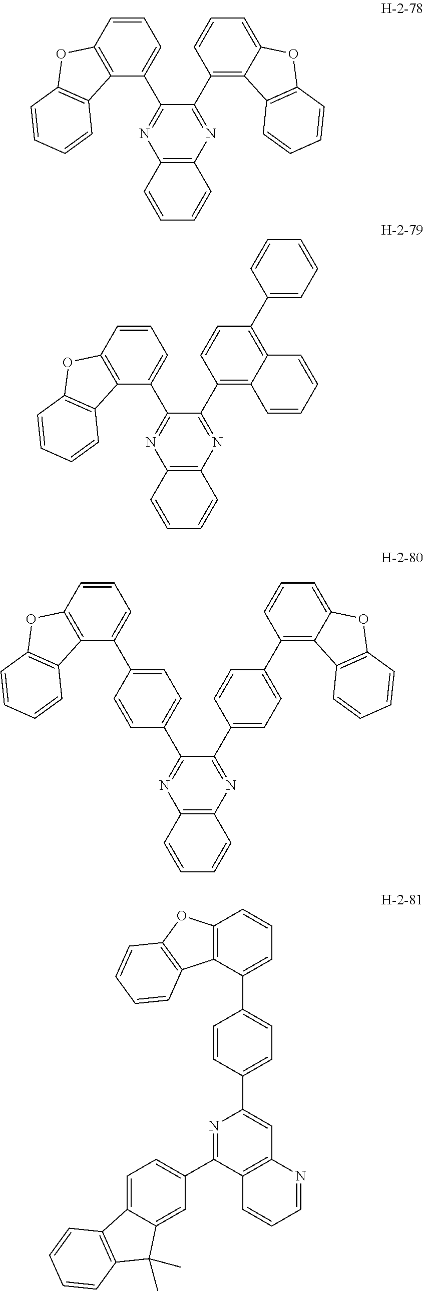

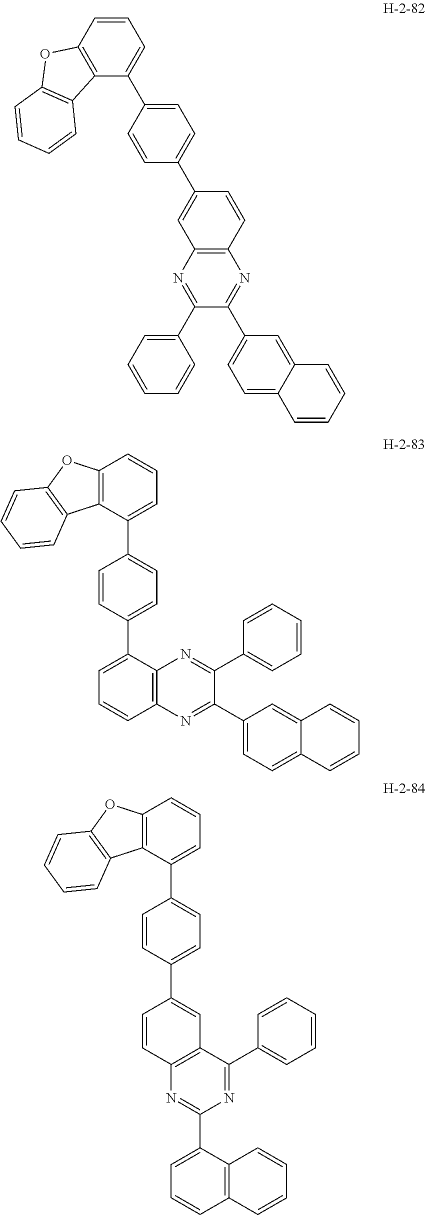 [SMILES: c1ccc(-c2nc3cc(-c4ccc(-c5cccc6oc7ccccc7c56)cc4)ccc3nc2-c2ccc3ccccc3c2)cc1 ; c1ccc(-c2nc3c(-c4ccc(-c5cccc6oc7ccccc7c56)cc4)cccc3nc2-c2ccc3ccccc3c2)cc1 ; c1ccc(-c2nc(-c3cccc4ccccc34)nc3ccc(-c4ccc(-c5cccc6oc7ccccc7c56)cc4)cc23)cc1]

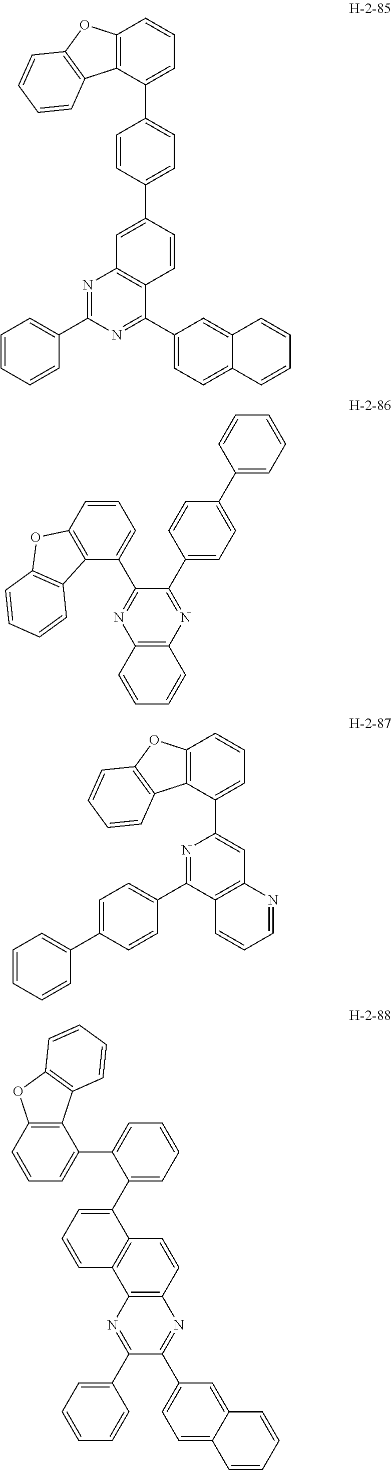

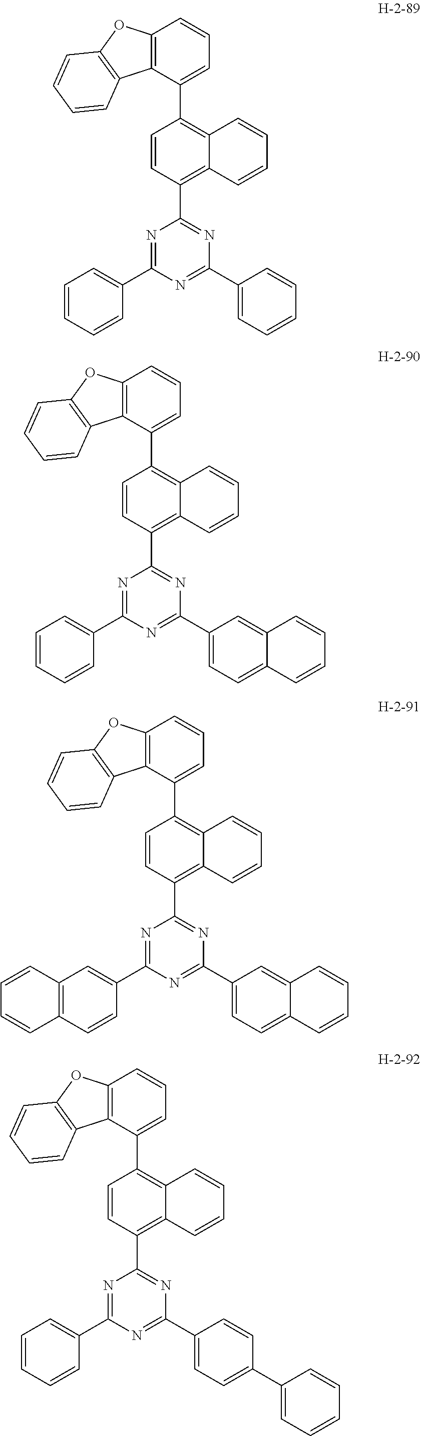

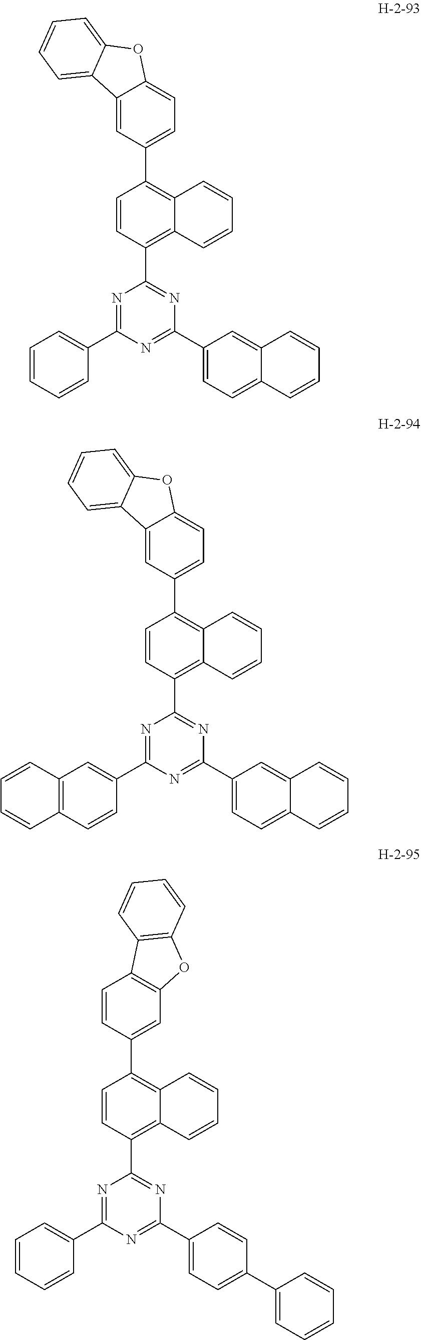 [SMILES: c1ccc(-c2nc(-c3ccc4ccccc4c3)nc(-c3ccc(-c4ccc5oc6ccccc6c5c4)c4ccccc34)n2)cc1 ; c1ccc2cc(-c3nc(-c4ccc5ccccc5c4)nc(-c4ccc(-c5ccc6oc7ccccc7c6c5)c5ccccc45)n3)ccc2c1 ; c1ccc(-c2ccc(-c3nc(-c4ccccc4)nc(-c4ccc(-c5ccc6c(c5)oc5ccccc56)c5ccccc45)n3)cc2)cc1]

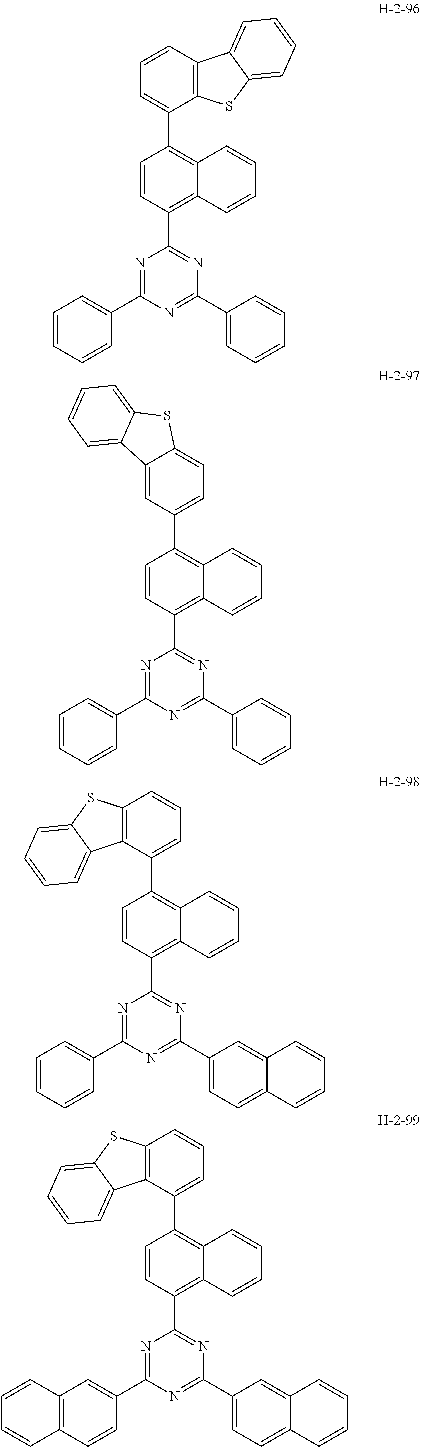

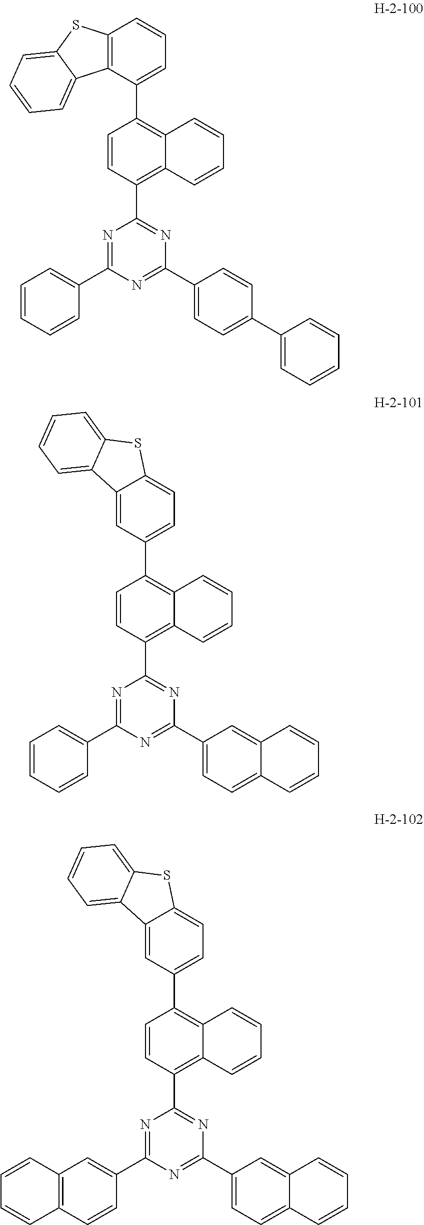 [SMILES: c1ccc(-c2ccc(-c3nc(-c4ccccc4)nc(-c4ccc(-c5cccc6sc7ccccc7c56)c5ccccc45)n3)cc2)cc1 ; c1ccc(-c2nc(-c3ccc4ccccc4c3)nc(-c3ccc(-c4ccc5sc6ccccc6c5c4)c4ccccc34)n2)cc1 ; c1ccc2cc(-c3nc(-c4ccc5ccccc5c4)nc(-c4ccc(-c5ccc6sc7ccccc7c6c5)c5ccccc45)n3)ccc2c1]

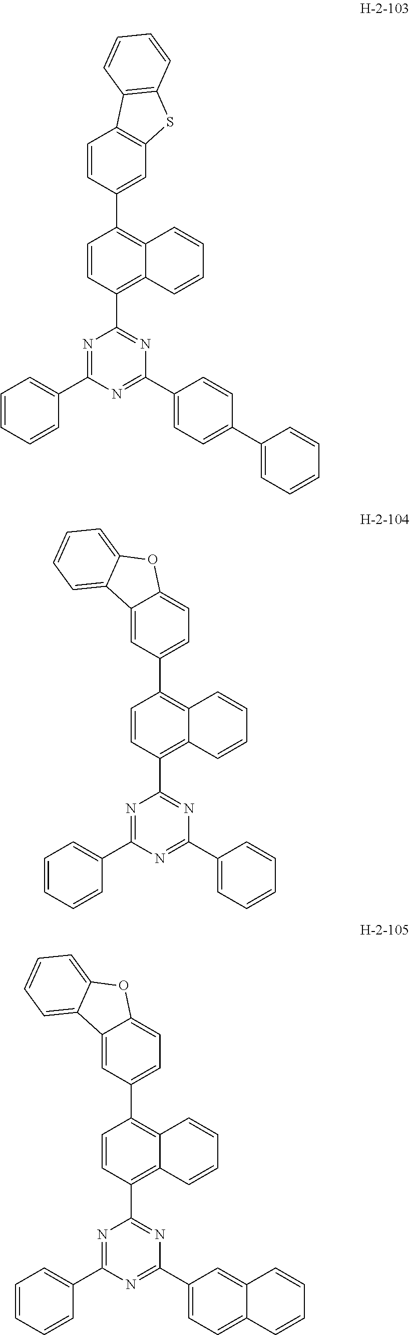

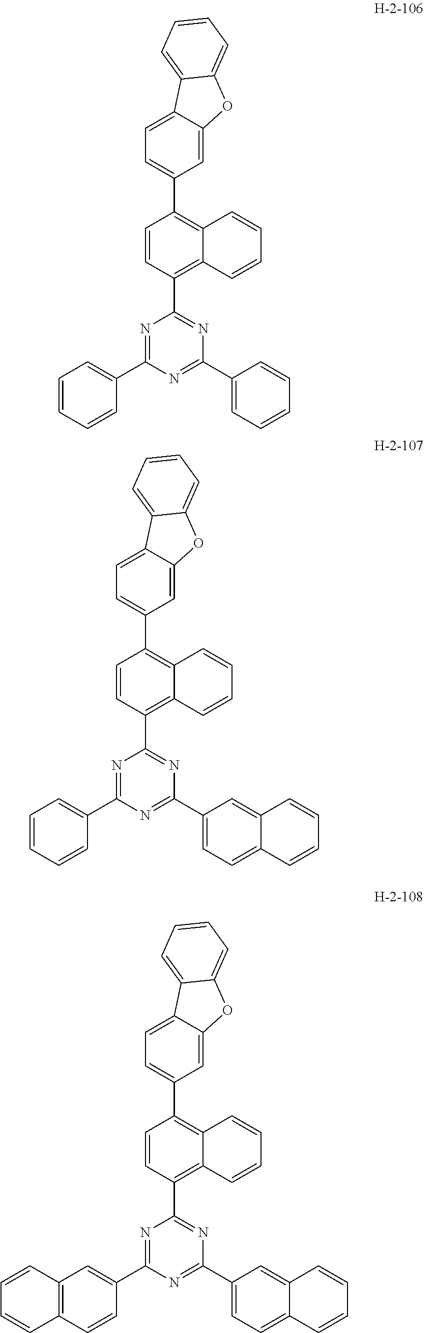 [SMILES: c1ccc(-c2nc(-c3ccccc3)nc(-c3ccc(-c4ccc5c(c4)oc4ccccc45)c4ccccc34)n2)cc1 ; c1ccc(-c2nc(-c3ccc4ccccc4c3)nc(-c3ccc(-c4ccc5c(c4)oc4ccccc45)c4ccccc34)n2)cc1 ; c1ccc2cc(-c3nc(-c4ccc5ccccc5c4)nc(-c4ccc(-c5ccc6c(c5)oc5ccccc56)c5ccccc45)n3)ccc2c1]

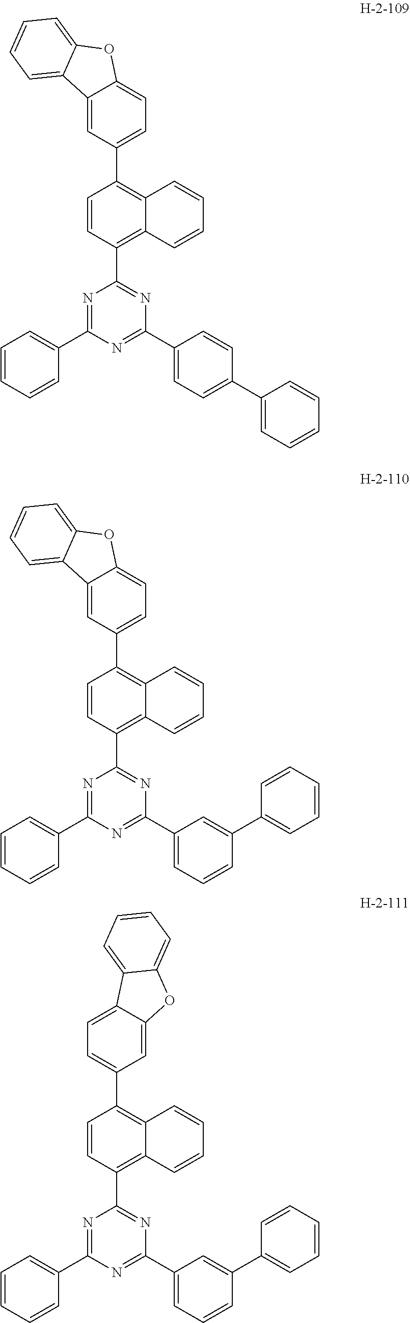 [SMILES: c1ccc(-c2ccc(-c3nc(-c4ccccc4)nc(-c4ccc(-c5ccc6oc7ccccc7c6c5)c5ccccc45)n3)cc2)cc1 ; c1ccc(-c2cccc(-c3nc(-c4ccccc4)nc(-c4ccc(-c5ccc6oc7ccccc7c6c5)c5ccccc45)n3)c2)cc1 ; c1ccc(-c2cccc(-c3nc(-c4ccccc4)nc(-c4ccc(-c5ccc6c(c5)oc5ccccc56)c5ccccc45)n3)c2)cc1]

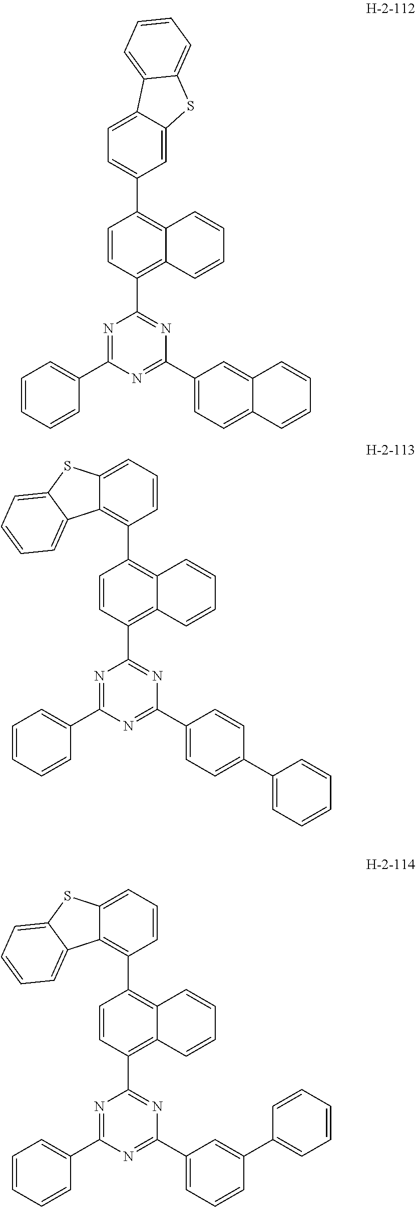

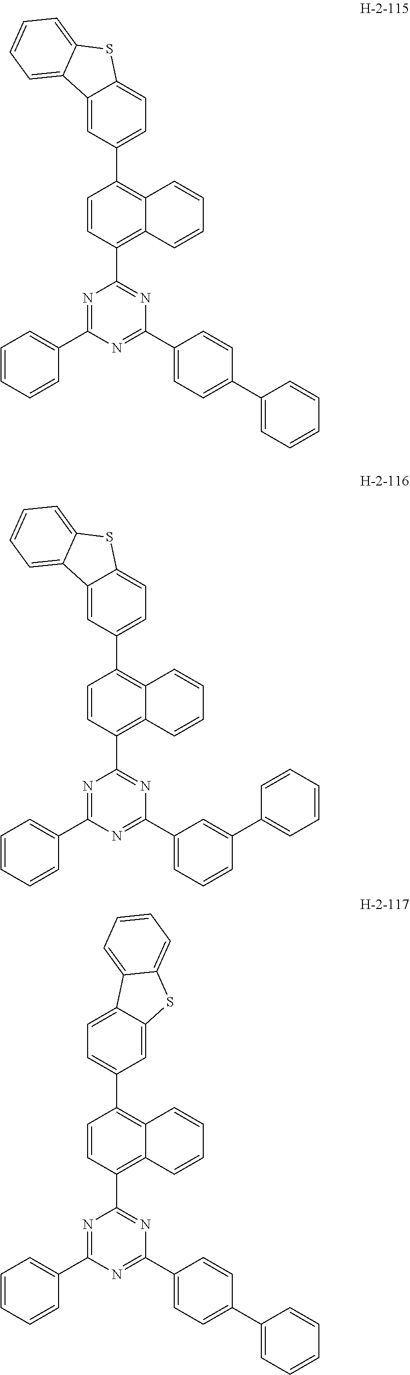 [SMILES: c1ccc(-c2ccc(-c3nc(-c4ccccc4)nc(-c4ccc(-c5ccc6sc7ccccc7c6c5)c5ccccc45)n3)cc2)cc1 ; c1ccc(-c2cccc(-c3nc(-c4ccccc4)nc(-c4ccc(-c5ccc6sc7ccccc7c6c5)c5ccccc45)n3)c2)cc1 ; c1ccc(-c2ccc(-c3nc(-c4ccccc4)nc(-c4ccc(-c5ccc6c(c5)sc5ccccc56)c5ccccc45)n3)cc2)cc1]

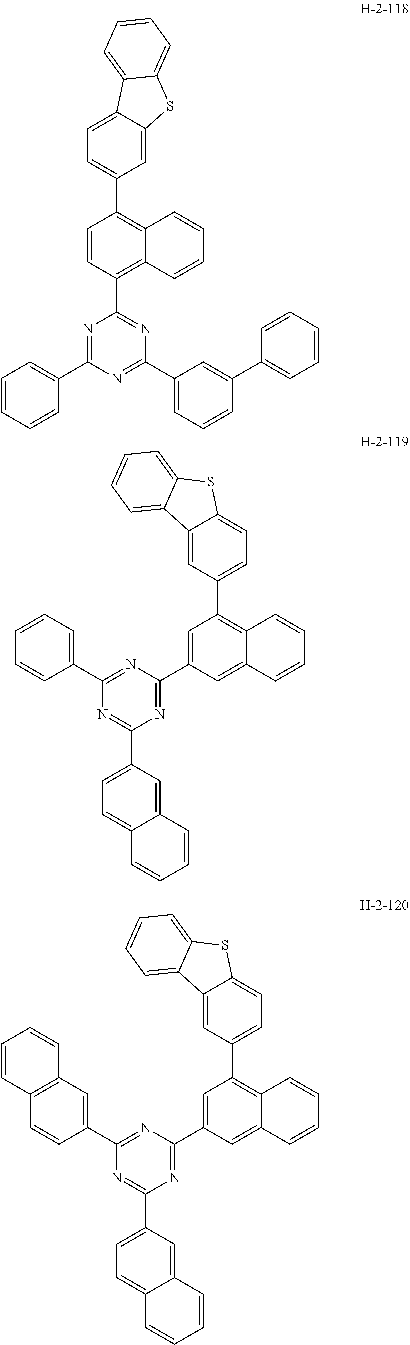

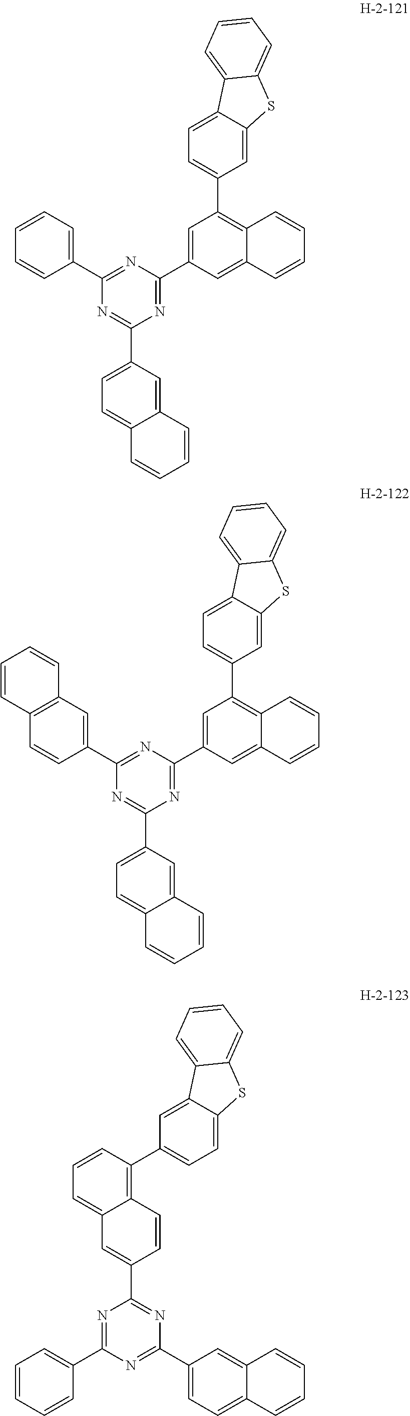 [SMILES: c1ccc(-c2nc(-c3ccc4ccccc4c3)nc(-c3cc(-c4ccc5c(c4)sc4ccccc45)c4ccccc4c3)n2)cc1 ; c1ccc2cc(-c3nc(-c4ccc5ccccc5c4)nc(-c4cc(-c5ccc6c(c5)sc5ccccc56)c5ccccc5c4)n3)ccc2c1 ; c1ccc(-c2nc(-c3ccc4ccccc4c3)nc(-c3ccc4c(-c5ccc6sc7ccccc7c6c5)cccc4c3)n2)cc1]

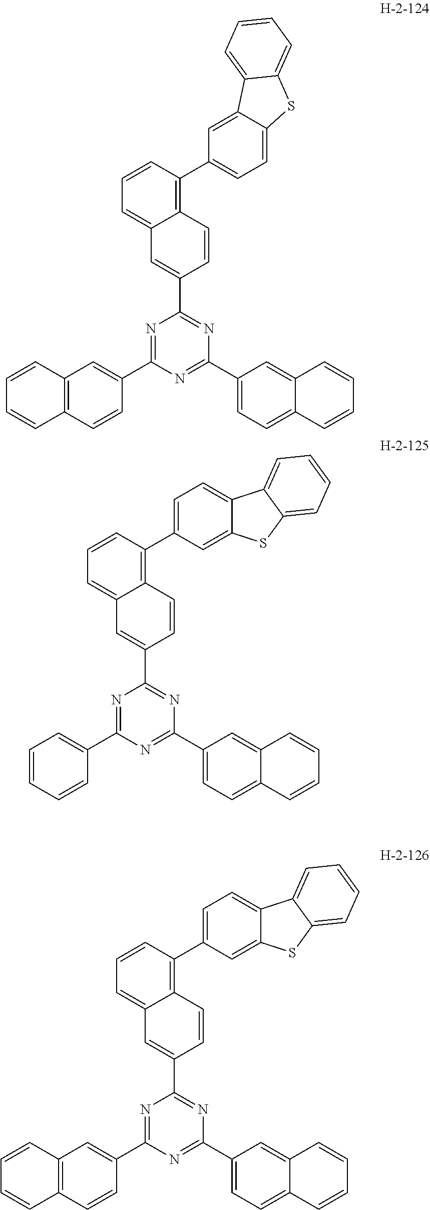

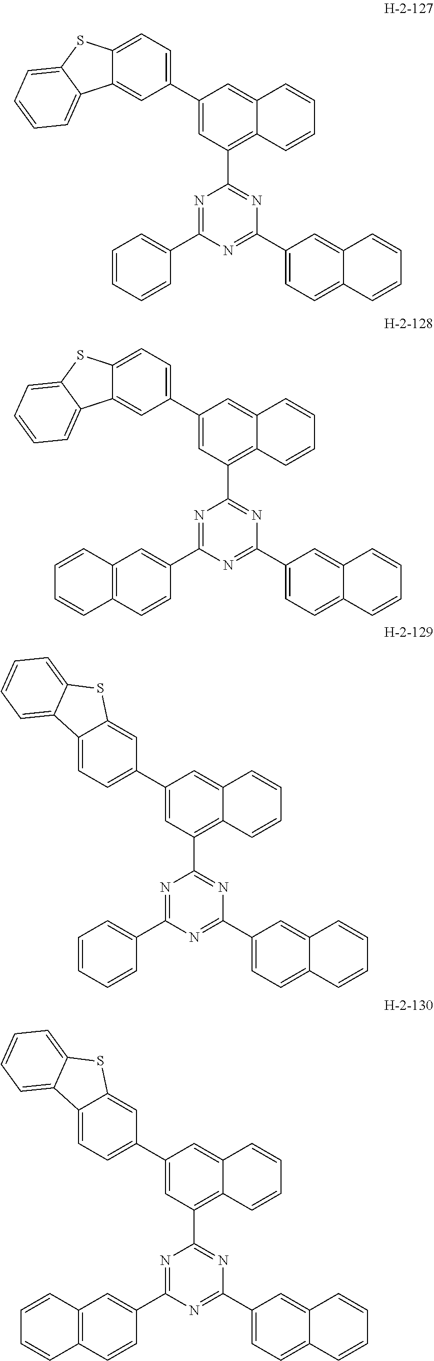 [SMILES: c1ccc(-c2nc(-c3ccc4ccccc4c3)nc(-c3cc(-c4ccc5sc6ccccc6c5c4)cc4ccccc34)n2)cc1 ; c1ccc2cc(-c3nc(-c4ccc5ccccc5c4)nc(-c4cc(-c5ccc6sc7ccccc7c6c5)cc5ccccc45)n3)ccc2c1 ; c1ccc(-c2nc(-c3ccc4ccccc4c3)nc(-c3cc(-c4ccc5c(c4)sc4ccccc45)cc4ccccc34)n2)cc1 ; c1ccc2cc(-c3nc(-c4ccc5ccccc5c4)nc(-c4cc(-c5ccc6c(c5)sc5ccccc56)cc5ccccc45)n3)ccc2c1]

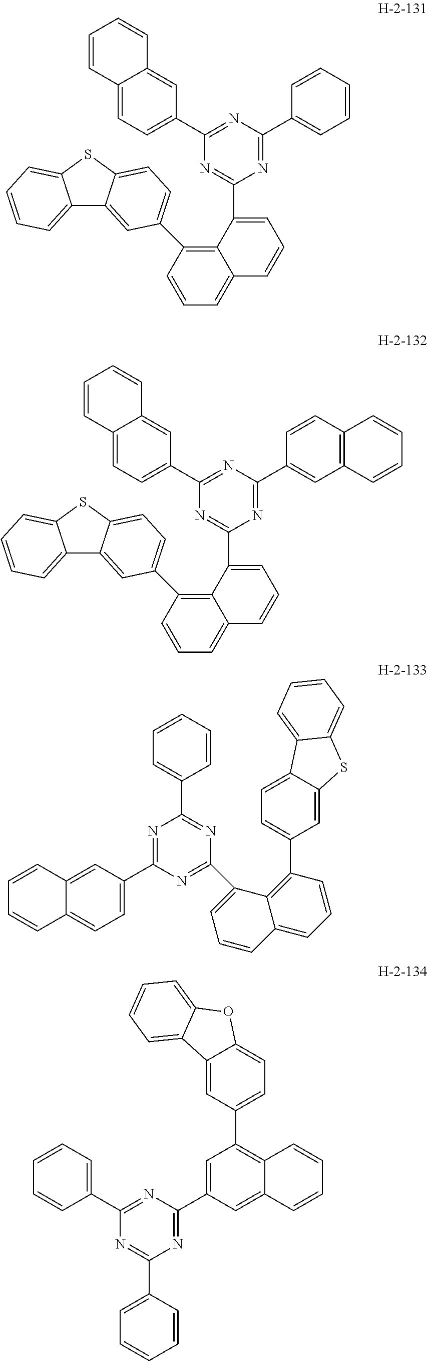 [SMILES: c1ccc(-c2nc(-c3ccc4ccccc4c3)nc(-c3cccc4cccc(-c5ccc6sc7ccccc7c6c5)c34)n2)cc1 ; c1ccc2cc(-c3nc(-c4ccc5ccccc5c4)nc(-c4cccc5cccc(-c6ccc7sc8ccccc8c7c6)c45)n3)ccc2c1 ; c1ccc(-c2nc(-c3ccc4ccccc4c3)nc(-c3cccc4cccc(-c5ccc6c(c5)sc5ccccc56)c34)n2)cc1 ; c1ccc(-c2nc(-c3ccccc3)nc(-c3cc(-c4ccc5oc6ccccc6c5c4)c4ccccc4c3)n2)cc1]

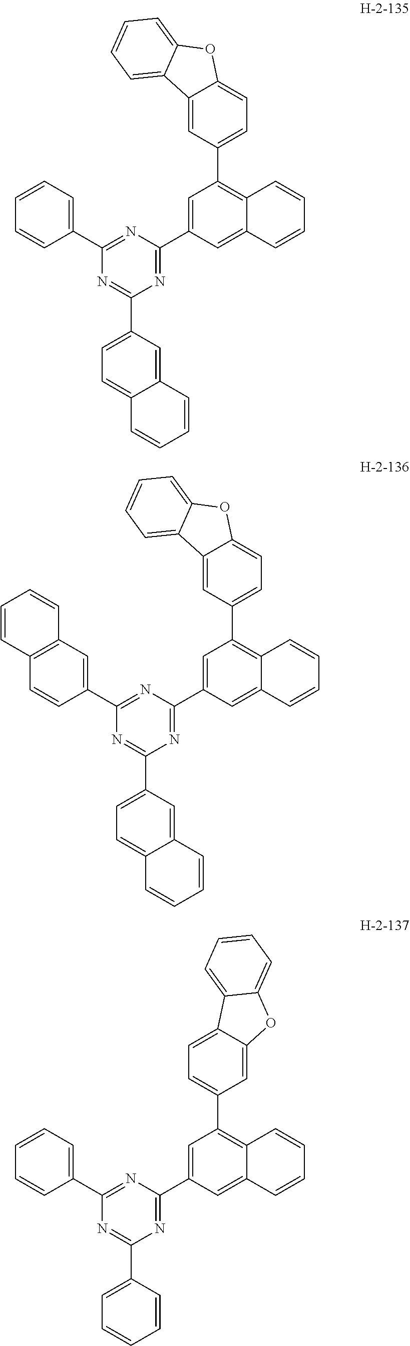

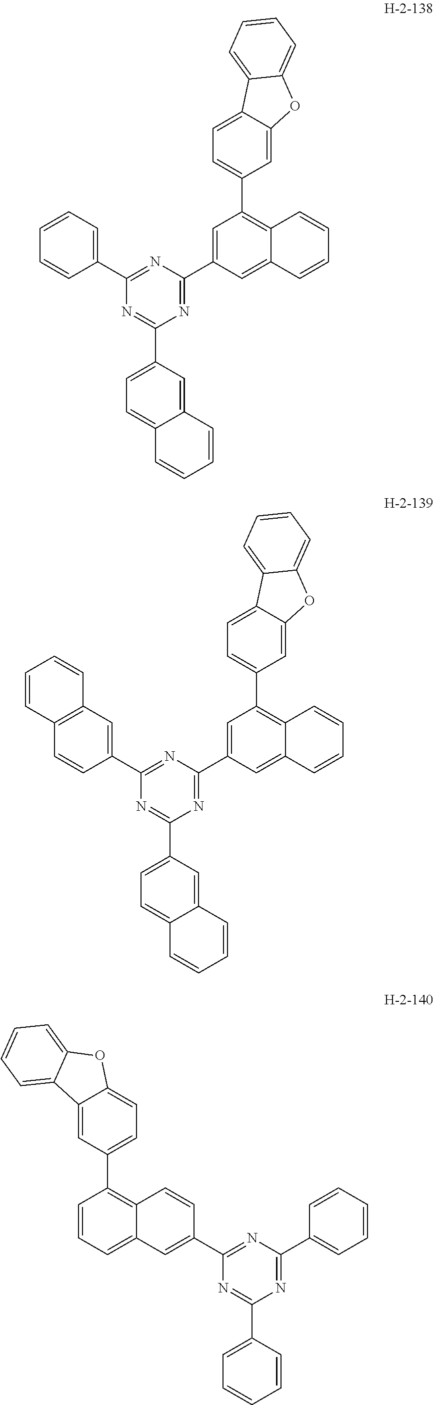 [SMILES: c1ccc(-c2nc(-c3ccc4ccccc4c3)nc(-c3cc(-c4ccc5c(c4)oc4ccccc45)c4ccccc4c3)n2)cc1 ; c1ccc2cc(-c3nc(-c4ccc5ccccc5c4)nc(-c4cc(-c5ccc6c(c5)oc5ccccc56)c5ccccc5c4)n3)ccc2c1 ; c1ccc(-c2nc(-c3ccccc3)nc(-c3ccc4c(-c5ccc6oc7ccccc7c6c5)cccc4c3)n2)cc1]

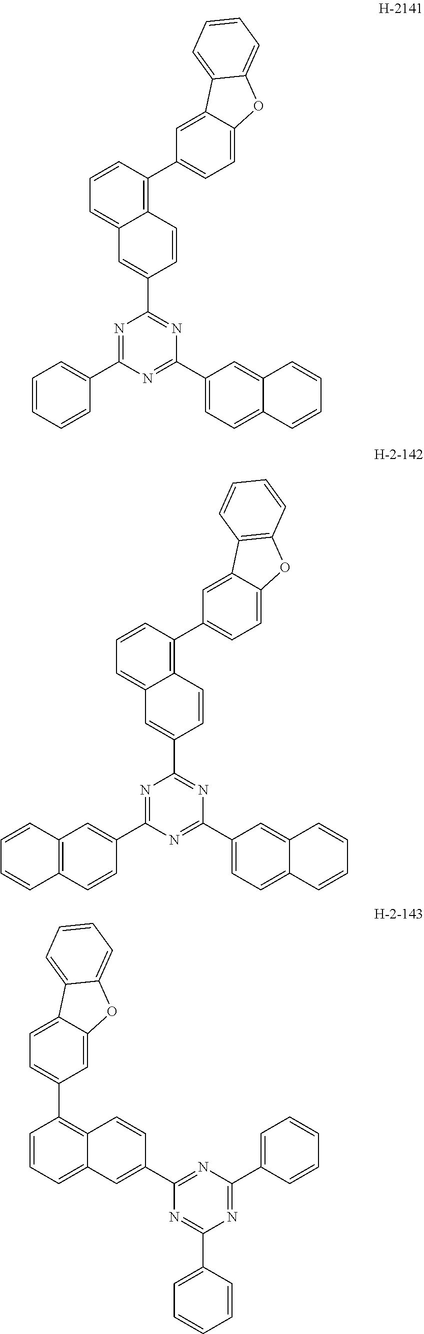

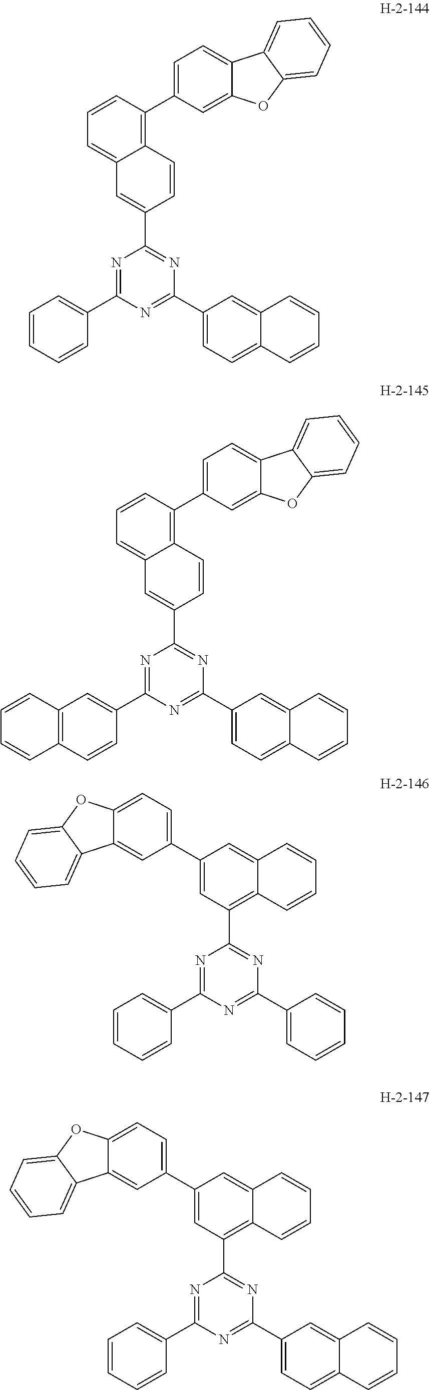

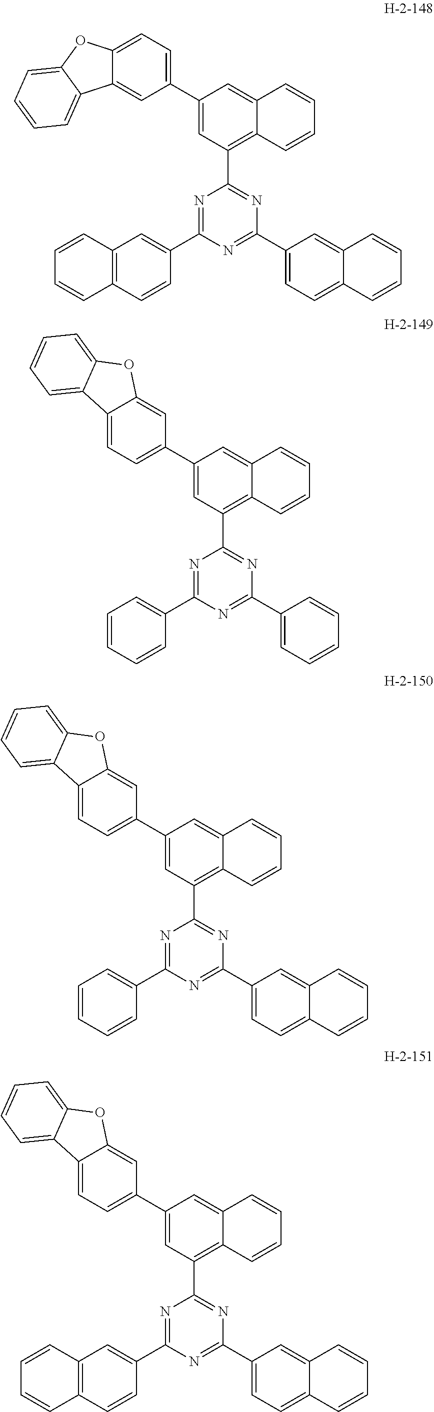

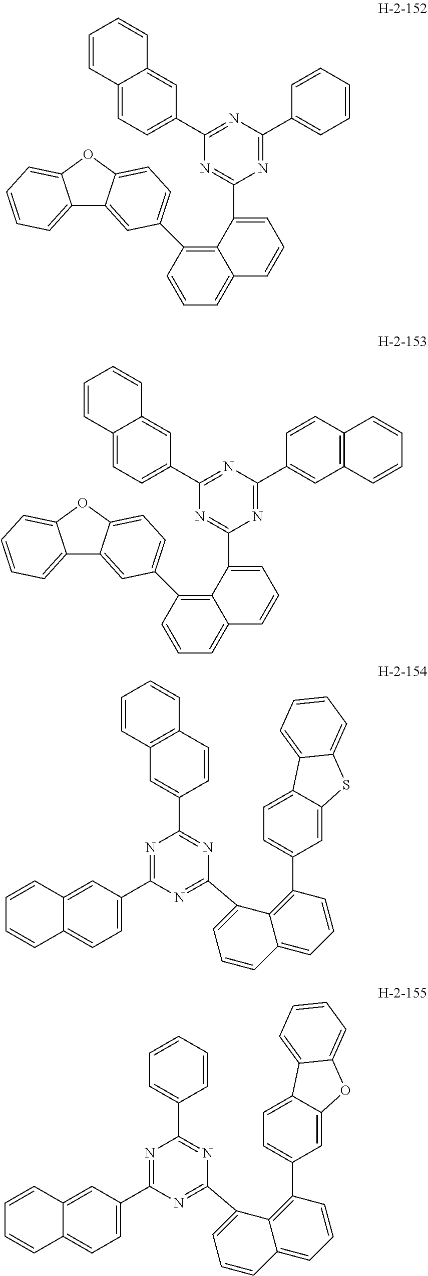

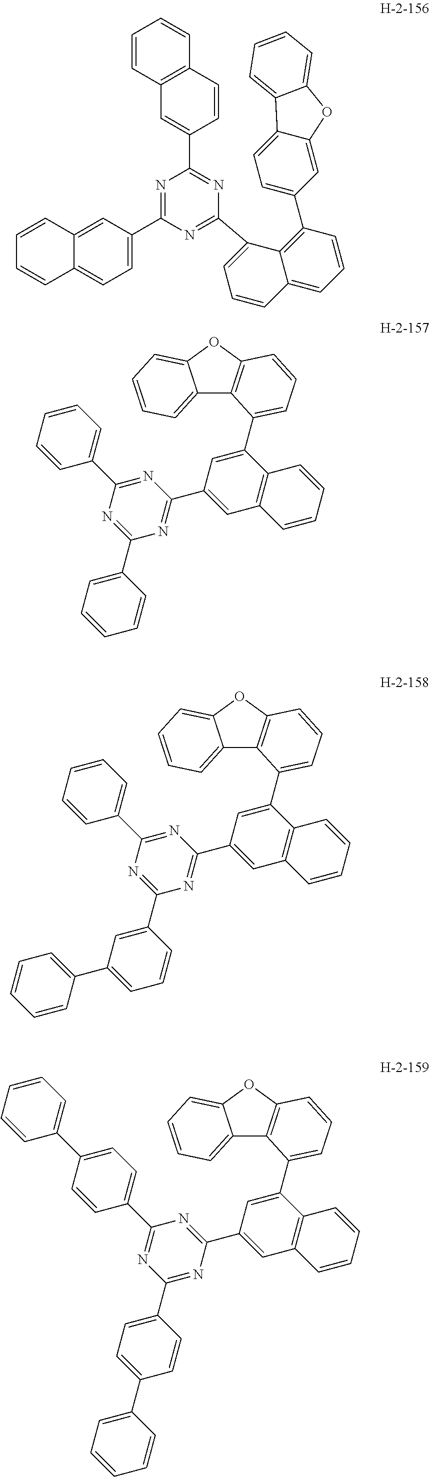 [SMILES: c1ccc2cc(-c3nc(-c4ccc5ccccc5c4)nc(-c4cccc5cccc(-c6ccc7c(c6)oc6ccccc67)c45)n3)ccc2c1 ; c1ccc(-c2nc(-c3ccccc3)nc(-c3cc(-c4cccc5oc6ccccc6c45)c4ccccc4c3)n2)cc1 ; c1ccc(-c2cccc(-c3nc(-c4ccccc4)nc(-c4cc(-c5cccc6oc7ccccc7c56)c5ccccc5c4)n3)c2)cc1 ; c1ccc(-c2ccc(-c3nc(-c4ccc(-c5ccccc5)cc4)nc(-c4cc(-c5cccc6oc7ccccc7c56)c5ccccc5c4)n3)cc2)cc1]

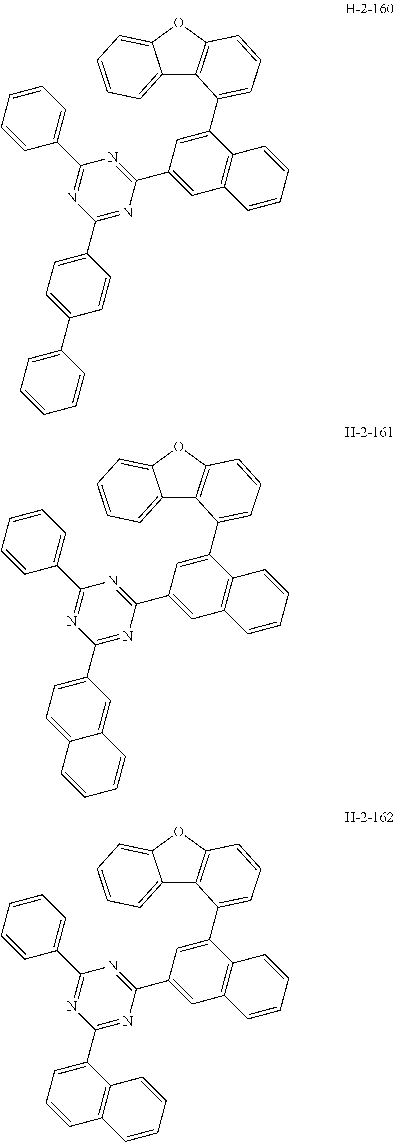 [SMILES: c1ccc(-c2ccc(-c3nc(-c4ccccc4)nc(-c4cc(-c5cccc6oc7ccccc7c56)c5ccccc5c4)n3)cc2)cc1 ; c1ccc(-c2nc(-c3ccc4ccccc4c3)nc(-c3cc(-c4cccc5oc6ccccc6c45)c4ccccc4c3)n2)cc1 ; c1ccc(-c2nc(-c3cc(-c4cccc5oc6ccccc6c45)c4ccccc4c3)nc(-c3cccc4ccccc34)n2)cc1]

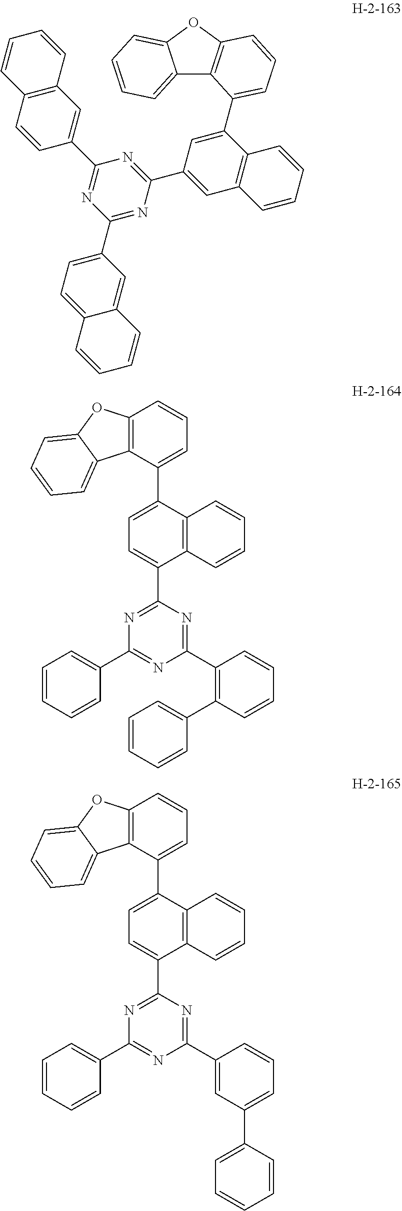

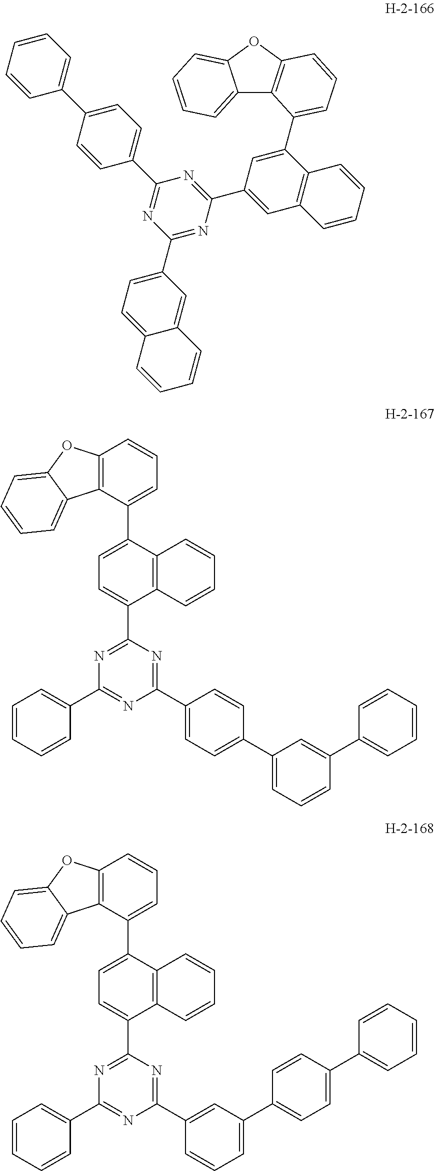 [SMILES: c1ccc(-c2ccc(-c3nc(-c4ccc5ccccc5c4)nc(-c4cc(-c5cccc6oc7ccccc7c56)c5ccccc5c4)n3)cc2)cc1 ; c1ccc(-c2cccc(-c3ccc(-c4nc(-c5ccccc5)nc(-c5ccc(-c6cccc7oc8ccccc8c67)c6ccccc56)n4)cc3)c2)cc1 ; c1ccc(-c2ccc(-c3cccc(-c4nc(-c5ccccc5)nc(-c5ccc(-c6cccc7oc8ccccc8c67)c6ccccc56)n4)c3)cc2)cc1]

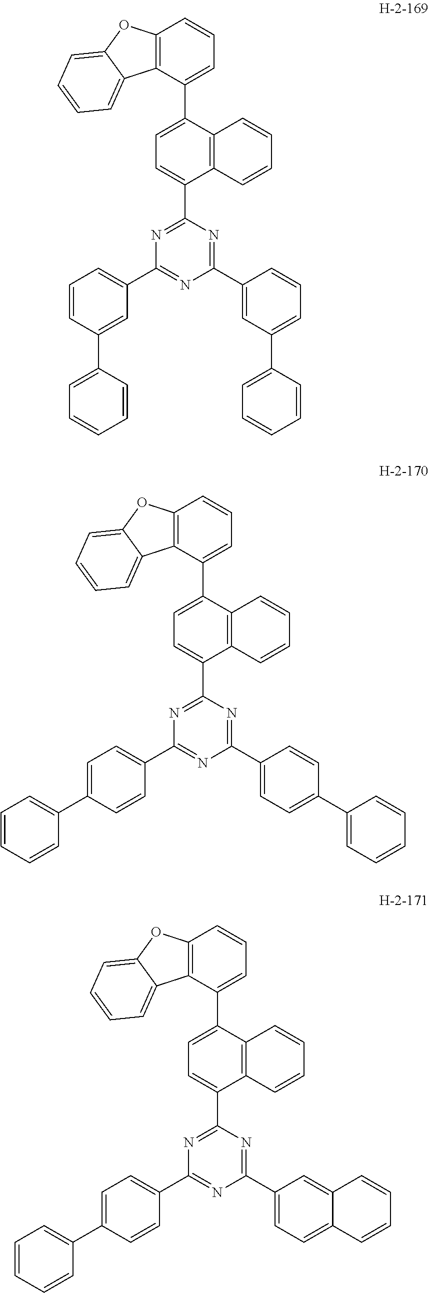 [SMILES: c1ccc(-c2cccc(-c3nc(-c4cccc(-c5ccccc5)c4)nc(-c4ccc(-c5cccc6oc7ccccc7c56)c5ccccc45)n3)c2)cc1 ; c1ccc(-c2ccc(-c3nc(-c4ccc(-c5ccccc5)cc4)nc(-c4ccc(-c5cccc6oc7ccccc7c56)c5ccccc45)n3)cc2)cc1 ; c1ccc(-c2ccc(-c3nc(-c4ccc5ccccc5c4)nc(-c4ccc(-c5cccc6oc7ccccc7c56)c5ccccc45)n3)cc2)cc1]

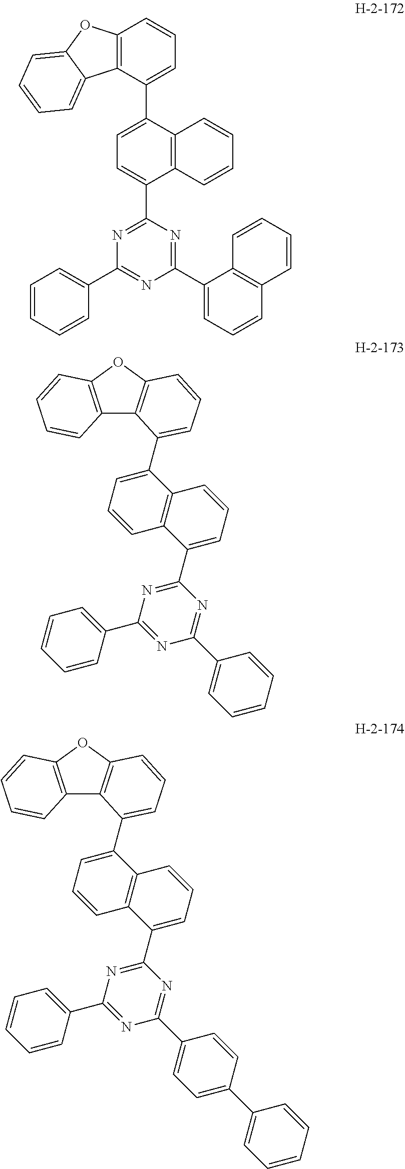

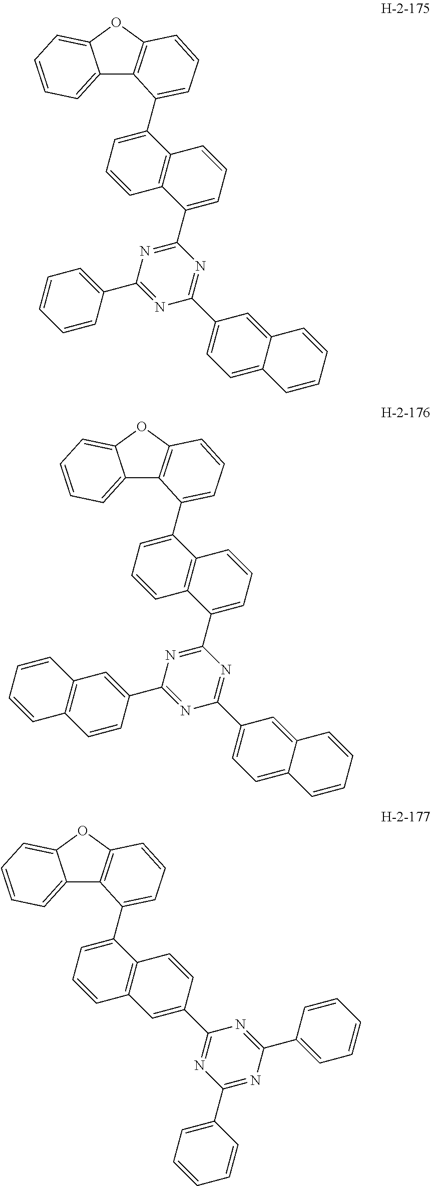 [SMILES: c1ccc(-c2nc(-c3ccc4ccccc4c3)nc(-c3cccc4c(-c5cccc6oc7ccccc7c56)cccc34)n2)cc1 ; c1ccc2cc(-c3nc(-c4ccc5ccccc5c4)nc(-c4cccc5c(-c6cccc7oc8ccccc8c67)cccc45)n3)ccc2c1 ; c1ccc(-c2nc(-c3ccccc3)nc(-c3ccc4c(-c5cccc6oc7ccccc7c56)cccc4c3)n2)cc1]

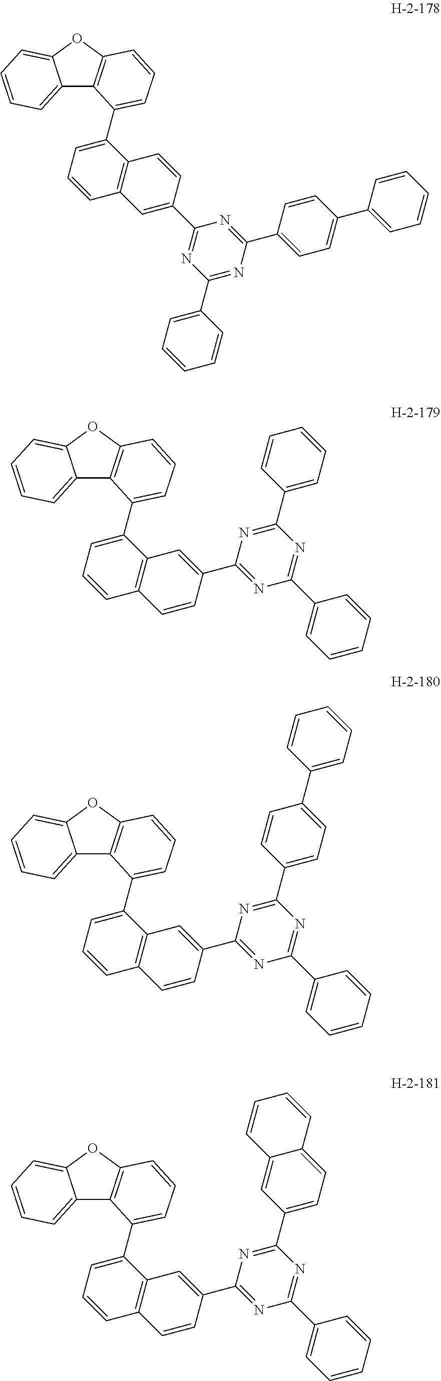 [SMILES: c1ccc(-c2ccc(-c3nc(-c4ccccc4)nc(-c4ccc5c(-c6cccc7oc8ccccc8c67)cccc5c4)n3)cc2)cc1 ; c1ccc(-c2nc(-c3ccccc3)nc(-c3ccc4cccc(-c5cccc6oc7ccccc7c56)c4c3)n2)cc1 ; c1ccc(-c2ccc(-c3nc(-c4ccccc4)nc(-c4ccc5cccc(-c6cccc7oc8ccccc8c67)c5c4)n3)cc2)cc1 ; c1ccc(-c2nc(-c3ccc4ccccc4c3)nc(-c3ccc4cccc(-c5cccc6oc7ccccc7c56)c4c3)n2)cc1]

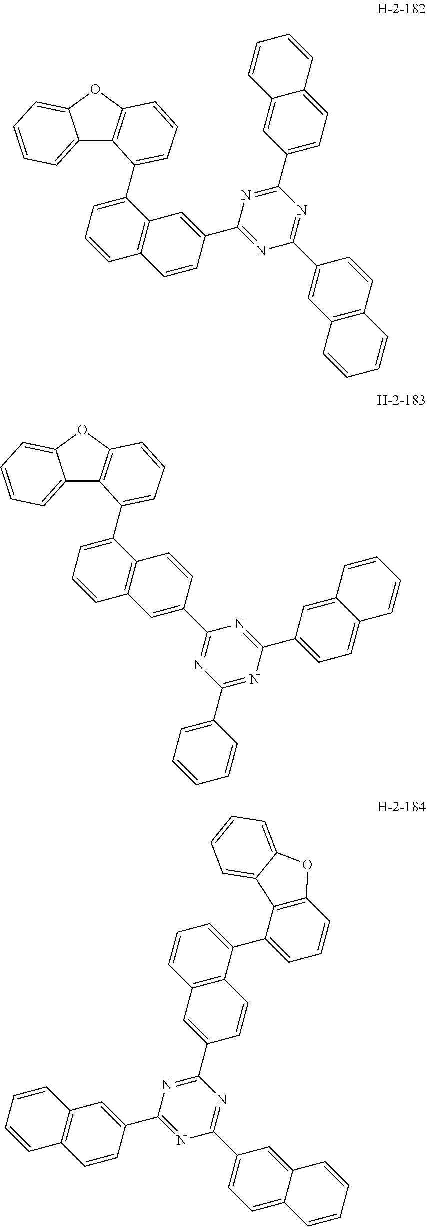

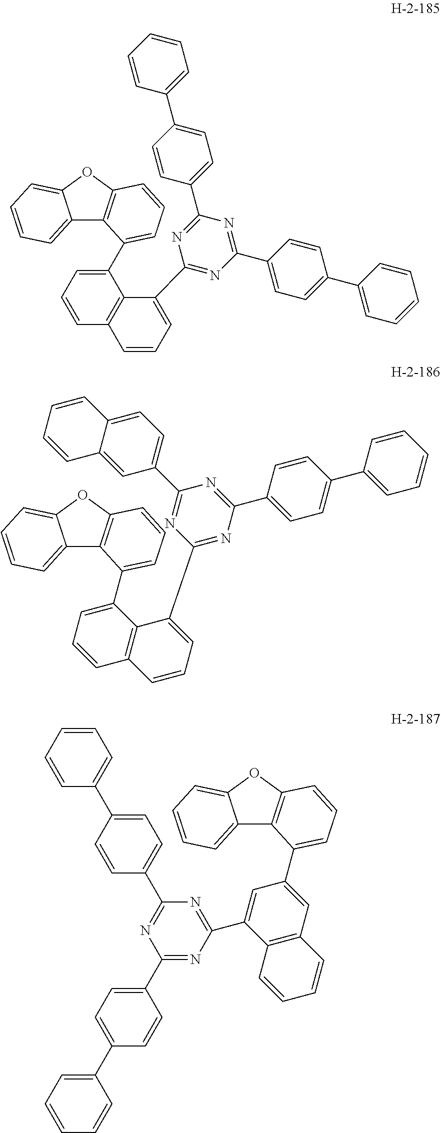 [SMILES: c1ccc(-c2ccc(-c3nc(-c4ccc(-c5ccccc5)cc4)nc(-c4cccc5cccc(-c6cccc7oc8ccccc8c67)c45)n3)cc2)cc1 ; c1ccc(-c2ccc(-c3nc(-c4ccc5ccccc5c4)nc(-c4cccc5cccc(-c6cccc7oc8ccccc8c67)c45)n3)cc2)cc1 ; c1ccc(-c2ccc(-c3nc(-c4ccc(-c5ccccc5)cc4)nc(-c4cc(-c5cccc6oc7ccccc7c56)cc5ccccc45)n3)cc2)cc1]

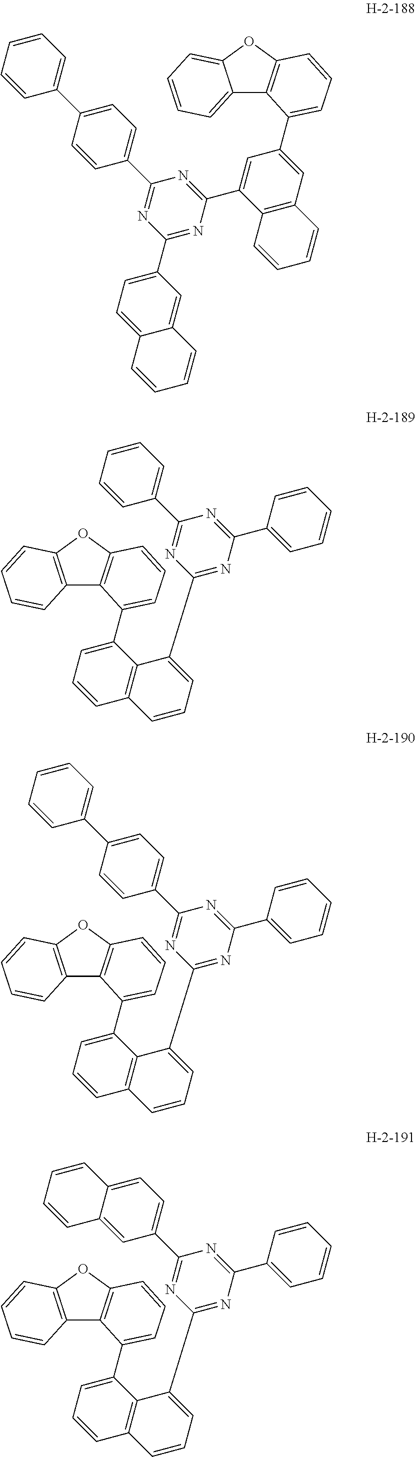 [SMILES: c1ccc(-c2ccc(-c3nc(-c4ccc5ccccc5c4)nc(-c4cc(-c5cccc6oc7ccccc7c56)cc5ccccc45)n3)cc2)cc1 ; c1ccc(-c2nc(-c3ccccc3)nc(-c3cccc4cccc(-c5cccc6oc7ccccc7c56)c34)n2)cc1 ; c1ccc(-c2ccc(-c3nc(-c4ccccc4)nc(-c4cccc5cccc(-c6cccc7oc8ccccc8c67)c45)n3)cc2)cc1 ; c1ccc(-c2nc(-c3ccc4ccccc4c3)nc(-c3cccc4cccc(-c5cccc6oc7ccccc7c56)c34)n2)cc1]

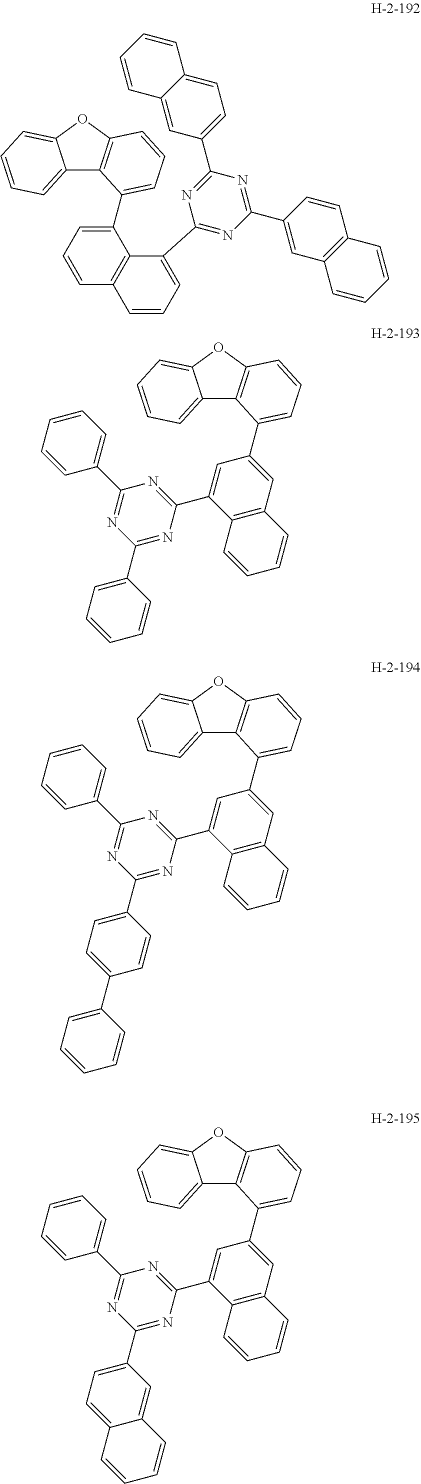

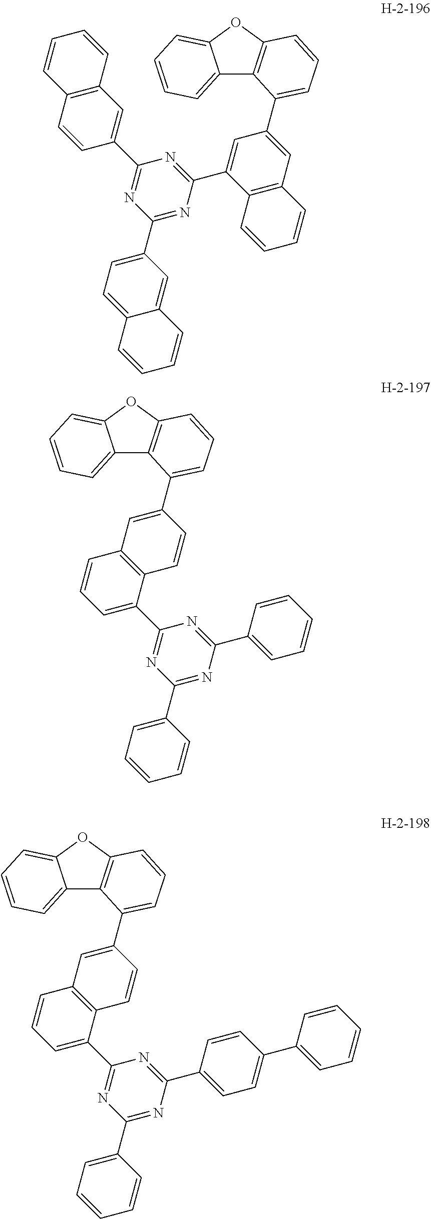

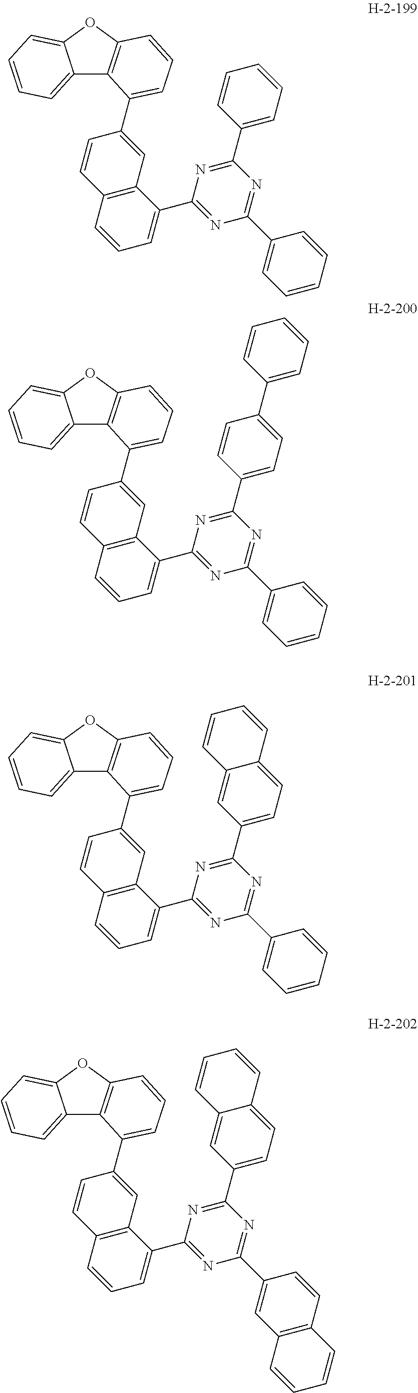

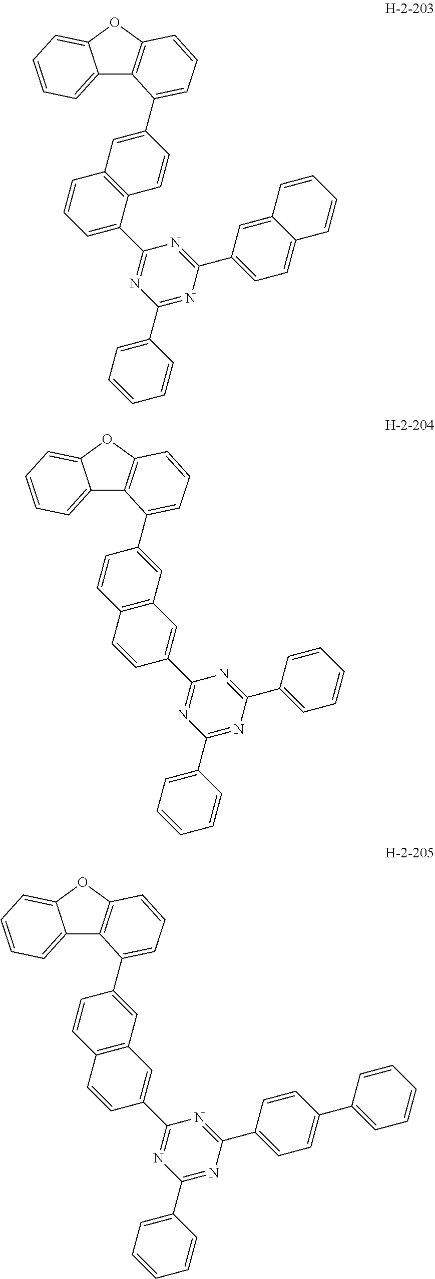

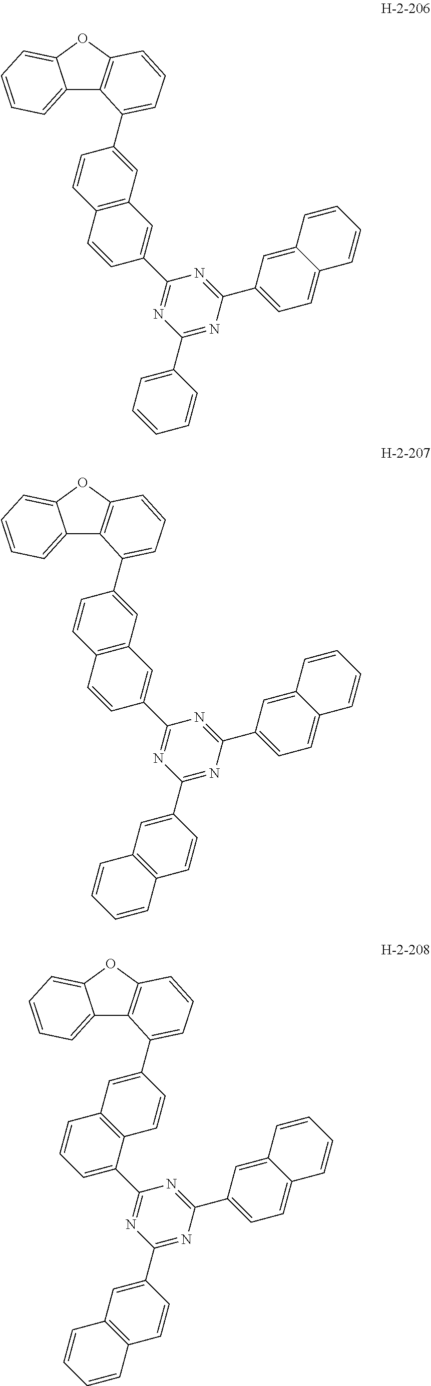

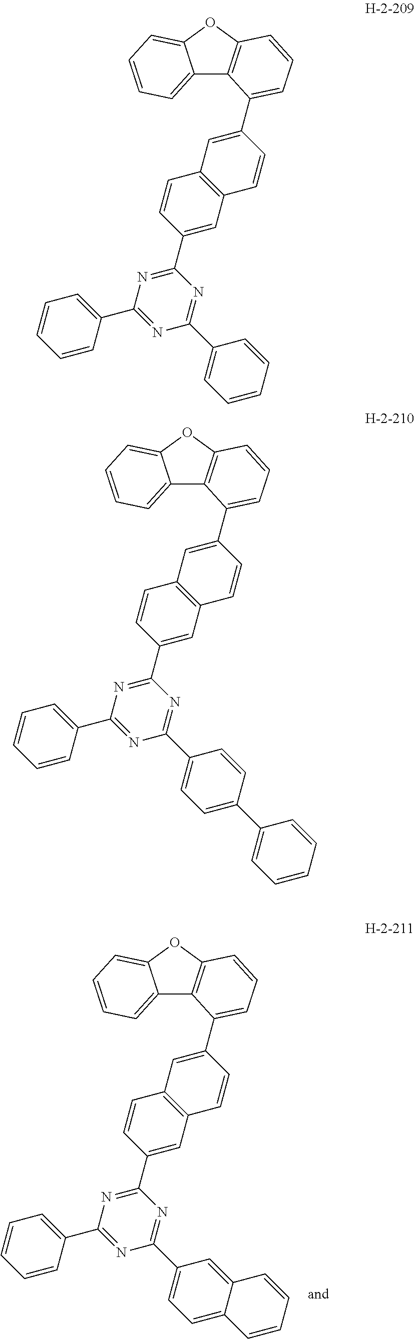

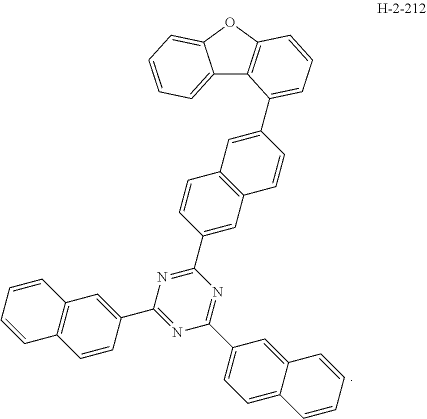 [SMILES: c1ccc2cc(-c3nc(-c4ccc5ccccc5c4)nc(-c4ccc5cc(-c6cccc7oc8ccccc8c67)ccc5c4)n3)ccc2c1]

9. An organic electroluminescent device comprising an anode, a cathode, and at least one light-emitting layer between the anode and the cathode, wherein at least one of the light-emitting layers comprises the plurality of host materials according to claim 1.
